(19) United States
(12) Patent Application Publication
(10) Pub. No.: US 2021/0214749 A1
(43) Pub. Date: Jul. 15, 2021

(54) DIRECTED EVOLUTION

(71) Applicant: VOYAGER THERAPEUTICS, INC., Cambridge, MA (US)

(72) Inventors: Jinzhao Hou, Lexington, MA (US); Hongxing Wang, Cambridge, MA (US); Robert Ng, Jamaica Plain, MA (US); Kye Il Joo, Arlington, MA (US)

(21) Appl. No.: 17/055,842

(22) PCT Filed: May 16, 2019
(86) PCT No.: PCT/US2019/032566

§ 371 Date: Nov. 16, 2020

Related U.S. Application Data
(60) Provisional application No. 62/672,450, filed on May 16, 2018.
(60) Provisional application No. 62/729,645, filed on Sep. 11, 2018.

(51) Int. Cl. C12N 15/86; C07K 14/435; C12N 15/11

(52) U.S. Cl. CPC C12N 15/86; C07K 14/43563; C12N 15/111; C12N 2750/14143; C12N 2750/14152; C12N 2330/51; C12N 2750/14122; C12N 2310/14; C12N 2750/14145

(57) ABSTRACT

The disclosure provides compositions and methods for the preparation, manufacture, formulation and therapeutic use of adeno-associated virus (AAV) particles for the prevention and/or treatment of diseases.

### REFERENCE TO RELEVANT APPLICATIONS

This application claims the benefit of U.S. Provisional Patent Application No. 62/672,450, entitled “Compositions and methods for delivery of AAV”, filed May 16, 2018, and U.S. Provisional Patent Application No. 62/729,645, entitled “Directed Evolution” filed Sep. 11, 2018, the contents of each of which are herein incorporated by reference in their entirety.

### REFERENCE TO THE SEQUENCE LISTING

The present application is being filed along with a Sequence Listing in electronic format as an ASCII text file. The Sequence Listing is provided as an ASCII text file entitled 2057-1029PCTSEQLST.txt, created on May 16, 2019, which is 588,258 bytes in size. The Sequence Listing is incorporated herein by reference in its entirety.

### FIELD OF THE DISCLOSURE

The present disclosure relates to compositions, methods and processes for the design, preparation, manufacture, use and/or formulation of adeno-associated virus capsids for improved biodistribution.

### BACKGROUND

Adeno-associated virus (AAV) vectors are a promising candidate for therapeutic gene delivery and have proven safe and efficacious in clinical trial.

Delivery of AAV to some systems in the body has proven to be particularly challenging, requiring invasive surgeries for sufficient levels of gene transfer. For some body systems, intravenous delivery has historically resulted in limited gene transfer, in part due to inefficient transduction into cells. There remains a need in the art for AAV vectors that may be administered by intravenous delivery and yet are able to efficiently target regions critical for treating a multitude of diseases.

One example of a system where delivery is challenging is the central nervous system. Delivery of AAV to regions of the central nervous system (CNS) has proven to be particularly challenging, requiring invasive surgeries for sufficient levels of gene transfer (See e.g., Bevan et al. Mol. Ther. 2011 November; 19(11): 1971-1980). Intravenous delivery has historically resulted in limited gene transfer to the CNS, in part due to the presence of the blood brain barrier (BBB). There remains a need in the art for AAV vectors that may be administered by intravenous delivery and yet are able to efficiently cross the blood brain barrier and target regions of the CNS critical for treating a multitude of CNS diseases.

The present disclosure addresses this need by providing novel AAV particles with engineered capsid proteins that allow for efficient transduction of CNS tissues. Further, the viral genomes of these AAV particles may be altered to suit the needs of any number of CNS diseases, providing platform capsids for targeting of CNS tissues.

### SUMMARY

In one aspect, provided herein are capsid proteins (e.g., chimeric AAV capsid proteins). The capsid proteins provided herein have been selected for their ability to infect tissues (e.g., central nervous system tissues) as well as specific cell types (e.g., neurons and/or astrocytes). The capsid proteins described herein can comprise an amino acid sequence of any one of the capsid proteins described in Tables 1-3, or a variant thereof. In some embodiments, the capsid protein can comprise an amino acid sequence of SEQ ID NO: 6, SEQ ID NO: 8, SEQ ID NO: 9, SEQ ID NO: 1-5, SEQ ID NO: 7, SEQ ID NO: 10-24, SEQ ID NO: 49-72, and SEQ ID NO: 97-120, or a fragment or variant thereof. In a specific embodiment, the capsid protein provided herein comprises the amino acid sequence of SEQ ID NO: 6, 8 or 9, or a fragment or variant thereof. In some embodiments, the capsid protein comprises SEQ ID NO: 6, or a fragment or variant thereof. In some embodiments, the capsid protein comprises SEQ ID NO: 8, or a fragment or variant thereof. In some embodiments, the capsid protein comprises SEQ ID NO: 9, or a fragment or variant thereof.

In one aspect, provided herein is a nucleic acid molecule that comprises a polynucleotide sequence that encodes a capsid protein described herein. Accordingly, in some embodiments, the nucleic acid molecule comprises a polynucleotide sequence that encodes any one of the capsid proteins described in Tables 1-3, or a variant thereof. In a specific embodiment, the nucleic acid molecule comprises a polynucleotide sequence that encodes the amino acid sequence of SEQ ID NO: 6, 8 or 9. Accordingly, in some embodiments, the polynucleotide sequence comprises SEQ ID NO: 30, 32 or 33.

In one aspect, provided herein is an insect cell (e.g., an Sf9 cell) comprising a polynucleotide sequence provided herein. Such an insect cell, in some embodiments, can comprises a polynucleotide sequence that promotes expression of a capsid protein provided herein in the insect cell. In another embodiment, the insect cell can comprise a polynucleotide sequence that encodes a Rep protein. Accordingly, in some embodiments, the insect cell provided herein can comprise a polynucleotide sequence encoding a Rep78, Rep68, Rep52 or Rep40 protein. In some embodiments, the insect cell comprises a polynucleotide sequence encoding Rep78. In some embodiments, the insect cell comprises a polynucleotide sequence encoding Rep52. In some embodiments, the insect cell comprises a polynucleotide sequence encoding Rep78 and Rep52.

In one aspect, provided herein is an AAV particle comprising a capsid protein as described herein and a viral genome described herein. An AAV particle provided herein can further comprise a viral genome that comprises at least one inverted terminal repeat (ITR) and at least one polynucleotide sequence that encodes a payload molecule. In some embodiments, an AAV particle provided herein comprises a capsid protein as described herein and a viral genome that comprises a nucleic acid sequence position between two ITRs. An AAV particle provided herein can comprise a capsid protein described herein, or a variant thereof. In one aspect, the AAV particle comprises one or more capsid proteins of Tables 1-3, or a variant thereof. In a specific embodiment, the AAV particle provided herein comprises a capsid protein that comprises the amino acid sequence of SEQ ID NO: 6, 8 or 9.

AAV particles comprising one or more capsid proteins described herein may transduce CNS structures following administration. Non-limiting examples of CNS structures include brain, spinal cord (cervical, thoracic, lumbar), hippocampus, putamen, brainstem nuclei, dentate nuclei, cerebellum, frontal cortex, motor cortex, occipital cortex, cingulate cortex, purkinje fibers, caudate nucleus, thalamus, cervical spinal cord, thoracic spinal cord, lumbar spinal cord, striatum, substantia nigra, and/or cerebral cortex.

In one aspect, AAV particles comprising one or more capsid proteins described herein transduce peripheral nervous system (PNS) structures following administration. Non-limiting examples of PNS structures include the sensory nervous system (e.g., dorsal root ganglia, trigeminal ganglia), the autonomous nervous system (e.g., parasympathetic and sympathetic ganglia), the enteric nervous system and nerve cell clusters in tissues and organs.

In one aspect, an AAV particle described herein penetrates the blood brain barrier following delivery of the AAV particle that comprises a capsid protein described herein. The delivery may be by any method known in the art, such as, but not limited to, intravenous administration or intracarotid artery delivery.

In one aspect, the AAV particle comprising one or more capsid proteins described herein comprises a viral genome that comprises a nucleic acid sequence (e.g., modulatory polynucleotide or siRNA) that, when expressed, inhibits or suppresses the expression of a gene of interest (e.g., superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A) and voltage-gated sodium channel alpha subunit 10 (SCN10A)) in a cell. The nucleic acid sequence comprises a sense strand sequence and an antisense strand sequence that may be independently 30 nucleotides or less and, the sense and/or antisense strands may comprise a 3′ overhang of at least 1 or at least 2 nucleotides. The sense sequence and antisense strand sequence may share a region of complementarity of at least four nucleotides in length (e.g., at least 17 nucleotides in length, between 19 and 21 nucleotides in length, or 19 nucleotides in length). The antisense strand may be excised from the AAV particle at a rate of at least 80%, 90%, 95% or more than 95%. The antisense strand may be excised from the AAV particle at a rate greater than the excision of the sense strand (e.g., 2 times, 5 times, 10 times or more than 10 times greater).

In one aspect, the AAV particle comprises a viral genome that comprises a nucleic acid sequence that, when expressed, inhibits or suppresses the expression of one or more genes of interest (e.g., SOD1, C9ORF72, TARDBP, ATXN3, HTT, APP, APOE, MAPT, SNCA, SCN9A and/or SCN10A) in a cell. For each gene of interest, the nucleic acid sequence comprises a sense strand sequence and an antisense strand sequence which may be independently 30 nucleotides or less and, the sense and/or antisense strands may comprise a 3′ overhang of at least 1 or at least 2 nucleotides. For each gene of interest, the sense sequence and antisense strand sequence may share a region of complementarity of at least four nucleotides in length (e.g., at least 17 nucleotides in length, between 19 and 21 nucleotides in length, or 19 nucleotides in length). For each gene of interest, the antisense strand may be excised from the AAV particle at a rate of at least 80%, 90%, 95% or more than 95%. The antisense strand may be excised from the AAV particle at a rate greater than the excision of the sense strand (e.g., 2 times, 5 times, 10 times or more than 10 times greater).

In one embodiment, the nucleic acid when expressed inhibits or suppresses the expression of two genes in a cell. In one embodiment, the nucleic acid when expressed inhibits or suppresses the expression of three genes in a cell. In one embodiment, the nucleic acid when expressed inhibits or suppresses the expression of four genes in a cell. In one embodiment, the nucleic acid when expressed inhibits or suppresses the expression of five genes in a cell. In one embodiment, the nucleic acid when expressed inhibits or suppresses the expression of six genes in a cell. In one embodiment, the nucleic acid when expressed inhibits or suppresses the expression of seven genes in a cell. In one embodiment, the nucleic acid when expressed inhibits or suppresses the expression of eight genes in a cell. In one embodiment, the nucleic acid when expressed inhibits or suppresses the expression of nine genes in a cell.

In one aspect, an AAV particle comprising one or more capsid proteins described herein comprises a viral genome that comprises a nucleic acid sequence that encodes a polypeptide, for example, an antibody that, when expressed, inhibits or suppresses the activity of a polypeptide of interest (e.g., SOD1, C9ORF72, TARDBP, ATXN3, HTT, APP, APOE, MAPT, SNCA, SCN9A and/or SCN10A) in a cell.

In one aspect, the AAV particle comprises a viral genome which comprises a nucleic acid sequence that expresses a gene of interest (e.g., an antibody, Aromatic L-Amino Acid Decarboxylase (AADC), APOE2, Frataxin, survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1), or gigaxonin (GAN)).

Provided herein are compositions (e.g., pharmaceutical compositions) comprising AAV particles. The AAV particles may comprise a viral genome comprising a nucleic acid sequence encoding a protein of interest (e.g., an antibody, AADC, APOE2, Frataxin, SMN, GCase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, ASPA, GRN, MeCP2, GLB1, and/or GAN). The AAV particles may comprise a viral genome comprising nucleic acid sequences that when expressed, inhibit or suppress the expression of one or more genes of interest (e.g., SOD1, C9ORF72, TARDBP, ATXN3, HTT, APP, APOE, MAPT. SNCA. SCN9A and/or SCN10A) in a cell. For example, the AAV particles may comprise a viral genome comprising nucleic acid sequences that when expressed, inhibits or suppresses the expression of two genes of interest in a cell. For example, the AAV particles may comprise a viral genome comprising nucleic acid sequences that when expressed, inhibits or suppresses the expression of three, four, five, six, seven, eight, or nine genes of interest in a cell.

Provided herein are methods of using AAV particles comprising one or more capsid proteins described herein. Accordingly, in one aspect, provided herein are methods of delivering a payload molecule to a cell. Such a method can comprise the steps of contacting the cell with an AAV particle described herein, wherein the AAV particle comprises a viral genome that encodes the payload molecule, such that the payload molecule is expressed in the cell, thereby delivering the payload molecule to the cell. As such, also provided herein are methods of inhibiting the expression of a target gene in a cell (e.g., mammalian cell, or mammalian cell of the CNS) by delivering to the cell an AAV particle comprising one or more capsid proteins described herein.

In one aspect, provided are methods for treating and/or ameliorating a neurological disease in a subject by administering a therapeutically effective amount of a composition comprising an AAV particle comprising one or more capsid proteins described herein. In some embodiments, the administration may be by intravenous or intracarotid artery delivery. In another embodiment, the administration is by direct administration into the CNS. The methods may be used to increase the expression of a protein of interest (e.g., an antibody, AADC, APOE2, Frataxin, SMN, GCase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, ASPA, GRN, MeCP2, GLB1, and/or GAN). The methods may be used to decrease the amount of expression and/or activity of a gene or polypeptide of interest (e.g., SOD1, C9ORF72, TARDBP, ATXN3, HTT, APP, APOE, MAPT, SNCA, SCN9A and/or SCN10A).

In one aspect, provided herein are methods for altering the level of a protein or gene of interest by administration of an AAV particle comprising one or more capsid proteins described herein. In some embodiments, the administration may be by intravenous or intracarotid artery delivery. In another embodiment, the administration may be by direct CNS delivery. In one aspect, the methods may be used to increase the expression of a protein of interest (e.g., an antibody, AADC, APOE2, Frataxin, SMN, GCase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, ASPA, GRN, MeCP2, GLB1, and/or GAN). In another aspect, the methods may be used to decrease the amount of expression or activity of a gene or polypeptide of interest (e.g., SOD1, C9ORF72, TARDBP, ATXN3, HTT, APP, APOE, MAPT, SNCA, SCN9A and/or SCN10A).

1. A capsid protein comprises a sequence selected from the group consisting of SEQ ID NO: 6, SEQ ID NO: 8, and SEQ ID NO: 9, or a fragment or variant thereof.

2. A nucleic acid molecule comprising a polynucleotide sequence that encodes the capsid protein of embodiment 1.

3. The nucleic acid molecule of embodiment 2, wherein the polynucleotide sequence is linked to a second polynucleotide sequence that promotes expression of the polynucleotide sequence in insect cells.

4. An insect cell comprising the polynucleotide sequence of any embodiments 2 or 3.

5. The insect cell of embodiment 4, further comprising a rep-encoding polynucleotide sequence that encodes at least one Rep protein, wherein the Rep protein is Rep78, Rep68, Rep40 and/or Rep52.

6. The insect cell of embodiment 5, wherein the Rep protein is Rep78.

7. The insect cell of embodiment 5, wherein the Rep protein is Rep68.

8. The insect cell of embodiment 5, wherein the Rep protein is Rep40.

9. The insect cell of embodiment 5, wherein the Rep protein is Rep52.

10. The insect cell of embodiment 5, wherein the rep-encoding polynucleotide sequence encodes Rep78 and Rep52.

11. The insect cell of any one of embodiments 5-10, wherein the rep-encoding polynucleotide sequence is part of the same nucleic acid molecule that encodes the capsid protein.

12. The insect cell of any one of embodiments 5-11, wherein the rep-encoding polynucleotide sequence is linked to a sequence that promotes expression in insect cells.

13. The insect cell of any one of embodiments 4-12, wherein the insect cell is an Sf9 insect cell.

14. An adeno-associated viral (AAV) particle, comprising the capsid protein of embodiment 1 and a viral genome, wherein the viral genome comprises at least one inverted terminal repeat (ITR) and at least one polynucleotide sequence that encodes at least one payload molecule.

15. The AAV particle of embodiment 14, wherein the at least one polynucleotide sequence that encodes at least one payload molecule is positioned between two ITRs.

16. The AAV particle of embodiment 14 or 15, wherein at least one of the at least one payload molecules is an siRNA duplex.

17. The AAV particle of embodiment 16, wherein the siRNA duplex, when expressed, inhibits or suppresses the expression of a gene of interest in a cell.

18. The AAV particle of embodiment 17, wherein the gene of interest is selected from the group consisting of superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A), and voltage-gated sodium channel alpha subunit 10 (SCN10A).

19. The AAV particle of embodiment 18, wherein the gene of interest is SOD1.

20. The AAV particle of embodiment 18, wherein the gene of interest is C9ORF72.

21. The AAV particle of embodiment 18, wherein the gene of interest is TARDBP.

22. The AAV particle of embodiment 18, wherein the gene of interest is ATXN3.

23. The AAV particle of embodiment 18, wherein the gene of interest is HTT.

24. The AAV particle of embodiment 18, wherein the gene of interest is APP.

25. The AAV particle of embodiment 18, wherein the gene of interest is APOE.

26. The AAV particle of embodiment 18, wherein the gene of interest is MAPT.

27. The AAV particle of embodiment 18, wherein the gene of interest is SNCA.

28. The AAV particle of embodiment 18, wherein the gene of interest is SCN9A.

29. The AAV particle of embodiment 18, wherein the gene of interest is SCN10A.

30. The AAV particle of embodiment 14 or 15, wherein at least one of the at least one payload molecule is a protein of interest.

31. The AAV particle of embodiment 30, wherein the protein of interest is selected from the group consisting of an antibody, Aromatic L-Amino Acid Decarboxylase (AADC), ApoE, Frataxin, survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8. CLN8, aspartoacylase (ASPA), progranulin (GRN). MeCP2, beta-galactosidase (GLB1), and gigaxonin (GAN).

32. The AAV particle of embodiment 18, wherein the protein of interest is an antibody.

33. The AAV particle of embodiment 18, wherein the protein of interest is AADC.

34. The AAV particle of embodiment 18, wherein the protein of interest is APOE.

35. The AAV particle of embodiment 18, wherein the protein of interest is Frataxin.

36. The AAV particle of embodiment 18, wherein the protein of interest is SMN.

37. The AAV particle of embodiment 18, wherein the protein of interest is GCase.

38. The AAV particle of embodiment 18, wherein the protein of interest is N-sulfoglucosamine sulfohydrolase.

39. The AAV particle of embodiment 18, wherein the protein of interest is N-acetyl-alpha-glucosaminidase.

40. The AAV particle of embodiment 18, wherein the protein of interest is iduronate 2-sulfatase.

41. The AAV particle of embodiment 18, wherein the protein of interest is alpha-L-iduronidase.

42. The AAV particle of embodiment 18, wherein the protein of interest is palmitoyl-protein thioesterase 1.

43. The AAV particle of embodiment 18, wherein the protein of interest is tripeptidyl peptidase 1.

44. The AAV particle of embodiment 18, wherein the protein of interest is battenin.

45. The AAV particle of embodiment 18, wherein the protein of interest is CLN5.

46. The AAV particle of embodiment 18, wherein the protein of interest is CLN6.

47. The AAV particle of embodiment 18, wherein the protein of interest is MFSD8.

48. The AAV particle of embodiment 18, wherein the protein of interest is CLN8.

49. The AAV particle of embodiment 18, wherein the protein of interest is ASPA.

50. The AAV particle of embodiment 18, wherein the protein of interest is GRN.

51. The AAV particle of embodiment 18, wherein the protein of interest is MeCP2.

52. The AAV particle of embodiment 18, wherein the protein of interest is GLB1.

53. The AAV particle of embodiment 18, wherein the protein of interest is GAN.

54. The AAV particle of embodiment 34, wherein the ApoE is ApoE2.

55. A method of delivering a payload molecule to a cell, comprising contacting the cell with the AAV particle of any one of embodiments 14-54, wherein the AAV particle comprises a viral genome that encodes the payload molecule, such that the payload molecule is expressed in the cell, thereby delivering the payload molecule to the cell.

56. The method of embodiment 55, wherein the payload molecule encodes a protein of interest.

57. The method of embodiment 55, wherein the payload molecule is a modulatory polynucleotide.

58. The method of any one of embodiments 55-57, wherein the cell is a mammalian cell.

59. The method of embodiment 58, wherein the mammalian cell is a human cell.

60. The method of embodiment 58 or 59, wherein the cell is a nervous system cell.

61. The method of embodiment 60, wherein the cell is a cell of the caudate, hippocampus, thalamus, putamen, brain stem, cortex, purkinje fiber, substantia nigra, spinal cord, dorsal root ganglion, cerebellum, or striatum.

62. The method of embodiment 61, wherein the cell of the cortex is a frontal, motor, occipital or cingulate cell of the cortex.

63. The method of embodiment 61, wherein the cell of the spinal cord is a cervical, thoracic, lumbar cell of the spinal cord.

64. The method of embodiment 60, wherein the cell is a medium spiny neuron.

65. The method of embodiment 60, wherein the cell is a neuron.

66. The method of embodiment 65, wherein the neuron is a cortical neuron.

67. The method of embodiment 60, wherein the cell is an astrocyte.

The details of various embodiments of the disclosure are set forth in the description below. Other features, objects, and advantages of the disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1 shows a flow diagram of a method for generating the chimeric capsid proteins described herein.

FIG. 2 shows a Venn diagram of the performance of the capsid proteins described herein in the four systems tested (NHP CNS, mouse CNS, neurons and astrocytes). Capsid protein HW01 was one of the best performing chimeric capsid proteins in all four screening systems.

FIG. 3 shows a Guide tree of the complexity of the chimeric VP1 capsid protein described herein.

### DETAILED DESCRIPTION

The details of one or more embodiments are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### I. COMPOSITIONS

### Adeno-Associated Viruses (AAVs), AAV Particles and Capsid Proteins

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism.

The parvoviruses and other members of the Parvoviridae family are generally described in Kenneth I. Bems, “Parvoviridae: The Viruses and Their Replication,” Chapter 69 in FIELDS VIROLOGY (3d Ed. 1996), the contents of which are incorporated by reference in their entirety.

The Parvoviridae family includes the Dependovirus genus, which includes AAV, capable of replication in vertebrate hosts including, but not limited to, human, primate, bovine, canine, equine, and ovine species.

The AAV viral genome is a linear, single-stranded DNA (ssDNA) molecule approximately 5,000 nucleotides (nt) in length. The AAV viral genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV viral genome typically comprises two ITR sequences. The AAV viral genome comprises a characteristic T-shaped hairpin structure defined by the self-complementary terminal 145 nt of the 5′ and 3′ ends of the ssDNA that form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV particles described herein can comprise one or more capsid proteins described herein and can comprise a viral genome, in whole or in part, of any naturally occurring and/or recombinant AAV serotype nucleotide sequence or variant.

In some embodiments, AAV particles comprising one or more capsid protein described herein are recombinant AAV particles that are replication defective, lacking sequences encoding functional Rep and Cap proteins within their viral genome. These defective AAV particles may have a viral genome lacking most or all parental coding sequences and essentially carry only one or two AAV ITR sequences and the nucleic acid of interest for delivery to a cell, a tissue, an organ or an organism.

In some embodiments, the viral genome of the AAV particles comprising one or more capsid proteins described herein comprise at least one control element that provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles comprising one or more capsid proteins described herein for use in therapeutics and/or diagnostics comprise a virus that has been distilled or reduced to the minimum components necessary for transduction of a nucleic acid payload or cargo of interest. In this manner, AAV particles comprising one or more capsid proteins described herein are engineered as vehicles for specific delivery while lacking the deleterious replication and/or integration features found in wild-type viruses.

AAV particles comprising one or more capsid proteins described herein may be produced recombinantly and may be based on AAV parent or reference sequences.

In addition to single stranded AAV viral genomes (e.g., ssAAVs), the present disclosure also provides for self-complementary AAV (scAAVs) viral genomes, scAAV viral genomes contain DNA strands that anneal together to form double stranded DNA. By skipping second strand synthesis, scAAVs allow for rapid expression in the cell.

In some embodiments, an AAV particle comprising one or more capsid proteins described herein is an scAAV.

In some embodiments, an AAV particle comprising one or more capsid proteins described herein is an ssAAV.

Methods for producing and/or modifying AAV particles are disclosed in the art such as pseudotyped AAV particles (PCT Patent Publication Nos. WO200028004; WO200123001; WO2004112727; WO 2005005610 and WO 2005072364, the content of each of which is incorporated herein by reference in its entirety).

In some embodiments, the AAV particles comprising one or more capsid proteins described herein comprise a payload region encoding the polypeptides or polynucleotides described herein and may be introduced into mammalian cells.

### Capsid Proteins

In some embodiments, described herein is a capsid protein as found in Table 1, or variant thereof. In some embodiments, described herein are capsid proteins encoded by a polynucleotide sequence as found in Table 1.

In some embodiments, described herein is a capsid protein as found in Table 2, or variant thereof. In some embodiments, described herein is a capsid protein encoded by a polynucleotide sequence as found in Table 2.

In some embodiments, described herein is a capsid protein as found in Table 3, or variant thereof. In some embodiments, described herein is a capsid protein encoded by a polynucleotide sequence as found in Table 3.

In one aspect, AAV particles are described herein that comprise one or more capsid proteins, or variants thereof, described herein.

In some embodiments, a capsid protein described herein may be selected from any of those capsid proteins (VP1) found in Table 1. In some embodiments, the capsid protein may be a variant of any of the capsid proteins found in Table 1. In some embodiments, AAV particles are described herein that comprise such a capsid protein or proteins, or variants thereof.

In some embodiments, a capsid protein or proteins may be encoded by a polynucleotide sequence found in Table 1. In some embodiments, the capsid protein or proteins may be encoded by a polynucleotide sequence that is a codon optimized form of a polynucleotide sequence of Table 1. For example, the capsid protein or proteins may be encoded by a polynucleotide sequence that is codon optimized for expression in insect cells, such as Sf9 insect cells. In some embodiments, the capsid protein or proteins may be encoded by a polynucleotide sequence that differs from a polynucleotide sequence of Table 1 due to amino acid code degeneracy. In some embodiments, AAV particles are described herein that comprise a capsid protein or proteins, or variants thereof, encoded by such a polynucleotide. In some embodiments, AAV particles are described herein that comprise capsid proteins, or variants thereof, encoded by such a polynucleotide and an RNA splice variant or variants of such a polynucleotide.

In any of the DNA and RNA sequences referenced and/or described herein, the single letter symbol has the following description: A for adenine; C for cytosine; G for guanine; T for thymine; U for Uracil: W for weak bases such as adenine or thymine; S for strong nucleotides such as cytosine and guanine; M for amino nucleotides such as adenine and cytosine; K for keto nucleotides such as guanine and thymine: R for purines adenine and guanine; Y for pyrimidine cytosine and thymine: B for any base that is not A (e.g., cytosine, guanine, and thymine); D for any base that is not C (e.g., adenine, guanine, and thymine); H for any base that is not G (e.g., adenine, cytosine, and thymine): V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine: P (Pro) for Proline; V (Val) for Valine; I (Ile) for Isoleucine: C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

TABLE 1
Capsid Proteins (VP1)

| Capsid | | |
|---|---|---|
| Protein | Amino Acid | Representative Polynucleotide |
| KJ01 | MAADGYLPDWLEDNLSEGIREWWDLKP | 5′-atggctgccgacggctatcttccagattggctcgaggacaacctctctgagg |
| (VP1) | GAPKPKANQQKQDDGRGLVLPGYKYLG | gcattcgcgagtggtgggacttgaaacctggagccccgaaacccaaagccaacca |
| | PFNGLDKGEPVNAADAAALEHDKAYDQ | gcaaaagcaggacgacggccggggtccggtgctccctggccacaagtacctcgga |
| | QLQAGDNPYLRYNHADAEFQERLQEDT | cccttcaacggactcgacaagggggagcccgtcaacgcggcggacgcagcggccc |
| | SFGGNLGRAVFQAKKRVLEPLGLVEEG | tcgagcacgacaaggcctacgaccagcagctgcaggcgggtgacaatccgtacct |
| | AKTAPGKKRPVEPSPQRSPDSSTGIGK | gtggtataaccacgctgacgccgagtttcaggagcgtctgcaagaagatacgtct |
| | KGQQPARKRLNFGQTGDADSVPDPQPL | tttgggggcaacctcgggcgagcagtcttccaggccaagaagcgggttctcgaac |
| | GEPPAAPSSVGSGTMAAGGGAPMADNN | ctctcggtctggttgaggaaggcgctaagacggctcctggaaagaagagaccggt |
| | EGADGVGSSSGNWHCDSTWLGDRVITT | agagccatcaccccagcgttctccagactcctctacgggcatcggcaagaaaggc |
| | STRTWALPTYNNHLYKQISNGTSGGST | caacagcccgcaagaaaaagattgaattttggtcagactggagacgcagactcag |
| | NDNTYFGYSTPWGYFDFNRFHCHFSPR | tacctgacccacaacctctcggagaacctccagcagcgccctctagtgtgggatc |
| | DWQRLINNNWGFRPKRLSFKLFNIQVK | tggtacaatggctgcaggcggtggcgctccaatggcagacaataacgaaggcgcc |
| | EVTQNEGTKTIANNLTSTIQVFTDSEY | gacggagtgggtagttcctcaggaaattggcattgcgattccacatggctgggcg |
| | QLFYVLGSAHQGCLPPFPADVFMIPQY | acagagtcatcaccaccagcacccgaacctgggccctgcccacctacaacaacca |
| | GYLTLNNGSQAVGRSSFYCLEYFPSQM | cctctacaagcaaatctccaacggcacctcgggaggaagcaccaacgacaacacc |
| | LRTGNNFQFTYTFEDVPFHSSYAHSQS | tattttggctacagcaccccctgggggtattttgactttaacagattccactgcc |
| | LDRLMNPLIDQYLYYLSRTQSTGGTAG | tattttggctacagcaccccctgggggtattttgactttaacagattccactgcc |
| | TQQLLFSQAGPNNMSAQAKNWLPGPCY | acttttcaccacgtgactggcagcgactcatcaacaacaactggggattccggcc |
| | RQQRVSTTLSQNNNSNFAWTGATKYHL | caagagactcagcttcaagctcttcaacatccaggtcaaggaggtcacgcagaat |
| | NGRDSLVNPGVAMATHKDDEERFFPSS | gaaggcaccaagaccatcgccaataacctcaccagcaccatccaggtgtttacgg |
| | GVLMFGKQGAGKDNVDYSSVMLTSEEE | actcggagtaccagctgccgtacgttctcggctctgcccaccagggctgcctgcc |
| | IKTTNPVATEQYGVVADNLQQQNAAPI | tccgttcccggcggacgtgttcatgattccccagtacggctacctaacactcaac |
| | VGAVNSQGALPGMVWQNRDVYLQGPIW | aacggtagtcaggccgtgggacgctcctccttctactgcctggaatactttcctt |
| | AKIPHTDGNFHPSPLMGGFGLKHPPPQ | cgcagatgctgagaaccggcaacaacttccagtttacttacaccttcgaggacgt |
| | ILIKNTPVPADPPTTFSQAKLASFITQ | gcctttccacagcagctacgcccacagccagagcttggaccggctgatgaatcct |
| | YSTGQVSVEIEWELQKENSKRWNPEIQ | ctgattgaccagtacctgtactacctgtctcggactcagtccacgggaggtaccg |
| | YTSNYYKSTNVDFAVNTDGTYSEPRPI | caggaactcagcagttgctattttctcaggccgggcctaataacatgtcggctca |
| | GTRYLTRNL SEQ ID NO: 1 | ggccaaaaactggctacccgggccctgctaccggcagcaacgcgtctccacgaca |
| | | ctgattgaccagtacctgtactacctgtctcggactcagtccacgggaggtaccg |
| | | caggaactcagcagttgctattttctcaggccgggcctaataacatgtccggctc |
| | | ggccaaaaactggctacccgggccctgctaccggcagcaacgcgtctccacgaca |
| | | ctgtcgcaaaataacaacagcaactttgcctggaccggtgccaccaagtatcatc |
| | | tgaatggcagagactctctggtaaatcccggtgtcgctatggcaacccacaagga |
| | | cgacgaagagcgattttttccgtccagcggagtcttaatgtttgggaaacaggga |
| | | gctggaaaagacaacgtggactatagcagcgttatgctaaccagtgaggaagaaa |
| | | ttaaaaccaccaacccagtggccacagaacagtacggcgtggtggccgataacct |
| | | gcaacagcaaaacgccgctcctattgtaggggccgtcaacagtcaaggagcctta |
| | | cctggcatggtctggcagaaccgggacgtgtacctgcagggtcctatctgggcca |
| | | agattcctcacacggacggaaactttcatccctcgccgctgatgggaggctttgg |
| | | actgaaacacccgcctcctcagatcctgattaagaatacacctgttcccgcggat |
| | | cctccaactaccttcagtcaagctaagctggcgtcgttcatcacgcagtacagca |
| | | ccggacaagtcagcgtggaaatcgagtgggagctgcagaaggaaaacagcaaacg |
| | | ctggaatccagagattcagtacacttcaaactactacaaatctacaaatgtggac |
| | | tttgctgttaacacagatggcacttattctgagcctcgccccatcggcacccgtt |
| | | acctcacccgtaatctg-3′ SEQ ID NO: 25 |
| KJ02 | MAADGYLPDWLEDTLSEGIRQWWKLKP | 5′-atggctgccgatggttatcttccagattggctcgaggacactctctctgaag |
| (VP1) | GPPPPKPAERHKDDGRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacggccggggtctggtgcttcctggctacaagtacctcgga |
| | QLKAGDNPYLRYNHADAEFQERLQEDT | cccttcaacggactcgacaagggggagcccgtcaacgcggcggacgcagcggccc |
| | SFGGNLGRAVFQAKKRVLEPLGLVEEG | tcgagcacgacaaggcctacgaccagcagctcaaagcgggtgacaatccgtacct |
| | AKTAPGKKRPVEQSPQEPDSSSGIGKT | gcggtataaccacgccgacgccgagtttcaggagcgtctgcaagaagatacgtct |
| | GQQPAKKRLNFGQTGDSESVPDPQPIG | tttgggggcaacctcgggcgagcagtcttccaagccaagaagcgggttctcgaac |
| | EPPAGPSGLGSGTMAAGGGAPMADNNE | ctctcggtctggttgaggaaggcgctaagacggctcctggaaagaaacgtccggt |
| | GADGVGNASGNWHCDSTWLGDRVITTS | agagcagtcgccacaagagccagactcctcctcgggcatcggcaagacaggccaa |
| | TRTWALPTYNNHLYKQISSETAGSTND | cagcccgctaaaaagagactcaattttggtcagactggcgactcagagtcagtcc |
| | NTYFGYSTPWGYFDFNRFHCHFSPRDW | ccgaccctcaaccaatcggagaaccaccagcaggcccctctggtctgggatctgg |
| | QRLINNNWGFRPKRLNFKLLNIQVKEV | tacaatggctgcaggcggtggcgctccaatggcagacaataacgagggcgccgac |
| | TDNNGVKTIANNLTSTIQVFTDSEYQL | ggagtgggtaatgcctcaggaaattggcattgcgattccacatggctgggcgaca |
| | PYVLGSAHQGCPPPFPADVFMIPQYGY | gagtcattaccaccagcacccgaacctgggccctgcccacctacaacaaccacct |
| | LTLNNGSQAVGRSSFYCLEYFPSQMLR | ctacaagcaaatctccagtgaaactgcaggtagtaccaacgacaacacctacttc |
| | TGNNFEFSYSFEDVPFHSSYAHSQSLD | ggctacagcaccccctgggggtattttgacttcaacagattccactgccacttct |
| | RLMNPLIDQYLYYLSRTQSTGGTAGTQ | caccacgtgactggcagcgactcatcaacaacaactggggattccgacctaagcg |
| | QLLFSQAGPNNMSAQAKNWLPGPCYRQ | actcaacttcaagctcctcaacattcaggtcaaagaagttacggacaacaatgga |
| | QRVSTTLSQNNNSNFAWTGATKYHLNG | gtcaagaccatcgccaataacctcaccagcaccatccaggtgtttacggactcgg |
| | RDSLVNPGVAMATHKDDEERFFPSSGV | agtaccagctgccgtacgttctcggctctgcccaccagggctgcccgcctccgtt |
| | LMFGKQGAGKDNVDYSSVMLTSEEEIK | cccggcggacgtcttcatgattcctcagtacggctacctgactctcaacaatggc |
| | TTNPVATEQYGVVADNLQQQNTAPQIG | agtcaggccgtgggccgttcctccttctactgcctggagtactttccttctcaaa |
| | TVNSQGALPGMVWQNRDVYLQGPIWAK | tgctgagaacgggcaacaactttgagttcagctacagcttcgaggacgtgccttt |
| | IPHTDGHFHPSPLMGGFGLKHPPPQIL | ccacagcagctacgcacacagccagagcctggaccggctgatgaatcccctcatc |
| | IKNTPVPANPSTTFNQSKLNSFITQYS | gaccagtacctgtactacctgtctcggactcagtccacgggaggtaccgcaggaa |
| | TGQVSVEIEWELQKENSKRWNPEIQTY | ctcagcagttgctattttctcaggccgggcctaataacatgtcggctcaggccaa |
| | SNYYKSTSVDFAVNTEGVYSEPRPIGT | aaactggctacccgggccctgctaccggcagcaacgcgtctccacgacactgtcg |
| | HYLTRNL SEQ ID NO: 2 | caaaataacaacagcaactttgcctggaccggtgccaccaagtatcatctgaatg |
| | | gcagagactctctggtaaatcccggtgtcgctatggcaacccacaaggacgacga |
| | | agagcgattttttccgtccagcggagtcttaatgtttgggaaacagggagctgga |
| | | aaagacaacgtggactatagcagcgttatgctaaccagtgaggaagaaattaaaa |
| | | ccaccaacccagtggccacagaacagtacggcgtggtggccgataacctgcaaca |
| | | gcaaaacacggctcctcaaattggaactgtcaacagccagggggccttacccggt |
| | | atggtctggcagaaccgggacgtgtacctgcagggtcccatctgggccaagattc |
| | | cacacacggacggacattttcacccctctcccctcatgggtggattcggacttaa |
| | | acaccctcctccacagattctcatcaagaacaccccggtacctgcgaatccttcg |
| | | accaccttcaaccagtcaaagctgaactctttcatcacgcaatacagcaccggac |
| | | aggtcagcgtggaaattgaatgggagctgcagaaggaaaacagcaagcgctggaa |
| | | ccccgagatccagtacacctccaactactacaaatctacaagtgtggactttgct |
| | | gttaatacagaaggcgtgtactctgaaccccgccccattggcacccattacctca |
| | | cccgcaacctg-3′ SEQ ID NO: 26 |
| KJ03 | MAADGYLPDWLEDTLSEGIRQWWKLKP | 5′-atggctgccgatggttatcttccagattggctcgaggacactctctctggag |
| (VP1) | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctggctacaagtacctcgga |
| | QLKAGDNPYLRYNHADAEFQERLKEDT | cccttcaacggactcgacaagggggagcccgtcaacgcggcggacgcagcggccc |
| | DFGGNLGRAVFQAKKRVLEPLGLVEEG | tcgagcacgacaaggcctacgaccagcagctcaaagcgggtgacaatccgtacct |
| | AKTAPAKKRPVEPSPQRSPDSSTGIGK | gcggtataaccacgccgacgccgagtttcaggagcgccttaaagaagatacgtct |
| | KGQQPARKRLNFGQTGDADSVPDPQPL | tttgggggcaacctcggacgagcagtcttccaggcgaaaaagagggttctcgaac |
| | GEPPAAPSGVGPNTMASGGGAPMADNN | ctctcggtctggttgaggaaggcgctaagacggctcctgcaaagaagagaccggt |
| | EGADVGVSSSGNWHCDSTWLGDRVITT | agagccgtcacctcagcgttcccccgactcctccacgggcatcggcaagaaaggc |
| | STRTWALPTYNNHLYKQISSASTGASN | cagcagcctgcaagaaaaagattgaattttggtcagactggagacgcagactcag |
| | DNHYFGYSTPWGYFDFNRFHCHFSPRD | tacctgacccccagcctctcggagaacctccagcagcgccctctggtgtgggacc |
| | WQRLINNNWGFRPKRLSFKLFNIQVKE | taatacaatggcttcaggcggtggcgcaccaatggcagacaataacgaaggcgcc |
| | VTQNEGTKTIANNLTSTIQVFTDSEYQ | gacggagtgggtagttcctcaggaaattggcattgcgattccacatggctgggcg |
| | LPYVLGSAHQGCLPPFPADVFMIPQYG | acagagtcatcaccaccagcacccgaacatgggccttgcccacctataacaacca |
| | YLTLNNGSQAVGRSSFYCLEYFPSQML | cctctacaagcaaatctccagtgcttcaacgggggccagcaacgacaaccactac |
| | RTGNNFTFSYTFEDVPFHSSYAHSQSL | ttcggctacagcaccccctgggggtattttgacttcaacagattccactgccact |
| | DRLMNPLIDQYLYYLSRTQSTGGTAGT | tttcaccacgtgactggcaaagactcatcaacaacaactggggattccggcccaa |
| | QQLLFSQAGPNNMSAQAKNWLPGPCYR | gagactcagcttcaagctcttcaacatccagatcaaggaggtcacgcagaataaa |
| | QQRVSTTLSQNNNSNFAWTGGTKYHLN | ggcaccaagaccatcgccaataacctcaccagcaccatccaggtgtttacggact |
| | GRNSLANPGIAMATHKDDEERFFPSNG | cggagtaccagctgccgtacgttctcggctctgcccaccagggctgcctgcctcc |
| | ILIFGKQNAARDNADYSDVMLTSEEEI | gttcccggcggacgtcttcatgattcctcagtacggctacctgacgctcaacaat |
| | KTTNPVATEEGIVADNLQQQNTAAPQI | ggcagccaggcagtgggacggtcatccttttactgcctggaatatttcccatcgc |
| | GTVNSQGALPGMVWQNRDVYLQGPIWA | agatgctgagaacgggcaataactttaccttcagctacaccttcgaggacgtgcc |
| | KIPHTDGNFHPSPLMGGFGLKHPPPQI | tttccacagcagctacgcgcacagccaaagcctggaccggctgatgaaccccctc |
| | LIKNTPVPADPPTTFNQSKLNSFITQY | atcgaccagtacctgtactacatgtctcggactcagtccacgggaggtaccgcag |
| | STGQVSVEIEWELQKENSKRWNPEIQY | gaactcagcagttgctattttctcaggccgggcctaataacatgtcggctcaggc |
| | TSNYYKSTSVDFAVNTEGVYSEPRPIG | taagaactggctacctggaccttgctaccggcagcagcgagtctctacgacactg |
| | TRYLTRNL SEQ ID NO: 3 | tcgcaaaacaacaacagcaactttgcttggactggtgggaccaaataccatctga |
| | | atggaagaaattcattggctaatcctggcatcgctatggcaacacacaaagacga |
| | | cgaggagcgtttttttcccagtaacgggatcctgatttttggcaaacaaaatgct |
| | | gccagagacaatgcggattacagcgatgtcatgctcaccagcgaggaagaaatca |
| | | aaaccactaaccctgtggctacagaggaatacgatatcgtggcagataacttgca |
| | | gcagcaaaacacggctcctcaaattggaactgtcaacagccagggggccttaccc |
| | | ggtatggtctggcagaaccgggacgtgtacctgcagggtcctatctgggccaaga |
| | | ttcctcacacggacggaaactttcatccctcgccgctgatgggaggctttggact |
| | | gaaacacccgcctcctcagatcctgattaagaacacgcctgtacctgcggatcct |
| | | ccgaccaccttcaaccagtcaaagctgaactctttcatcacgcaatacagcaccg |
| | | gacaggtcagcgtggaaattgaatgggagctacagaaggaaaacagcaagcgctg |
| | | gaaccccgagatccagtacacctccaactactacaaatctacaagtgtggacttt |
| | | gctgttaatacagaaggcgtgtactctgaaccccgccccattggcacccgttacc |
| | | tcacccgtaatctg-3′ SEQ ID NO: 27 |
| KJ04 | MAADGYLPDWLEDTLSEGIRQWWDLKP | 5'-atggctgccgatggttatcttccagattggctcgaggacactctctctgaag |
| (VP1) | GAPKPKANQQKQDDGRGLVLPGYKLYG | gaataagacagtggtgggacttgaaacctggagccccgaaacccaaagccaacca |
| | PFNGLDKGEPVNAADAEALEHDKAYDQ | gcaaaagcaggacgacggccggggcctggtgcttcctggctacaagtacctcgga |
| | QLKAGDNPYLKYNHADAEFQERLKEDT | cccttcaacggactcgacaagggggagcccgtcaacgcggcagacgcggaggccc |
| | SFGGNLGRAVFQAKKRVLEPLGLVEEA | tcgagcacgacaaggcctacgaccagcagctcaaggccggagacaacccgtacct |
| | AKTAPGKKRPVEPSPQRSPDSSTGIGK | caagtacaaccacgccgacgccgagttccaggagcggctcaaagaagatacgtct |
| | KGQQPAKKRLNFGQTGDSESVPDPQPI | tttgggggcaacatcgggcgagcagtattccaggccaagaagcgggttctcgaac |
| | GEPPAAPSGLGPNTMAAGGGAPMADNN | ctctcggtctggttgaggaagctgctaagacggctcctggaaagaagagaccggt |
| | EGADGVGSSSGNWHCDSTWLGDRVITT | agaaccgtcacctcagcgttcccccgactcctctacgggcatcggcaagaaaggc |
| | STRTWALPTYNNHLYKQISNGTSGGAT | cagcagcccgcgaaaaagagactcaactttgggcagactggcgactcagagtcag |
| | NDNTYFGYSTPWGYFDFNRFHCHFSPR | tgcccgaccctcaaccaatcggagaacctccagcagccccctcaggtctgggacc |
| | DWQRLINNNWGFRPKRLSFKLFNIQVK | taatacaatggctgcaggcggtggcgcaccaatggcagacaataacgaaggcgcc |
| | EVTQNEGTKTIANNLTSTIQVFTDSEY | gacggagtgggtagttactcgggaaattggcattgcgattccacatggctgggcg |
| | QLPYVLGSAHQGCLPPFPADVFMIPQY | acagagtcatcaccaccagcacccgaacctgggccctgcccacctacaacaacca |
| | GYLTLNNGSQAVGRSSFYCLEYFPSQM | cctctacaagcaaatctccaacgggacatcgggaggagccaccaacgacaacacc |
| | LRTGNNFQFTYTFEDVPFHSSYAHSWS | tacttcggctacagcaccccctgggggtattttgacttcaacagattccactgcc |
| | LDRLMNPLIDQYLYYLSRTQTTGGTAN | acttctcaccacgtgactggcaacgactcatcaacaacaattggggattccgacc |
| | TQTLGFSQGGPNTMANQAKNWLPGPCY | caaaagactcagcttcaagctcttcaacatccaggtcaaggaggtcacgcagaat |
| | RQQRVSTTTGQNNNSNFAWTAGTKYHL | gaaggcaccaagaccatcgccaataacctcaccagcaccatccaggtgtttacgg |
| | NGRNSLANPGIAMATHKDDEEFFFPSN | actcggagtaccagctgccgtacgttctcggctctgcgcaccagggctgcctgcc |
| | GILIGGKQNAARDNADYSDVMLTSEEE | tccgttcccggcggacgtcttcatgattcctcagtacgggtacctgactctgaac |
| | IKTTNPVATEEYGIVADNLQQQNTAPQ | aatggcagtcagaccgtggaccgttcctccttctactgcctggaatattttccat |
| | IGTVNSQGALPGMVWQNRDVYLQGPIW | cgcagatgctgagaaccggcaacaacttccagtttacttacaccttcgaggacgt |
| | AKIPHTDGNFHPSPLMGGFGLHKPPPQ | gcctttccacagcagctacgcccacagccagagcttggaccggctgatgaatcct |
| | ILIKNTPVPANPPEVFTPAKFASFITQ | ctgattaaccagtacctgtactacttatctcggactcaaacaacaggaggcacga |
| | YSTGQVSVEIEWELQKENSKRWNPEIQ | caaatacgcagactctgggcttcagccaaggtgggcctaatacaatggccaatca |
| | YTSNFEKQTGVDFAVDSQGVYSEPRPI | ggcaaagaactggctgccaggaccctgttaccgccaacaacgcgtctcaacgaca |
| | GTRYLTRNL SEQ ID NO: 4 | accgggcaaaacaacaatagcaactttgcctggactgctgggaccaaataccatc |
| | | tgaatggaagaaattcattggctaatcctggcatcgctatggcaacacacaaaga |
| | | cgacgaggagcgtttttttcccagtaacgggatcctgatttttggcaaacaaaat |
| | | gctgccagagacaatgcggattacagcgatgtcatgctcaccagcgaggaagaaa |
| | | tcaaaaccactaaccctgtggctacagaggaatacggtatcgtggcagataactt |
| | | gcagcagcaaaacacggctcctcaaattggaactgtcaacagccagggggcctta |
| | | cccggtatggtctggcagaaccgggacgtgtacctgcagggtcccatctgggcca |
| | | agattcctcacacggacggcaacttccacccgtctccgctgatgggcggctttgg |
| | | cctgaaacatcctccgcctcagatcctgatcaagaacacgcccgttcccgctaat |
| | | cctccggaggtgtttactcctgccaagtttgcttcgttcatcacacagtacagca |
| | | ccggacaagtcagcgtggaaatcgagtgggagctgcagaaggaaaacagcaagcg |
| | | ctggaacccggagattcaatacacctccaactttgaaaaacagactggtgtggac |
| | | tttgccgttgacagccagggtgtttactctgagcctcgccctattggcactcgtt |
| | | acctcacccgcaacctg-3′ SEQ ID NO 28 |
| KJ05 | MAADGYLPDWLEDNLSEGIREWWDLKP | 5′-atggctgccgatggttatcttccagattggctcgaggacaacctctctgagg |
| (VP1) | GAPKPKANQQKQDNGRGLVLPGYKYLG | gcattcgcgagtggtgggacctgaaacctggagccccgaaacccaaagccaacca |
| | PFNGLDKGEPVNAADAAALEHDKAYDQ | gcaaaagcaggacaacggccggggtcttgtgcttcctgggtacaagtacctcgga |
| | QLKAGDNPYLRYNHADAEFQERLQEDT | cccttcaacggactcgacaagggggagcccgtcaacgcggcggacgcagcggccc |
| | SFGGNLGRAVFQAKKRVLEPLGLVEEG | tcgagcacgacaaggcctacgaccagcagctcaaagcgggtaacaacccgtacct |
| | AKTGPGKKRPVEPSPQRSPDSSTGIGK | gcggtacaaccacgccgacgcggagtttcaggagcgtctgcaagaagatacgtct |
| | KGQQPAKKRLNFGQTGDTESVPDPQPI | tttgggggcaacctcgggcgagcagtcttccaggccaagaagcgggttctcgaac |
| | GEPPAAPSGVGSLTMASGGGAPVADNN | ctctcggtctggttgaggaaggcgctaagacggctcctggaaagaagagaccggt |
| | EGADGVGNASGNWHCDSTWLGDRVITT | agagccatcaccccagcgttctccagactcctctacgggcatcggcaagaaaggc |
| | STRTWALPTYNNHLYKQISNSTSGGSS | cagcagcccgctaaaaagagactcaatttcggtcagactggcgacacagagtcag |
| | NDNAYFGYSTPWGYFDFNGFHCHFSPR | tcccagaccctcaaccaatcggagaacctcccgcagccccctcaggtgtgggatc |
| | DWQRLINNNWGFRPKRLGFKLFNIQVK | tcttacaatggcttcaggtggtggcgcaccagtggcagacaataacgaaggtgcc |
| | EVTQNEGTKTIANNLTSTIQVFTDSEY | gacggagtgggtaatgcctcaggaaattggcattgcgattccacatggctgggcg |
| | QLPYVLGSAHQGCLPPFPADVFMIPQY | acagagtcatcaccaccagcacccgaacatgggccctgcccacctacaacaacca |
| | GYLTLNNGSQAVGRSSFYCLEYFPSQM | cctctacaagcaaatctccaacagcacatctggaggatcttcaaatgacaacgcc |
| | LRTGNNFEFSYTFEDVPFHSSYAHSQS | tacttcggctacagcaccccctgggggtattttgacttcaacggattccactgcc |
| | LDRLMNPLIDYYLYYLSRTQTTGGTAN | atttctcaccacgtgactggcagcgactcatcaacaaaaattggggattccggcc |
| | TQTLGFSQGGPNTMANQAKNWLPGPCY | caagagactcggcttcaagctcttcaacatccaggtcaaggaggtcacgcagaat |
| | RQQRVSTTTGQNNNSNFAWTAGTKYHL | gaaggcaccaagaccatcgccaataacctcaccagcaccatccaggtgtttacgg |
| | NGRNSLANPGIAMATHKDDEERFFPSN | actcggagtaccagctgccgtacgttctcggctctgcccaccagggctgcctgcc |
| | GILIFGKQNAARDNADYSDMVLTSEEE | tccgttcccggcggacgtgttcatgattcctcagtacgggtacctgactctgaac |
| | IKTTNPVATEEYGIVADNLQQQNTAPQ | aatggcagtcaggccgtgggccgttcctccttctactgcctggagtactttcctt |
| | IGTVDSQGALPGMVWQNRDVYLQGPIW | ctcaaatgctgcgaactggaaacaattttgaattcagctacaccttcgaggacgt |
| | AKIPHTDGNFHPSPLMGGFGLKHPPPQ | gcctttccacagcagctacgcgcacagccagagcctggacaggctgatgaatccc |
| | ILIKNTPVPADPPTTFNQSKLNSFITQ | ctcatcgactactacctgtactacttgtctcggactcaaacaacaggaggcacgg |
| | YSTGQVSVEIEWELQKENSKRWNPEIQ | caaatacgcagactctgggcttcagccaaggtgggcctaatacaatggccaatca |
| | YTSNYYKSTSVDFAVNTEGVYSEPRPI | ggcaaagaactggctgccaggaccctgttaccgccaacaacgcgtctcaacgaca |
| | GTRYLTRNL SEQ ID NO: 5 | accgggcaaaacaacaatagcaactttgcctggactgctgggaccaaataccatc |
| | | tgaatggaagaaattcattggctaatcctggcatcgctatggcaacacacaaaga |
| | | cgacgaggagcgtttttttcccagtaacgggatcctgatttttggcaaacaaaat |
| | | gctgccagagacaatgcggattacagcgatgtcatgctcaccagcgaggaagaaa |
| | | tcaaaaccactaacectatggctacagaggaatacggtatcgtggcagataactt |
| | | gcagcagcaaaacacggctcctcaaattggaactgtcgacagccagggggcctta |
| | | cccggtatggtctggcagaaccgggacgtgtacctgcagggtcccatctgggcca |
| | | agattcctcacacggacggcaacttccacccgtccccgctgatgggcggctttgg |
| | | cctgaaacatcctccgcctcagatcctgatcaagaacacgcctgtacctgcggat |
| | | cctccgaccaccttcaaccagtcaaagctgaactctttcatcacgcaatacagca |
| | | ccggacaggtcagcgtggaaattgaatgggagctacagaaggaaaacagcaagcg |
| | | ctggaaccccgagatccagtacacctccaactactacaaatctacaagtgtggac |
| | | tttgctattaatacagaagacgtttactctgagcctcgccctattgggactcgtt |
| | | acctcacccgtaatctg-3′ SEQ ID NO: 29 |
| HW01 | MAADGYLPDWLEDTLSEGIRQWWKLKP | 5′-atggctgccgatggttatcttccagattggctcgaggacactctctctgaag |
| (VP1) | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctggctacaagtacctcgga |
| | QLKAGDNPYLRYNHADAEFQERLQEDT | cccttcaacggactcgacaagggggagcccgtcaacgcggcggacgcagcggccc |
| | SFGGNLGRAVFQAKKRLLEPLGLVEEA | tcgagcacgacaaggcctacgaccagcagctcaaagcgggtgacaatccgtacct |
| | AKTAPGKKRPVEQSPQEPDSSAGIGKS | gcggtataaccacgccgacgccgagtttcaggagcgtctgcaagaagatacgtct |
| | GAQPAKKRLNFGQTGSDESVPDPQPLG | tttgggggcaacctcgggcgagcagtcttccaggccaaaaagaggcttcttgaac |
| | EPPATPAAVGPTTMAAGGGAPMADNNE | ctcttggtctggttgaggaagcggctaagacggctcctggaaagaagaggcctgt |
| | GADGVGNSSGNWHCDSTWLGDRVITTS | agagcagtctcctcaggaaccggactcctccacgggtattggcaaatcgggtaca |
| | TRTWALPTYNNHLYKQISNSTSGGSSN | cagcccgctaaaaagagactcaattttggtcagactggcgactcagagtcagtcc |
| | DNAYFGYSTPWGYFDFNRFHCHFSPRD | ccgatccacaacctctcggagaacctccagcaacccccgctgctgtgggacctac |
| | WQRLINNNWGFRPKRLNFKLFNIQVKE | tacaatggctgcaggcggtggcgctccaatggcagacaataacgaaggcgccgac |
| | VTQNEGTKTIANNLTSTIQVFTDSEYQ | ggagtgggtaattcctcgggaaattggcattgcagattccacatggctggggaca |
| | LPYVLGSAHQGCLPPFPADVFMIPQYG | gagtcatcaccaccagcacccgaacctgggccctacccacctacaacaaccacct |
| | YLTLNNGSQAVGRSSFYCLEYFPSQML | ctacaagcaaatctccaacagcacatctggaggatcttcaaatgacaacgcctac |
| | RTGNNFQFTYTFEDVPFHSSYAHSQSL | ttcggctacagcaccccctgggggtattttgacttcaacagattccactgccact |
| | DRLMNPLIDQYLYYLSRTQSTGGTAGT | tctcaccacgtgactggcagcgactcatcaacaacaactggggattccggcccaa |
| | QQLLFSQAGPNNMSAWAKNWLPGPCYR | gagactcaacttcaagctcttcaacatccaggtcaaggaggtcacgcagaatgaa |
| | QQRVSKTSADNNNSEYSWTGATKYHLN | ggcaccaagaccatcgccaataacctcaccagcaccatccaggtgtttacggact |
| | GRDSLVNPGPAMASHKDDEEKFFPQSG | cggagtaccagctgccgtacgttctcggctctgcccaccagggctgcctgcctcc |
| | VLIFGKQGSEKTNVDIEKVMITDEEEI | gttcccggcggacgtgttcatgattcccaagtacggctacctaacactcaacaac |
| | RTTNPVATEQYGSVSTNLQRGNRQAAT | ggtagtcaggccgtgggacgctcctccttctactgcctggaatactttccttcgc |
| | ADVNTQGVLPGMVWQDRDVYLQGPIWA | agatgctgagaaccggcaacaacttccagtttacttacaccttcgaggacgtgcc |
| | KIPHTDGHFHPSPLMGGFGLKHPPPQI | tttccacagcagctacgcccacagccagagcttggaccggctgatgaaccccctc |
| | LIKNTPVPADPPTTFNQSKLNSFITQY | atcgaccagtacctgtactacctgtctcggactcagtccacgggaggtaccgcag |
| | STGQVSVEIEWELQKENSKRWNPEIQY | gaactcagcagttgctattttctcaggccgggcctaataacatgtcggctcaggc |
| | TSNYYKSTSVDFAVNTEGVYSEPRPIG | caaaaactggctacccgggccctgctaccggcagcagcgagtatcaaagacatct |
| | TRYLTRNL SEQ ID NO: 6 | gcggataacaacaacagtaaatactcgtggactggagctaccaagtaccacctca |
| | | atggcagagactctctggtgaatccgggcccggccatggcaagccacaaggacga |
| | | tgaagaaaagttttttcctcagagcggggttctcatctttgggaagcaaggctca |
| | | gagaaaacaaatgtggacattgaaaaggtcatgattacagacgaagaggaaatca |
| | | ggacaaccaatcccgtggctacggagcagtatggttctgtatctaccaacctcca |
| | | gagaggcaacagacaagcagctaccgcagatgtcaacacacaaggcgttcttcca |
| | | ggcatggtctggcaggacagagatgtgtaccttcaggggcccatctgggcaaaga |
| | | ttccacacacggacggacattttcacccctctcccctcatgggtggattcggact |
| | | taaacaccctcctccacagatcctgatcaagaacacgcctgtacctgcggatcct |
| | | ccgaccaccttcaaccagtcaaagctgaactctttcatcacgcaatacagcaccg |
| | | gacaggtcagcgtggaaattgaatgggagctgcagaaggaaaacagcaagcgctg |
| | | gaaccccgagatccagtacacctccaactactacaaatctacaagtgtggacttt |
| | | gctgttaatacagaaggcgtgtactctgaaccccgccccattggcacccgttacc |
| | | tcacccgcaacctg-3′ SEQ ID NO: 30 |
| HW02 | MAADGYLPDWLEDTLSEGIRQWWKLKP | 5'-atggctgccgatggttatcttccagattggctcgaggacactctctctgaag |
| (VP1) | GPPPPKPAERHKDDSRGLVLPGYKYLG | gaataagacagtggtggaagctcaaacctggcccaccaccaccaaagcccgcaga |
| | PFNGLDKGEPVNEADAAALEHDKAYDR | gcggcataaggacgacagcaggggtcttgtgcttcctgggtacaagtacctcgga |
| | QLDSGDNPYLKYNHADAEFQERLKEDT | cccttcaacggactcgacaagggagagccggtcaacgaggcagacgccgcggccc |
| | SFGGNLGRAVFQAKKRVLEPLGLVEEP | tcgagacacgacaaagcctacgaccagcagctcgacagcggagacacccgtacct |
| | VKTAPGKKRPVEHSPVEPDSSSGTGKA | caagtacaaccacgccgacgcggagtttcaggagcgccttaaagaagatacgtct |
| | GQQPARKRLNFGQTGDADSVPDPQPLG | tttgggggcaacctcggacgagcagtcttccaggcgaaaaagagggttcttgaac |
| | QPPAAPSGLGTNTMATGSGAPMADNNE | ctctgggcctggttgaggaacctgttaagacggctccgggaaaaaagaggccggt |
| | GADGVGNSSGNWHCDSTWMGDRVITTS | agagcactctcctgtggagccagactcctcctcgggaaccggaaaggcgggccag |
| | TRTWALPTYNNHLYKQISSQSGASNDN | cagcctgcaagaaaaagattgaattttggtcagactggagacgcagactcagtac |
| | HYFGYSTPWGYFDFNRFHCHFSPRDWQ | ctgacccccagcctctcggacagccaccagcagccccctctggtctgggaactaa |
| | RLINNNWGFRPKRLNFKLFNIQVKEVT | tacgatggctacaggcagtggcgcaccaatggcagacaataacgagggcgccgac |
| | QNDGTTTIANNLTSTVQVFTDSEYQLP | ggagtaggtaattcctcgggaaattgacattgcgattccacatggataggcgaca |
| | YVLGSAHQGCLPPFPADVFMVPQYGYL | gagtcatcaccaccagcacccgaacctgggccctgcccacctacaacaaccacct |
| | TLNNGSQAVGRSSFYCLEYFPSQMLRT | ctacaaacaaatttccagccaatcaggagcctcgaacgacaatcactactttggc |
| | GNNFTFSYTFEDVPFHSSYAHSQSLDR | tacagcaccccttgggggtattttgacttcaacagattccactgccacttttcac |
| | LMNPLIDQYLYYLSRTNTPSGTTTQSR | cacgtgactggcaaagactcatcaacaacaactggggattccgacccaagagact |
| | LQFSQAGASDIRDQSRNWLPGPCYRQQ | caacttcaagctctttaacattcaagtcaaagaggtcacgcagaatgacggtacg |
| | RVSKTSADNNNSEYSWTGATKYHLNGR | acgacgattgccaataaccttaccagcacggttcaggtgtttactgactcggagt |
| | DSLVNPGPAMASHKDDEEKFFPQSGVL | accagctcccgtacgtcctcggctcggcgcatcaaggatgcctcccgccgttccc |
| | IFGKQGSEKTNVDIEKVMITDEEEIRT | agcagacgtcttcatggtgccacagtatggatacctcaccctgaacaacgggagt |
| | TNPVATEQYGSVSTNLQRGNRQAATAD | caggcagtaggacgctcttcattttactgcctggagtactttccttctcagatgc |
| | VNTQGVLPGMVWQDRDVYLQGPIWAKI | tgcgtaccggaaacaactttaccttcagctacacttttgaggacgttcctttcca |
| | PHTDGHFHPSPLMGGFGLKHPPPQILI | cagcagctacgctcacagccagagtctggaccgtctcatgaatcctctcatcgac |
| | KNTPVPADPPTTFNQSKLNSFITQYST | cagtacctgtattacttgagcagaacaaacactccaagtggaaccaccacgcagt |
| | GQVSVEIEWELQKENSKRWNPEIQYTS | caaggcttcagttttctcaggccggagcgagtgacattcgggaccagtctaggaa |
| | NYYKSTSVDFAVNTEGVYSEPRPIGTR | ctggcttcctggaccctgttaccgccagcagcgagtatcaaagacatctgcggat |
| | YLTRNL SEQ ID NO: 7 | aacaacaacagtgaatactcgtggactggagctaccaagtaccacctcaatggca |
| | | gagactctctggtgaatccgggcccggccatggcaagccacaaggatgatgaaga |
| | | aaagttttttcctcagagcggggttctcatctttgggaagcaaggctcagagaaa |
| | | acaaatgtggacattgaaaaggtcatgattacagacgaagaggaaatcaggacaa |
| | | ccaatcccgtggctacggagcagtatggttctgtatctaccaacctccagagagg |
| | | caacagacaagcagctaccgcagatgtcaacacacaaggcgttcttccaggcatg |
| | | gtctggcaggacagagatgtgtaccttcaggggcccatctgggcaaagattccac |
| | | acacggacggacattttcacccctctcccctcatgggtggattcggacttaaaca |
| | | ccctcctccacagatcctgatcaagaacacgcctgtacctgcggatcctccgacc |
| | | accttcaaccagtcaaagctgaactctttcatcacgcaatacagcaccggacagg |
| | | tcagcatggaaattgaatgggagctgcagaaggaaaacagcaagcgctggaaccc |
| | | cgagatccagtacacctccaactactacaaatctacaagtgtggactttgctgtt |
| | | aatacagaaggcgtgtactctgaaccccgccccattggcacccgttacctcaccc |
| | | gcaacctg-3′ SEQ ID NO: 31 |
| HW03 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacttgaaacctggagccccgaaacccaaagccaacca |
| | PFNGLDKGEPVNAADAAALEHDKAYDQ | gcaaaagcaggacgacggccggggtctggtgcttcctggctacaagtacctcgga |
| | QLQAGDNPYLRYNHADAEFQERLQEDT | cccttcaacggactcgacaagggggagcccgtcaacgcggcggacgcagcggccc |
| | SFGGNLGRAVFQAKKRLLEPLGLVEEA | tcgagcacgacaaggcctacgaccagcagctgcaggcgggtgacaatccgtacct |
| | AKTAPGKKRPVEQSPQEPDSSAGIGKS | gcggtataaccacgccgacgccgagtttcaggagcgtctgcaagaagatacgtct |
| | GAQPAKKRLNFGQTGDSESVPDPQPLG | tttgggggcaacctcgggcgagcagtcttccaggccaaaaagaggcttcttgaac |
| | EPPATPAAVGPTTMAAGGGAPMADNNE | ctcttggtctggttgaggaagcggctaagacggctcctggaaagaagaggcctgt |
| | GADGVGNSSGNWHCDSTWLGDRVITTS | agagcagtctcctcaggaaccggactcctccgcgggtattggcaaatcgggtgca |
| | TRTWALPTYNNHLYKQISNSTSGGSSN | cagcccgctaaaaagagactcaattttggtcagactggcgactcagagtcagtcc |
| | DNAYFGYSTPWGYFDFNRFHCHRSPRD | ccgatccacaacctctcggagaacctccagcaacccccgctgctgtgggacctac |
| | WQRLINNNWGFRPKRLNFKLFNIQVKE | tacaatggctgcaggcggtggcgctccaatggcagacaataacgaagacgccgac |
| | VTQNEGTKTIANNLTSTIQVFTDSEYQ | ggagtgggtaattcctcgggaaattggcattgcgattccacatggctgggggaca |
| | LPYVLGSAHQGCLPPFPADVFMIPQYG | gagtcatcaccaccagcacccgaacctgggccctgcccacctacaacaaccacct |
| | YLTLNNGSQAVGRSSFYCLEYFPSQML | ctacaagcaaatctccaacagcacatctggaggatcttcaaatgacaacgcctac |
| | RTGNNFQFTYTFEDVPFHSSYAHSQSL | ttcggctacagcaccccctgggggtattttgacttcaacagattccactgccact |
| | DRLMNPLIDQYLYYLSRTQSTGGTAGT | tctcaccacgtgactggcagcgactcatcaacaacaactggggattccggcccaa |
| | QQLLFSQAGPNNMSAQAKNWLPGPCYR | gagactcaacttcaagctcttcaacatccaggtcaaggaggtcacgcagaatgaa |
| | GRDSLVNPGPAMASHKDDEEKFFPQSG | ggcaccaagaccatcgccaataacctcaccagcaccatccaggtgtttacggact |
| | VLIFGKQGSEKTNVDIEKVMITDEEEI | cggagtaccagctgccgtacgttctcggctctgcccaccagggctgcctgcctcc |
| | RTTNPVATEQYGSVSTNLQRGNRQAAT | gttcccggcggacgtgttcatgattccccagtacggctacctaacactcaacaac |
| | ADVNTQGVLPGMVWQDRDVYLQGPIWA | ggtagtcaggccgtgggacgctcctccttctactgcctggaatactttccttcgc |
| | KIPHTDGHFHPSPLMGGFGLKHPPPQI | agatgctgagaaccggcaacaacttccagtttacttacaccttcgaggacgtgcc |
| | LIKNTPVPADPPTTFNQSKLNSFITQY | tttccacagcagctacgcccacagccagagcttggaccggctgatgaaccccctc |
| | STGQVSVEIEWELQKENSKRWNPEIQY | atcgaccagtacctgtactacctgtctcggactcagtccacgggaggtaccgcag |
| | TSNYYKSTSVDFAVNTEGVYSEPRPIG | gaactcagcagttgctattttctcaggccgggcctaataacatgtcggctcaggc |
| | TRYLTRNL SEQ ID NO: 8 | caaaaactggctacccgggccctgctaccggcagcagcgagtatcaaagacatct |
| | | gcggataacaacaacagtgaatactcgtggactggagctaccaagtaccacctca |
| | | atggcagagactctctggtgaatccgggcccggccatggcaagccacaaggacga |
| | | tgaagaaaagttttttcctcagagcggggttctcatctttgggaagcaaggctca |
| | | gagaaaacaaatgtggacattgaaaaggtcatgattacagacgaagaggaaatca |
| | | ggacaaccaatcccgtggctacggagcagtatggttctgtatctaccaacctcca |
| | | gagaggcaacagacaagcagctaccgcagatgtcaacacacaaggcgttcttcca |
| | | ggcatggtctggcaggacagagatgtgtaccttcaggggcccatctgggcaaaga |
| | | ttccacacacggacggacattttcacccctctcccctcatgggtggattcggact |
| | | taaacaccctcctccacagatcctgatcaagaacacgcctgtacctgcggatcct |
| | | ccgaccaccttcaaccagtcaaagctgaactctttcatcacgcaatacagcaccg |
| | | gacaggtcagcgtggaaattgaatgggagctgcagaaggaaaacagcaagcgctg |
| | | gaaccccgagatccagtacacctccaactactacaaatctacaagtgtggacttt |
| | | gctgttaatacagaaggcgtgtactctgaaccccgccccattggcacccgttacc |
| | | tcacccgcaacctg-3′ SEQ ID NO: 32 |
| HW04 | MAADGYLPDWLEDTLSEGIRQWWKLKP | 5′-atggctgccgatggttatcttccagattggctcgaggacactctctctgaag |
| (VP1) | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctggctacaagtacctcgga |
| | QLKAGDNPYLKYNHADAEFQERLKEDT | cccttcaacggactcgacaagggggagcccgtcaacgcggcggacgcagcggccc |
| | SFGGNLGRAVFQAKKRVLEPLGLVEEA | tcgagcacgacaaggcctacgaccagcagctcaaagcgggtgacaacccgtacct |
| | AKTAPGKKRPVEPSPQRSPDSSSGIGK | caagtacaaccacgccgacgcggagtttcaggagcgccttaaagaagatacgtct |
| | KGQQPARKRLNFGQTGDSESVPDPQPL | tttgggggcaacctcggacgagcagtcttccaggccaagaagcgggttctcgaac |
| | GEPPAAPSGVGPNTMASGGGAPMADNN | ctcttaatctggttgaggaaacggctaagacggctcctggaaagaagagaccggt |
| | EGADGVGNASGNWHCDSTWLGDRVITT | agagccatcaccccagcgttctccagactcctcctcgggcatcggcaagaaaggc |
| | STRTWALPTYNNHLYKQISNSTSGGSS | caacagcccgccagaaaaagactcaattttggccagactggcgactcagagtcag |
| | NDNAYFGYSTPWGYFDFNRFHCHFSPR | ttccagaccctcaacctctcggagaacctccagcagcgccctctggtgtgggacc |
| | DWQRLINNNWGFRPKRLNFKLFNIQVK | taatacaatggcttcaggcggtggcgcaccaatggcagacaataacgaaggcgcc |
| | EVTTNDGVTTIANNLTSTVQVFSDSEY | gacggagtgggtaatgcctcaggaaattggcattgcgattccacatggctgggcg |
| | QLPYVLGSAHQGCLPPFPADVFMIPQY | atagagtcatcaccaccagcacccgaacctgggccctccccacctacaacaatca |
| | GYLTLNNSGQSVGRSSFYCLEYFPSQM | cctctacaagcaaatctccaacagcacatctggaggatcttcaaatgacaacgcc |
| | LRTGNNFTFSYTFEDVPFHSSYAHSQS | tacttcggctacagcaccccctggaggtattttgacttcaacagattccactacc |
| | LDRLMNPLIDQYLYYLSRTQSTGGTQG | atttctcaccacgtgactggcagcgactcatcaacaacaattggggattccggcc |
| | TQQLLFSQAGPANMSAQAKNWLPGPCY | caagagactcaacttcaagctcttcaacatccaagtcaaggaggtcacgacgaat |
| | RQQRVSKTSADNNNSEYSWTGATKYHL | gatggcgtcacgaccatcgctaataaccttaccagcacggttcaagtcttctcgg |
| | NGRDSLVNPGPAMASHKDDEEKFFPQS | actcggagtaccagttgccgtacgtcctcggctctgcgcaccagggctgcctgcc |
| | GVLIFGKQGSEKTNVDIEKVMITDEEE | tccgttcccggcggacgtgttcatgattcctcagtacggctacctgactctcaac |
| | IRTTNPVATEQYGSVSTNLQRGNRQAA | aatggcagtcagtctgtgggccgttcctccttctactgcctggaatatttcccat |
| | TADVNTQGVLPGMVWQDRDVYLQGPIW | cgcagatgctgagaacgggcaataactttaccttcagctacaccttcgaggacgt |
| | AKIPHTDGHFHPSPLMGGFGLKHPPPQ | gcctttccacagcagctacgcccacagccagagcttggaccggctgatgaatcct |
| | ILIKNTPVPANPSTTFNQSKLNSFITQ | ctcatcgaccagtacctgtactacttatccagaactcagtccacaggaggaactc |
| | YSTGQGSVEIEWELQKENSKRWNPEIQ | aaggtacccagcaattgttattttctcaagctgggcctgcaaacatgtcggctca |
| | YTSNYYKSTNVDFAVNTEGVYSEPRPI | ggctaagaactggctacctggaccttgctaccggcagcagcgagtatcaaagaca |
| | GTRYLTRNL SEQ ID NO: 9 | tctgcggataacaacaacagtgaatactcgtggactggagctaccaagtaccacc |
| | | tcaatggcagagactctctggtgaatccgggcccggccatggcaagccacaagga |
| | | cgatgaagaaaagttttttcctcagagcggggttctcatctttgggaagcaaggc |
| | | tcagagaaaacaaatgtggacattgaaaaggtcatgattacagacgaagaggaaa |
| | | tcaggacaaccaatcccgtggctacggagcagtatggttctgtatctaccaacct |
| | | ccagagaggcaacagacaagcagctaccgcagatgtcaacacacaaggcgttctt |
| | | ccaggcatggtctggcaggacagagatgtgtaccttcaggggcccatctgggcaa |
| | | agattccacacacggacggacattttcacccctctcccctcatgggtggattcgg |
| | | acttaaacaccctcctccacagattctcatcaagaacaccccggtacctgcgaat |
| | | ccttcgaccaccttcaaccagtcaaagctgaactctttcatcacgcaatacagca |
| | | ccggacaagtcagcgtggaaatcgagtgggagctgcagaaggaaaacagcaagcg |
| | | ctggaatccagagattcaatacacttccaactactacaaatctacaaatgtggac |
| | | tttgctgtcaacacggagggggtttatagtgagcctcgccccattggcacccgtt |
| | | acctcacccgtaatctg-3′ SEQ ID NO: 33 |
| HW05 | MAADGYLPDWLEDTLSEGIRQWWKLKP | 5′-atggctgccaatggttatcttccagattggctcgaggacactctctctgaaa |
| (VP1) | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDR | gcggcataaggacgacagcaggggtcttgtgcttcctgggtacaagtacctcgga |
| | QLDSGDNPYLKYNHADAEFQERLKEDT | cccttcaacggactcgacaagggggagcccgtcaacgcggcggatgcagcggccc |
| | SFGGNLGRAVFQAKKRVLEPLGLVEEA | tcgagcacgacaaggcctacgaccggcagctcgacagcggagacaacccgtacct |
| | AKTAPGKKRPVEPSPQRSPDSSTGIGK | caagtacaaccacgccgacgcggagtttcaggagcgccttaaagaagatacgtct |
| | KGQQPAKKRLNFGQTGDSESVPDPQPI | tttgggggcaacctcggacgagcagtcttccaggccaagaagcgggttctcgaac |
| | GEPPAAPSGVGPNTMAAGGGAPMADNN | ctcttggtctggttgaggaagcggctaagacggctcctggaaagaagagaccggt |
| | EGADGVGSSSGNWHCDSTWLGDRVITT | agagccgtcacctcagcgttcccccgactcctccacgggcatcggcaagaaaggc |
| | STRTWALPTYNNHLYKQISSASTGASN | cagcagcccgctaaaaagagactgaactttggtcagactggcgactcagagtcag |
| | DNTYFGYSTPWGYFDFNRFHCHFSPRD | tgcccgaccctcaaccaatcggagaacctccagcagcgccctctggtgtgggacc |
| | WQRLINNNWGFRPKKLRFKLFNIQVKE | taatacaatggctgcaggcggtggcgcaccaatggcagacaataacgaaggcgcc |
| | VTQNEGTKTIANNLTSTVQVFTDSEYQ | gacggagtgggtagttcctcgggaaattggcattgcgattccacatggctgggcg |
| | LPYVLGSAHQGCLPPFPADVFMVPQYG | acagagtcatcaccaccagcacccgaacatgggccttgcccacctacaacaacca |
| | YLTLNNGSQALGRSSFYCLEYFPSQML | cctctacaagcaaatctccagtgcttcaacgggggccagcaacgacaacacctac |
| | RTGNNFQFTYTFEDVPFHSSYAHSQSL | ttcggctacagcaccccctgggggtattttgacttcaacagattccactgtcact |
| | DRLMNPLIDQYLYYLSRTQSTGGTAGT | tttcaccacgtgactggcaacgactcatcaacaacaactggggattccggcccaa |
| | QQLLFSQAGPNNMSAQAKNWLPGPCYR | gaagctgcggttcaagctcttcaacatccaggtcaaggaggtcacgcagaatgaa |
| | QQRVSKTSADNNNSEYSWTGATKYHLN | ggcaccaagaccatcgccaataatctcaccagcaccgtgcaggtctttacggact |
| | GRDSLVNPGPAMASHKDDEEKFFPQSG | cggagtaccagttaccgtacgtgctaggatccgctcaccagggatgtctgcctcc |
| | VLIFGKQGSEKTNVDIEKVMITDEEEI | gttcccggcggacgtcttcatggttcctcagtacggctatttaactttaaacaat |
| | RTTNPVATEQYGSVSTNLQRGNRQAAT | ggaagccaagccctgggacgttcctccttctactgtctggagtatttcccatcgc |
| | ADVNTQGVLPGMVWQDRDVYLQGPIWA | agatgctgagaaccggcaacaactttcagtttacttacaccttcgaggacgtgcc |
| | KIPHTDGNFHPSPLMGGFGMKHPPPQI | tttccacagcagctacgcgcacagccagagcctggacaggctgatgaatcccctc |
| | LIKNTPVPADPPTTFSQAKLASFITQY | atcgaccagtacctgtactacctgtctcggactcagtccacgggaggtaccgcag |
| | STGQVSVEIEWELQKENSKRWNPEIQY | gaactcagcagttactattttctcagaccgggcctaataacatgtcgactcaggc |
| | TSNYYKSTNVDFAVNTEGTYSEPRPIG | caaaaactggctacccgggccctgctaccggcagcagcgagtatcaaagacatct |
| | TRYLTRNL SEQ ID NO: 10 | gcggataacaacaacagtgaatactcgtggactggagctaccaagtaccacctca |
| | | atggcagagactctctggtgaatccgggcccggccatggcaagccacaaggacga |
| | | tgaagaaaagttttttcctcagagcggggttctcatctttgggaagcaaggctca |
| | | gagaaaacaaatgtggacattgaaaaggtcatgattacagacgaagaggaaatca |
| | | ggacaaccaatcccgtggctacggagcagtatggttctgtatctaccaacctcca |
| | | gagaggcaacagacaagcagctaccgcagatgtcaacacacaaggcgttcttcca |
| | | ggcatggtctggcaggacagagatatgtaccttcaggggcccatctgggcaaaga |
| | | ttccacacacggacggcaactttcacccttctccgctgatgggagggtttggaat |
| | | gaagcacccacctcctcagatcctgatcaagaacacgccggtacctgcggatcct |
| | | ccaacaacgttcagccaggcgaaattggcttccttcattacgcagtacagcaccg |
| | | gacaggtcagcgtggaaatcgagtgggagctgcagaaggagaacagcaaacgctg |
| | | gaacccagagattcagtacacttcaaactactacaaatctacaaatgtggacttt |
| | | gctgtcaatacagagggaacttattctgagcctcgccccattggtactcgttacc |
| | | tcacccgtaatctg-3′ SEQ ID NO: 34 |
| HW06 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacctgaaacctggagccccgaagcccaaagccaacca |
| | PFNGLDKGEPVNAADAAALEHDKAYDQ | gcaaaagcaggacgacggccggggcctggtgcttccttgctacaagtacctcgga |
| | QLQAGDNPYLKYNHADAEFQERLQEDT | cccttcaacggactcgacaagggggagcccgtcaacgcggcggacgcagcggccc |
| | SFGGNLGRAVFQAKKRVLEPLGLVEEG | tcgagcacgacaaggcctacgaccagcagctgcaggcgggtgacaatccgtacct |
| | AKTAPGKKRPVEQSPQEPDSSSGIGKK | caagtacaaccacgccgacgcggagtttcaggagcggctgcaagaagatacgtct |
| | GQQPAKKRLNFGQTGSDESVPDPQPIG | tttgggggcaacctcgggcgagcagtcttccaggccaagaagcgggttctcgaac |
| | EPPAAPSGVGPNTMAAGGGAPMADNNE | ctctcggtctggttgaggaaggcgctaagacggctcctggaaagaagagaccagt |
| | GADGVGSSSGNWHCDSTWLGDRVITTS | agagcagtcgccacaagagccagactcctcctcgggcatcggcaagaaaggccag |
| | TRTWALPTYNNHLYKQISSASTGASND | cagcccgcgaaaaagagactcaactttgggcagactggcgactcagagtcagtgc |
| | NHYFGYSTPWGYFDFNRFHCHFSPRDW | ccgaccctcaaccaatcggagaacctccagcagcgccctctggtgtgggacctaa |
| | QRLINNNWGFRPKKLRFKLFNIQVKEV | tacaatggctgcaggcggtggcgcaccaatggcagacaataacgaaggcgccgac |
| | TDNNGVKTIANNLTSTIQVFTDSEYQL | ggagtgggtagttcctcgggaaattggcattgcgattccacatggctgggcgaca |
| | PYVLGSAHQGCLPPFPADVFMIPQYGY | gagtcatcaccaccagcacccgaacatgggccttgcccacctataacaaccacct |
| | LTLNNGSQAVGRSSFYCLEYFPSQMLR | ctacaagcaaatctccagtgcttcaacgggggccagcaacgacaaccactacttc |
| | TGNNFQFSYEFENVPFHSSYAHSQSLD | ggctacagcaccccctggggatattttaacttcaacagattccactgccactttt |
| | RLMNPLIDQYLYYLSRTQTTGGTANTQ | caccacgtgactggcagcgactcatcaacaacaactggggattccggcccaagaa |
| | TLGFSQGGPNTMANQAKNWLPGPCYRQ | gctgcggttcaagctcttcaacattcaggtcaaagaggttacggacaacaatgga |
| | QRVSTTTGQNNNSNFAWTAGTKYHLNG | gtcaagaccatcgccaataacctcaccagcaccatccaggtatttacggactcga |
| | RNSLANPGIAMATHKDDEERFFPSNGI | agtaccagctgccgtacgttctcggctctgcccaccagggctgcctgcctccgtt |
| | LIFGKQNAARDNADYSDVMLTSEEEIK | cccggcggacgtcttcatgattccgcagtacggctaccttacactgaacaatgga |
| | TTNPVATEEYGIVADNLQQQNTAPQIG | agtcaagccgtaggccgttcctccttctactgcctggaatactttccttcgcaga |
| | TVNSQGALPGMVWQNRDVYLQGPIWAK | tgctgagaaccggcaacaacttccagttcagctacgagtttgagaacgtaccttt |
| | IPHTDGNFHPSPLMGGFGLKHPPPQIL | ccatagcagctacgctcacagccaaagcctggaccgactaatgaatccactgatt |
| | IKNTPVPANPPEVFTPAKFASFITQYS | gaccagtacctgtactacttgtctcggactcaaacaacaggaggcacggcaaata |
| | TGQVSVEIEWELQKENSKRWNPEIQYT | cgcagactctgggcttcagccaaggtgggcctaatacaatggccaatcaggcaaa |
| | SNFEKQTGVDFAVDSQGVYSEPRPIGT | gaactggctgccaggaccctgttaccgccaacaacgcgtctcaacgacaaccggg |
| | RYLTRNL SEQ ID NO: 11 | caaaacaacaatagcaactttgcctggactgctgggaccaaataccatctgaatg |
| | | gaagaaattcattggctaatcctggcatcgctatggcaacacacaaagacgacga |
| | | ggagcgtttttttcccagtaacgggatcctgatttttggcaaacaaaatgctacc |
| | | agagacaatgcggattacagcgatgtcatgctcaccagcgaggaagaaatcaaaa |
| | | ccactaaccctgtggctacagaggaatacggtatcgtggcagataacttgcagca |
| | | gcaaaacacggctcctcaaattggaactgtcaacagccagggggccttacccggt |
| | | atggtctggcagaaccgggacgtgtacctgcagggtcccatctgggccaagattc |
| | | ctcacacggacggcaactttcacccttctccgctgatgggcggctttggcctgaa |
| | | acacccgcctccctcagatcctgattaagaatacactgttcccgctaatcctccg |
| | | gaggtgtttactcctgccaagtttgcttcgttcatcacacagtacggcaccggac |
| | | aagtcaacgtggaaatcgaatgggagctgcagaaggaaaacagcaagcgctggaa |
| | | cccggagattcagtacacctccaactttgaaaagcagactggtgtggactttgcc |
| | | gttgacagccagggtgtttactctgagcctcgccctattggcactcgttacctca |
| | | cccgtaatctg-3′ SEQ ID NO: 35 |
| HW07 | MAADGYLPDWLEDNLSEGIRQWWDLKP | 5′-atggctgccgatggttatcttccagattggctcgaggacaacctctctgaag |
| (VP1) | GAPKPKANQQKQDDGRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctgggtacaagtacctcgga |
| | QLKAGDNPYLRYNHADAEFQERLQEDT | cccttcaacggactcgacaagggggagcccgtcaacgcggcggacgcagcggccc |
| | SFGGNLGRAVFQAKKRVLEPLGLVEEA | tcgagcacgacaaggcctacgaccagcagctcgaagcgggtgacaatccgtacct |
| | AKTAPGKKRPVEPSPQRSPDSSTGIGK | gcggtataaccacgccgacgccgagtttcaggagcgtctgcaagaagatacgtct |
| | KGQQPAKKRLNFGQTGDSESVPDPQPI | tttggaggcaacctcgggcgagcagtcttccagaccaaaaagaggcttcttgaac |
| | GEPPAGPSGLGSGTMAAGGGAPMADNN | ctctcggtctggttgaggaagctgctaagacggctcctggaaagaagagaccggt |
| | EGADGVGSSSGNWHCDSTWLGDRVITT | agaaccgtcacctcagcgttcccccgactcctccacgggcatcggcaagaaaggc |
| | STRTWALPTYNNHLYKQISSQSGASND | cagcccgctaaaaagagactcaatttcggtcagactggcgacacagagtcagtcc |
| | NHYFGYSTPWGYFDFNRFHCHFSPRDW | cagaccctcaaccaatcggagaacctcccgcagccccctctagtgtgggatctgg |
| | QRLINNNWGFRPKRLNFKLFNIQVKEV | tacagtggctgcaggcggtggcgcaccaatggcagacaataacgaaggtgccgac |
| | TDNNGVKTIANNLTSTIQVFTDSEYQL | ggagtgggtaatgcctcaggaaattggcattgcgattccacatggctgggcgaca |
| | PYVLGSAHQGCLPPFPADVFMIPQYGY | gagtcattaccaccagcacccgaacctgggccctgcccacctacaacaaccacct |
| | LTLNNGSQSVGRSSFYCLEYFPSQMLR | ctacaagcaaatctccagtgaaactgcaggtagtaccaacgacaacacctacttc |
| | TGNNFEFSYTFEDVPFHSSYAHSQSLD | ggctacagcaccccctgggggtattttgacttcaacagattccactgtcactttt |
| | RLMNPLIDQYLYYLSRTQSTGGTAGTQ | caccacgtgactggaaacgactcatcaaaaacaattggggattccggccaaaaag |
| | QLLFSQAGPNNMSAQAKNWLPGPCYRQ | actcaacttcaagctgttcaacatccaggtcaaggaagtcacgacgaacgaaggc |
| | QRVSKTSADNNNSEYSWTGATKYHLNG | accaagaccatcgccaataatctcaccagcaccgtgcaggtctttacggactcgg |
| | RDSLVNPGPAMASHKDDEEKFFPQSGV | aataccagctcccgtacgtcctcggctctgcgcaccagggctgcctgcctccgtt |
| | LIFGKQGSEKTNVDIEKVMITDEEEIR | cccggcggacgtcttcatgattcctcagtacgggtacctgactctgaacaatgga |
| | TTNPVATEQYGSVSTNLQRGNRQAATA | agtcaagccgtaggccgttcctccttctactgcctggaatatttcccatcgcaga |
| | DVNTQGVLPGMVWQDRDVYLQGPIWAK | tgctgagaacgggcaacaactttgagttcagctacagcttcgaggacgttccttt |
| | IPHTDGHFHPSPLMGGFGLKHPPPQIL | ccacagcagctacgctcacagccagagtctggaccgtctcatgaatcctctcatc |
| | TKNTPVPANPSTTFSAAKFASFITQYS | gaccagtacctgtattacttgagcagaacaaacactccaagtggaaccaccacgc |
| | TGQVSVIEEWELQKENSKRWNPEIQYT | agccaaggcctcagttctcccaggcccgagcgactgacatccgggaccagtctac |
| | SNYNKSVNVDFTVDTNGVYSEPHPIGT | gaactggcttcctggaccctgttaccgccagcagcgagtatcaaagacatctgcg |
| | RYLTRNL SEQ ID NO: 12 | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttgggaagcaaggctcagag |
| | | aaaacaaatgtggacattgaaaaggtcatgattacagacgaagaggaaatcagga |
| | | caaccaatcccgtggctacggagcagtatggttctgtatctaccaacctccagaa |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggacattttcacccctctcctctcatgggcggctttggacttaa |
| | | gcacccgcctcctcagatcctaatcaaaaacacgcctgttcccgcggatcctcca |
| | | actaccttcagtcaagctaagctggcgtcgttcatcacgcagtacagcaccggac |
| | | aggtcagcgtggaaatcgagtgggagctgcagaaggagaacagcaaacgctggaa |
| | | cccagagattcaatacacttccaaactactacaaatctacaaatgtggactttgt |
| | | gttaatacagaaggcgtatactctaaaccccaccccattggcacccgttacctca |
| | | cccgtaatctg-3′ SEQ ID NO: 36 |
| HW08 | MAADGYLPDWLEDNLSEGIRQWWDLKP | 5′-atggctgccgatggttatcttccagattggctcgaggacaacctctctgaag |
| (VP1) | GAPKPKANQQKQDDGRGLVLPGYKYLG | gaataagacagtggtgggacttgaaacctggagccccgaaacccaaagccaacca |
| | PFNGLDKGEPVNAADAAALEHDKAYDQ | gcaaaagcaggacgacggccggggtctggtgcttcctggctacaagtacctcgga |
| | QLKAGDNPYLRYNHADAEEQERLQEDT | cccttcaacggactcgacaagggggagcccgtcaacgcggcggacgcagcggccc |
| | SFGGNLGRAVFQAKKRVLEPLGLVEEA | tcgagcacgacaaggcctacgaccagcagctcaaagcgggtgacaatccgtacct |
| | AKTAPGKKRPVEPSRQRSPDSSTGIGK | gcggtataaccacgccgacgccgagtttcaggagcgtctgcaagaagatacgtct |
| | KGQQPAKKRLNFGQTGDSESVPDPQPI | tttgggggcaacctcgggcgagcagtcttccaggccaagaagcgggttctcgaac |
| | GEPPAGPSGLGSGTMAAGGGAPMADNN | ctctcggtctggttgaggaagctgctaagacggctcctggaaagaagagaccggt |
| | EGADGVGSSSGNWHCDSTWLGDRVITT | agaaccgtcacctcagcgttcccccgactcctccacgggcatcggcaagaaaggc |
| | STRTWALPTYNNHLYKQISSQSGASND | cagcagcccgcgaaaaagagactcaactttgggcagactggcgactcagagtcag |
| | NHYFGYSTPWGYFDFNRFHCHFSPRDW | tgcccgaccctcaaccaatcggagaaccccccgcaggcccctctggtctgggatc |
| | QRLINNNWGFRPKRLNFKLFNIQVKEV | tggtacaatggctgcaggcggtggcgctccaatggcagacaataacgaaggcgcc |
| | TDNNGVKTIANNLTSTIQVFTDSEYQL | gacggagtgggtagttcctcaggaaattggcattgcgattccacatggctgggcg |
| | PVYLGSAHQGCLPPFPADVFMIPQYGY | acagagtcatcaccaccagcacccgaacctgggccctccccacctacaacaacca |
| | LTLNNGSQSVGRSSFYCLEYFPSQMLR | cctctacaaacaaatttccagccaatcaggagcctcgaacgacaatcactacttt |
| | TGNNFEFSYTFEDVPFHSSYAHSQSLD | ggctacagcaccccttgggggtattttgacttcaacagattccactgccactttt |
| | OLMNPLIDQYLYYLSRTQSTGGTAGTQ | caccacgtgactggcagcgactcatcaacaacaactggggattccggcctaagcg |
| | QLLFSQAGPNNMSAQAKNWLPGPCYRQ | actcaacttcaagctcttcaacattcaggtcaaagaggttacggacaacaatgga |
| | QRVSKTSADNNNSEYSWTGATKYHLNG | gtcaagaccatcgccaataacctcaccagcaccatccaggtgtttacggactcgg |
| | RDSLVNPGPAMASHKDDEEKFFPQSGV | agtaccagctgccgtacgtcctcggctctgcgcaccagggctgcctgcctccgtt |
| | LIFGKQGSEKTNVDIEKVMITDEEEIR | cccggcggacgtcttcatgattcctcagtacggctacctgactctcaacaatggc |
| | TTNPVATEQYGSVNSTNLQRGNRQAAT | agtcagtctgtgggacgttcctccttctactgcctggaatattttccatctcaaa |
| | DVNTQGVLPGMVWQDRDVYLQGPIWAK | tgctgcgaactggaaacaattttgaattcagctacaccttcgaggacgtgccttt |
| | IPHTDGHFHPSPLMGGFGLKHPPPQIL | ccacagcagctacgcacacagccagagcttggaccgactgatgaatcctctcatc |
| | IKNTPVPANPSTTFSAAKFASFITQYS | gaccagtacctgtactacttatccagaactcagtccacgggaggtaccgcaggaa |
| | TGQVSVEIEWELQKENSKRWNPSIQYT | ctcagcagttgctattttctcaggccgggcctaataacatgtcggctcaggccaa |
| | SNYNKSVNVDFTVDTNGVYSEPHPIGT | aaactggctacccgggccctgctaccgccagcagcgagtatcaaagacatctgcg |
| | RYLTRNL SEQ ID NO: 13 | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttgggaagcaaggctcagag |
| | | aaaacaaatgtggacattgaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggacattttcacccctctcccctcatgggtggattcggacttaa |
| | | acaccctcctccacagattctcatcaagaacaccccggtacctgcgaatccttcg |
| | | accaccttcagtgcggcaaagtttgcttccttcatcacacagtactccacgggac |
| | | aggtcagcgtggagatcgagtgggagctgcagaaggaaaacagcaaacgctggaa |
| | | ccccgagatccagtacacttccaactacaacaagtctgttaatgtggactttact |
| | | gtggacactaatggcgtgtattcagagcctcaccccattggcaccagatacctca |
| | | cccgtaatctg-3′ SEQ ID NO: 37 |
| HW09 | MAADGYLPDWLEDNLSEGIRQWWDLKP | 5'-atggctgccgatggttatcttccagattggctcgaggacaacctctctgagg |
| (VP1) | GAPKPKANQQKQDDGRGLVLPGYKYLG | gcattcgcgagtggtgggacttgaaacctggagccccgaagcccaaagccaacca |
| | PFNGLDKGEPVNAADAAALEHDKAYDQ | gcaaaagcaggacgacggccggggtctggtgcttcctggctacaagtacctcgga |
| | QLKAGDNPYLRYNHADAEEQERLQEDT | cccttcaacggactcgacaagggggagcccgtcaacgcggcggatgcagcggccc |
| | SFGGNLGRAVFQAKKRVLEPLGLVEEA | tcgagcacgacaaggcctacgaccagcagctcaaagcgggtgacaatccgtacct |
| | AKTAPGKKRPVEPSRQRSPDSSTGIGK | gcggtataaccacgccgacgccgagtttcaggagcgtctgcaagaagatacgtct |
| | KGQQPAKKRLNFGQTGDSESVPDPQPI | tttgggggcaacctcgggcgagcagtcttccaggccaagaagcgggttctcgaac |
| | GEPPAGPSGLGSGTMAAGGGAPMADNN | ctctcggtctggttgaggaagctgctaagacggctcctggaaagaagagaccggt |
| | EGADGVGSSSGNWHCDSTWLGDRVITT | agagccatcaccccagcgttctccagactcctctacgggcatcggcaagaaaggc |
| | STRTWALPTYNNHLYKQISSQSGASND | caacagcctgcaagaaaaagattgaattttggtcagactggagacgcagactcag |
| | NHYFGYSTPWGYFDFNRFHCHFSPRDW | tacctgacccccagcctctcggacagccaccagcagccccctctggtctgggaac |
| | QRLINNNWGFRPKRLNFKLFNIQVKEV | taatacgatggctacaggcagtggcgcaccaatggcagacaataacgagggcgcc |
| | TDNNGVKTIANNLTSTIQVFTDSEYQL | gacggagtgggtaattcctcgggaaattggcattgcgattccacatggctgggcg |
| | PVYLGSAHQGCLPPFPADVFMIPQYGY | acagagtcattaccaccagcacccgaacctgggccctgcccacctacaacaacca |
| | LTLNNGSQSVGRSSFYCLEYFPSQMLR | cctctacaaacaaatttccagccaatcnnagcctcgaacgacaatcactactttg |
| | TGNNFEFSYTFEDVPFHSSYAHSQSLD | gctacagcaccccctggggggtattttgactttaacagattccactgccactttt |
| | OLMNPLIDQYLYYLSRTQSTGGTAGTQ | caccacgtgactggcagcgactcatcaacaacaactggggattccggccaaaaag |
| | QLLFSQAGPNNMSAQAKNWLPGPCYRQ | actcagcttcaagctcttcaacatccaggtcaaggaggtcacgcagaatgaaggc |
| | QRVSKTSADNNNSEYSWTGATKYHLNG | accaagaccatcgccaataaccttaccagcacgattcaggtatttacggactcgg |
| | RDSLVNPGPAMASHKDDEEKFFPQSGV | aataccagctgccgtacgtcctcggctctgcgcaccagggctgcctgcctccgtt |
| | LIFGKQGSEKTNVDIEKVMITDEEEIR | cccggcggacgtcttcatggttcctcagtacggctatttaactttaaacaatgga |
| | TTNPVATEQYGSVNSTNLQRGNRQAAT | agccaagccctgggacgttcctccttctactgtctggagtatttcccatcgcaga |
| | DVNTQGVLPGMVWQDRDVYLQGPIWAK | tgctgagaaccggcaacaactttcagttcagctacaccttcgaggacgtgccttt |
| | IPHTDGHFHPSPLMGGFGLKHPPPQIL | ccacagcagctacgctcacagccagagtctggaccggctgatgaaccccctcatc |
| | IKNTPVPANPSTTFSAAKFASFITQYS | gaccagtacctgtactacctgtctcggactcagtccacgggaggtaccgcaggaa |
| | TGQVSVEIEWELQKENSKRWNPSIQYT | ctcagcagttgctattttctcaggccggagcgagtgacattcgggaccagtctag |
| | SNYNKSVNVDFTVDTNGVYSEPHPIGT | gaactggcttcctggaccctgttaccgccagcagcgagtatcaaagacatctacg |
| | RYLTRNL SEQ ID NO: 14 | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttggggagcaaggctcagag |
| | | aaaacaaatgtggacattgaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggcaacttccacccgtctccactgatgggcggctttggcctgaa |
| | | acatcctccgcctcagatcctgatcaagaacacgcctgtacctgcggatcctccg |
| | | accaccttcaaccagtcaaagctgaactccttcattacgcagtacagcaccggac |
| | | aggtcagcgtggaaatcgagtgggagctgcagaaggagaacagcaaacgctggaa |
| | | cccagagattcagtacacttcaaactactacaaatctacaaatgtggactttgct |
| | | gtcaacacggagggggtttatagcgagcctcgccccattggcacccgttacctca |
| | | cccgcaacctg-3′ SEQ ID NO: 38 |
| HW10 | MAADGYLPDWLEDTLSEGIRQWWKLKP | 5′-atggctgccaatggttatcttccagattggctcgaggacactctctctgaaa |
| | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctggctacaagtacctcgga |
| | QLKAGDNPYLKYNHADAEFQERLKEDT | cccttcaacggactcgacaagggggagcccgtcaacgcggcggacgcagcggccc |
| | SFGGNLGRAVFQAKKRVLEPLGLVEEF | tcgagcacgacaaggcctacgaccagcagctcaaagcgggagacaacccgtacct |
| | VKTAPGKKRPVEHSPVEPDSSSGTGKA | caagtacaaccacgccgacgcggagtttcaggagcgccttaaagaagatacgtct |
| | GQQPARKRLNFGQTGDAESVPDPQPLG | tttgggggcaacctcggacgagcagtcttccaggcgaaaaagagggttcttgaac |
| | EPPATPAAVGPTTMASGGGAPMADNNE | ctctgggcctggttgaggaacctgttaagacggctccgggaaaaaagaggccggt |
| | GADGVGNASGNWHCDSTWMGDRVITTS | agagcactctcctgtggaaccagactcctcctcgggaaccggaaaggcgggccag |
| | TRTWALPTYNNHLYKQISSQSGASNDN | cagcctgcaagaaaaagattgaattttggtcagactggagacgcagagtcagtcc |
| | HYFGYSTPWGYFDFNRFHCHFSPRDWQ | ccgacccacaacctctcggagaacctccagcaacccccgctgctgtgggacctac |
| | RLINNNWGFRPKRLSFKLENIQVKEVT | tacaatggcttcaggcggtggcgcaccaatggcagacaataacgaaggcgccgac |
| | QNEGTKTIANNLTSTIQVFTDSEYQLP | ggagtgggtaatgcctcaggaaattggcattgcgattccacatggatgggcgaca |
| | YVLGSAHQGCLPPFPADVFMIPQYGYL | gagtcatcaccaccagcacccgaacctgggccctacccacctacaacaaccacct |
| | TLNNGSQAVGRSSFYCLEYFPSQMLRT | ctacaaacaaatttccagccaatcaggagcctcgaacgacaatcactactttggc |
| | GNNFEFSYTFEDVPFHSSYAHSQSLDR | tacagcaccccctgggggtattttgactttaacagattccactgccacttttcac |
| | LMNPLIDQYLYYLSRTNTPSGTTTQSR | cacgtaactggcaacgactcatcaacaacaactagggattccggcccaagagact |
| | LQFSQAGASDIRDQSRNWLPGPCYRQQ | cagcttcaagctcttcaacatccaggtcaaggaggtcacgcagaatgaaggcacc |
| | RVSKTSADNNNSEYSWTGATKYHLNGR | aagaccatcgccaataacctcaccagcaccatccaggtgtttacggactcggagt |
| | DSLVNPGPAMASHKDDEEKFFPQSGVL | accagctcccgtacgtcctcggctctgcgcaccagggctgcctgcctccgttccc |
| | IFGKQGSEKTNVKIEKVMITDEEEIRT | ggcggacgtgttcatgattcctcagtacgggtacctgactctgaacaatggcagt |
| | TNPVATEQYGSVSTNLQRGNRQAATAD | caggccgtgggccgttcctccttctactgcctggaatattttccatctcaaatgc |
| | VNTQGVLPGMVWQDRDVYLQGPIWAKI | tgcgaactggaaacaattttgaattcagctacaccttcgaggacgtgcctttcca |
| | PHTDGHFHPSPLMGGFGLKHPPPQILI | cagcagctacgcacacagccagagcttggaccgactgatgaatcctctcatcgac |
| | KNTPVPADPPLTFNQAKLNSFITQYST | cagtacctgtattacttgagcagaacaaacactccaagtggaaccaccacgcagt |
| | GQVSVEIEWELQKENSKRWNPEIQYTS | caaggcttcagttttctcaggccggagcgagtgacattcgggaccagtctaggaa |
| | NYYKSTSVDFAVNTEGVYSEPRPIGTR | ctggcttcctggaccctgttaccgccagcagcgagtatcaaagacatctgcggat |
| | YLTRNL SEQ ID NO: 15 | aacaacaacagtgaatactcgtggactggagctaccaagtaccacctcaatggca |
| | | gagactctctggtgaatccgggcccggccatggcaagccacaaggacgatgaaga |
| | | aaagttttttcctcagagcggggttctcatctttgggaagcaaggctcagagaaa |
| | | acaaatgtggacattgaaaaggtcatgattacagacgaagaggaaatcaggacaa |
| | | ccaatcccgtggctacggagcagtatggttctgtatctaccaacctccagagagg |
| | | caacagacaagcagctaccgcagatgtcaacacacaaggcgttcttccaggcatg |
| | | gtctggcaggacagagatgtgtaccttcaggggcccatctgggcaaagattccac |
| | | acacggacggacattttcacccctctcccctcatgggtggattcggacttaaaca |
| | | ccctcctccacagattctcatcaagaacacaccggttccagcggacccgccgctt |
| | | accttcaaccaggccaagctgaactctttcatcacgcagtacagcaccggacagg |
| | | tcagcgtggaaatcgagtgggagctgcagaaagaaaacagcaagcgctggaaccc |
| | | ggagattcagtacacctccaactactacaaatctacaagtgtggactttgctgtt |
| | | aatacagaaggcgtgtactctgaaccccgccccattggcacccgttacctcaccc |
| | | gtaatctg-3′ SEQ ID NO: 39 |
| HW11 | MAADGYLPDWLEDTLSEGIRQWWKLKP | 5′-atggctgccgatggttatcttccagattggctcgaggacactctctctgaag |
| (VP1) | GPPPPKPAERHKDDSRGLVLPGYKYLG | gaataagacagtggtggaagctcaaacctggcccaccaccaccaaagcccgcaga |
| | PFNGLDKGEPVNAADAAALEHDKAYDQ | gcggcataaggacgacagcaggggtcttgtgcttcctgggtacaagtacctcgga |
| | QLKAGDNPYLKYNHADAEFQERLKEDT | cccttcaacggactcgacaagggggagcccgtcaacgcggcggatgcagcggccc |
| | SFGGNLGRAVFQAKKRVLEPLGLVEEF | tcgagcacgacaaagcctacgaccggcagctcgacagcggagacaacccgtacct |
| | VKTAPGKKRPVEHSPVEPDSSSGTGKA | caagtacaaccacgccgacgcggagtttcaggagcgccttaaagaagatacgtct |
| | GQQPARKRLNFGQTGDAESVPDPQPLG | tttgggggcaacctcggacgagcagtcttccaggcgaaaaagagggttctcgaac |
| | EPPATPAAVGPTTMASGGGAPMADNNE | ctctcggtctggttgaggaaggcgctaagacggctcctggaaagaaacgtccggt |
| | GADGVGNASGNWHCDSTWMGDRVITTS | agagcagtcgccacaagagccagactcctcctcgggcatcggcaagacaggccag |
| | TRTWALPTYNNHLYKQISSQSGASNDN | cagcccgctaaaaagagactcaattttggtcagactggcgactcagagtcagtcc |
| | HYFGYSTPWGYFDFNRFHCHFSPRDWQ | ccgatccacaacctctcggagaacctccagcagccccctctggtctgggatctgg |
| | RLINNNWGFRPKRLSFKLENIQVKEVT | tacagtggctgcaggcggtggcgcaccaatggcagacaataacgaaggtgccgac |
| | QNEGTKTIANNLTSTIQVFTDSEYQLP | ggagtaggtaatacctcaggaaattgacattgcgattccacatggataggcgaca |
| | YVLGSAHQGCLPPFPADVFMIPQYGYL | ggagtgggtaatgcctcaggaaattggcattgcgattccacatggatgggcgaca |
| | TLNNGSQAVGRSSFYCLEYFPSQMLRT | gagtcatcaccaccagcacccgaacctgggccctgcccacctacaacaaccacct |
| | GNNFEFSYTFEDVPFHSSYAHSQSLDR | ctacaaacaaatttccagccaatcaggagcctcgaacgacaatcactactttggc |
| | LMNPLIDQYLYYLSRTNTPSGTTTQSR | tacagcaccccctgggggtattttgacttcaacagattccactgccatttctcac |
| | LQFSQAGASDIRDQSRNWLPGPCYRQQ | cacgtgactggcagcgactcatcaacaacaactggggattccggcctaagcgact |
| | RVSKTSADNNNSEYSWTGATKYHLNGR | caacttcaagctcttcaacattcaggtcaaaaaggttacggacaacaatggagtc |
| | DSLVNPGPAMASHKDDEEKFFPQSGVL | aagaccatcgccaataaccttaccagcacggtccaggtcttcacggactcagact |
| | IFGKQGSEKTNVKIEKVMITDEEEIRT | atcagctcccgtacgtgctcgggtcggctcacgagggctgcctcccgccgttcca |
| | TNPVATEQYGSVSTNLQRGNRQAATAD | agcggacgttttcatgattcctcagtacgggtatctgacgcttaatgatggaagc |
| | VNTQGVLPGMVWQDRDVYLQGPIWAKI | caggccgtgggtcgttcgtccttttactgcctggaatatttcccatcgcagatgc |
| | PHTDGHFHPSPLMGGFGLKHPPPQILI | tgagaaccggcaacaacttccagtttacttacaccttcgaggacgtgcctttcca |
| | KNTPVPADPPLTFNQAKLNSFITQYST | cagcagctacgcccacagccagagcttggaccggctgatgaatcctctgattgac |
| | GQVSVEIEWELQKENSKRWNPEIQYTS | cagtacctgtactacttgagcagaacaaacactccaagtggaaccaccacgcagt |
| | NYYKSTSVDFAVNTEGVYSEPRPIGTR | caaggcttcagttttctcaggccggagcgagtgacattcgggaccagtctaggaa |
| | YLTRNL SEQ ID NO: 16 | ctggcttcctggaccctgttaccgccagcagcgagtatcaaagacatctgcggat |
| | | aacaacaacagtgaatactcgtggactggagctaccaagtaccacctcaatggca |
| | | gagactctctggtgaatccgggcccggccatggcaagccacaaggacgatgaaga |
| | | aaagttttttcctcagagcggggttctcatctttgggaagcaaggctcagagaaa |
| | | acaaatgtggacattgaaaaggtcatgattacagacgaagaggaaatcaggacaa |
| | | ccaatcccgtggctacggagcagtatgattctgtatctaccaacctccagagagg |
| | | caacagacaagcagctaccgcagatgtcaacacacaaggcgttcttccaggcatg |
| | | gtctggcaggacagagatgtgtaccttcaggggcccatctgggcaaagattccac |
| | | acacggacggacattttcacccctctcccctcatgggtggattcggacttaaaca |
| | | ccctcctccacagattctcatcaagaacaccccggtacctgcgaatccttcgacc |
| | | accttcagtgcggcaaagtttgcttccttcatcacacagtactccacgggacagg |
| | | tcagcgtggagatcgagtgggagctgcagaaggaaaacagcaaacgctggaatcc |
| | | cgaaattcagtacacttccaactacaacaagtctgttaatgtggactttactgtg |
| | | gacactaatggcgtgtattcagagcctcgccccattggcacccgttacctcaccc |
| | | gtaatctg-3′ SEQ ID NO: 40 |
| HW12 | VAAGGGAPMADNNEGADGVGNASGNWH | 5′-atggctgccgatggttatcttccagattggctcgaggacaacctctctgagg |
| (VP1) | CDSTWMGDRVITTSTRTWALPTYNNHL | gcattcgcgagtggtgggacctgaaacctggagccccgaaacccaaagccaacca |
| | YKQISSQSGASNDNHYFGYSTPWGYFD | gcaaaagcaggacaacggccggggtctggtgcttcctggctacaagtacctcgga |
| | FNRFHCHFSPRDWQRLINNNWGFRPKR | cccttcaacggactcgacaagggggagcccgtcaacgcggcggacgcagcggccc |
| | LNFKLFNIQVKEVTDNNGVKTIANNLT | tcgagcacgacaaggcctacgaccagcagctcaaagcgggtgacaatccgtacct |
| | STVQVFTDSDYQLPYVLGSAHEGCLPP | gcggtataaccacgccgacgccgagtttcaggagcgtctgcaagaagatacgtca |
| | FPADVFMIPQYGYLTLNDGSQAVGRSS | tttgggggcaacctcgggcgagcagtcttccaggccaagaagcgggttctcgaac |
| | PFHSSYAHSQSLDRLMNPLIDQYLQQL | ctctcggtctggttgaggaaggcgctaagacggctcctgcaaagaagagaccagt |
| | SRTNTPSGTTTQSRLQFSQAGASDIRD | agagcaatcaccccaagaaccagactcctcctcgggcatcggcaagaaaggccaa |
| | QSRNWLPGPCYRQQRVSKTSADNNNSE | cagcccgccagaaaaagactcaattttggccagactggcgactcagagtcagttc |
| | YSWTGATKYHLNGRDSLVNPGPAMASH | cagaccctcaacctctcggagaacctccagcaacccccgctgctgtgggacctac |
| | KDDEEKFFPQSGVLIFGKQGSEKTNVD | tacaatggcttcaggcggtggcgcaccaatggcagacaataacgaaggcgccgac |
| | IEKVMITDEEEIRTTNPVATEQYGSVS | ggagtgggtaattcctcgggaaattggcattgcgattccacatggctgggggaca |
| | TNLQRGNRQAATADVNTQGVLPGMVWQ | gagtcatcaccaccagcacccgaacctgggccctgcccacctacaacaaccacct |
| | DRDVYLQGPIWAKIPHTDGHFHPSPLM | ctacaaacaaatttccagccaatcaggagcctcgaacgacaatcactactttggc |
| | GGFGLKHPPPQILIKNTPVPANPSTTF | tacagcaccccttgggggtattttgacttcaacagattccactgccacttttcac |
| | SAAKFASFITQYSTGQVSVEIEWELQK | cacgtgactggcaaagactcatcaacaacaactggggattccgacccaagagact |
| | ENSKRWNPEIQYTSNYNKSVNVDFTVD | caacttcaagctctttaacattcaagtcaaagaggtcacgcagaatgacggtacg |
| | TNGVYSEPRPIGTRYLTRNL SEQ ID | acgacgattgccaataaccttaccagcacggttcaggtgtttacggactcggagt |
| | NO: 17 | accagctgccgtacgttctcggctctgcccaccagggctgcctgcctccgttccc |
| | | ggcggacgtgttcatgattccccagtacggctacctaacactcaacaacggtagt |
| | | caggccgtgggacggtcatccttttactgcctggaatatttcccatctcagatgc |
| | | tgagaacgggcaataactttaccttcagctacaccttcgaggacgtgcctttcca |
| | | cagcagctacgctcacagccagagtctggaccgtctcatgaatcctctcatcgac |
| | | cagtacctgtattacttgagcagaacaaacactccaagtggaaccaccacgcagt |
| | | caaggcttcagttttctcaggccggagcgagtgacattcgggaccagtctaggaa |
| | | ctggcttcctggaccctgttaccgccagcagcgagtatcaaagacatctgcggat |
| | | aacaacaacagtgaatactcatggactggagctaccaagtaccacctcaatggca |
| | | gagactctctggtgaatccgggcccggccatggcaagccacaaggacgatgaaga |
| | | aaagttttttcctcagagcggggttctcatctttgggaagcaaggctcagagaaa |
| | | acaaatgtggacattgaaaaggtcatgattacagacgaagaggaaatcaggacaa |
| | | ccaatcccgtggctacggagcagtatggttctgtatctaccaacctccagagagg |
| | | caacagacaagcagctaccgcagatgtcaacacacaaggcgttcttccaggcatg |
| | | gtctggcaggacagagatgtgtaccttcaggggcccatctgggccaagattcctc |
| | | acacggacggcaacttccacccctctcccctcatgggtggattcggacttaaaca |
| | | ccctcctccacagattctcatcaagaacaccccggtacctgcgaatccttcgacc |
| | | accttcagtgcggcaaagtttgcttccttcatcacacagtactccacgggacagg |
| | | tcagcgtggagatcgagtgggagctgcagaaggaaaacagcaaacgctggaatcc |
| | | cgaaattcagtacacttccaactacaacaagtctgttaatgtggactttactgtg |
| | | gacactaatggcgtgtattcagagcctcgccccattggtactcgttacctcaccc |
| | | gtaatctg-3′ SEQ ID NO: 41 |
| HW13 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacctgaaacctggagccccgaagcccaaagccaacca |
| | PENGLDKGEPVNEADAAALEHDKAYDR | gcagaagcaggacgacggccggggtctggtgcttcctggctacaagtacctcgga |
| | QLKAGDNPYLRYNHADAEFQERLQEDT | cccttcaacggactcgacaagggagagccggtcaacgaggcagacgccgcggccc |
| | SFGGNLGRAVFQAKKRVLEPLGLVEEA | tcgagcacgacaaagcctacgaccggcagctcaaagcgggtaacaatccgtacct |
| | AKTAPGKKRPVEPSPQRSPDSSTGIGK | gcggtataaccacgccgacgccgagtttcaggagcgtctgcaagaagatacgtct |
| | KGQQPAKKRLNFGQTGDSESVPDPQPL | tttgggggcaacctcgggcgagcagtcttccaggccaagaagcgggttctcgaac |
| | GEPPAAPSGLGPNTMASGGGAPMADNN | ctctcggtctggttgaggaagctgctaagacggctcctggaaagaagagaccggt |
| | EGADGVGNASGNWHCDSTWLGDRVITT | agaaccgtcacctcagcgttcccccgactcctccacgggcatcggcaagaaaggc |
| | STRTWALPTYNNHLYKQISSQSGASND | cagcagcccgctaaaaagagactgaactttggtcagactggcgactcagagtcag |
| | NHYFGYSTPWGYFDFNRFHCHFSPRDW | tccccgacccacaacctctcggagaacctccagcagccccctcaggtctgggacc |
| | QRLINNNWGFRPKRLSFKLFNIQVKEV | taatacaatggcttcaggcggtggcgctccaatggcagacaataacgaaggcgcc |
| | TQNEGTKTIANNLTSTIQVFTDSEYQL | gacggagtgggtaatgcctcaggaaattggcattgcgattccacatggctgggcg |
| | PVYLGSAHQGCLPPFPADVFMIPQYGY | acagagtcatcaccaccagcacccgaacctgggccctgcccacctacaacaacca |
| | LTLNNGSQSVGRSSFYCLEYFPSQMLR | cctctacaaacaaatttccagccaatcaggagcctcgaacgacaatcactacttt |
| | TGNNFTFSYTFEDVPFHSSYAHSQSLD | ggctacagcaccccttgagggtatttcgacttcaacagattccactaccactttt |
| | TLMNPLIDQYLYYLSRTNTPSGTTTQS | caccacgtgactggcagcgactcatcaacaacaactggggattccggcccaagag |
| | RLQFSQAGASDIRDQSRNWLPGPCYRQ | actcagcttcaagctcttcaacatccaggtcaaggaggtcacgcagaatgaaggc |
| | QRVSKTSADNNNSEYSWTGATKYHLNG | accaagaccatcgccaataaccttaccagcacgattcaggtctttacggactcgg |
| | RDSLVNPGPAMASHKDDEEKFFPQSGV | aataccagctcccgtacgtcctcggctctgcgcaccagggctgcctccctccgtt |
| | LIKGKQGSEKTNVDIEKVMITDEEEIR | cccggcggacgtgttcatgattcctcagtacggctacctgactctcaacaatggc |
| | TTNPVATEQYGSVSTNLQRGNRQAATA | agtcagtctgtgggacgttcctccttctactgcctggagtacttcccctctcaga |
| | DVNTQGVLPGMVWQDRDVYLQGPIWAK | tgctgcgtaccggaaacaactttaccttcagctacacttttgaggacgttccttt |
| | IPHTDGNFHPSPLMGGFGLKHPPPQIL | ccacagcagctacgctcacagccagagtctggaccgtctcatgaatcctctcatc |
| | IKNTPVPADPPTTFNQSKLNSFITQYS | gaccagtacctgtattacttgagcagaacaaacactccaagtggaaccaccacgc |
| | TGQVSVEIEWELQKENSKRWNPEIQYT | agtcaaggcttcagttttctcaggccggagcgagtgacattcgggaccagtctag |
| | SNYYKSTSVDFAVNTEGVYSERPRIGT | gaactggcttcctggaccctgttaccgccagcagcgagtatcaaagacatctgcg |
| | RYLTRNL SEQ ID NO: 18 | gataacaacaacagtgaatactcgtggactggagctaccaagtaccacctcaatg |
| | | gcagagactctctggtgaatccgggcccggccatggcaagccacaaggacgatga |
| | | agaaaaattttttcctcagagcggggttctcatctttgggaagcaagactcagag |
| | | aaaacaaatgtggacattgaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggccaaaattc |
| | | ctcacacggacggcaacttccacccatctcccctgatgggcggctttggactaaa |
| | | gcacccgcctcctcagatcctgatcaagaacacgcctgtacctgcggatcctccg |
| | | accaccttcaaccagtcaaagctgaactctttcatcacgcaatacagcaccggac |
| | | aggtcagcgtggaaattgaatgggagctgcagaaagaaaacagcaagcgctggaa |
| | | ccccgagatccagtacacctccaactactacaaatctacaagtgtggactttgct |
| | | gtcaacacggagggggtttatagcgagcctcgccccattggcacccgttacctca |
| | | cccgtaatctg-3′ SEQ ID NO: 42 |
| HW14 | MAADGYLPDWLEDNLSEGIREWWDLKP | 5'-atggctgccaatggttatcttccagattggctcgaggacaacctctctgaga |
| (VP1) | GAPKPKANQQKQDNGRGLVLPGYKYLG | gcattcgcgagtggtgggacctgaaacctggagccccgaaacccaaagccaacca |
| | PFNGLDKGEPVNAADAAALEHDKAYDQ | gcaaaagcaggacaacggccggggtctggtgcttcctggctacaagtacctcgga |
| | QLKAGDNPYLRYNHADAEFQERLQEDT | cccttcaacggactcgacaagggggagcccgtcaacgcggcggatgcagcggccc |
| | SFGGNLGRAVFQAKKRVLEPLGLVEEG | tcgagcacgacaaggcctacgaccagcagctcaaagcgggtgacaatccgtacct |
| | AKTAPGKKRPVEQSPQEPDSSSGIGKK | gcggtataaccacgccgacgccgagtttcaggagcgtctgcaagaagatacgtct |
| | GQQPARKRLNFGQTGDSESVPDPQPIG | tttgggggcaacctcgggcgagcagtcttccaggccaagaagcgggttctcgaac |
| | EPPAAPSGVGPNTMAAGGGAPMADNNE | ctctgggcctggttgaggaaggcgctaagacggctcctggaaagaagagaccagt |
| | GADGVGNASGNWHCDSTWLGDRVITTS | agagcagtcaccccaagaaccagactcctcctcgggcatcggcaagaaaggccaa |
| | TRTWALPTYNNHLYKQISSETAGSTND | cagcccgccagaaaaagactcaattttggccagactggcgactcagagtcagtgc |
| | NTYFGYSTPWGYFDFNRFHCHFSPRDW | ccgaccctcaaccaatcggagaacctccagcagcgccctctggtgtgggacctaa |
| | QRLINNNWGFRPKRLSFKLFNIQVKEV | tacaatggctgcaggcggtggcgcaccaatggcagacaataacgaaggcgccgac |
| | TTNGDVTTIANNLTSTVQVFSDSEYQL | ggagtgggtaatgcctcaggaaattggcattgcgattccacatggctgggctaca |
| | PYVLGSAHQGCLPPFPADVFMIPQYGY | gagtcatcaccaccagcacccgaacatgggccttgcccacctataacaaccacct |
| | LTLNNGSQAVGRSSFYCLEYFPSQMLR | ctacaagcaaatctccagtgaaactccaggtaataccaacgacaacacctacttc |
| | TGNNFQFSYTFEDVPFHSSYAHSQSLD | ggatacagcaccccatgggggtattttgactttaacagattccactgccacttct |
| | RLMNPLIDQYLYYLSRTNTPSGTTTQS | caccacgtgactggcagcgactcatcaacaacaactggggattccggccaaaaag |
| | RLQFSQAGASDIRDQSRNWLPGPCYRQ | actcagcttcaagctcttcaacatccaagtcaaggaggtcacgacgaatgatggc |
| | QRVSKTSADNNNSEYSWTGATKYHLNG | gtcacgaccatcgctaataaccttaccagcacggttcaagtcttctcggactcgg |
| | RDSLVNPGPAMASHKDDEEKFFPQSGV | agtaccagttgccgtacgtcctcggctctgcgcaccagggctgcctgcctccgtt |
| | LIFGKQGSEKTNVDIEKMVITDEEEIR | cccggcggacgtcttcatgattccccagtacggctacctaacactcaacaacggt |
| | TTNFVATEQYGSVSTNLQRGNRQAATA | agtcaggccgtgggacgctcctccttctactgtctggagtatttcccatcgcaga |
| | DVNTQGVLPGMVWQDRDVYLQGPIWAK | tgctgagaaccggcaacaactttcagttcagctacaccttcgaggacgtgccttt |
| | IPHTDGHFHPSPLMGGFGLKHPPPQIL | ccacagcagctacgcgcacagccagagcctggacaggctgatgaatcccctcatc |
| | IKNTPVPANPSTTFSAAKFASFITQYS | gaccagtacctgtattacttgagcagaacaaacactccaagtggaaccaccacgc |
| | TGQVSVEIEWELQKENSKRWNPEIQYT | agtcaaggcttcagttttctcaggccggagcgagtgacattcgggaccagtctag |
| | SNYYKSTSVDFAVNTEGVYSEPRPIGT | gaactggcttcctggaccctgttaccgccagcagcgagtatcaaagacatctgcg |
| | RYLTRNL SEQ ID NO: 19 | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttgggaagcaaggctcagag |
| | | aaaacaaatgtggacattgaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggacattttcacccctctcccctcatgggtggattcggacttaa |
| | | acaccctcctccacagatcctcatcaaaaacacacctgtacctgcgaatccttcg |
| | | accaccttcagtgcggcaaagtttacttccttcatcacacagtactccacgggac |
| | | aggtcagcgtggaaatcgagtgggagctgcagaaagaaaacagcaaacgctggaa |
| | | cccagagattcaatacacttccaactactacaaatctacaagtgtggactttgct |
| | | gttaatacagaaggcgtgtactctgaaccccgccccattggcacccgttacctca |
| | | cccgtaatctg-3′ SEQ ID NO: 43 |
| HW15 | MAADGYLPDWLEDNLSEGIREWWDLKP | 5′-atggctgccgatggttatcttccagattggatcgaggacaacctctctgagg |
| (VP1) | GAPKPKANQQKQDNGRGLVLPGYKYLG | gcattcgcgagtggtgggacttgaaacctggagccccgaagcccaaagccaacca |
| | PFNGLDKGEPVNAADAAALEHDKAYDQ | gcaaaagcaggacgacggccggggtctggtgcttccttgctacaagtacctcgga |
| | QLKAGDNPYLRYNHADAEFQERLQEDT | ccattcaacggactcgacaagggggagcccgtcaacgcggaggacgcagcggcca |
| | SFGGNLGRAVFQAKKRVLEPLGLVEEG | tcgagcacgacaaggcctacgaccagcagctgcaggcgggtgacaatccgtacct |
| | AKTAPGKKRPVEQSPQEPDSSSGIGKK | gcggtataaccacgccgacgccgagtttcaggagcgtctgcaagaagatacgtct |
| | GQQPARKRLNFGQTGDSESVPDPQPIG | tttgggggcaacatcgggcgagcagtcttccaggccaagaagcgggttctcgaac |
| | EPPAAPSGVGPNTMAAGGGAPMADNNE | ctctcggtctggttgaggaaggcgctaagacggctcctggaaagaaacgtccggt |
| | GADGVGNASGNWHCDSTWLGDRVITTS | agagcagtctcctcaggaaccggactcctccgcgggtattggcaaatcgggtgca |
| | TRTWALPTYNNHLYKQISSETAGSTND | cagcccgctaaaaagagactcaattttggtcagactggcgactcagagtcagtcc |
| | NTYFGYSTPWGYFDFNRFHCHFSPRDW | ccgatccacaacctctcggagaaccaccagcaggcccctctggtctgggatctgg |
| | QRLINNNWGFRPKRLSFKLFNIQVKEV | tacaatggctgcaggcggtggagctccaatggcagacaataacgaaggcgccgac |
| | TTNGDVTTIANNLTSTVQVFSDSEYQL | ggagtgggtaattcctcgggaaattggcattgcaattccacatggctgggcgaca |
| | PYVLGSAHQGCLPPFPADVFMIPQYGY | gagtcatcaccaccagcacccgaacctgggccctgcccacctacaacaaccacct |
| | LTLNNGSQAVGRSSFYCLEYFPSQMLR | ctacaagcaaatctccagtgcttcaacgggggccagcaacgacaacacctacttc |
| | TGNNFQFSYTFEDVPFHSSYAHSQSLD | ggctacagcaccccctgggggtattttaactttaacagattccactgccactttt |
| | RLMNPLIDQYLYYLSRTNTPSGTTTQS | caccacgtgactggcagcgactcatcaacaacaactggggattccggccaaaaag |
| | RLQFSQAGASDIRDQSRNWLPGPCYRQ | actcagcttcaagctcttcaacatccaggtcaaggaggtcacgcagaatgaaggc |
| | QRVSKTSADNNNSEYSWTGATKYHLNG | accaagaccatcgccaataaccttaccagcacgattcaggtctttacggactcga |
| | RDSLVNPGPAMASHKDDEEKFFPQSGV | aataccagctgccgtacgtcctcggctctgcccaccagggctgcatgactccgtt |
| | LIFGKQGSEKTNVDIEKMVITDEEEIR | cccggcggacgtgttcatgattccccagtacggttacctaacactcaacaacggt |
| | TTNFVATEQYGSVSTNLQRGNRQAATA | agtcaggccgtgggacgctcctccttctactgcctggaatactttccttcgcaga |
| | DVNTQGVLPGMVWQDRDVYLQGPIWAK | tgctgagaacgggcaacaactttgagttcagctaccagtttgaggacgtgccttt |
| | IPHTDGHFHPSPLMGGFGLKHPPPQIL | tcacagcagctacgcgcacagccagagcctggaccggctgatgaatcctctcatc |
| | IKNTPVPANPSTTFSAAKFASFITQYS | gaccagtacctgtactacttgtctcggactcaaacaacaggaggcacggcaaata |
| | TGQVSVEIEWELQKENSKRWNPEIQYT | cgcagactctgggcttcagccaaggtgggcctaatacaatggccaatcaggcaaa |
| | SNYYKSTSVDFAVNTEGVYSEPRPIGT | gaactggctgccaggaccctgttaccgccaacaacgcgtctcaacgacaaccggg |
| | RYLTRNL SEQ ID NO: 20 | caaaacaacaatagcaactttgcctggactgctgggaccaaataccatctgaatg |
| | | gaagaaattcattggctaatcctggcatcgctatggcaacacacaaagacgacga |
| | | ggagcgtttttttcccagtaacgggatcctgatttttggcaaacaaaatgctgcc |
| | | agagacaatgcggattacagcgatgtcatgctcaccagcgaggaagaaatcaaaa |
| | | ccactaaccctgtggctacagaggaatacggtatcgtggcagataacttgcagca |
| | | aaccaatacggggcctattgtgggaaatgtcaacagccaaggagccttacctggc |
| | | atggtctggcagaaccgagacgtgtacctgcagggtcccatctgggccaagattc |
| | | ctcacacggacggcaacttccacccgtctccgctgatgggcggctttggcctgaa |
| | | acatcctccgcctcagatcctgatcaagaacacgcctgtacctgcggatcctccg |
| | | accaccttcaaccagtcaaagctgaactctttcatcacgcaatacagcaccggac |
| | | aggtcagcgtggaaattgaatgggagctgcagaaggaaaacagcaagcgctggaa |
| | | cccggagatccagtacacttccaactattacaagtctaataatgttgaatttgct |
| | | gttaatacagaaggcgtgtactctgaaccccgcccaattggcacccgttacctca |
| | | cccgtaatctg-3′ SEEQ ID NO: 44 |
| HW16 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacttgaaacctggagccccgaaacccaaagccaacca |
| | PFNGLDKGEPVNAADAAALEHDKAYDQ | gcaaaagcaggacgacggccggggtctggtgcttcctggctacaagtacatcgga |
| | QLKAGDNPYLRYNHADAEFQERLQEDT | cccttcaacggactcgacaagggggagcccgtcaacgcggcggacgcagcggccc |
| | SFGGNLGRAVFQAKKRVLEPLGLVEEA | tcgagcacgacaaggcctacgaccagcagctcaaagcgggtgacaatccgtacct |
| | AKTAPGKKRPVEPSPQRSPDSSTGIGK | gcggtataaccacgccgacgccgagtttcaggagcgtctgcaagaagatacgtct |
| | KGQQPARKRLNFGQTGDADSVPDPQPL | tttggaggcaacctcgggcgagcagtcttccagaccaagaagcgggttctcgaac |
| | GQPPAAPSGLGTNTMATGSGAPMADNN | ctctcggtctggttgaggaagctgctaagacggctcctggaaagaagagaccggt |
| | EGADGVGNSSGNWHCDSTWLGNRVITT | agaaccgtcacctcagcgttcccccgactcctccacgggcatcggcaagaaaggc |
| | STRTWALPTYNNHLYKQISNGTSGGST | cagcagcctgcaagaaaaagattgaattttggtcagactggagacgcagactcag |
| | NDNTYFGYSTPWGYFDFNRFHCHRSPR | tacctgacccccagcctctcggacagccaccagcagccccctctggtctgggaac |
| | DWQRLINNNWGFRPKRLSFKLFNIQVK | taatacgatggctacaggcagtggcgcaccaatggcagacaataacgagggcgcc |
| | EVTQNEGTKTIANNLTSTVQVFTDSDY | gacggagtgggtaattcctcgggaaattggcattgcgattccacatggctgggca |
| | QLPYVLGSAHEGCLPPFPADVFMIPQY | acagagtcatcaccaccagcacccgaacctgggccctgcccacctacaaaaacca |
| | GYLTLNDGSQAVGRSSFYCLEYFPSQM | cctctacaagcaaatatccaatgggacatcgggaggaagcaccaacgacaacacc |
| | LRTGNNFQFTYTFEDVPFHSSYAHSQS | tattttggctacagcaccccctgggggtattttgacttcaacagattccactgtc |
| | LDRLMNPLIDQYLYYLSRTQTTGGTAN | acttttcaccacgtgactggcaacgactcatcaacaacaactggggattccggcc |
| | TQTLGFSQGGPNTMANQAKNWLPGPCY | caagagactcagcttcaagctcttcaacattcaggtcaaggaggtcacgcagaat |
| | RQQRVSTTTGQNNNSNFAWTAGTKYHL | gaaggcaccaagaccatcgccaataaccttaccagcacggtccaggtcttcacgg |
| | NGRNSLANPGIAMATHKDDEERFFPSN | actcagactatcagctcccgtacgtgctcgggtcggctcacgagggctgcctccc |
| | GILIFGKQNAARDNADYSDVMLTSEEE | gccgttcccagcggacgttttcatgattcctcagtacgggtatctgacgcttaat |
| | IKTTNPVATEEYGIVADNLQQRNTAPQ | gatggaagccaggccgtgggtcgttcgtccttttactgcctggaatatttcccgt |
| | IGTVNSQGALPGMVWQNRDVYLQGPIW | cgcaaatgctaagaacgggtaacaacttccagtttacttacaccttcgaggacgt |
| | AKIPHTDGNFHPSPLMGGFGLKHPPPQ | gcctttccacagcagctacgcccacagccagagcttggaccggctgatgaatcct |
| | ILIKNTPVPADPPTTFSAAKFASFITQ | ctgattgaccagtacctgtactacttgtctcggactcaaacaacaggaggcacgg |
| | YSTGQVSVEIEWELQKENSKRWNPEIQ | caaatacgcagactctgggcttcagccaaggtgagcctaatacaatggccaatca |
| | YTSNYYKSNNVEFAVNTEGVYSEPRPI | ggcaaagaactggctgccaggaccctgttaccgccaacaacgcgtctcaacgaca |
| | GTRYLTRNL SEQ ID NO: 21 | accgggcaaaacaacaatagcaactttgcctggactgctgggaccaaataccatc |
| | | tgaatggaagaaattcattggctaatcctggcatcgctatggcaacacacaaaga |
| | | cgacgaggagcgtttttttcccagtaacgggatcctgatttttggcaaacaaaat |
| | | gctgccagagacaatgcggattacagcgatgtcatgctcaccagcgaggaagaaa |
| | | tcaaaaccactaaccctgtggctacagaggaatacggtatcgtggcagataactt |
| | | gcagcagcgaaacacggctcctcaaattggaactgtcaacagccagggggcctta |
| | | cccggtatggtctggcagaaccgggacgtgtacctgcagggtcccatctgggcca |
| | | agattcctcacacggacggcaacttccacccgtctccgctgatgggcggctttgg |
| | | cctgaaacatcctccgcctcagatcctgatcaagaacacgcctgtacctgcggat |
| | | cctccaactaccttcagtgcggcaaagtttgcttccttcatcacacagtactccc |
| | | cgggacaggtcagcgtggagatcgagtgggagctgcagaaggaaaacagcaaacg |
| | | ctggaatcccgaaattcagtacacttccaactattacaagtctaataatgttgaa |
| | | tttgctgttaatactgaaggtgtatatagtgaaccccgccccattggcaccagat |
| | | acctgactcgtaatctg-3′ SEQ ID NO: 45 |
| HW17 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacctgaaacctggagcccccaagcccaaggccaacca |
| | PFNGLDKGEPVNAADAAALEHDKAYDQ | gcagaagcaggacgacggccggggtctggtgcttccgggttacaaataccttgga |
| | QLKAGDNPYLRYNHADAEFQERLQEDT | cccggcaacggactcgacaagggggagcccgtcaacgcggcggacgcagcggccc |
| | SFGGNLGRAVFQAKKRVLEPLGLVEEA | tcgagcacgacaaggcctacgaccagcagctcaaagcgggtgacaatccgtacct |
| | AKTAPGKKRPVEPSPQRSPDSSTGIGK | gcggtataaccacgccgacgccgagtttcaggagcgtctgcaagaagatacgtca |
| | KGQQPARKRLNFGQTGDADSVPDPQPL | tttgggggcaacctcgggcgagcagtcttccaggccaagaagcgggttctcgaac |
| | GQPPAAPSGLGTNTMATGSGAPMADNN | ctctcggtctggttgaggaaggcgctaagacggctcctggaaagaagagaccggt |
| | EGADGVGNSSGNWHCDSTWLGNRVITT | agagccatcaccccagcgttctccagactcctcctcgggcatcggcaaatcgggt |
| | STRTWALPTYNNHLYKQISNGTSGGST | gcacagcccgctaaaaagagactcaatttcggtcagactggcgacccagagtcag |
| | NDNTYFGYSTPWGYFDFNRFHCHRSPR | tcccagaccctcaaccaatcggagaacctcccgcagccccctcaggtgtgggatc |
| | DWQRLINNNWGFRPKRLSFKLFNIQVK | tcttacaatggcttcaggtggtggcgcaccagtggcagacaataacgaaggtgcc |
| | EVTQNEGTKTIANNLTSTVQVFTDSDY | gatggagtgggtagttcctcgggaaattggcattgcgattccacatggctgggcg |
| | QLPYVLGSAHEGCLPPFPADVFMIPQY | acagagtcatcaccaccagcacccgaacctgggccctgcccacctacaacaacca |
| | GYLTLNDGSQAVGRSSFYCLEYFPSQM | cctctacaagcaaatatccaatgggacatcgggaggaagcaccaacgacaacacc |
| | LRTGNNFQFTYTFEDVPFHSSYAHSQS | tacttcggctacagcaccccctgggggtattttgactttaacagattccactgcc |
| | LDRLMNPLIDQYLYYLSRTQTTGGTAN | acttttcaccacgtgactggcagcgactcatcaacaacaactggggattccgacc |
| | TQTLGFSQGGPNTMANQAKNWLPGPCY | caagagactcagcttcaagctcttcaacatccaggtcaaagaggtcacgcagaat |
| | RQQRVSTTTGQNNNSNFAWTAGTKYHL | gacggtacgacgacgattgccaataaccttaccagcacggttcaggtgtttactg |
| | NGRNSLANPGIAMATHKDDEERFFPSN | actcggagtaccagctcccgtacgtcctcggctcggcgcatcaaggatgcctccc |
| | GILIFGKQNAARDNADYSDVMLTSEEE | gccgttcccggcggacgtcttcatgattcctcagtacgggtacctgactctgaac |
| | IKTTNPVATEEYGIVADNLQQRNTAPQ | aatggcagtcagaccgtggaccgttcctccttctactgcctggagtactttcctt |
| | IGTVNSQGALPGMVWQNRDVYLQGPIW | ctcaaatgctgagaacgggcaacaactttgagttcagctacaccttcgaggacgt |
| | AKIPHTDGNFHPSPLMGGFGLKHPPPQ | gcctttccacagcagctacgcacacagccagagcttggaccgactgatgaatcct |
| | ILIKNTPVPADPPTTFSAAKFASFITQ | ctcatccaccagtacctgtactacttatctcggactcaaacaacaggaggcacga |
| | YSTGQVSVEIEWELQKENSKRWNPEIQ | caaatacgcagactctgggctttagccaaggtgggcctaatacaatggccaatca |
| | YTSNYYKSNNVEFAVNTEGVYSEPRPI | ngcaaagaactggctgccaggaccctgttaccggcagcagcgagtctctacgaca |
| | GTRYLTRNL SEQ ID NO: 22 | accgggcaaaacaacaacagcaactttgcttggactggtgccaccaaatatcacc |
| | | tgaacggaagagactctctggtaaatcccggtgtcgctatggcaacccacaagga |
| | | tgacgacgaccgcttcttcccttcgagcggggtcctgatttttggcaagcaagga |
| | | gccgggaacgatggagtggattacagccaagtgctgattacagacgaagaggaaa |
| | | tcaggacaaccaatcccgtggctacggagcagtatggttctgtatctaccaacct |
| | | ccagagaggcaacagacaagcagctaccgcagatgtcaacacacaaggcgttctt |
| | | ccaggcatggtctggcaggacagagatgtgtaccttcaggggcccatctgggcaa |
| | | agattccacacacggacggacattttcacccctctcccctcatgggtggattcgg |
| | | acttaaacaccctcctccacagattctcatcaagaacaccccggtacctgcgaat |
| | | ccttcgaccaccttcagtgcggcaaagtttgcttccttcatcacacagtactcca |
| | | cgggacaggtcagcgtggagategagtgggagctgcagaaggaaaacagcaagcg |
| | | ctggaacccggagattcaatacacctccaactttgaaaaacagactggtgtggac |
| | | tttgctgtcaatacagagggaacttattctgagcctcgccccattggtactcgtt |
| | | acctcacccgtaatctg-3′ SEQ ID NO 46 |
| HW18 | MAADGYLPDWLEDNLSEGIREWWDLKP | 5′-atggctgccgatggttatcttccagattggctcgaggacaacctctctgagg |
| (VP1) | GAPKPKANQQKQDDGRGLVLPGYKYLG | gcattcgcgagtggtgggacctgaaacctggagccccgaaacccaaagccaacca |
| | PFNGLDKGEPVNAADAAALEHDKAYDQ | gcaaaagcaggacgacggccggggtctggtgcttcctggctacaagtacctcgga |
| | QLKAGDNPYLRYNHADAEFQERLQEDT | cccttcaacggactcgacaagggggagcccgtcaacgcggcggatgcagcggccc |
| | SFGGNLGRAVFQAKKRVLEPLGLVEEA | tcgagcacgacaaggcctactaccagcagctcaaagcgggtaacaatccgtacct |
| | AKTAPGKKRPVEPSPQRSPDSSTGIGK | gcggtataaccacgccgacgccgagtttcaggagcgtctgcaagaagatacgtct |
| | KGQQPARKRLNFGQTGDADSVPDPQPL | tttgggggcaacctcgggcgagcagtcttccaggccaaaaagaggcttcttgaac |
| | GQPPAAPSGLGTNTMATGSGAPMADNN | ctcttggtctggttgaggaagcggctaagacggctcctggaaagaagaggcctgt |
| | EGADGVGNSSGNWHCDSTWLGNRVITT | agagcagtctcctcaggaaccagactcctcctcgggcatcggcaagaaaggccag |
| | STRTWALPTYNNHLYKQISNGTSGGST | cagcccgccagaaagagactcaatttcggtcagactggcgactcagagtcagtcc |
| | NDNTYFGYSTPWGYFDFNRFHCHRSPR | ccgaccctcaacctctcggagaacctccagcaacccccgctgctgtgggacctac |
| | DWQRLINNNWGFRPKRLSFKLFNIQVK | tacaatggcttcaggcggtggcgcaccaatggcagacaataacgaangtgccgat |
| | EVTQNEGTKTIANNLTSTVQVFTDSDY | ggagtgggtagttcctcaggaaattggcattacgattcccaatggctgggggaca |
| | QLPYVLGSAHEGCLPPFPADVFMIPQY | gagtcatcaccaccagcacccgaacctgggccctgcccacctacaacaatcacct |
| | GYLTLNDGSQAVGRSSFYCLEYFPSQM | ctacaagcaaatctccagtgaaactgcaggtagtaccaacgacaacacctacttc |
| | LRTGNNFQFTYTFEDVPFHSSYAHSQS | tgctacagcaccccctgggggtattttgactttaacagattccactgccacttct |
| | LDRLMNPLIDQYLYYLSRTQTTGGTAN | caccacgtgaactggcagcgactcatcaacaacaatggggattccggcccaagag |
| | TQTLGFSQGGPNTMANQAKNWLPGPCY | actcagcttcaagctctttaacattcaagtcaaagaggtcacgcagaatgacggt |
| | RQQRVSTTTGQNNNSNFAWTAGTKYHL | acgacgacgattgccaataaccttaccagcacggttcaggtgtttactgactcgg |
| | NGRNSLANPGIAMATHKDDEERFFPSN | agtaccagctcccgtacgtcctcggctctgcgcaccagggctgcctccctccgtt |
| | GILIFGKQNAARDNADYSDVMLTSEEE | cccggcggacgtattcatgattccgcaatacggctacctaacgctcaacaatggc |
| | IKTTNPVATEEYGIVADNLQQRNTAPQ | agccaggcagtgggacggtcatccttttactgcctggaatatttcccatcgcaga |
| | IGTVNSQGALPGMVWQNRDVYLQGPIW | tgctgagaacgggcaataactttaccttcagctacaccttcgaggacgtgccttt |
| | AKIPHTDGNFHPSPLMGGFGLKHPPPQ | ccacagcagctacgcccacagccagagtctggaccgtctcatgaatcctctcatc |
| | ILIKNTPVPADPPTTFSAAKFASFITQ | gaccagtacctgtattacttgagcagaacaaacactccaagtggaaccaccacgc |
| | YSTGQVSVEIEWELQKENSKRWNPEIQ | agtcaaggcttcagttttctcaggccggagcgagtgacattcgggaccagtctag |
| | YTSNYYKSNNVEFAVNTEGVYSEPRPI | gaactggcttcctggaccctgttaccgccagcagcgagtatcaaagacatctgcg |
| | GTRYLTRNL SEQ ID NO: 23 | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttgggaagcaaggctcagag |
| | | aaaacaaatgtggacattgaaaaggtcatgattacagacgaagaggaaatcagga |
| | | caaccaatcccatggctacggagcaatatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggacattttcacccctctcccctcatgggtggattcggacttaa |
| | | acaccctcctccacagattctcatcaagaacaccccggtacctgcgaatccttcg |
| | | accaccttcagtgcggcaaagtttgcttccttcatcacacagtactccacgggac |
| | | aggtcagcgtggaaattgaatgggagctgcagaaggaaaacagcaagcgctggaa |
| | | ccccgagatccagtacacctccaactactacaaatctacaagtgtggactttgct |
| | | gttaatacagaaggcgtgtactctgaaccccgccccattggcacccgttacctca |
| | | cccgtaatctg-3′ SEQ ID NO: 47 |
| HW19 | MAADGYLPDWLEDNLSEGIREWWDLKP | 5'-atggctgccaatggttatcttccagattggctcgaggacaacctctctgaga |
| (VP1) | GAPKPKANQQKQDDGRGLVLPGYKYLG | gcattcgcgagtggtgggacttgaaacctggagccccgaaacccaaagccaacca |
| | PFNGLDKGEPVNAADAAALEHDKAYDQ | gcaaaagcaggacgacggccggggtctggtgcttcctggctacaagtacctcgga |
| | QLKAGNDPYLRYNHADAEFQERLKEDT | cccttcaacggactcgacaagggggagcccgtcaacgcggcggatgcagcggccc |
| | SFGGNLGRAVFQAKKRVLEPLGLVEEP | tcgagcacgacaaggcctacgaccagcagctcaaagcgggtgacaatccgtacct |
| | VKTAPGKKRPVEHSPVEPDSSSGTGKA | gcggtataaccacgccgacgcggagtttcaggagcgccttaaagaagatacgtct |
| | GQQPARKRLNFGQTGDADSVPDPQPLG | tttgggggcaacctcggacgagcagtcttccaggcgaaaaagagggttcttgaac |
| | QPPAAPSGLGTNTMATGGGAPMADNNE | ctctgggcctggttgaggaacctgttaagacggctccgggaaaaaagaggccggt |
| | GADGVGNASGNWHCDSTWLGDRVITTS | agagcactctcctgtggaaccagactcctcctcgggaaccggaaaggcgggccag |
| | TRTWALPTYNNHLYKQISSETAGSTND | cagcctgcaagaaaaagattgaattttggtcagactggagacgcagactcagtac |
| | NTYFGYSTPWGYFDFNRFHCHFSPRDW | ctgacccccagcctctcggacagccaccagcagccccctctggtctgggaactaa |
| | QRLINNNWGFRPKRLSFKLFNIQVKEV | tacgatggctacaggcggtggcgcaccaatggcagacaataacgaaggtgccgac |
| | TQNEGTKTIANNLTSTIQVFTDSEYQL | ggagtgggtaatgcctcaggaaattggcattgcgttccacattggctgggcgaca |
| | PYVLGSAHQGCLPPFPADVFMIPQYGY | gagtcattaccaccagcacccgaacctgggccctacccacctacaacaaccacct |
| | LTLNNGSQSVGRSSFYCLEYFPSQMMR | ctacaagcaaatctccagtgaaactgcaggtagtaccaacgacaacacctacttc |
| | TGNNFEFSYSFEDVPFHSSYAHSQSLD | ggatacagcaccccctgggggtattttgactttaacagattccactgccactttt |
| | RLMNPLIDQYLYYLSRTQSTGGTAGTQ | caccacgtgactggcagcgactcatcaacaacaactggggattccggcccaagag |
| | QLLFSQAGASDIRDQSRNWLPGPCYRQ | actcagcttcaagctcttcaacatccaggtcaaggaggtcacgcagaatgaaggc |
| | QRVSKTSADNNNSEYSWTGATKYHLNG | accaagaccatcgccaataaccttaccagcacgattcaggtgtttacggactcgg |
| | RDSLVNPGPAMASHKDDEEKFFPQSGV | agtaccagctgccatacgttctcggctctgcccaccagggctgcctgcctccgtt |
| | LIFGKQGSEKTNVDIEKVMITDEEEIR | cccggcggacgtgttcatgattcctcagtacggctacctgactctcaacaatggc |
| | TTNPVATEQYGSVSTNLQRGNRQAATA | agtcagtctgtgggacgttcctccttctactgcctggagtacttcccctctcaga |
| | DVNTQGVLPGMVWQDRDVYLQGPIWAK | tgatgagaacgggcaacaactttgagttcagctacagcttcgaggacgtgccttt |
| | IPHTDGNFHPSPLMGGFGLKHPPPQIL | ccacagcagctacgcacacagccagagcctggaccggctgatgaatcccctcatc |
| | IKNTPVPADPPTTFSQAKLASFITQYS | gaccagtacctgtactacctgtctcggactcagtccacgggaggtaccgcaggaa |
| | TGQVSVEIEWELQKENSKRWNPEIQYT | ctcagcagttgctattttctcaggccggagcgagtgacattcgggaccagtctag |
| | SNYYKSTNVDFAVNTEGVYSEPRPIGT | gaactggattcctggaccctgttaccgccagcagcgagtatcaaagacatctgcg |
| | RYLTRNL SEQ ID NO: 24 | gataacaacaacagtgaatactcgtggactggagctaccaagtaccacctcaatg |
| | | agaaaagttttttcctcagagcggggttctcatctttgggaagcaaggctcagag |
| | | aaaacaaatgtggacattaaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | ctcacacggacggcaacttccacccttcaccgctaatgggaggatttggactgaa |
| | | gcacccacctcctcagatcctgatcaagaacacgccggtacctgcggatcctcca |
| | | acaacgttcagtcaagctaagctggcgtcgttcatcacgcagtacagcaccggac |
| | | aggtcagcgtggaaattgaatgggagctgcagaaggaaaacagcaagcgctggaa |
| | | cccggagattcaatacacttccaactactacaaatctacaaatgtggactttgct |
| | | gtcaacacggagggggtttatagcgagcctcgccccattggcacccgttacctca |
| | | cccgcaacctg-3′ SEQ ID NO: 48 |

In some embodiments, a capsid protein may be the VP protein KJ01. In some embodiments, a capsid protein may be the VP1 protein KJ02. In some embodiments, a capsid protein may be the VP1 protein KJ03. In some embodiments, a capsid protein may be the VP1 protein KJ04. In some embodiments, a capsid protein may be the VP1 protein KJ05. In some embodiments, a capsid protein may be the VP protein HW01. In some embodiments, a capsid protein may be the VP1 protein HW02. In some embodiments, a capsid protein may be the VP1 protein HW03. In some embodiments, a capsid protein may be the VP1 protein HW04. In some embodiments, a capsid protein may be the VP1 protein HW05. In some embodiments, a capsid protein may be the VP1 protein HW06. In some embodiments, a capsid protein may be the VP1 protein HW07. In some embodiments, a capsid protein may be the VP1 protein HW08. In some embodiments, a capsid protein may be the VP1 protein HW09. In some embodiments, a capsid protein may be the VP1 protein HW10. In some embodiments, a capsid protein may be the VP1 protein HW11. In some embodiments, a capsid protein may be the VP1 protein HW12. In some embodiments, a capsid protein may be the VP1 protein HW13. In some embodiments, a capsid protein may be the VP1 protein HW14. In some embodiments, a capsid protein may be the VP1 protein HW15. In some embodiments, a capsid protein may be the VP1 protein HW16. In some embodiments, a capsid protein may be the VP1 protein HW17. In some embodiments, a capsid protein may be the VP1 protein HW18. In some embodiments, a capsid protein may be the VP1 protein HW19.

In some embodiments, a capsid protein described herein may be selected from any of those capsid proteins (VP2) found in Table 2. In some embodiments, the capsid protein may be a variant of any of the capsid proteins found in Table 2. In some embodiments, AAV particles are described herein that comprise such a capsid protein or proteins, or variants thereof. In some embodiments AAV particles are described herein that comprise such a capsid protein or proteins, or variants thereof, and a VP1 protein of Table 1, or variants thereof.

In some embodiments, a capsid protein or proteins may be encoded by a polynucleotide sequence found in Table 2. In some embodiments, the capsid protein or proteins may be encoded by a polynucleotide sequence that is a codon optimized form of a polynucleotide sequence of Table 2. For example, the capsid protein or proteins may be encoded by a polynucleotide sequence that is codon optimized for expression in insect cells, such as Sf9 insect cells. In some embodiments, the capsid protein or proteins may be encoded by a polynucleotide sequence that differs from a polynucleotide sequence of Table 2 due to amino acid code degeneracy. In some embodiments, AAV particles are described herein that comprise a capsid protein or proteins, or variants thereof, encoded by such a polynucleotide.

TABLE 2
Capsid Proteins (VP2)

| Capsid | | |
|---|---|---|
| Protein | Amino Acid | Representative Polynucleotide |
| KJ01 | TAPGKKRPVEPSPQRSPDSSTGIGKK | 5′-acggctcctggaaagaagagaccggtagagccatcaccccagcgttctcca |
| (VP2) | GQQPARKRLNFGQTGDADSVPDPQPL | gactcctctacgggcatcggcaagaaaggccaacagcccgcaagaataagattg |
| | GEPPAAPSSVGSGTMAAGGGAPMADN | gactcctctacgggcatcggcaagaaaggccaacagcccgcaagaaaaagattg |
| | NEGADGVGSSSGNWHCDSTWLGDRVI | aattttggtcagactggagacgcagactcagtacctgacccacaacctctcgga |
| | TTSTRTWALPTYNNHLYKQISNGTSG | gaacctccagcagcgccctctagtgtgggatctggtacaatggctgcaggcggt |
| | GSTNDNTYFGYSTPWGYFDFNRFECM | ggcgctccaatggcagacaataacgaaggccccgacggagtgggtagttcctca |
| | FSPRDWQRLINNNWGFRPKRLSFKLF | ggaaattggcattgcgattccacatggctgggcgacagagtcatcaccaccagc |
| | NIQVKEVTQNEGTKTIANNLTSTIQV | acccgaacctgggccctgcccacctacaacaaccacctctacaagcaaatctcc |
| | FTDSEYQLPYVLGSAHQGCLPPFPAD | aacggcacctcgggaggaagcaccaacgacaacacctattttggctacagcacc |
| | VFMIPQYGYLTLNNGSQAVGRSSFYC | ccctgggggtatcttgactctaacagattccaccgccacttttcaccacgtgac |
| | LEYFPSQMLRTGNNFQFTYTFEDVFF | tggcagcgactcatcaacaacaactcgggattccggcccaagagactcaacttc |
| | HSSYAHSQSLDRLMNPLIDQYLYYLS | aagctcttcaacatccaggtcaaggaggtcacgcagaatgaaggcaccaagacc |
| | RTQSTGGTAGTQQLLFSQAGPNNMSA | atcgccaataacctcaccagcaccatccaggtgcttacggactcggagtaccag |
| | QAKNWLPGPCYRQQRVSTTLSQNNNS | ctgccgtacgttctcggctctgcccaccagggctgcctgcctccgttcccggcg |
| | NFAWTGATKYHLNGRDSLVNPGVAMA | gacgtgttcatgattccccagtacggctacctaacactcaacaacggtagtcag |
| | THKDDEERFFPSSGVLMFGKQGAGKD | gccgtgggacgctcctccttctactgcctggaatactttccttcgcagatgctg |
| | NVDYSSVMLTSEEEIKTTNPVATEQY | agaaccggcaacaacttccagtttacttacaccttcgaggacgtgcccttccac |
| | GVVADNLQQQNAAPIVGAVNSQGALP | agcagctacgcccacagccagagctcggaccggctgatgaatcctctgactgac |
| | GMVWQNRDVYLQGPIWAKIPHTDGNF | caatacctgtactacctgtctcggactcagtccacggaaggtaccgcaggaact |
| | HPSPLMGGFGLKHPPPQILIKNTPVP | cagcagttgctattttctcaggccgggcctaataacatgtcggctcaggccaaa |
| | ADPPTTFSQAKLASFITQYSTGQVSV | aactggccacccgggccccgccaccggcagcaacgcgtctccacgacaccgtcg |
| | EIEWELQKENSKRWNPEIQYTSNYYK | caaaataacaacagcaactttgcctggaccggtgccaccaagtatcatctgaat |
| | STNVDFAVNTDGTYSEPRPIGTRYLT | ggcagagactctctggtaaatcccggtgtcgctatggcaacccacaaggacgac |
| | RNL SEQ ID NO: 49 | gaagagcgatttttcccgtccagcggagccttaatgtttgggaaacagggagct |
| | | ggaaaagacaacgtggactatagcagcgttatgctaaccagtgaggaagaaatt |
| | | aaaaccaccaacccagtggccacagaacagtacggcgtggtggccgataacctg |
| | | caacagcaaaacgccgctcctattgtaggggccgtcaacagtcaaggagcctta |
| | | cctggcatggtctggcagaaccgggacgtgtacctgcagggtcctatctgggcc |
| | | aagattcctcacacggacggaaactttcatccctcgccgctgatgggaggcttt |
| | | ggactgaaacacccgcctcctcagatcctgattaagaatacacctgttcccgcg |
| | | gatcctccaactaccttaagtcaagctaagccggcgtcgttcatcacgcagtac |
| | | agcaccggacaagtcaccgtggaaatcgagtgggagccgcagaaggaaaacagc |
| | | aaacgctggaatccagagattcagtacacttcaaactactacaaatctacaaat |
| | | gtggactttgctgtcaacacagatggcacttattctgagcctcgccccatcggc |
| | | acccgttacctcacccgtaatctg-3′ SEQ ID NO: 73 |
| KJ02 | TAPGKKRPVEQSPQEPDSSSGIGKTG | 5′-acggctcctggaaagaaacgtccggcagagcagtcgccacaagagccagac |
| (VP2) | QQPAKKRLNFGQTGDSESVPDPQPIG | tcctcctcgggcatcggcaagacaggccaacagcccgctaaaaagagactcaat |
| | EPPAGPSGLGSGTMAAGGGAPMADNN | tttggtcagactggcgactcagagtcagtccccgaccctcaaccaatcggagaa |
| | EGADGVGNASGNWHCDSTWLGDRVIT | ccaccagcaggcccctctggtctgggatctggtacaatggctgcaggcggtggc |
| | TSTRTWALPTYNNHLYKQISSETAGS | gctccaatggcagacaataacgagggcgccgacggagtgggtaatgcctcagga |
| | TNDNTYFGYSTPWGYPDFNRFHCHFS | aattggcattgcgattccacatggctgggcgacagagtcattaccaccagcacc |
| | PRDWQRLINNNWGFRPKRLNFKLLNI | cgaacctgggccctgcccacctacaacaaccacctctacaagcaaatctccagt |
| | QVKEVTDNNGVKTIANNLTSTIQVFT | gaaactgcaggtagtaccaacgacaacacctacttcggctacagcaccccctgg |
| | DSEYQLPYVLGSAHQGCPPPFPADVF | gggtactttcacctcaacagattccactgccacttctcaccacgtgactggcag |
| | MIPQYGLTLNNGSQAVGRSSSFYCLE | cgactcatcaacaacaactggggattccgatctaagcgactcaacttcaagctc |
| | YFPSQMLRTGNNFEFSYSFEDVPFHS | ctcaacattcaggtcaaagaagttacggacaacaatggagtcaagaccatcgcc |
| | SYAHSQSLDRLMNPLIDQYLYYLSRT | aataacctcaccagcaccatccaggtgtttacggactcggagtaccagctgccg |
| | QSTGGTAGTQQLLFSQAGPNNMSAQA | tacgttctcggctctgcccaccagggctgcccgcctccgttcccggcggacgtc |
| | KNWLPGPCYRQQRVSTTLSQNNNSNF | ttcatcattcctcagtacggctacctgactctcaacaatggcagtcaggccgtg |
| | AWTGATKYHLNGRDSLVNPGVAMATH | ggccgttcctccttctactgcctggagtactttccttctcaaatgctgagaacg |
| | KDDEERFFPSSGVLMFGKQGAGKDNV | ggcaacaactttgagttcagctacagcttcgaggacgtgcctttccacagcagc |
| | DYSSVMLTSEEEIKTTNPVATEQYGV | tacgcacacagccagagcctggaccggctgatgaatcccctcatcgaccagtac |
| | VADNLQQQNTAPQIGTVNSQGALPGM | ctgtactacctgtctcggactcagtccacgggaggtaccgcaggaactcagcag |
| | VWQNRDVYLQGPIWAKIPHTDGHFHP | ttgctattttctcaggccgggcctaataacatgtcggctcaggccaaaaactgg |
| | SPLMGGFGLKHPPPQILIKNTPVPAN | ctacccgggccctgctaccggcagcaacgcgtctccacgacactgtcgcaaaat |
| | PSTTFNQSKLNSFITQYSTGQVSVEI | aacaacagcaactttgcctggaccggtgccaccaagtatcatctgaatggcaga |
| | EWELQKENSKRWNPEIQYTSNYYKST | gactctctggtaaatcccggtgtcgctatggcaacccacaaggacgacgaagag |
| | SVDFAVNTEGVYSEPRPIGTHYLTRN | cgattttttccgtccagcggagtcttaatgtttgggaaacagggagctggaaaa |
| | L SEQ ID NO: 50 | gacaacgtggactatagcagcgttatgctaaccagtgaggaagaaattaaaacc |
| | | accaacccagtggccacagaacagtacggcgtggtggccgataacctgcaacag |
| | | caaaacacggctcctcaaattggaactgtcaacagccagggggccttacccggt |
| | | atggtctggcagaaccgggacgtgtacctgcagggtcccatctgggccaagatt |
| | | ccacacacggacggacattttcacccctctcccctcatgggtggattcggactt |
| | | aaacaccctcctccacagattctcatcaagaacaccccggtacctgcgaatcct |
| | | tcgaccaccctcaaccagtcaaagctgaactctctcaccacgcaatacagcacc |
| | | cgacaggtcagcgtcgaaattgaatcggagctgcagaaggaaaacaacaagcgc |
| | | tggaaccccgagatccagtacacctccaactactacaaatctacaagtgcggac |
| | | tttgccgttaatacagaaggcgtctactctgaacccccccccattggcacccat |
| | | tacctcacccgcaacctg-3′ SEQ ID NO: 74 |
| KJ03 | TAPAKKRPVEPSPQRSPDSSTGIGKK | 5′-acggctcctgcaaagaagagaccggtagagccgtcacctcagcgttccccc |
| (VP2) | GQQPARKRLNFGQTGDADSVPDPQPL | gactcctccacgggcatcggcaagaaaggccagcagcctgcaagaaaaagattg |
| | GEPPAAPSGVGPNTMASGGGAPMADN | aattttggtcagaccggagacgcagactcagtacctgacccccagcctcccgga |
| | NEGADGVGSSSGNWHCDSTWLGDRVI | gaacctccagcagcgccctctggtgtgggacctaatacaatggcttcaggcggt |
| | TTSTRTWALPTYNNHLYKQISSASTG | ggcgcaccaatggcagacaataacgaaggcgccgacggagtgggtagttcctca |
| | ASNDNHYFGYSTPWGYFDFNRFHCHF | ggaaattggcattgcgattccacatggctgggcgacagagtcatcaccaccagc |
| | SPRDWQRLINNNWGFRPKRLSFKLFN | acccgaacatgggccttgcccacctataacaaccacctctacaagcaaatctcc |
| | IQVKEVTQNEGTKTIANNLTSTIQVF | agtgcttcaacgggggccagcaacgacaaccactacttcggctacagcaccccc |
| | TDSEYQLPYVLGSAHQGCLPPFPADV | tgggggtattttgacttcaacagattccactgccacttttcaccacgtgactgg |
| | FMIPQYGYLTLNNGSQAVGRSSFYCL | caaagactcatcaacaacaactggggattccggcccaagagactcagcttcaag |
| | EYFPSQMLRTGNNFTFSYTFEDVPFH | ctcttcaacatccaggccaaggaggtcacgcagaatgaaggcaccaagaccatc |
| | SSYAHSQSLDRLMNPLIDQYLYYLSR | gccaataacctcaccagcaccatccaggtgtttacggactcggagtaccagctg |
| | TQSTGGTAGTQQLLFSQAGPNNMSAQ | ccgtacgttctcggctctgcccaccagggctgcctgcctccgttcccggcggac |
| | AKNWLPGPCYRQQRVSTLLSQNNNSN | gtcttcatgattcctcagtacggctacctgacgctcaacaatggcagccaggca |
| | FAWTGGTKYHLNGRNSLANPGIAMAT | gtgggacggtcatccttttactgcctggaatatttcccatcgcagatgctgaga |
| | HKDDEERFFPSNGILIFGKQNAARDN | acgggcaataactttaccttcagctacaccttcgaggacgtgcctttccacagc |
| | ADYSDVMLTSEEEIKTTNPVATEEYG | agctacgcgcacagccaaagcctcgaccggctgatgaaccccctcatcgaccag |
| | IVADNLQQQNTAPQIGTVNSQGALPG | tacctgtactacctgtctcggactcagtccacgggaggtaccgcaggaactcag |
| | MVWQNRDVYLQGPIWAKIPHTDGNFH | cagttgctattttctcaggccgggcctaataacatgtcggctcaggctaagaac |
| | PSPLMGGFGLKHPPPQILIKNTPVPA | tgcctacctcgaccttgctaccgacagcagcgagtctctacgacactgtcgcaa |
| | DPPTTFNQSKLNSFITQYSTGQVSVE | aacaacaacagcaactttgcttggactggtgggaccaaataccatctgaatgga |
| | IEWELQKENSKRWNPEIQYTSNYYKS | agaaattcattggccaatcctggcaccgctatggcaacacacaaagacgacgag |
| | TSVDFAVNTEGVYSEPRPIGTRYLTR | gagcgtttttttcccactaacggcatcctgatttttgccaaacaaaatgctgcc |
| | NL SEQ ID NO: 51 | agagacaatgcggattacagcgatgtcatgctcaccagcgaggaagaaatcaaa |
| | | accactaaccctgtggctacagaggaatacggtatcgtggcagataacttgcag |
| | | cagcaaaacacggctcctcaaattggaactgtcaacagccagggggccttaccc |
| | | ggtatggtctggcagaaccgggacgtgtacctgcagggtcctatctgggccaag |
| | | attcctcacacggacggaaactttcatccctcgccgctgatgggaggctttgga |
| | | ctgaaacacccgcctcctcagatcatgattaagaacacgcctgtacctgcggat |
| | | cccccgaccaccttcaaccagtcaaagctgaactcttccatcacgcaatacagc |
| | | accggacaggtcagcgtggaaattgaatgggagctacagaaggaaaacagcaag |
| | | cgctggaaccccgagatccagtacacctccaactactacaaatctacaagtgtg |
| | | gacttcgctcttaatacagaaggcgtgtactctgaaccccgccccatcggcacc |
| | | cgttacctccacccgtcaatctg-3′ SEQ ID NO: 75 |
| KJ04 | TAPGKKRPVEPSPQRSPDSSTGIGKK | 5′-acggctcctggaaagaagagaccggtagaaccgtcacctcagcgttccccc |
| (VP2) | GQQPAKKRLNFGQTGDSESVPDPQPI | gactcctctacgggcatoggcaagaaaggccagcagcccgcgaaaaagagactc |
| | GEPPAAPSGLGPNTMAAGGGAPMADN | aactttgggcagaccggcgactcagagtcagtgcccgaccctcaaccaaccgga |
| | NEGADGVGSSSGNWHCDSTWLGDRVI | gaacctccagcagccccctcaggtctgggacctaatacaatggctgcaggcggt |
| | TTSTRTWALPTYNNHLYKQISNGTSG | ggcgcaccaatggcagacaataacgaaggagccgacggagcgggtagttcctcg |
| | GATNDNTYFGYSTPWGYFDFNRFHCH | ggaaattggcattgcgattccacatggctgggcgacagagtcatcaccaccagc |
| | PFPRDWQRLINNNWGFRPKRLSPKLF | acccgaacctgggccctgcccacctacaacaaccacctctacaagcaaatctcc |
| | NIQVKEVTQNEGTKTIANNLTSTIQV | aacgggacatcgggaggagccaccaacgacaacacctacttcggctacagcacc |
| | FTDSEYQLPYVLGSAHQGCLPPFPAD | ccctgggggtatttcgacctcaacagatcccactgccacttctcaccacgtgac |
| | VFMIPQYGYLTLNNGSQAVGRSSFYC | cggcaacgactcatcaacaacaattggggattccggcccaaaagactcagcttc |
| | LEYFPSQMLRTGNNFQFTYTFEDVPF | aagctcttcaacatccaggtcaaggaggtcacgcagaatgaaggcaccaagacc |
| | HSSYAHSQSLDRLMNPLIDQYLYYLS | atcgccaataacctcaccagcaccatccaggtgtttacgaactcggagtaccag |
| | RTQTTGGTANTQTLGFSQGGPNTMAN | ctgccgtacgttctcggctctgcgcaccagggctgcctggctccgttcccggcg |
| | QAKNWLPGPCYRQQRVSTTTGQNNNS | gacgtcttcatgattcctcagtacgggtacctgactccgaacaacggcagtcag |
| | NFAWTAGTKYHLNGRNSLANFGIAMA | gccgtgggccgttcctccttctactgcctggaatattttccatcgcagatgctg |
| | THKDDEERFFPSNGILIFGKQNAARD | agaaccggcaacaacttccagtttacttacaccttcgaggacgtgcctttccac |
| | NADYSDVMLTSEEEIKTTNPVATEEY | agcagctacgcccacagccagagcttggaccggctgacgaatcctctgattgac |
| | GIVADNLQQQNTAPQIGTVNSQGALP | cagtacctgtactacttgtctcggactcaaacaacaggaggcacggcaaatacg |
| | GMVWQNRDVYLQGPIWAKIPHTDGNF | cagactctgggcttcagccaaggtgggcctaatacaatggccaatcaggcaaag |
| | HPSPLMGGFGLKHPPPQILIKNTPVP | aactggctgccaggaccctgttaccgccaacaacgcgtctcaacgacaacccgg |
| | ANPPEVFTPAKFASFITQYSTGQVSV | caaaacaacaatagcaactttgcctggactgctgggaccaaataccacctgaat |
| | EIEWELQKENSKRWNPEIQYTSNFEK | ggaagaaattcattggctaatcctggcatcgctatggcaacacacaaagacgac |
| | QTGVDFAVDSQGVYSEPRPIGTRYLT | gacgagcgtttttttcccagtaacgggatcctgatttttggcaaacaaaatgct |
| | RNL SEQ ID NO: 52 | gccagagacaatgcggattacagcgatgtcatgctcaccagcgaggaagaaatc |
| | | aaaaccactaaccccgtggctacagaggaatacggcatcgtggcagataacttg |
| | | cagcagcaaaacacggctcctcaaattggaactgtcaacagccagggggcctta |
| | | cccggtatggtctggcagaaccgggacgtgtacctgcagggtcccatctgggcc |
| | | aagattcctcacacggacggcaactcccacccgtctccgctgatgggcggcttt |
| | | ggcctgaaacatcctccgcctcagatcctgatcaagaacacgcccgttcccgct |
| | | aatcctccggaggtgtttactcctgccaagtttgcttcgttcatcacacagtac |
| | | agcaccggacaagtcagcgtggaaaccgagtgggagctgcagaaggaaaacagc |
| | | aagcgctggaacccggagattcagtacacctccaactttgaaaagcagactggt |
| | | gtggactttgccgttgacagccagggtgtttactctgagcctcgccctattggc |
| | | actcgttacctcacccgcaacctg-3′ SEQ ID NO: 76 |
| KJ05 | TAPGKKRPVEPSPQRSPDSSTGIGKK | 5′-acggctcctggaaagaagagaccggtagagccatcaccccagcgctctcca |
| (VP2) | GQQPAKKRLNFGQTGDTESVPDPQPI | gactcctctacgggcatcggcaagaaaggccagcagcccgccaaaaagagactc |
| | GEPPAAPSGVGSLTMASGGGAPVADN | aatttcggtcagactggcgacacagagtcagtcccagaccctcaaccaatcgga |
| | NEGADGVGNASGNWHCDSTWLGDRVI | gaacctcccgcagccccctcaggtgtgggatctcttacaatggcttcaggtggt |
| | TTSTRTWALPTYNNHLYKQISNSTSG | ggcgcaccagtggcagacaataacgaaggtgccgacggagtgggtaatgcctca |
| | GSSNDNAYFGYSTPWGYFDFNGFHCH | ggaaattggcattgcgattccacatggccgggcgacagagtcatcaccaccagc |
| | FSPRDWQRLINNNWGFRPKRLGFKLF | acccgaacatgggccctgcccacctacaacaaccacctctacaagcaaatctcc |
| | NIQVKEVTQNEGTKTIANNLTSTIQV | aacagcacatctggaggatcttcaaatgacaacgcctacttcggctacagcacc |
| | FTDSEYQLPYVLGSAHQGCLPPFPAD | ccctgggggtatttcgacctcaacggattccactgccatttctcaccacgtgac |
| | VFMIPQYGYLTLNNGSQAVGRSSFYC | tggcagcgactcatcaacaacaattggggattccggcccaagagactcggcttc |
| | LEYFPSQMLRTGNNFEFSYTFEDVFP | aagctcttcaacatccaggtcaaggaggtcacgcagaatgaaggcaccaagacc |
| | HSSYAHSQSLDRLMNPLIDYYLYYLS | atcgccaataacctcaccagcaccacccaggtgtttacggactcggagtaccag |
| | RTQTTGGTANTQTLGFSQGGPNTMAN | ctgccgtacgttctcggctctgcccaccagggctgcctgcctccgttcccggcg |
| | QAKNWLPGPCYRQQRVSTTTGQNNNS | gacgtgttcatgattcctcagtacgggtacctgactctgaacaatggcagtcag |
| | NFAWTAGTKYHLNGRNSLANPGIAMA | gccgtgggccgttcctccttctactccctggagtactttccttctcaaatgctg |
| | THKDDEERFFPSNGILIFGKQNAARD | cgaactggaaacaattttgaactcagctacaccttcgaggacgtgcctttccac |
| | NADYSDMVLTSEEEIKTTNPVATEEY | agcagctacgcgcacacccagagcctggacaggctgacgaatcccctcatccac |
| | GIVADNLQQQNTAPQIGTVDSQGALP | tactacctgtactacttgtctcggactcaaacaacaggaggcacggcaaatacg |
| | GMVWQNRDVYLQGPIWAKIPHTDGNF | cagactctgggcttcagccaaggtgggcctaatacaatggccaatcaggcaaag |
| | HPSPLMGGFGLKHPPPQILIKNTPVP | aactggctgccaggaccctgttaccgccaacaacgcgcctcaacgacaaccggg |
| | ADPPTTFNQSKLNSFITQYSTGQVSV | caaaacaacaatagcaactttgcctggactgctgggaccaaataccatctgaat |
| | EIEWELQKENSKRWNPEIQYTSNYYK | ggaagaaattcattggctaatcctggcaccgctatggcaacacacaaagacgac |
| | STSVDFAVNTEGVYSEPRPIGTRYLT | gaggagcgtcttcttcccagtaacgggatcctgatttctggcaaacaaaatgct |
| | RNL SEQ ID NO: 53 | gccagagacaatgcggattacagcgatgtcatgctcaccagcgaggaagaaatc |
| | | aaaaccactaaccctgtggctacagaggaatacggtatcgtggcagataacttg |
| | | caccagcaaaacacggctcctcaaattggaactgtcgacagccacggggcctta |
| | | cccggtatggtctggcagaaccgggacgtgtacctgcagggtcacatctgggcc |
| | | aagattcctcacacggaccgcaactcccacccgtccccgctgatgggcggcttt |
| | | ggcctgaaacatcctccgcctcacatcctgatcaagaacacgcctgtacctgcg |
| | | gatcctccgaccaccttcaaccagtcaaagctgaactctttcatcacgcaatac |
| | | agcaccggacaggtcagcgtggaaactgaatgggagctacagaaggaaaacagc |
| | | aagcgctggaaccccgagatccagtacacctccaactactacaaacctacaagt |
| | | gtggactttgctgttaatacagaaggcgtttactctgagcctcgccctattggg |
| | | actcgttacctcacccgtaatctg-3′ SEQ ID NO: 77 |
| HW01 | TAPGKKRPVEQSPQEPDSSAGIGKSG | 5′-acggctcctggaaagaagaggcccgtagagcagtctcctcaggaaccggac |
| (VP2) | AQPAKKRLNFGQTGDSESVPDPQPLG | tcctccgcgggtattggcaaatcgggtgcacagcccgctaaaaagagactcaat |
| | EPPATPAAVGPTTMAAGGGAPMADNN | tttggtcagactggcgactcagagtcagtccccgatccacaacctctcggagaa |
| | EGADGVGNSSGNWHCDSTWLGDRVIT | cctccagcaacccccgctgctgtgggacctactacaatggctgcaggcggtggc |
| | TSTRTWALPTYNNHLYKQISNSTSGG | gctccaatggcagacaataacgaaggcgccgacggagtgggtaattcctcggga |
| | SSNDNAYFGYSTPWGYFDFNRFHCHF | aattggcattgcgattccacatggctgggggacagagtcatcaccaccagcacc |
| | SPRDWQRLINNNWGFRPKRLNFKLFN | cgaacctgggccctgcccacctacaacaaccacctctacaagcaaatctccaac |
| | IQVKEVTQNEGTKTIANNLTSTIQVF | agcacatctggaggatcttcaaatgacaacgcctacttcggctacagcaccccc |
| | TDSEYQLPYVLGSAHQGCLPPFPADV | tgggggtattttgacttcaacagatcccactgccacttctcaccacgtgactgg |
| | FMIPQYGYLTLNNGSQAVGRSSFYCL | caccgactcatcaacaacaactgcggattccggcccaagagactcaacttcaag |
| | EYFPSQMLRTGNNFQFTYTFEDVPFH | ctcttcaacatccaggtcaaggaggtcacgcagaatgaaggcaccaagaccatc |
| | SSYAHSQSLDRLMNPLIDQYLYYLSR | gccaataacctcaccagcaccatccaggcgtttacggactcggagtaccagctg |
| | TQSTGGTAGTQQLLFSQAGPNNMSAQ | ccgtacgttctcggctctgcccacaagggctgcctgcctccgttcacggcggac |
| | AKNWLPGPCYRQQRVSKTSADNNNSE | gtgttcatgattccccagtacggctacctaacactcaacaacggtagtcaggcc |
| | YSWTGATKYHLNGRDSLVNPGPAMAS | gtgggacgctcctccttctaccgcctggaatacttcccttcgcagatgccgaga |
| | HKDDEEKFFPQSGVLIFGKQGSEKTN | accggcaacaacttccagtttacttacaccttcgaggacgtgcctttccacagc |
| | VDIEKVMITDEEEIRTTNPVATEQYG | agctacgcccacagccagagcttggaccggctgatgaaccccctcatcgaccag |
| | SVSTNLQRGNRQAATADVNTQGVLPG | tacctgtactacctgtctcggactcagtccacgggaggtaccgcaggaactcag |
| | MVWQDRDVYLQGPIWAKIPHTDGHFH | cagttggcattttctcaggccgggcctaataacatgtcggctcaggccaaaaac |
| | PSPLMGGFGLKHPPPQILIKNTPVPA | tggctacccgggccctgctaccggcagcagcgagtatcaaagacatctgcggat |
| | DPPTTFNQSKLNSFITQYSTGQVSVE | aacaacaacagtgaatactcgtggactggagctaccaagtaccacctcaatggc |
| | IEWELQKENSKRWNPEIQYTSNYYKS | agagactctctggtgaatccgggcccggccatggcaagccacaaggacgatgaa |
| | TSVDFAVNTEGVYSEPRPIGTRYLTR | gaaaagtttcttcctcagagcggggtcctcatctttgcgaagcaaggctcagag |
| | NL SEQ ID NO: 54 | aaaacaaatgtggacattgaaaaggtcatgattacagacgaagaggaaatcagg |
| | | acaaccaatcccgtggctacggagcagtatggttctgtatctaccaacctccag |
| | | agaggcaacagacaagcagctaccgcagatctcaacacacaaggcgttcttcca |
| | | ggcatggtatggcaggacagagatgagtaccttcaggggcccatctgggcaaag |
| | | attccacacacggacggacattttcacccctctcccctcatgggtggattcgga |
| | | cttaaacaccctcctccacagatcctgatcaagaacacgcctgtacctgcggat |
| | | cctccgaccaccttcaaccagtcaaagctgaactctttcaccacgcaatacagc |
| | | accggacaggtcagcgtgcaaattgaatgggagctgcagaaggaaaacagcaag |
| | | cgctggaaccccgagatccagtacacctccaactactacaaatctacaagtctg |
| | | gactttgctgttaatacagaaggcgtgtactctgaaccccgccccattggcacc |
| | | cgttacctcacccgcaacctg-3′ SEQ ID NO: 73 |
| HW02 | TAPGKKRPVEHSPVEPDSSSGTGKAG | 5′-acggctccgggaaaaaagaggccggtagagcactctcctgtggagccagac |
| (VP2) | QQPARKRLNFGQTGDADSVPDPQPLG | tcctcctcgggaaccggaaaggcggcccagcagcctgcaagaaaaagattgaat |
| | QPPAAPSGLGTNTMATGSGAPMADNN | tttggtcagactggagacgcagactcagtacctgacccccagcctctcggacag |
| | EGADGVGNSSGNWHCDSTWMGDRVIT | ccaccagcacccccctctggtctgggaactaatacgatggctacaggcagtcgc |
| | TSTRTWALPTYNNHLYKQISSQSGAS | gcaccaatggcagacaataacgaggccgccgacggagtgggtaattcctcggga |
| | NDNHYFGYSTPWGYFDFNRFHCHFSP | tattggcattgcgattccacatggacgggcgacagagtcatcaccaccagcacc |
| | RDWQRLINNNWGFRPKRLNFKLFNIQ | cgaacctgggccctgcccacctacaacaaccacctctacaaacaaatctccagc |
| | VKEVTQNDGTTTIANNLTSTVQVFTD | caatcaggagcctcgaacgacaatcactactttggctacagcaccccttggggg |
| | SEYQLPYVLGSAHQGCLPPFPADVFM | tattttgacttcaacagactccactgccacttttcaccacgtgactggcaaaga |
| | VPQYGYLTLNNGSQAVGRSSFYCLEY | ctcatcaacaacaactcgggattccgacccaagagactcaacttcaagctcttt |
| | FPSQMLRTGNNFTFSYTFEDVPFHSS | aacattcaagtcaaagaggtcacgcagaatgacggtacgacgacgattgccaat |
| | YAHSQSLDRLMNPLIDQYLYYLSRTN | aaccttaccagcacggttcaggtgtctactgactcggagtaccagctcccgtac |
| | TPSGTTTQSRLQFSQAGASDIRDQSR | gtcctcggctcggcgcatcaaggatgcctcccgccgttcccagcagacgtcttc |
| | NWLPGPCYRQQRVSKTSADNNNSEYS | atggtgccacagtatggatacctcaccctgaacaacgggagtcaggcagtagga |
| | WTGATKYHLNGRDSLVNPGPAMASHK | cgctcttcattttactgcctggagtactttccttcccagatgctgcgtaccgga |
| | DDEEKFFPQSGVLIFGKQGSEKTNVD | aacaactttaccttcacctacacttttgaggacgttcctttccacagcagctac |
| | IEKVMITDEEEIRTTNPVATEQYGSV | gctcacagccagagtctggaccgtctcatgaatcctctcatcgaccagtacctg |
| | STNLQRGNRQAATADVNTQGVLPGMV | cattacttgagcagaacaaacactccaagtggaaccaccacgcagtcaaggctt |
| | WQDRDVYLQGPIWAKIPHTDGHFHPS | cagttttctcaggccggagcgagtgacattcgggaccagtctaggaactggctt |
| | PLMGGFGLKHPPPQILIKNTPVPADF | cctggaccccgttaccgccagcagcgagtatcaaagacatctgcggataacaac |
| | PTTFNQSKLNSFITQYSTGQVSVEIE | aacagtgaatactcgtggactggagctaccaagtaccacctcaatggcacagac |
| | WELQKENSKRWNPEIQYTSNYYKSTS | tctctggtgaatccgggcccggccatggcaagccacaaggacgatgaagaaaag |
| | VDFAVNTEGVYSEPRPIGTRYLTRNL | ttctttcctcagagcgcggttctcatctttcggaagcaaggctcagagaaaaca |
| | SEQ ID NO: 55 | aatgtggacattgaaaaggtcatgattacagacgaagaggaaatcaggacaacc |
| | | aatcccgtggctacggagcagtatggttctgtatctaccaacctccagagaggc |
| | | aacagacaagcagctaccgcagatgtcaacacacaaggcgttctcccaggcatg |
| | | gtctggcaggacagagatgtgtaccttcaggggcccatctgggcaaagattcca |
| | | cacacggacggacattttcacccctctcccctcatgggtggattcggacttaaa |
| | | caccctcctccacagatcctgatcaagaacacgcctgcacctgcggatcctccg |
| | | accaccttcaaccagtcaaagctgaactctttcatcacgcaatacagcaccgga |
| | | caggtcagcgtggaaattgaatgggagccgcagaaggaaaacagcaagcgctgg |
| | | aaccccgagatccagtacacctccaactactacaaatctacaagtgtggacttt |
| | | gctgttaatacagaaggcgtgtactctgaaccccgccccattggcacccgttac |
| | | ctcacccgcaacctg-3′ SEQ ID NO: 73 |
| HW03 | TAPGKKRPVEQSPQEPDSSAGIGKSG | 5′-acggctcctggaaagaagaggcctgtagagcagtctcctcaggaaccggac |
| (VP2) | AQPAKKRLNFGQTGDSESVPDPQPLG | tcctccgcgggtattggcaaatcgggtgcacagcccgctaaaaagagactcaat |
| | EPPATPAAVGPTTMAAGGGAPMADNN | tttggtcagactggcgactcagagtcagtccccgatccacaacctctoggagaa |
| | EGADGVGNSSGNWHCDSTWLGDRVIT | cccccagcaacccccgctgctgtcggacctactacaacggctgcaggcggtggc |
| | TSTRTWALPTYNNHLYKQISNSTSGG | gctccaatggcagacaataacgaaggcgccgacggagtgggtaattcctcggga |
| | SSNDNAYFGYSTPWGYFDFNRFHCHF | aattggcattgcgattccacatggctgggggacagagtcatcaccaccagcacc |
| | SPRDWQRLINNNWGFRPKRLNFKLFN | cgaacctggcccctgcccacctacaacaaccacctctacaagcaaatctccaac |
| | IQVKEVTQNEGTKTIANNLTSTIQVF | agcacatctggaggatcttcaaatgacaacgcctacttcggctacagcaccccc |
| | TDSEYQLPYVLGSAHQGCLPPFPADV | tgggggtattttgacttcaacagattccactgccacttctcaccacgtgactgg |
| | FMIPQYGYLTLNNGSQAVGRSSFYCL | cagcgactcatcaacaacaactgcggattccggcccaagagactcaacttcaag |
| | EYFPSQMLRTGNNFQFTYTFEDVPFH | ctcttcaacatccaggtcaaggaggtcacgcagaatgaaggcaccaagaccatc |
| | SSYAHSQSLDRLMNPLIDQYLYYLSR | gccaataacctcaccagcaccatccaggtgtttacggactcggagtaccagctg |
| | TSSTGGTAGTQQLLFSQAGPNNMSAQ | ccgtacgttctcggctctgcccaccagggctgcctgcctccgttcccggcggac |
| | AKNWLPGPCYRQQRVSKTSADNNNSE | gtgttcatgattccccagtacggctacctaacactcaacaacggtagtcaggcc |
| | YSWTGATKYHLNGRDSLVNPGPAMAS | gtgggacgctcctccttctaccgcccggaatacttcccttcgcagatgccgaga |
| | HKDDEEKFFPQSGVLIFGKQGSEKTN | accggcaacaacttccagtttacttacaccttcgaggacgtgcctttccacagc |
| | VDIEKMVITDEEEIRTTNPVATEQYG | agctacgcccacagccagagcttggaccggctgatgaaccccctcatcgaccag |
| | SVSTNLQRGNRQAATADVNTQGVLPG | cacctgtactacctgtctcggactcagtccacgggaggtaccgcaggaactcag |
| | MVWQDRDVYLQGPIWAKIPHTDGHFH | cagttgctattttctcaggccgggcctaataacatgtcggctcaggccaaaaac |
| | PSPLMGGFGLKMPPPQILIKNTPVPA | tggctacccgggccctgctaccggcagcagcgagtatcaaagacatctgcggat |
| | DPPTTFNQSKLNSFITQYSTGQVSVE | aacaacaacagtgaatactcgtggactggagctaccaagtaccacctcaatggc |
| | IEWELQKENSKRWNPEIQYTSNYYKS | agagcctctctggtgaatccgggcccggccatggcaagccacaaggacgatgaa |
| | TSVDFAVNTEGVYSEPRPIGTRYLTR | gaaaagttttttcctcagagcggggttctcatctttgggaagcaaggctcagag |
| | NL SEQ ID NO: 56 | aaaacaaatgtagacattcaaaaggtcatgattacagacgaagaggaaatcagg |
| | | acaaccaatcccgtggctacggagcagtatggttctgtatctaccaacctccag |
| | | agaggcaacagacaagcagctaccgcagatgtcaacacacaaggcgttcttcca |
| | | ggcatggtctggcaggacagagatgtgtaccttcaggggcccatctgggcaaag |
| | | attccacacacggacggacattttcacccctctcccctcatgggtggattcgga |
| | | ctcaaacaccctcctccacagatcctgatcaagaacacgcctgtacctgcggat |
| | | cctccgaccaccttcaaccagtcaaagctgaactctttcatcacgcaatacagc |
| | | accggacaggtcagcgtggaaattgaatgggagctgcagaaggaaaacagcaag |
| | | cgctggaaccccgagacccagtacacctccaaccactacaaatctacaagtctg |
| | | gactttgctgttaatacagaaggcgtgtactctgaaccccgccacatcggcacc |
| | | cgttacctcacccgcaacctg-3′ SEQ ID NO: 80 |
| HW04 | TAPGKKRPVEPSPQRSPDSSSGIGKK | 5′-acggctcctggaaagaagagaccggtagagccatcaccccagcgttctcca |
| (VP2) | GQQPARKRLNFGQTGDSESVPDPQPL | gactcctcctcgggcatcggcaagaaaggccaacagcccgccagaaaaaaactc |
| | GEPPAAPSGVGPNTMASGGGAPMADN | aatttgggccagactggcgactcagagtcagttccagaccctcaacctctcgga |
| | NEGADGVGNASGNWHCDSTWLGDRVI | gaacccccagcagcgccctctggtgtgggacctaatacaatggcttcaggcggt |
| | TTSTRTWALPTYNNHLYKQISNSTSG | ggcgcaccaatggcagacaataacgaaggcgccgacggagtgggtaatgcctca |
| | GSSNDNAYFGYSTPWGYFDFNRFHCH | ggaaattggcattgcgattccacatggctgggcgatagagtcatcaccaccagc |
| | FSPRDWQRLINNNWGFRPKRLNFKLF | acccgaacctgggccctccccacctacaacaatcacctctacaagcaaatctcc |
| | NIQVKEVTTNDGVTTIANNLTSTVQV | aacagcacatctggaggatcttcaaatgacaacgcctacttcggctacagcacc |
| | FSDSEYQLPYVMLGSHQGCLPPFPAD | ccctgggggtattttgacttcaacagattccactgccatttctcaccacgtgac |
| | VFMIPQYGYLTLNNGSQSVGRSSFYC | tggcagcgactcatcaacaacaattggggattccggcccaagagactcaacttc |
| | LEYFPSQMLRTGNNFTFSYTFEDVPF | aagctcttcaacatccaactcaaggaggtcacgacgaataatggcgtcacgacc |
| | HSSYAHSQSLDRLMNPLIDQYLYYLS | atcgctaataaccttaccagcacggttcaagtcttctcggactcggagtaccag |
| | RTQSTGGTQGTQQLLFSQAGPANMSA | ttgccgtacgtcctcggctctgcgcaccagggctgcccgcctccgttcccggcg |
| | QAKNWLPGPCYRQQRVSKTSADNNNS | gacgtgttcatgattcctcagtacggctacctgactctcaacaacggcagtcag |
| | EYSWTGATKYHLNGRDSLVNPGPAMA | tctgtgggccgttcctccttctactgcctggaatatttcccatcgcagatgctg |
| | SHKDDEEKFFPQSGVLIFGKQGSEKT | agaacgggcaataactttaccttcagctacaccttcgaggacgtgcctttccac |
| | NVDIEKVMITDEEEIRTTNPVATEQY | agcagctacgcccacagccagagcttggaccggctgatgaatcctctcatcgac |
| | GSVSTNLQRGNRQAATADVNTQGVLP | cagtacctgtactacttatccagaactcagtccacaggaggaactcaaggtacc |
| | GMVWQDRDVYLQGPIWAKIPHTDGHF | cagcaattgttattttctcaagctgggcctgcaaacatgtcggctcaggctaag |
| | HPSPLMGGFGLKHPPPQILIKNTPVP | aactggctacctggaccttgctaccggcagcagcgagcatcaaagacatctgcg |
| | ANPSTTFNQSKLNSFITQYSTGQVSV | gataacaacaacagtgaatactcgtcgactggagctaccaagtaccacctcaat |
| | EIEWELQKENSKRWNPEIQYTGQVSV | ggcagagactctctggtgaatccgggcccgcccatggcaagccacaaggaccat |
| | EIEWELQKENSKRWNPEIQYTSNYYK | gaagaaaagcttcttcctcagagcggggttctcatctctgggaagcaaggccca |
| | STNVDFAVNTEGVYSEPRPIGTRYLT | gagaaaacaaatgtggacattgaaaaggtcatgattacagacgaagaggaaatc |
| | RNL SEQ ID NO: 57 | aggacaaccaatcccgtggctacggagcagtatggttctgtatctaccaacctc |
| | | cagagaggcaacagacaagcagccaccgcagatgtcaacacacaaggcgttctt |
| | | ccaggcatggtctggcagcacagagatgtgtaccttcaggggcccatctcggca |
| | | aagattccacacacggacggacattttcaccactctcccctcatgggtggattc |
| | | ggacttaaacaccctcctccacacattctcatcaagaacacccccgtacctgcg |
| | | aatccttcgaccaccttcaaccagtcaaagctgaactctttcatcacgcaatac |
| | | agcaccggacaagtcagcgtggaaaccgagtgggagctgcagaaggaaaacagc |
| | | aagcgctggaatccagagattcaatacacttccaactactacaaatccacaaat |
| | | gtggactttgctgtcaacacggagggggtttatagtgagcctcgccccattggc |
| | | acccgttacctcacccgtaatctg-3′ SEQ ID NO: 81 |
| HW05 | TAPGKKRPVEPSPQRSPDSSTGIGKK | 5′-acggctcctggaaagaagagaccggtagagccgtcacctcagcgttccccc |
| (VP2) | GQQPAKKRLNFGQTGDSESVPDPQPI | gactcctccacgggcatcggcaagaaaggccagcagcccgctaaaaagagactg |
| | GEPPAAPSGVGPNTMAAGGGAPMADN | aactttggtcagactggcgactcagagtcagtgcccgaccctcaaccaatcgga |
| | NEGADGVGSSSGNWHCDSTWLGDRVI | gaacctccagcagcgccctctggtgtgggacctaatacaatggccgcaggcggt |
| | TTSTRTWALPTYNNHLYKQISSASTG | ggcgcaccaatggcagacaataacgaaggcgccgacggagtgggtaattcctcg |
| | ASNDNTYFGYSTPWGYFDFNRFHCHF | ggaaattggcattgcgatcccacatggctgggcgacagagtcatcaccaccagc |
| | SPRDWQRLINNNWGFRPKKLRFKLFN | acccgaacacgggcctcgcccacctacaacaaccacccctacaagcaaatcccc |
| | IQVKEVTQNEGTKTIANNLTSTVQVF | agtgcttcaacgggggccagcaacgacaacacctacttcggctacagcaccccc |
| | TDSEYQLPYVLGSAHQGCLPPFPADV | tgggggtattttgacttcaacagatcccactgtcacttttcaccacgtgactgg |
| | FMVPQYGYLTLNNGSQALGRSSFYCL | caacgactcatcaacaacaactgcggattccggcccaagaagctgcggttcaag |
| | EYFPSQMLRTGNNFQFTYTFEDVPFH | ctcttcaacatccaggtcaaggaggtcacgcagaatgaaggcaccaagaccatc |
| | SSYAHSQSLDRLMNPLIDQYLYYLSR | gccaataatctcaccagacccgtgaaggtctttacggactcggagtaccagtta |
| | TQSTGGTAGTQQLLFSQAGPNNMSAQ | ccgtacgtgctaggatccgctcaccagggatgtctgcctccgttcccggcggac |
| | AKNWLPGPCYRQQRVSKTSADNNNSE | gtcttcatggttcctcagtacggctatttaactttaaacaatggaagccaagcc |
| | YSWTGATKYHLNGRDSLVNPGPAMAS | ctgggacgttcctccttccaccgtctggagtatttcccatcgcagatgccgaga |
| | HKDDEEKFFPQSGVLIFGKQGSEKTN | accggcaacaactttcagtttacctacaccttcgaggacgcgcctttccacagc |
| | VDIEKVMITDEEEIRTTNPVATEQYG | agctacgcgcacagccagagcctggacaggctgatgaatcccctcatcgaccag |
| | SVSTNLQRGNRQAATADVNTQGVLPG | cacctgtactacctgtctcggactcagtccacgggaggtaccgcaggaactcag |
| | MVWQDRDVYLQGPIWAKIPHTDGNFH | cacttgctattttctcaggccgggcctaataacatgtcggctcaggccaaaaac |
| | PSPLMGGFGMKHPPPQILIKNTPVPA | tggctacccgggccctgctaccggcagcagcgagtatcaaagacatctgcggat |
| | DPPTTFSQAKLASFITQYSTGQVSVE | aacaacaacagtgaatactcgcggactggagctaccaagtaccacctcaatggc |
| | IEWELQKENSKRWNPEIQYTSNYYKS | agagactctctggtgaatccgggcccggccatggcaagccacaacgacgatcaa |
| | TNVDFAVNTEGTYSEPRPIGTRYLTR | gaaaagtttcttcctcagagcggggttctcatctttgggaagcaaggctcagag |
| | NL SEQ ID NO: 58 | aaaacaaatgtggacattcaaaaggccatgattacagacgaagaggaaaccagg |
| | | acaaccaatcccgtggctacggagcagtatggttctgtatctaccaacctccag |
| | | agaggcaacagacaagcagctaccgcagatgtcaacacacaaggcgtccttcca |
| | | ggcatggtctggcaggacagagatgtgtaccttcaggggcccatctgggcaaag |
| | | attccacacacggacggcaactttcacccttctccgctgatgggagggtttggc |
| | | atgaagcacccacctcctcagatcctgatcaagaacacgccggtacctgcggat |
| | | cctccaacaacattcagccaggcgaaattggcttccttcattacgcagtacagc |
| | | accggacaggtcagcgtggaaatcgagtgggagctgcagaaggagaacagcaaa |
| | | cgctggaacccagagactcagtacacttcaaactactacaaatccacaaatgtg |
| | | gactttgctgtcaatacagagggaacttattctgagcctcgccccattggtact |
| | | cgttaccccacccgcaatctg-3′ SEQ ID NO: 82 |
| HW06 | TAPGKKRPVEQSPQEPDSSSGIGKKG | 5′-acggctcctggaaagaagaggcctgtagagcagtctcctcaggaaccggac |
| (VP2) | QQPAKKRLNFGQTGDSESVPDPQPIG | tcctccgcgggtattggcaaatcggctgcacagcccgctaaaaagagactcaat |
| | EPPAAPSSVGSGTVAAGGGAPMADNN | ttcggtcagactggcgacacagagtcagtcccagaccctcaaccaatcggagaa |
| | EGADGVGNASGNWHCDSTWLGDRVIT | ccccccggagccccctctagtgtgggatctggtacagcggctgcaggcggtggc |
| | TSTRTWALPTYNNHLYKQISSETAGS | gcaccagcggcagacaataacgaaggtgccgacggagtgggtaatgcctcagga |
| | TNDNTYFGYSTPWGYFDFNRFHCHFS | aattggcattgcgattccacatggctgggcgacagagtcattaccaccagcacc |
| | PRDWQRLINNNWGFRPKRLNFKLFNI | cgaacctggaccctgcccacctacaacaaccacctctacaagcaaatctccagt |
| | QVKEVTTNEGTKTIANNLTSTVQVFT | gaaactgcaggtagtaccaacgacaacacctacttcggctacagcaccccctgg |
| | DSEYQLPYVLGSAHQGCLPPFPADVF | gggtattttgacttcaacagattccactgtcacttttcaccacgtgactggcaa |
| | MIPQYGYLTLNNGSQAVGRSSFYCLE | cgactcatcaacaacaattggggattccggcccaaaacactcaacttcaagctg |
| | YFPSQMLRTGNNFEFSYSFEDVPFHS | ttcaacatccaggtcaaggaagtcacgacgaacgaaggcaccaagaccatcgcc |
| | SYAHSQSLDRLMNPLIDQYLYYLSRT | aataatctcaccagcaccgtgcaggtctttacggactcggaataccagctcccg |
| | NTPSGTTTQSRLQFSQAGASDIRDQS | tacgtcctcggctctgcgcaccagggctgcctgcctccgttcccggcggacgtc |
| | RNWLPGPCYRQQRVSKTSADNNNSEY | ttcatgattcctcagtacgggtacctgactctgaacaatggaagtcaagccgta |
| | SWTGATKYHLNGRDSLVNPGPAMASH | ggccgttcctccttctactgcctggaatatttcccatcgcagatgctgagaacg |
| | KDDEEKFFPQSGVLIFGKQGSEKTNV | ggcaacaactttgagttcagctacagcttcgaggacgttcctttccacagcagc |
| | DIEKVMITDEEEIRTTNPVATEQYGS | tacgctcacagccagagtctggaccgtctcatgaatcctctcatcgaccagtac |
| | VSTNLQRGNRQAATADVNTQGVLPGM | ctgtattacttaagcagaacaaacactccaagtggaaccaccacgcagtcaagg |
| | VWQDRDVYLQGPIWAKIPHTDGHFHP | cttcagttttctcaggccggagcgagtgacattcaggaccagtctaggaactgg |
| | SPLMGGFGLKHPPPQILIKNTPVPAD | ctccctggaccctgttaccgccagcagcgagtatcaaagacatctgcggataac |
| | PPTTFSQAKLASFITQYSTGQVSVEI | aacaacagtgaatactcgtggactggagctaccaagtaccacctcaatggcaga |
| | EWELQKENSKRWNPEIQYTSNYYKST | gactctctggtgaatccgggcccggccatggcaagccacaaggacgatgaagaa |
| | NVDFAVNTEGVYSEPRPIGTRYLTRN | aagttctttcctcagagcggggtcctcatccttgggaagcaaggctcagagaaa |
| | L SEQ ID NO: 60 | acaaatgtggacattgaaaaggtcatgattacagccgaagaggaaatcacgaca |
| | | accaatcccgtggctacggagcagtatggttctgtatctaccaacctccagaga |
| | | ggcaacagacaagcagctaccgcagatgtcaacacacaaggcgttctcccaggc |
| | | atggtctggcaggacagagatgtgtaccttcaggggcccatctgggcaaagatt |
| | | ccacacacggacggacattttcacccctctcctctcaagggcggctttggactt |
| | | aagcacccgcctcctcagatcctcatcaaaaacacgcctgttcccgcggatcct |
| | | ccaactaccttcagtcaagctaagctggcgtcgttcaccacgcagtacagcacc |
| | | ggacaggtcagcgtggaaatcgagtgggagctgcagaaggagaacagcaaacgc |
| | | tggaacccagagattcaatacacttccaactactacaaatctacaaatgtggac |
| | | tttgctgttaatacagaaggcgtgtactctgaaccccgccccattggcacccgt |
| | | tacctcacccgtaacctg-3′ SEQ ID NO: 84 |
| HW08 | TAPGKKRPVEPSPQRSPDSSTGIGKK | 5′-acggctcctggaaagaagagaccggtagaaccgtcacctcagcgctccccc |
| (VP2) | GQQPAKKRLNFGQTGDSESVPDPQPI | gactcctccacgggcatcggcaagaaaggccagcagcccgcgaaaaagacactc |
| | GEPPAGPSGLGSGTMAAGGGAPMADN | aactttgggcagactggcgactcagagtcagtgcccgaccctcaaccaatcgga |
| | NEGADGVGSSSGNWHCHSTWLGDRVI | gaaccccccgcaggcccctctggtctgggatctggtacaatggctgcaggcggt |
| | TTSTRTWALPTYNNHLYKQISSQSGA | ggcgctccaatggcagacaataacgaaggcgccgacggagtgggtagttcctca |
| | SNDNHYFGYSTPWGYFDFNRFHCHFS | ggaaattggcattgcgattccacatggctgggcgacagagtcatcaccaccagc |
| | PRDWQRLINNNWGFRPKRLNFKLFNI | acccgaacccgggccccccccacctacaacaaccacccctacaaacaaattccc |
| | QVKEVTDNNGVKTIANNLTSTIQVFT | agccaatcaggagcctcgaacgacaatcactactttggctacagcaccccttgg |
| | DSEYQLPYVLGSAHQGCLPPFPADVF | gggtattttgacttcaacagattccactgccacttttcaccacgtgactggcag |
| | MIPQYGYLTLNNGSQSVGRSSFYCLE | cgactcatcaacaacaactggggattccggcctaagcgactcaacttcaagctc |
| | YFPSQMLRTGNNFEFSYTFEDVPFHS | ttcaacattcaggtcaaagaggttacggacaacaatggagtcaagaccatcgcc |
| | SYAHSQSLDRLMNPLIDQYLYYLSRT | aataacctcaccagcaccatccaggtgtttacggactcggagtaccagctgccg |
| | QSTGGTAGTQQLLFSQAGPNNMSAQA | tacgtcctcagctctgcgcaccacggctgcctgcctccgttcccggcggacgtc |
| | KNWLPGPCYRQQRVSKTSADNNNSEY | ttcatgattcctcagtacggctacctgactctcaacaatggcagtcagtctgtg |
| | SWTGATKYHLNGRDSLVNPGPAMASH | ggacgttcctccttctaccgcctggaatattttccatctcaaatgctgccaact |
| | KDDEEKFFPQSGVLIFGKQGSEKTNV | ggaaacaattttgaattcagctacaccttcgaggacgtgcctttccacagcagc |
| | DIEKVMITDEEEIRTTNPVATEQYGS | tacgcacacagccagagcttggaccgactgatgaatcctctcatcgaccagtac |
| | VSTNLQRGNRQAATADVNTQGVLPGM | ctgtactacttatccagaactcagtccacgggaggtaccgcaggaactcagcag |
| | VWQDRDVYLQGPIWAKIPHTDGHFHP | ctgctattttctcaggccgggcctaataacatgtcggctcaggccaaaaactgg |
| | SPLMGGFGLKHPPPQILIKNTPVPAN | ctacccgggccctgctaccgccagcagcgagtatcaaagacatctgcggataac |
| | PSTTFSAAKFASFITQYSTGQVSVEI | aacaacagtgaatactcgTggactgcagctaccaagtaccacctcaatgccaga |
| | EWELQKENSKRWNPEIQYTSNYNKSV | gactctctggtgaatccgggcccggccatggcaagccacaaggacgatgaagac |
| | NVDFTVDTNGVYSEPHPIGTRYLTRN | aagttttttcctcagagcggggttctcatctttgggaagcaaggctcagagaaa |
| | L SEQ ID NO: 61 | acaaatgtggacattgaaaaggtcatgattacagacgaagaggaaatcaggaca |
| | | accaatcccgtggctacggagcagtatggttctgtatctaccaacctccagaga |
| | | ggcaacagacaagcagctaccgcagacgtcaacacacaaggcgttctcccaggc |
| | | atggtctggcaggacagagatgtgtaccttcaggggcccatctgggcaaagatt |
| | | ccacacacggacggacattttcacccctctcccctcatgggtggattcggactt |
| | | aaacaccctcctccacagattctcatcaagaacaccccggtacccgcgaatcct |
| | | tcgaccaccttcagtgcggcaaagtttgcttccttcatcacacagtactccacg |
| | | ggacaggtcagcgtggagatcgagtgggagctgcagaaggaaaacagcaaacgc |
| | | tgcaaccccgagatccagtacacttccaaccacaacaagtctgtcaatgtggac |
| | | tttactgtggacactaatggcgtgtattcagagcctcaccccattggcaccaga |
| | | tacctcacccgtaatctg-3′ SEQ ID NO: 85 |
| HW09 | TAPGKKRPVEPSPQRSPDSSTGIGKK | 5′-acggctcctggaaagaagagaccggtagagccatcaccccagcgttctcca |
| (VP2) | GQQPARKRLNFGQTGDADSVPDPQPL | gactcctctacgggcatcggcaagaaaggccaacagcctgcaagaaaaagattg |
| | GQPPAAPSGLGTNTMATGSGAPMADN | aattttcgtcagactggagacgcagactcagtacctgacccccagcctcccgga |
| | NEGADGVGNSSGNWHCDSTWLGDRVI | cagccaccagcagccccctctggtctgggaactaatacgacggctacaggcagt |
| | TTSTRTWALPTYNNHLYKQISSQXXP | ggcgcaccaatggcagacaataacgagggcgccgacggagtgggtaattcctcg |
| | RTTITTLATAPPGGYFDFNRFHCHFS | ggaaattggcattgcgattccacatggctgggcgacagagtcattaccaccagc |
| | PRDWQRLINNNWGFRPKRLSFKLFNI | acccgaacctgggccctgcccacctacaacaaccacccctacaaacaaatttcc |
| | QVKEVTQNEGTKTIANNLTSTIQVFT | agccaatcnnagcctcgaacgacaatcactactttggctacagcaccccctggg |
| | DSEYQLPYVLGSAMQGCLPPFPADVF | gggtattttgactttaacagattccactgccacttttcaccacgtgactggcag |
| | MVPQYGYLTLNNGSQALGRSSFYCLE | cgactcatcaacaacaactggggattccggccaaaaagactcagcttcaagctc |
| | YFPSQMLRTGNNFQFSYTFEDVPFHS | ttcaacatccaggtcaaggaggtcacgcagaatgaaggcaccaagaccatcgcc |
| | SYAHSQSLDRLMNPLIDQYLYYLSRT | aataaccttaccagcacgattcaggtatttacggactcggaataccagctgccg |
| | QSTGGTAGTQQLLFSQAGASDIRDQS | tacgtcctccgctctgcgcaccagggctgcctgcctccgttccccgcggacctc |
| | RNWLPGPCYRQQRVSKTSTDNNNSEY | ttcatggttcctcagtacggctatttaactttaaacaatggaagccaagccctg |
| | SWTGATKYHLNGRDSLVNPGPAMASH | ggacgttcctccttctactgtctggagtatttcccatcgcagatgctgagaacc |
| | KDDEEKFFPQSGVLIFGEQGSEKTNV | ggcaacaactttcagttcagctacaccttcgaggacgtgcctttccacagcagc |
| | DIEKVMITDEEEIRTTNPVATEQYGS | tacgctcacagccagagtctggaccggctgatgaaccccctcatcgaccagtac |
| | VSTNLQRGNRQAATADVNTQGVLPGM | ctgtactacctgtcccggactcagtccacgggaggcaccgcaggaactcagcag |
| | VWQDRDVYLQGPIWAKIPHTDGNFHP | ttcctattttctcaggccggagccagtaacattcgggaccagtctaggaactgg |
| | SPLMGGFGLKHPPPQILIKNTPVPAD | cttcctggaccctgttaccgccagcagcgagtatcaaagacatctacggataac |
| | PPTTFNQSKLNSFITQYSTGQVSVEI | aacaacagtgaatactcgtggactggagctaccaagtaccacctcaatggcaga |
| | EWELQKENSKRWNPEIQYTSNYYKST | gactctctggtgaatccgggcccggccatggcaagccacaaggacgatgaagaa |
| | NVDFAVNTEGVYSEPRPIGTRYLTRN | aagttttttcctcagagcggggtcctcatctttggggagcaaggctcagagaaa |
| | L SEQ ID NO: 62 | acaaatgtggacattgaaaaggtcatgattacagacgaaaaggaaatcacgaca |
| | | accaatcccgtggctacggagcagtatggttctgtatctaccaacctccagaga |
| | | ggcaacagacaagcagctaccgcagatgtcaacacacaaggcgttcttccaggc |
| | | atggtctggcaggacagagatgtgtaccttcaggggcccatctgggcaaagatt |
| | | ccacacacggacggcaacttccacccgtctccgctgatgggcggctttggcctg |
| | | aaacaccctccgcctcagatcctcatcaagaacacgcctgcacccgcggatcct |
| | | ccgaccaccttcaaccagtcaaagctgaactccttcattacgcagtacaccacc |
| | | ggacaggtcagcgtggaaatcgagtgggagctgcagaaggagaacagcaaacgc |
| | | tggaacccagagattcagtacacctcaaactactacaaatctacaaatgtggac |
| | | tttgctgtcaacacggagggggtttatagcgagcctcgccccattggcacccgt |
| | | tacctcacccgcaacctg-3′ SEQ ID NO: 86 |
| HW10 | TAPGKKRPVEHSPVEPDSSSGTGKAG | 5′-acggctccgggaaaaaagaggccggtagagcactctcctgtggagccagac |
| (VP2) | QQPARKRLNFGQTGDAESVPDPQPLG | tcctcctcgggaaccggaaaggcggcccagcagcccgcaagaaaaagatcgaat |
| | EPPATPAAVGPTTMASGGGAPMADNN | tttggtcagactggagacgcagagtcagtccccgacccacaacctctcggagaa |
| | EGADGVGNASGNWHCDSTWMGDRVIT | ccTccagcaacccccgctgctgtgggacctactacaatggcttcaggcggtggc |
| | TSTRTWALPTYNNHLYKQISSQSGAS | gcaccaacggcagacaataacgaaggcgccgacggagtgggtaatgcctcagga |
| | NDNHYFGYSTPWGYFDFNRFHCHFSP | aattggcattgcgattccacatggatgggcgacagagtcatcaccaccagcacc |
| | RDWQRLINNNWGFRPKRLSFKLFNIQ | cgaacctgggccctgcccacctacaacaaccacctctacaaacaaatctccagc |
| | VKEVTQNEGTKTIANNLTSTIQVFTD | caatcaggagcctcgaacgacaatcactactttggctacagcaccccctggggg |
| | SEYQLPYMLGSAHQGCLPPFPADVFM | tattttgactttaacagattccactgccacttttcaccacgtgactggcagcga |
| | IPQYGYLTLNNGSQAVGRSSFYCLEY | ctcatcaacaacaactggggattccggcccaagagacccagcttcaagctcttc |
| | FPSQMLRTGNNFEFSYTFEDVPFHSS | aacatccaggtcaaggaggtcacgcagaatgaaggcaccaagaccatcgccaat |
| | YAHSQSLDRLMNPLIDQYLYYLSRTN | aacatcaccagcaccatccaggtgtttacggacgcggagtaccagctcccgtac |
| | TPSGTTTQSRLQFSQAGASDIRDQSR | gtcctcggctctgcgcaccagggctgcctgcctccgttcccggcggacgtgctc |
| | NWLPGPCYRQQRVSKTSADNNNSEYS | atgattcctcagtacgggtacctgactctgaacaatggcagtcaggccgtgggc |
| | WTGATKYHLNGRDSLVNPGPAMASHK | cgttcctccttctactgcctggaacattttccatctcaaatgctgcgaactgga |
| | DDEEKFFPQSGVLIFGKQGSEKTNVD | aacaactttgaactcagctacaccttcgagcacgtgcctttccacagcagccac |
| | IEKVMITDEEEIRTTNPVATEQYGSV | gcacacagccagagctcggaccgactgatgaatcctctcatcgaccagtacctg |
| | STNLQRGNRQAATADVNTQGVLPGMV | cattactcgagcagaacaaacactccaagtggaaccaccacgcagtcaaggctt |
| | WQDRDVYLQGPIWAKIPHTGGHFHPS | cagttttctcaggccggagcgagtgacattcgggaccagtctaggaactggctt |
| | PLMGGFGLKHPPPQILIKNTPVPADF | cctggaccctgttaccgccagcagcgagtatcaaagacatctgcggataacaac |
| | PLTFNQAKLNSFITQYSTGQVSVEIE | aacagtgaatactcgtggactggagctaccaagtaccacctcaatggcagagac |
| | WELQKENSKRWNPEIQYTSNYYKSTS | tctctggtgaatccggccccggccatgacaagccacaaggacgatgaagaaaag |
| | VDFAVNTEGVYSEPRPIGTRYLTRNL | ttttttcctcagagcggggctctcatctttgggaagcaaggctcagagaaaaca |
| | SEQ ID NO: 63 | aatgtggacattgaaaaggtcatgactacagacgaagaggaaatcaggacaacc |
| | | aatcccgtggctacggagcagtatggttctgtatctaccaacctccagagaggc |
| | | aacagacaagcagctaccgcagatgtcaacacacaaggcgttcttccggcatg |
| | | gtctggcaggacagagatctgcaccttcaggggcccatctgggcaaagactcca |
| | | cacacggacggacactttcacccctctcccctcatgggtggactcggacctaaa |
| | | cacccccctccacagactctcaccaagaacacaccggctccagcggacccgccg |
| | | cttaccttcaaccaggccaagctgaactctttcatcacgcagtacagcaccgga |
| | | caggtcagcgtggaaatcgagtgggagctgcagaaagaaaacagcaagcgctgg |
| | | aacccggagattcagtacacctccaactactacaaatctacaagcgtggacctt |
| | | gctgttaatacagaaggcctgtactctgaaccccgccccattggcacccgttac |
| | | ctcacccgtaatctg-3′ SEQ ID NO: 87 |
| HW11 | TAPGKKRPVEQSPQEPDSSSGIGKTG | 5′-acggctcctggaaagaaacgtccggtagagcagtcgccacaagagccagac |
| (VP2) | QQPAKKRLNFGQTGDSESVPDPQPLG | tcctcctcgggcatcggcaagacaggccagcagcccgctaaaaagagacccaat |
| | EPPAAPSGLGSGTVAAGGGAPMADNN | tttggtcagactggcgactcagagtcagtccccgatccacaacctctcggagaa |
| | EGADGVGNASGNWHCDSTWMGDRVIT | cctccagcagccccctctggtctgggatctggtacagtggctgcaggcggtggc |
| | TSTRTWALPTYNNHLYKQISSQSGAS | gcaccaacggcagacaataacgaaggtgccgacggagtgggtaatgcctcagga |
| | NDNHYFGYSTPWGYFDFNRFHCHFSP | aattggcattgcgattccacatggatgggcgacagagtcatcaccaccagcacc |
| | RDWQRLINNNWGFRPKRLNFKLFNIQ | cgaagctgggccctgcccacctacaacaaccacctctacaaacaaatttccagc |
| | VKEVTDNNGVKTIANNLTSTVQVFTD | caatcaggagcctcgaacgacaatcactactttggctacagcaccccctggggg |
| | SDYQLPVYLGSAHEGCLPPFPADVFM | tattttgacttcaacagattccactgccatttctcaccacgtgactggcagcga |
| | IPQYGYLTLNDGSQAVGRSSFYCLEY | cccatcaacaacaactggggattccggcctaagcgactcaacttcaagctcttc |
| | FPSQMLRTGNNFQFTYTFEDVPFHSS | aacattcaggtcaaagaggttacggacaacaatggagtcaagaccatcgccaat |
| | YAHSQSLDRLMNPLIDQYLYYLSRTN | aaccttaccagcacggtccaggtcttcacggactcagactatcagctcccgtac |
| | TPSGTTTQSRLQFSQAGASDIRDQSR | gtcctcgggtcggctcacgagggctgcctcccgccgtccccagcggacgttctc |
| | NWLFGPCYRQQRVSKTDADNNNSEYS | atgattcctcagtacgggtatctgacgcttaatgatggaagccaggccgtgggt |
| | WTGATKYHLNGRDSLVNPGPAMASHK | cgttcgtccttttactgcctggaatatttcccatcgcagatgctgagaaccggc |
| | DDEEKFFPQSGVLIFGKQGSEKTNVD | aacaacttccagcttacttacaccttcgaggacgtgcctttccacagcagccac |
| | IEKVMITDEEEIRTTNPVATEQYGSV | gcccacagccagagcttggaccggctgatgaatcctctgattgaccagtacctg |
| | STNLQRGNRQAATADVNTQGVLPGMV | tactacttgagcagaacaaacactccaagtggaaccaccacgcagtcaaggctt |
| | WQDRDVYLQGPIWAKIPHTDGHFHPS | cagttttctcaggccggagcgagtgacattcgggaccagtctaggaactggctt |
| | PLMGGFGLKHPPPQILIKNTPVPANP | cctggaccctgttaccgccagcagcgagtatcaaagacatctgcggataacaac |
| | STTFSAAKFASFITQYSTGQVSVEIE | aacagtgaatactcgtggactggagctaccaagtaccacctcaatggcagagac |
| | WELQKENSKRWNPEIQYTSNYNKSVN | tctctggtgaatccgggcccggccatggcaagccacaaggacgatgaagaaaag |
| | VDVTVDTNGVYSEPRPIGTRYLTRNL | ttttttcctcagagcggggttctcatctttgggaagcaaggctcagagaaaaca |
| | SEQ ID NO: 64 | aatgtggacattgaaaaggccatgattacagacgaagaggaaatcaggacaacc |
| | | aatcccgtggctacggagcagtatggttctgtatctaccaacctccagagaggc |
| | | aacagacaagcagctaccgcagatgtcaacacacaaggcgttcttccaggcatg |
| | | gtctggcaggacagagatgtgcaccctgaggggcccatctgggcaaagactgca |
| | | cacacggacggacattttcacccctctcccctcatgggtggattcggacttaaa |
| | | caccctcctccacagactctcatcaagaacaccccggcacctgcgaatccttcg |
| | | accaccttcagtgcggcaaagtttgcttccttcatcacacagtactccacggga |
| | | caggtcagcgtggagatcgagtgggagctgcagaaggaaaacagcaaacgctgg |
| | | aaccccgaaattcagtacacttccaactacaacaagtctgttaatgtggacttt |
| | | actgtggacactaatggcgtgtattcagagcctcgccccattggcacccgttac |
| | | ctcacccgtaatctg-3′ SEQ ID NO: 68 |
| HW12 | TAPAKKRPVEQSPQEPDSSSGIGKKG | 5′-acggctcctgcaaagaagagaccagtagagcagtcaccccaagaaccagac |
| (VP2) | QQPARKRLNFGQTGDSESVPDPQPLG | tcctcctcgggcatcggcaagaaaggccaacagcccgccagaaaaagactcaat |
| | EPPATPAAVGPTTMASGGGAPMADNN | tttggccagactggcgactcagagtcagttccagaccctcaacctctcggagaa |
| | EGADGVGNSSGNWHCDSTWLGDRVIT | cctccagcaacccccgctgctgtgggacctactacaatggctttaggcggtggc |
| | TSTRTWALPTYNNHLYKQISSQSGAS | gcaccaatggcagacaataacgaagacgccgacggagtgggtaattcctcggga |
| | NDNHYFGYSTPWGYFDFNRFHCHFSP | aattggcattgcgattccacatgcctgggggacagagtcatcaccaccagcacc |
| | RDWQRLINNNWGFRPKRLNFKLFNIQ | cgaacctgggccctgcccacctacaacaaccacctctacaaacaaatttccagc |
| | VKEVTQNDGTTTIANNLTSTVQVFTD | caatcaggagcctcgaaccacaatcactactttggctacagcaccccttcgggg |
| | SEYQLPYMLGSAHQGCLPPEPADVFM | tattttgacttcaacagattccactgccacttttcaccacgtgactggcaaaga |
| | IPQYGYLTLNNGSQAVGRSSFYCLEY | ctcatcaacaacaactggggattccgacccaagagactcaacttcaagctcttt |
| | FPSQMLRTGNNFTFSYTFEDVPFHSS | aacattcaagtcaaagaggtcacgcagaatgacggtacgacgacgattgccaat |
| | YAHQSWLDRLMNPLIDQYLYYLSRTN | aaccttaccagcacggttcaggtgtttacggactcggagtaccagctgccgtac |
| | TPSGTTTQSRLQFSQAGASDIRDQSR | gttctcggctctgcccaccagggctgcctgcctccgttcccggcggacgtgttc |
| | NWLPGPCYRQQRVSKTSADNNNSEYS | atgattccccaatacggctacctaacactcaacaacggtagtcaggccgtggga |
| | WTGATKYHLNGRDSLVNPGPAMASHK | cggtcatccttttactgcctggaatatttcccatctcagatgctgagaacgggc |
| | DDEEKFFPQSGVLIFGKQGSEKTNVD | aataactttaccttcagctacaccttcgaggacgtgcctttccacagcagctac |
| | IEKVMITDEEEIRTTNPVATEQYGSV | gctcacagccagagtctggaccgtctcatgaatcctctcatcgaccagtacctg |
| | STNLQRGNRQAATADVNTQGVLPGMV | cattacttgagcagaacaaacactccaagtggaaccaccacgcagtcaaggctt |
| | WQDRDVYLQGPIWAKIPHTDGNFHPS | cagttttctcaggccggagcgagcgacattcgggaccagtctaggaactggctt |
| | PLMGGFGLKHPPPQILIKNTPVPANP | cctggaccctgttaccgccagcagcgagtatcaaagacatctgcggataacaac |
| | STTFSAAKFASFITQYSTGQVSVEIE | aacagtgaatactcgtggactggagctaccaagtaccacctcaatggcagagac |
| | WELQKENSKRWNPEIQYTSNYNKSVN | tctctggtgaatccgggcccggccatggcaagccacaaggacgatgaagaaaag |
| | VDFTVDTNGVYSEPRPIGTRYLTRNL | ttttttcctcagagcggggttctcatctttgggaagaaaggctcagagaaaaca |
| | SEQ ID NO: 65 | aatgtggacattgaaaaggtcatgattacagacgaagaggaaatcaggacaacc |
| | | aatcccgtggctacggagcagtatggttctgtatctaccaacctccagagaggc |
| | | aacagacaagcagctaccgcagatgtcaacacacaaggcgttcttccaggcatg |
| | | gtctggcaggacagagatgtgcaccctcaggggcccatctgggccaagattcct |
| | | cacacggaccgcaacttccacccctctcccctcatggctggattcggacttaaa |
| | | caccctcctccacagattctcatcaagaacaccccggtacctgcgaatccttcg |
| | | accaccttcagtgcggcaaagtttgcttccttcatcacacagtactccacggga |
| | | caggtcagcgtggagatcgagtgggagctgcagaaggaaaacagcaaacgctgg |
| | | aatcccgaaattcagtacacttccaactacaacaagtctgttaatgtggacttt |
| | | actgtggacactaacggcctgcattcagagcctcgccccattggtactcgttac |
| | | ctcacccgtaatctg-3′ SEQ ID NO: 89 |
| HW13 | TAPGKKRPVEPSPQRSPDSSTGIGKK | 5′-acggctcctggaaagaagagaccggtagaaccgtcacctcagcgttccccc |
| (VP2) | GQQPAKKRLNFGQTGDSKSVPDPQPL | gactcctccacgggcatcggcaagaaaggccagcagcccgctaaaaagagactg |
| | GEPPAAPSGLGPNTMASGGGAPMADN | aactttggtcagactggcgactcagagtcagtccccgacccacaacctctcgga |
| | NEGADGVGNASGNWHCDSTWLGDRVI | gaacctccagcagccccctcaggtctgggacctaatacaatggcttcaggcggt |
| | TTSTRTWALPTYNNHLYKQISSQSGA | ggcgctccaatggcagacaataacgaaggcgccgacggagtgggtaatgcctca |
| | SNDNHYFGYSTPWGYFDFNRFHCHFS | ggaaattggcattgcgattccacatgcctgggcgacacagtcatcaccaccagc |
| | PRDWQRLINNNWGFRPKRLSFKLFNI | acccgaacctgggccctgcccacctacaacaaccacctctacaaacaaatttcc |
| | QVKEVTQNEGTKTIANNLTSTIQVFT | agccaatcaggagcctcgaacgacaatcactactttggctacagcaccccttgg |
| | DSEYQLPYVLGSAHQGCLPPFPADVT | ggctatttcgacttcaacagattccactgccacttttcaccacgtgactggcag |
| | MIPQYGYLTLNNGSQSVGRSSFYCLE | cgactcatcaacaacaactggggattccggcccaagagactcagcttcaagctc |
| | YFPSQMLRTGNNFTFSYTFEDVPFHS | ctcaacatccaggtcaaggaggtcacgcagaatgaaggcaccaagaccatcgcc |
| | SYAHSQSLDRLMNPLIDQYLYYLSRT | aatacccttaccagcacgattcaggtctttacggactcggaataccagctcccg |
| | NTPSGTTTQSRLQFSQAGASDIRDQS | tacgtcctcggctctgcgcaccagggctgcctccctccgttcccggcggacgtg |
| | RNWLPGPCYRQQRVSKTSADNNNSEY | ttcatgattcctcagtacggctacccgactctcaacaatggcagtcagtctgtg |
| | SWTGATKYHLNGRDSLVNPGPAMASH | ggacgttcctccttctactgcctggagtacttcccctctcagatgctgcgtacc |
| | KDDEEKFFPQSGVLIFGKQGSEKTNV | ggaaacaactttaccttcagctacacttttgaggacgttcctttccacagcagc |
| | DIEKVMITDEEEIRTTNPVATEQYGS | tacgctcacagccagagtctggaccctctcatgaatcctctcatcgaccagtac |
| | VSTNLQRGNRQAATADVNTQGVLPGM | ctgtattacttgagcagaacaaacactccaagtggaaccaccacgcagtcaagg |
| | VWQDRDVYLQGPIWAKIPHTDGNFHP | cttcagttttctcaggccggagcgagtgacattcgggaccagtctaggaactgg |
| | SPLMGGFGLKHPPPQILIKNTPVPAD | cttcctggaccctgttaccgccagcagcgagtatcaaagacatctgcggataac |
| | PPTTFNQSKLNSFITQYSTGQVSVEI | aacaacagtgaatactcgtggactggagctaccaagtaccacctcaatggcaga |
| | EWELQKENSKRWNPEIQYTSNYYKST | gactccctggtgaatccgggcccggccatggcaagccacaaggacgacgaacaa |
| | SVDFAVNTEGVYSEPRPIGTRYLTRN | aagttttttcctcagagcggggttctcatctttgggaagcaaggctcagagaaa |
| | L SEQ ID NO: 66 | acaaatgtggacattgaaaaggtcatgactacagacgaagaggaaatcaggaca |
| | | accaaccccgtggctacggagcagtacggtcctgtatctaccaacctccagaga |
| | | ggcaacagacaagcagctaccgcagatgtcaacacacaaggcgttcttccaggc |
| | | atggtctggcaggacagagatgtgtaccttcaggggcccatctgggccaaaatt |
| | | cctcacacgcacggcaacttccacccgtctcccctgatgggcggctttggactg |
| | | aagcacccgcctcctcagatcctgatcaagaacacgcctgtacctgcggatcct |
| | | ccgaccaccttcaaccagtcaaagctgaactctttcatcacgcaatacagcacc |
| | | ggacaggtcagcgtggaaattgaatggaagctgcagaaagaaaacagcaagcgc |
| | | tggaaccccgagatccagtacacctccaactactacaaatctacaagtgtggac |
| | | cttgctgtcaacacggagggggtttatagcgagcctcgccccattggcacccgt |
| | | tacctcacccgtaatctg-3′ SEQ ID NO: 90 |
| HW14 | TAPGKKRPVEQSPQEPDSSSGIGKKG | 5′-acggctcctggaaagaagagaccagtagagcagtcaccccaagaaccagac |
| (VP2) | QQPARKRLNFGQTGDSESVPDPQPID | tcctcctcgggcatcggcaagaaaggccaacagcccgccagaaaaagactcaat |
| | EPPAAPSGVGPNTMAAGGGAPMADNN | ttcggccagactggcgactcagagtcagtgcccgaccctcaaccaatcggagaa |
| | EGADGVGNASGNWHCDSTWLGDRVIT | cctccagcagcgccctctggtgtgggacctaatacaatggctgcaggcggtggc |
| | TSTRTWALPTYNNHLYKQISSETAGS | gcaccaatggcagacaataacgaaggcgccgacggagtgggtaatgcctcagga |
| | TNDNTYFGYSTPWGYFDFNRFHCHFS | aactggcatcgcgattccacatggctgggcgacagagccaccaccaccagcacc |
| | PRDWQRLINNNWGFRPKRLSFKLFNI | cgaacatgggccttgcccacctataacaaccacgtctacaagcaaatctccagt |
| | QVKEVTTNDGVTTIANNLTSTVQVFS | gaaactgcaggtagtaccaacgacaacacctacttcggctacagcaccccctgg |
| | DSEYQLPYVLGSAHQGCLPPFPADVF | ggctattttgactttaacagattccactgccacttctcaccacgtgactggcag |
| | MIPQYGYLTLNNGSQAVGRSSFYCLE | cgactcatcaacaacaactggggattccggccaaaaagactcagcttcaagctc |
| | YFPSQMLRTGNNFQFSYTFEDVPFHS | ttcaacatccaagtcaaggaggtcacgacgaatgacggcgtcacgaccaccgct |
| | SYAHSQSLDRLMNPLIDQYLYYLSRT | aataaccttaccagcacggttcaagtcttctcggactcggagtaccagttgccg |
| | NTPSGTTTQSRLQFSQAGASDIRDQS | tacgtcctcggctctgcgcaccagggctgcctgcctccgttcccggcggacgtc |
| | RNWLPGPCYRQQRVSKTSADNNNSEY | ttcatgattccccagtacggctacctaacactcaacaacggtagtcaggccgtg |
| | SWTGATKYHLNGRDSLVNPGPAMASH | ggacgctcctccttctactgtctggagtatttcccatcgcagatgctgagaacc |
| | KDDEEKFFPQSGVLIFGKQGSEKTNV | ggcaacaactttcagttcagctacaccttcgaggacgtgcctttccacagcagc |
| | DIEKVMITDEEEIRTTNPVATEQYGS | cacgcgcacagccagagcctggacaggctgatgaaccccctcatcgaccagtac |
| | VSTNLQRGNRQAATADVNTQGVLPGM | ctgtattacttgagcagaacaaacactccaagtggaaccaccacgcagtcaagg |
| | VWQDRDVYLQGPIWAKIPHTDGHFHP | cttcagttttctcaggccggagcgagtgacattcgggaccagtctaggaactgg |
| | SPLMGGFGLKHPPPQILIKNTPVPAN | cttcctggaccctgttaccgccagcagcgagtatcaaagacatctgcggataac |
| | PSTTFSAAKFASFITQYSTGQVSVEI | aacaacagtgaatactcgtggactggagctaccaagtaccacctcaatggcaga |
| | EWELQKENSKRWNPEIQYTSNYYKST | gactctctggtgaatccgggcccggccatggcaagccacaaggacgatgaagaa |
| | SVDFAVNTEGVYSEPRPIGTRYLTRN | aagttttttcctcagagcggggttctcatctttgggaagcaaggctcagagaaa |
| | L SEQ ID NO: 67 | acaaatgtggacattgaaaaggtcatgattacagacgaagaggaaatcaggaca |
| | | accaaccccgtggctacggagcagtatggttctgtatctaccaacctccagaga |
| | | ggcaaccgacaagcagctaccgcagatgtcaacacacaaggcgttcttccaggc |
| | | atggtctggcaggacagagatgtgtaccttcaggggcccatctgggcaaagatt |
| | | ccacacacggacggacattttcacccctctcccctcaagggtggattcggactt |
| | | aaacaccctcctccacagatcctcatcaaaaacacacctgtacctgcgaatcct |
| | | tcgaccaccttcagtgcggcaaagtttgcttccttcatcacacagtactccacg |
| | | ggacaggtcagcgtggaaatcgaatgggagctgcagaaagaaaacagcaaacgc |
| | | tggaacccagagattcaatacacttccaactactacaaacctacaagtgtggac |
| | | cttgctgctaatacagaaggcgtgtactctgaaccccgccccattggcacccgt |
| | | tacctcacccgtaatctg-3′ SEQ ID NO: 91 |
| HW15 | TAPGKKRPVEQSPQEPDSSAGIGKSG | 5′-acggctcctggaaagaaacgtccggtagagcagtctcctcaggaaccggac |
| (VP2) | AQPAKKRLNFGQTGDSESVPDPQPLG | tcctccgcgggtattggcaaatcgggtgcacagcccgctaaaaagagactcaat |
| | EPPAGPSGLGSGTMAAGGGAPMADNN | tttggtcagactggcgactcagagtcagtccccgatccacaacctctcggagaa |
| | EGADGVGNSSGNWHCDSTWLGDRVIT | ccaccagcaggcccctctcgtctgggatctggtacaatggctgcaggcggtggc |
| | TSTRTWALPTYNNHLYKQISSASTGA | gctccaatggcagacaataacgaaggcgccgacggagtgggtaattcctcggga |
| | SNDNTYFGYSTPWGYFDFHRFHCHFS | aattggcattgcgattccacatggctgggcaacagagccatcaccaccagcacc |
| | PRDWQRLINNNWGFRPKRLSFKLFNI | cgaacctgggccctgcccacctacaacaaccacctctacaagcaaatctccagt |
| | QVKEVTQNEGTKTIANNLTSTIQVFT | gcttcaacgggggccagcaacgacaacacctacttcggctacagcaccccctgg |
| | DSEYQLPYVLGSAHQGCLPPFPADVF | ggctattttgactttaacagattccactgccacttttcaccacgtgactggcag |
| | MIPQYGYLTLNNGSQAVGRSSFYCLE | cgactcatcaacaacaactggggattccggccaaaaagactcagcttcaagctc |
| | YFPSQMLRTGNNFEFSYQFEDVPFHS | ttcaacatccaggtcaaggaggtcacgcagaatgaaggcaccaagaccatcgcc |
| | SYAHSQSLDRLMNPLIDQYLYYLSRT | aataaccttaccagcacgattcaggtctttacggactcggaataccagctgccg |
| | QTTGGTANTQTLGFSQGGPNTMANQA | tacgtcctcggctctgcccaccagggctgcctgcctccgttcccggcggacgtg |
| | KNWLPGPCYRQQRVSTTTGQNNNSNF | ttcatgattccccagtacggttacccaacactcaacaacggtagtcaggccgtg |
| | AWTAGTKYHLNGRNSLANPGIAMATH | ggacgctcctccttctactgcctggaatactttccttcgcagatgctgagaacg |
| | KDDEERFFPSNGILIFGKQNAARDNA | ggcaacaactttgagttcagctaccagtttgaggacgtgccttttcacagcagc |
| | DYSDVMLTSEEEIKTTNPVATEEYGI | cacgcgcacagccagagcctggaccggccgatgaaccctctcatcgaccagtac |
| | VADNLQQTNTGPIVGNVNSQGALPGM | ctgtactacttgtctcggactcaaacaacaggaggcacggcaaatacgcagact |
| | VWQNRDVYLQGPIWAKIPHTDGNFHP | ctgggcttcagccaaggtgggcctaatacaatggccaatcaggcaaagaactgg |
| | SPLMGGFGLKHPPPQILIKNTPVPAD | ctgccaggaccctgttaccgccaacaacgcgtctcaacgacaaccgggcaaaac |
| | PPTTFNQSKLNSFITQYSTGQVSVEI | aacaatagcaactttgcctggactgctgggaccaaataccatctgaatggaaga |
| | EWELQKENSKRWNPEIQYTSNYYKSN | aattcattggctaatcctggcatcgctatggcaacacacaaagacgacgaggag |
| | NVEFAVNTEGVYSEPRPIGTRYLTRN | cgtttttttcccagtaacgggatcctgatttttggcaaacaaaatgctgccaga |
| | L SEQ ID NO: 68 | gacaatgcggattacagcgatgtcatgctcaccagcgaggaagaaatcaaaacc |
| | | accaaccctgtggctacagaggaatacggtatcgtggcagataacttgcagcaa |
| | | accaatacggggcctattgtgggaaatgtcaacagccaaggagccttacctggc |
| | | ttggtctggcagaaccgagacgtgtacctgcagggtcccatctgggccaagatt |
| | | cctcacacggacggcaacttccacccgtctccgctgacgggcggcttcggcctg |
| | | aaacatcctccgcctcagatcctgatcaagaacacgcctgtacctgcggatcct |
| | | ccgaccaccttcaaccagtcaaagctgaactctttcatcacgcaatacagcacc |
| | | ggacaggtcagcgtggaaattgaatgggagctgcagaaggaaaacagcaagcgc |
| | | tggaacccggagatccagtacacttccaactattacaagtctaataatgttgaa |
| | | tttgctgttaatacagaaggcgtgtactctgaaccccgccccattggcacccgt |
| | | tacctcacccgtaatctg-3′ SEQ ID NO: 92 |
| HW16 | TAPGKKRPVEPSPQRSPDSSTGIGKK | 5′-acggctcctggaaagaagagaccggtagaaccgtcacctcagcgttccccc |
| (VP2) | GQQPARKRLNFGQTGDADSVPDPQPL | gactcctccacgggcatcggcaagaaaggccagcagcctgcaagaaaaagattg |
| | GQPPAAPSGLGTNTMATGSGAPMADN | aattttggtcagactggagacgcagactcagtacctgacccccagcctctcgga |
| | NEGADGVGNSSGNWHCDSTWLGNRVI | cagccaccagcagccccctctggtctgggaactaatacgatggctacaggcagt |
| | TTSTRTWALPTYNNHLYKQISNGTSG | ggcgcaccaatggcagacaataacgagagcgccgacggagtgggtaattcctcg |
| | GSTNDNTYFGYSTPWGYFDFNRFHCH | ggaaactggcatcgcgattccacatggctgggcaacagagccatcaccaccagc |
| | FSPRDWQRLINNNWGFRPKRLSFKLF | acccgaacctgggccctgcccacctacaacaaccacctctacaagcaaatatcc |
| | NIQVKEVTQNEGTKTIANNLTSTVQV | aatgggacatcgggaggaagcaccaacgacaacacctattttggctacagcacc |
| | FTDSDYQLPYMLGSAHEGCLPPFPAD | gcctgggggcattttgacttcaacagattccactgtcacttttcaccacgtgac |
| | VFMIPQYGYLTLNDGSQAVGRSSFYC | tggcaacgactcatcaacaacaactggggattccggcccaagagactcagcttc |
| | LEYFPSQMLRTGNNFQFTYTFEDVPF | aagctcttcaacattcaggtcaaggaggtcacgcagaatgaaggcaccaagacc |
| | HSSYAHSQSLDRLMNPLIDQYLYYLS | atcgccaataaccttaccagcacggtccaggtcttcacggactcagactatcag |
| | RTQTTGGTANTQTLGFSQGGPNTMAN | ctcccgtacgtgctcgggtcggctcacgagggctgcctcccgccgttcccagcg |
| | QAKNWLPGPCYRQQRVSTTTGQNNNS | gacgttttcatgattcctcagtacgggtatctgacgcttaatgatggaagccag |
| | NFAWTAGTKYHLNGRNSLANPGIAMA | gccgtgggtcgttcgtccttttactgcctggaatatttcccgtcgcaaatgcta |
| | THKDDEERFFPSNGILIFGKQNAARD | agaacgggtaacaacttccagtttacttacaccttcgaggacgtgcctttccac |
| | NADYSDVMLTSEEEIKTTNPVATEEY | agcagctacgcccacagccagagctcggaccggctgatgaatcctctgactgac |
| | GIVADNLQQRNTAPQIGTVNSQGALP | cagtacctgtactacttgtctcggactcaaacaacaggaggcacggcaaatacg |
| | GMVWQNRDVYLQGPIWAKIPHTDGNF | cagactctgggcttcagccaaggtgggcctaatacaatggccaatcaggcaaag |
| | HPSPLMGGFGLKHPPPQILIKNTPVP | aactggctgccaggaccctgttaccgccaacaacgcgtctcaacgacaaccggg |
| | ADPPTTFSAAKFASFITQYSTGQVSV | caaaacaacaatagcaactttgcctggactgctgggaccaaataccatctgaat |
| | EIEWELQKENSKRWNPEIQYTSNYYK | ggaagaaattcattggctaatcctggcatcgctatggcaacacacaaagacgac |
| | SNNVEFAVNTEGVYSEPRPIGTRYLT | gaggagcgtttttttcccagtaacgggatcctgatttttggcaaacaaaatgct |
| | RNL SEQ ID NO: 69 | gccagagacaatgcggattacagcgatgtcatgctcaccagcgaggaagaaatc |
| | | aaaaccactaaccctgtggctacagaggaacacggtatcgtggcagataacttg |
| | | cagcagcgaaacacggctcctcaaattggaactgtcaacagccagggggcctta |
| | | cccggtatggtctggcagaaccgggacgtgtacctgcagggtcccatctgggcc |
| | | aagattcctcacacggacggcaacttccacccgtctccgctgatgggcggcttt |
| | | ggcctgaaacatcctccgcctcagatcctgatcaagaacacgcctgtacctgcg |
| | | gatcctccaactaccttcagtgcggcaaagtttgcttccttcatcacacagtac |
| | | tccacgggacaggtcagcgtggagatcgagtgggagctgcagaaggaaaacagc |
| | | aaacgctggaatcccgaaattcagtacacttccaactattacaagtctaataat |
| | | gttgaatttgctgttaatactgaaggtgtatatagtgaaccccgccccattggc |
| | | accagatacctgactcgtaatctg-3′ SEQ ID NO: 93 |
| HW17 | TAPGKKRPVEPSPQRSPDSSSGIGKS | 5′-acggctcctggaaagaagagaccggcagagccatcaccccagcgttctcca |
| (VP2) | GAQPAKKRLNFGQTGDTESVPDPQPI | gactcctcctcgggcatcggcaaatcgggtgcacagcccgctaaaaagagactc |
| | GEPPAAPSGVGSLTMASGGGAPVADN | aatttcggtcagactggcgacacagagtcagtcccagaccctcaaccaatcgga |
| | NEGADGVGSSSGNWHCDSTWLGDRVI | gaacctcccgcagcccccgcaggtgggggatctctcacaatggcttcaggtggt |
| | TTSTRTWALPTYNNHLYKQISNGTSG | ggcgcaccagtggcagacaataacgaaggtgccgatggagtgggtagttcctcg |
| | GSTNDNTYFGYSTPWGYFDFNRFHCH | ggaaattggcattgcgattccacatggctgggcgacagagtcatcaccaccagc |
| | FSPRDWQRLINNNWGFRPKRLSFKLF | acccgaacctgggccctgcccacctacaacaaccacctctacaagcaaatatcc |
| | NIQVKEVTQNDGTTTIANNLTSTVQV | aatgggacatcgggaggaagcaccaacgacaacacctacttcggctacagcacc |
| | FTDSEYQLPYVLGSAHQGCLPPFPAD | ccctgggggtattttgactttaacagattccactgccacttttcaccacgtgac |
| | VFMIPQYGYLTLNNGSQAVGRSSFYC | tggcagcgactcatcaacaacaactcgggattccggcccaagagactcagcttc |
| | LEYFPSQMLRTGNNFEFSYTFEDVPF | aagctcttcaacatccaggtcaaagagctcacgcagaatgacggtacgacgacg |
| | HSSYAHSQSLDRLMNPLIDQYLYYLS | attgccaataaccttaccagcacggttcagctgcttactgactcggagtaccag |
| | RTQTTGGTANTQTLGFSQGGPNTMAN | ctcccgtacgtcctcggctcggcgcatcaaggatgcctcccgccgttcccggcg |
| | XAKNWLPGPCYRQQRVSTTTGQNNNS | gacgtcttcatgattcctcagtacgggtacctgactctgaacaatggcagtcag |
| | NFAWTGATKYHLNGRDSLVNPGVAMA | gccgtgggccgtccctcctgctactgcctggagtactctccttctcaaatgctg |
| | THKDDDDRFFPSSGVLIFGKQGAGND | agaacgggcaacaactttgagttcagctacaccttcgaggacgtgcctttccac |
| | GVDYSQVLITDEEEIRTTNPVATEQY | agcagctacgcacacagccagagcttggaccgactgatgaatcctctcatcgac |
| | GSVSTNLQRGNRQAATADVNTQGVLP | cactacctgtactacttgtctcgcactcaaacaacagcaggcaccgcaaatacg |
| | GMVWQDRDVYLQGPIWAKIPHTDGHF | cagactctgggctttagccaaggtgcgcctaatacaacggccaatcatgcaaag |
| | HPSPLMGGFGLKHPPPQILIKNTPVP | aactggctgccaggaccctgttaccggcagcagcgagtctctacgacaaccggg |
| | ANPSTTFSAAKFASFITQYSTGQVSV | caaaacaacaacagcaactttgcttggactggtgccaccaaatatcacctgaac |
| | EIEWELQKENSKRWNPEIQYTSNFEK | ggaagagactctctggtaaatcccggtgtcgctatggcaacccacaaggatgac |
| | QTGVDFAVNTEGTYSEPRPIGTRYLT | gacgaccgcttcttcccttcgagcggggtcctgatctttggcaagcaaggagcc |
| | RNL SEQ ID NO: 70 | gggaacgatggagtggattacagccaaatgctgattacagacgaagaggaaatc |
| | | aggacaaccaatcccgtggctacggagcagtatggttctgtatctaccaacctc |
| | | cagagaggcaacagacaagcagctaccgcagatgtcaacacacaaggcgctctt |
| | | ccaggcatggtctggcaggacagagatgtgtaccttcaggggcccatctgggca |
| | | aagattccacacacggacggacattttcacccctctcccctcatgggtggattc |
| | | ggacttaaacaccctcctccacagattctcatcaacaacaccccggtacctgcg |
| | | aatccttcgaccaccttcagtgcggcaaagtttgcttccttcatcacacagtac |
| | | tccacgggacaggtcagcgcggagatcgagcgggagctgcagaaggaaaacagc |
| | | aagcgctggaacccggagattcagtacacctccaactttgaaaagcagactggt |
| | | gtggactttgctgtcaatacagagggaacttattctgagcctcgccccattggt |
| | | acccgctacctcacccgtaatctg-3′ SEQ ID NO: 94 |
| HW18 | TAPGKKRPVEQSPQEPDSSSGIGKKG | 5′-acggctcctggaaagaagaggcctgcagagcagtctcctcaggaaccagac |
| (VP2) | QQPARKRLNFGQTGDSESVPDPQPLG | tcctcctcgggcatcggcaaaaaaggccagcagcccgccagaaagagactcaat |
| | EPPATPAAVGPTTMASGGGAPMADNN | ttcggtcagactggcgactcagagtcagtccccgaccctcaacctctcggagaa |
| | EXADGVGSSSGNWHCDSQWLGDRVIT | cctccagcaacccccgctgctgtggcacctactacaatggcttcaggcggtggc |
| | TSTRTWALPTYNNHLYKQISSETAGS | gcaccaatggcagacaataacgaangtgccgatggagtgggtagttcctcggga |
| | TNDNTYFGYSTPWGYFDFNRFHCHFS | aattggcattgcgattcccaatggctgggggacagagtcatcaccaccagcacc |
| | PRDWQRLINNNWGFRPKRLSFKLFNI | cgaacctgggccctgcccacctacaacaatcacctctacaagcaaatctccagt |
| | QVKEVTQNDGTTTIANNLTSTVQVFT | gaaactgcaggtagtaccaacgacaacacctacttcggctacagcaccccctgg |
| | DESYQLPYVLGSAHQGCLPPFPADVF | gggtattttgactttaacagattccactgccacttctcaccacgtgactggcag |
| | MIPQYGYLTLNNGSQAVGRSSFYCLE | cgactcatcaacaacaactggggattccggcccaagagactcagcttcaagctc |
| | YFPSQMLRTGNNFTFSYTFEDVPFHS | tttaacattcaagtcaaagaggtcacgcagaatgacggtacgacgacgattgcc |
| | SYAHSQSLDRLMNPLIDQYLYYLSRT | aacaaccttaccagcacggttcaggtgtttactgactcggagtaccagctcccg |
| | NTPSGTTTQSRLQFSQAGASDIRDQS | tacgtcctcggctctgcgcaccagggctgcctccctccgttcccggcggacgtg |
| | RNWLPGPCYRQQRVSKTSADNNNSEY | ttcatgattccgcagtacggctacctaacgctcaacaatggcagcccgacagtg |
| | SWTGATKYHLNGRDSLVNPGPAMASH | ggacggtcatccttttactgcctagaatatttcccatcgcagatgctgagaacg |
| | KDDEEKFFPQSGVLIFGKQGSEKTNV | ggcaataactttaccttcagctacaccttcgaggacgtgcctttccacagcagc |
| | DIEKVMITDEEEIRTTNPVATEQYGS | tacgcccacagccagagtctggaccgtctcatgaatcctctcatcgaccagtac |
| | VSTNLQRGNRQAATADVNTQGVLPGM | ctgtattacttgagcacaacaaacaccccaagtggaaccaccacgcagtcaagg |
| | VWQDRDVYLQGPIWAKIPHTDGHFHP | cttcagttttctcaggccggagcgagtgacattcgggaccagtctaggaactgg |
| | SPLMGGFGLKHPPPQILIKNTPVPAN | cttcctggaccctgttaccgccagcagcgagtatcaaagacatctgcggataac |
| | PSTTFSAAKFASFITQYSTGQVSVEI | aacaacagtgaatactcgtggactggagctaccaagtaccacctcaatggcaga |
| | EWELQKENSKRWNPEIQYTSNYYKST | gactctctggtgaatccgggcccggccatggcaagccacaaggacgatgaagaa |
| | SVDFAVNTEGVYSEPRPIGTRYLTRN | aagttttttcctcagagcggggttcccacctttgggaagcaaggctcagagaaa |
| | L SEQ ID NO: 71 | acaaatgtggacattgaaaaggtcatgattacagacgaagaggaaatcaggaca |
| | | accaatcccgtggctacggagcagtatggttctgtatctaccaacctccagaga |
| | | ggcaacagacaagcagctaccgcagatgtcaacacacaaggcgttcttccaggc |
| | | atggtctggcaggacagagatgtgtaccttcaggggcccatctgggcaaagatt |
| | | ccacacacggacggacattttcacccctctcccctcatgggtggattcggactt |
| | | aaacaccctcctccacagattctcaccaagaacaccccggtacctgcgaatcct |
| | | tcgaccaccttcagtgcggcaaagtttgcttccttcatcacacagtactccacg |
| | | ggacaggtcagcgtggaaattgaatgggagctgcagaaggaaaacagcaagcgc |
| | | tggaaccccgagatccagtacacctccaactactacaaatctacaagtgtggac |
| | | tttgctgttaatacagaaggcgtgtactctgaaccccgccccattggcacccgt |
| | | tacctcacccgtaatctg-3′ SEQ ID NO: 55 |
| HW19 | TAPGKKRPVEHSPVEPDSSSGTGKAG | 5′-acggctccgggaaaaaagaggccggtagagcactctcctgtggagccagac |
| (VP2) | QQPARKRLNFGQTGDADSVPDPQPLG | tcctcctcgggaaccggaaaggcgggccagcagcctgcaagaaaaagattgaat |
| | QPPAAPSGLGTNTMATGGGAPMADNN | ttcggtcagactggagacgcagactcagtacctgacccccagcctctcggacag |
| | EGADGVGNASGNWHCDSTWLGDRVIT | ccaccagcagccccctctggtctgggaactaatacgatggctacaggcggtggc |
| | TSTRTWALPTYNNHLYKQISSETAGS | gcaccaatggcagacaataacgaaggtgccgacggagtgggtaatgcctcagga |
| | TNDNTYFGYSTPWGYFDFNRFHCHFS | aactggcattgcgattccacatggctgggccacagagccattaccaccagcacc |
| | PRDWQRLINNNWGFRPKRLSFKLFNI | cgaacctgggccctgcccacctacaacaaccacctctacaagcaaatctccagt |
| | QVKEVTQNEGTKTIANNLTSTIQVFT | gaaactgcaggtagtaccaacgacaacacctacttcggctacagcaccccctgg |
| | DSEYQLPYVLGSAHQGCLPPFPADVF | gggtattttgactttaacagattccactgccacttttcaccacgtgactggcag |
| | MIPQYGYLTLNNGSQSVGRSSFYCLE | cgactcatcaacaacaactggggattccggcccaagagactcagcttcaagctc |
| | YFPSQMMRTGNNFRFSYSFEDVPFHS | ctcaacatccaggtcaagcaggtcacgcagaatgaaggcaccaagaccatcgcc |
| | SYAHSQSLDRLMNPLIDQYLYYLSRT | aataaccttaccagcacgattcacgtgtttacggactcggagtaccagctgccg |
| | QSTGGTAGTQQLLFSQAGASDIRDQS | tacgttctcggctctgcccaccagggctgactgcctccgttcccggcggacgtg |
| | RNWLPGPCYRQQRVSKTSADNNNSEY | ttcatgattcctcagtacggctacccgactctcaacaatggcagtcagtctgtg |
| | SWTGATKYHLNGRDSLVNPGPAMASH | ggacgttcctccttctactgcctggagtacttcccctctcagatgatgagaacg |
| | KDDEEKFFPQSGVLIFGKQGSEKTNV | ggcaacaactttgagttcagctacagcttcgaggacgtgcctttccacagcagc |
| | DIEKVMITDEEEIRTTNPVATEQYGS | cacgcacacagccagagcctggaccggctgatgaaccccctcatcgaccagtac |
| | VSTNLQRGNRQAATADVNTQGVLPGM | ctctactacctgtctcggactcagtccacgggaggtaccgcaggaactcagcag |
| | WVQDRDVYLQGPIWAKIPHTDGNFHP | ttgctattttctcaggccggagcgagtgacattcgggaccagtctaggaactgg |
| | SPLMGGFGLKHPPPQILIKNTPVPAD | cttcctggaccctgttaccgccagcagcgagtatcaaagacatctgcggataac |
| | PPTTFSQAKLASFITQYSTGQVSVEI | aacaacagtgaatactcgtggactggagctaccaagtaccacctcaatggcaga |
| | EWELQKENSKRWNPEIQYTSNYYKST | gactccctggtgaatccgggcccggccatggcaagccacaaggacgacgaagaa |
| | NVDFAVNTEGVYSEPRPIGTRYLTRN | aagttttttcctcagagcggggttctcatctttggcaagcaaggctcagagaaa |
| | L SEQ ID NO: 72 | acaaatgtggacattgaaaaggtcatgattacagacgaagaggaaatcaggaca |
| | | accaatcccctggctacggagcactatggttctgtatctaccaacctccagaga |
| | | ggcaacagacaagcagctaccgcagatgtcaacacacaaggcgttcttccaggc |
| | | atggtctggcaggacagagatgtgtacattcaggggcccatctgggcaaagatt |
| | | ccccacacggacggcaacttccacccctcaccgctaatgggaggattcggactg |
| | | aagcacccacctcctcagatcctgatcaagaacacgccggtacctgcggatcct |
| | | ccaacaacgttcagtcaagctaagatggcgtcgttcatcacgcagtacagcacc |
| | | ggacaggtcagcgtggaaattgaatgggagctgcagaaggaaaacagcaagcgc |
| | | tggaacccggagattcaatacacttccaactactacaaatctacaaatgtggac |
| | | tttgctgtcaacacggagggggtttatagcgagcctcgccccattggcacccgt |
| | | tacctcacccgcaacctg-3′ SEQ ID NO: 95 |

In some embodiments, a capsid protein may be the VP2 protein KJ01. In some embodiments, a capsid protein may be the VP2 protein KJ02. In some embodiments, a capsid protein may be the VP2 protein KJ03. In some embodiments, a capsid protein may be the VP2 protein KJ04. In some embodiments, a capsid protein may be the VP2 protein KJ05. In some embodiments, a capsid protein may be the VP2 protein HW01. In some embodiments, a capsid protein may be the VP2 protein HW02. In some embodiments, a capsid protein may be the VP2 protein HW03. In some embodiments, a capsid protein may be the VP2 protein HW04. In some embodiments, a capsid protein may be the VP2 protein HW05. In some embodiments, a capsid protein may be the VP2 protein HW06. In some embodiments, a capsid protein may be the VP2 protein HW07. In some embodiments, a capsid protein may be the VP2 protein HW08. In some embodiments, a capsid protein may be the VP2 protein HW09. In some embodiments, a capsid protein may be the VP2 protein HW10. In some embodiments, a capsid protein may be the VP2 protein HW11. In some embodiments, a capsid protein may be the VP2 protein HW12. In some embodiments, a capsid protein may be the VP2 protein HW13. In some embodiments, a capsid protein may be the VP2 protein HW14. In some embodiments, a capsid protein may be the VP2 protein HW15. In some embodiments, a capsid protein may be the VP2 protein HW16. In some embodiments, a capsid protein may be the VP2 protein HW17. In some embodiments, a capsid protein may be the VP2 protein HW18. In some embodiments, a capsid protein may be the VP2 protein HW19.

In some embodiments, a capsid protein described herein may be selected from any of those found in Table 3. In some embodiments, the capsid protein may be a variant of any of the capsid proteins (VP3) found in Table 3. In some embodiments, AAV particles are described herein that comprise such a capsid protein or proteins, or variants thereof. In some embodiments, AAV particles are described herein that comprise such a capsid protein or proteins, or variants thereof, and a VP1 protein of Table 1, or variants thereof. In some embodiments, AAV particles are described herein that comprise such a capsid protein or proteins, or variants thereof, a VP1 protein of Table 1, or variants thereof, and a VP2 protein of Table 2, or variants thereof.

24 In some embodiments, a capsid protein or proteins may be encoded by a polynucleotide sequence found in Table 3. In some embodiments, the capsid protein or proteins may be encoded by a polynucleotide sequence that is a codon optimized form of a polynucleotide sequence of Table 3. For example, the capsid protein or proteins may be encoded by a polynucleotide sequence that is codon optimized for expression in insect cells, such as Sf9 insect cells. In some embodiments, the capsid protein or proteins may be encoded by a polynucleotide sequence that differs from a polynucleotide sequence of Table 3 due to amino acid code degeneracy. In some embodiments, AAV particles are described herein that comprise a capsid protein or proteins, or variants thereof, encoded by such a polynucleotide.

TABLE 3
Capsid Proteins (VP3)

| Capsid | | |
|---|---|---|
| Protein | Amino Acid | Representative Polynucleotide |
| KJ01 | MAAGGGAPMADNNEGADGVGSSSGNW | 5′-atggctgcaggcggtggcgctccaatggcagacaataacgaaggcgccgac |
| (VP3) | HCDSTWLGDRVITTSTRTWALPTYNN | ggagtgggtagtccctcaggaaactggcattgcgattccacatgcctcggcgac |
| | HLYKQISNGTSGGSTNDNTYFGYSTP | agagtcatcaccaccagcacccgaacctgggccctgcccacctacaacaaccac |
| | WGYFDFNRFHCHFSPRDWQRLINNNW | ctctacaagcaaatctccaacggcacctcgggaggaagcaccaacgacaacacc |
| | GFRPKRLSFKLFNIQVKEVTQNEGTK | taatttggctacagcaccccctgcgggtattttgactttaacagattccactgc |
| | TIANNLTSTIQVFTDSEYQLPYVLGS | cacttttcaccacgtgactggcagcgactcatcaacaacaactggggattccgg |
| | AHQGCLPPFPADVFMIPQYGYLTLNN | cccaagagactcagcttcaagctcctcaacatccaggtcaaggaggtcacgcag |
| | GSQAVGRSSFYCLEYFPSQMLRTGNN | aatgaaggcaccaagaccatcgccaataacctcaccaccaccatccaggtgttt |
| | FQFTYTFEDVFPHSSYAHSQSLDRLM | acggactcggagtaccagctgccgtacgttctcggctctgcccaccagggctgc |
| | HPLIDQYLYYLSRTQSTGGTAGTQQL | ctgcctccgttccccgcggacgtgttcatgattccccagtacggctacctaaca |
| | LFSQAGPNNMSAQAKNWLPGPCYRQQ | ctcaacaacggtagtcaggccgtcggacgctcctccttctactgcctggaatac |
| | RVSTTLSQNNNSNFAWTGATKYHLNG | tttccttcgcagatgctgagaaccggcaacaacttccagtttacttacaccttc |
| | RDSLVNPGVAMATHKDDEERFFPSSG | gaggacgtgcctttccacagcagctacgcccacagccagagcctggaccggctg |
| | VLMFGKQGAGKDNVDYSSVMLTSEEE | atgaatcctctgattgaccagtacctgtactacctgtctcggactcagtccacg |
| | IKTTNPVATEQYGVVADNLQQQNAAP | ggaggtaccgcaggaactcagcagttgctattttctcaggccgggcctaataac |
| | IVGAVNSQGALPGMVWQNRDVYLQGP | atgtcggctcaggccaaaaactggctacccgggccctgctaccggcagcaacgc |
| | IWAKIPHTDGNFHPSPLMGGFGLKHP | gtctccacgacactgtcgcaaaataacaacagcaactttgcctggaccggtgcc |
| | PPQILIKNTPVPADPPTTFSQAKLAS | accaagtatcatctgaatggcagagactctctggtaaatcccggcgtcgctatg |
| | FITQYSTGQVSVEIEWELQKENSKRW | gcaacccacaaggacgacgaacagcgattttttccctccagccgagtcttaatg |
| | NPEIQYTSNYYKSTNVDFAVNTDGTY | tttgggaaacagggagctggaaaagacaacgtggactatcgcagcgttacgcta |
| | SEPRPIGTRYLTRNL SEQ ID NO: | accagtgagcaagaaactaaaaccaccaacccagtggccacagaacagtaccgc |
| | 97 | gtggtggccgataacctgcaacagcaaaacgccgctcctattgtaggggccgtc |
| | | aacagtcaaggagccttacctggcatggtctggcagaaccgggacgtgtacctg |
| | | cagggtcctatccgggccaagatccctcacacggacggaaactttcacccctcg |
| | | ccgctgatgggaggctttggactgaaacacccgcctcctcagatcctgattaag |
| | | aatacacctgttcccgcggatcctccaactaccttcagtcaagctaagctggcg |
| | | tcgttcatcacgcagtacagcaccggacaagtcagcgtggaaatcgagtgggag |
| | | ctgcagaaggaaaacagcaaacgctggaatccagagattcagtacacttcaaac |
| | | tactacaaatctacaaatgtggactttgctgttaacacagatggcacttattct |
| | | gagcctcgccccatcggcacccgttacctcacccgtaatctg-3′ |
| KJ02 | MAAGGGAPMADNNEGADGVGNASGNW | 5′-atggctgcaggcggtggcgctccaatggcagacaattacgagggcgccgac |
| (VP3) | HCDSTWLGDRVITTSTRTWALPTYNN | ggagtgggtaatgcctcaggaaatcggcattgcgattccacatggctgggcgac |
| | HLYKQISSETAGSTNDNTYFGYSTPW | agagtcattaccaccagcacccgaacctgggccctgcccacctacaacaaccac |
| | GYFDFNRFHCHFSPRDWQRLINNNWG | ctctacaagcaaatctccagtgaaactgcaggtagtaccaacgacaacacctac |
| | FRPKRLNFKLLNIQVKEVTDNNGVKT | ttcggctacagcaccccctgggggcattttgacttcaacagattccactgccac |
| | IANNLTSTIQVFTDSEYQLPYVLGSA | ttctcaccacgtgactggcagcgactcatcaacaacaactggggactccgacct |
| | HQGCPPPRPADVFMIPQYGYLTLNNG | aagcgactcaacttcaagctcctcaacattcaggtcaaagaagttacggacaac |
| | SQAVGRSSFYCLEYFPSQMLRTGNNF | aatggagtcaagaccatcgccaataacctcaccagcaccatccaggtgtttacg |
| | EFSYSFEDVPFHSSYAHSQSLDRLMN | gactcggagtaccagctgccgtacgttctcggctctgcccaccagggctgcccg |
| | PLIDQYLYYLSRTQSTGGTAGTQQLL | cctccgttcccggcggacgtcttcatgattcctcagtacggctacctgactctc |
| | FSQAGPNNMSAQAKNWLPGPCYRQQR | aacaatggcagtcaggccgtgggccgttcctccttctactgcctggagtacttt |
| | VSTTLSQNNNSNFAWTGATKYHLNGR | ccttctcaaatgctgagaacgggcaacaactttgagttcagctacagcttcgag |
| | DSLVNPGNAMATHKDDEERFFPSSGV | gacgtgcctctccacagcagctacgcacacagccagagcctggaccggctgatg |
| | LMFGKQGAGKDNVDYSSVMLTSEEEI | aatcccctcatcgaccagtacctgtactacctgtctcggactcagtccacggga |
| | KTTNPVATEQYGVVADNLQQQNTAPQ | ggtaccgcaggaactcagcagttgctattttctcaggccgggccaaataacatg |
| | IGTVNSQGALPGMVWQNRDVYLQGPI | tccgctcagcccaaaaactggctacccgggccctgctaccggcaccaacgcgtc |
| | WAKIPHTDGHFHPSPLMGGFGLKHPP | tccacgacactgtcgcaaaataacaacagcaactttgcctggaccggtgccacc |
| | PQILKNTPVPANFPSTTFNQSKLNSF | aagtatcatctgaatggcagaaactctccggtaaaccccggtgtcgctatggca |
| | ITQYSTGQVSVEIEWELQKENSKRWN | acccacaaggacgacgaagagcgattttttccgtccagcggagtcttaatgttt |
| | PEIQYTSNYYKSTSVDFAVNTEGVYS | gggaaacagggagcaggaaaagacaacgaggactaaagcagcgttatgcaaacc |
| | EPRPIGTHYLTRNL SEQ ID NO: | agtgaggaagaaattaaaaccaccaacccagtggccacagaacagtacggcgtg |
| | 98 | gtggccgataacctgcaacagcaaaacacggctcctcaaattggaactgtcaac |
| | | agccagggggccttacccggtatggtctggcagaaccgggacgtgtacctgcag |
| | | ggtcccatctgggccaagattccacacacggacggacattttcacccctctccc |
| | | ctcatgggtggattcggacttaaacaccctcctccacagattctcatcaagaac |
| | | accccggtacctgcgaatccttcgaccaccttcaaccagtcaaagctgaactct |
| | | ttcatcacgcaatacagcaccggacaggtcagcgtggaaattgaatgggagctg |
| | | cagaaggaaaacagcaagcgctggaaccccgagatccagtacacctccaactac |
| | | tacaaatctacaagtgtggactttgccgttaatacagaaggcgtctactctgaa |
| | | ccccgccccattggcacccattacctcacccgcaacctg-3′ SEQ ID NO: |
| | | 122 |
| KJ03 | MAAGGGAPMADNNEGADGVGNASGNW | 5′-atggcttcaggcggtggcgcaccaatggcagacaataacgaaggcgccgac |
| (VP3) | HCDSTWLGDRVITTSTRTWALPTYNN | ggagtgggtagttcctcaggaaatcggcattgcgattccacatggctgggcgac |
| | HLYKQISSETAGSTNDNTYFGYSTPW | agagtcatcaccaccagcacccgaacatgggccttgcccacctataacaaccac |
| | GYFDFNRFHCHFSPRDWQRLINNNWG | ctctacaagcaaatctccagtgcttcaacgggggccagcaacgacaaccactac |
| | FRPKRLNFKLLNIQVKEVTDNNGVKT | ttcggctacagcaccccctgggggtattttgacttcaacagattccactgccac |
| | IANNLTSTIQVFTDSEYQLPYVLGSA | ttttcaccacgtgactggcaaagactcatcaacaacaactggggattccggccc |
| | HQGCPPPRPADVFMIPQYGYLTLNNG | aagagactcagcttcaagctcttcaacatccaggtcaaggaggtcacgcagaat |
| | SQAVGRSSFYCLEYFPSQMLRTGNNF | gaaggcaccaagaccatcgccaataacctcaccagcaccatccaggtgtttacg |
| | EFSYSFEDVPFHSSYAHSQSLDRLMN | gactcggagtaccagctgccgtacgttctcggctctgcccaccagggctgcctg |
| | PLIDQYLYYLSRTQSTGGTAGTQQLL | cctccgttcccggcggacgtcttcatgattcctcagtacggctacctgacgctc |
| | FSQAGPNNMSAQAKNWLPGPCYRQQR | aacaatggcagccacgcagtgggacggtcatccttttactgcctggaatatttc |
| | VSTTLSQNNNSNFAWTGATKYHLNGR | ccatcgcagatgctgagaacgggcaataactttaccttcagctacaccttcgag |
| | DSLVNPGNAMATHKDDEERFFPSSGV | gacgtgcctttccacagcagctacgcgcacagccaaagcctggaccggctgatg |
| | LMFGKQGAGKDNVDYSSVMLTSEEEI | aaccccctcatcgaccagtacctgtactacctgtctcggactcagtccacggga |
| | KTTNPVATEQYGVVADNLQQQNTAPQ | ggtaccgcaggaactcagcagttgctattttctcaggccgggcctaataacatg |
| | IGTVNSQGALPGMVWQNRDVYLQGPI | tcggctcagcctaagaactggctacctggaccttgctaccggcagcagcgagtc |
| | WAKIPHTDGHFHPSPLMGGFGLKHPP | tctacgacactgtcgcaaaacaacaacagcaactttgcttggactggtgggacc |
| | PQILKNTPVPANFPSTTFNQSKLNSF | aaataccatctgaatggaagaaattcattggctaatcctggcatcgctatggca |
| | ITQYSTGQVSVEIEWELQKENSKRWN | acacacaaagacgacgaggagcgtttctttcccagtaacgggatcctgattttt |
| | PEIQYTSNYYKSTSVDFAVNTEGVYS | ggcaaacaaaatgctgccagagacaatgcggattacagcgatgtcatgctcacc |
| | EPRPIGTHYLTRNL SEQ ID NO: | agcgaggaagaaatcaaaaccactaaccctgtggctacagaggaatacggtatc |
| | 99 | gtggcagataacttgcagcagcaaaacacggctcctcaaattggaactgtcaac |
| | | agccagggggccttacccggtatggtctggcagaaccgggacgtgtacctgcag |
| | | ggtcctatctgggccaagattcctcacacggacggaaactttcatccctcgccg |
| | | ctgatgggaggctttggactgaaacacccgcctcctcagatcctcattaagaac |
| | | acgcctgtacctgcggatcctccgaccaccttcaaccagtcaaagctgaactct |
| | | ctcatcacgcaatacagcaccggacaggtcagcgtggaaattgaatgggagcta |
| | | cagaaggaaaacagcaagcgctggaaccccgagatccagtacacctccaactac |
| | | tacaaatctacaagtgtggactttgctgttaatacagaaggcgtgtactctgaa |
| | | ccccgccccattggcacccgttacctcacccgtaacctg-3′ SEQ ID NO: |
| | | 123 |
| KJ04 | MAAGGGAPMADNNEGADGVGNASGNW | 5′-atggctgcaggcggtggcgcaccaatggcagacaataacgaaggcgccgac |
| (VP3) | HCDSTWLGDRVITTSTRTWALPTYNN | ggagtgggtagttcctcgggaaattggcattgcgattccacatggctgggcgac |
| | HLYKQISNGTSGGATNDNTYFGYSTP | agagtcatcaccaccagcacccgaacctgggccctgcccacctacaacaaccac |
| | GFRPKRLSFKLFNIQVKEVTQNEGTK | ctctacaagcaaatctccaacgggacatcgggaggagccaccaacgacaacacc |
| | TIANNLTSTIQVFTDSEYQLPYVLGS | tacttcggctacagcaccccctgggggtattttgacttcaacagattccactgc |
| | AHQGCLPPFPADVFMIPQYGYLTLNN | cacttctcaccacgtgactgacaacgactcatcaacaacaattgcggattccgg |
| | GSQAVGRSSFYCLEYFPSQMLRTGNN | cccaaaagactcagctccaagctcttcaacatccaggccaaggaggtcacgcag |
| | FQFTYTFEDVPFHSSYAHSQSLDRLM | aatgaaggcaccaagaccatcgccaataacctcaccagcaccatccaggtgttt |
| | NPLIDQYLYYSLRTQTTGGTANTQTL | acggactcggagtaccagctgccgtacgttctcggctctgcgcaccagggctgc |
| | GFSQGGPNTMANQAKNWLPGPCYRQQ | ctccctccgctcccggcggacgtcttcatgattcctcagtacgggtacctgact |
| | RVSTTTGQNNNSNFAWTAGTKYHLNG | ctgaacaatggcagtcaggccgtgggccgttcctccttctactgcctggaatat |
| | RNSLANPGIAMATHKDDEERFFPSNG | tttccatcgcagatgctgagaaccggcaacaacttccagtttacttacaccttc |
| | ILIFGKQNAARDNADYSDVMLTSEEE | gaggacgtgcctttccacagcagctacgcccacagccagagcttggaccggctg |
| | IKTTNPVATEEYGIVADNLQQQNTAP | atgaatcctctgattgaccagtacctgtactacttctctcggactcaaacaaca |
| | QIGTVNSQGALPGMVWQNRDVYLQGP | ggaggcacggcaaacacgcagactctgggcttcagccaaggtgggcctaataca |
| | IWAKIPHTDGNEHPSPLMGGFGLKHP | atggccaatcaggcaaagaactggctgccaggaccctgttaccgccaacaacgc |
| | PPQILIKNTPVPANPPEVFTPAKFAS | gtctcaacgacaaccgggcaaaacaacaatagcaactttgcctggactgctggg |
| | FITQYSTGQVSVEIEWELQKENSKRW | accaaataccatctcaatggaagaaattcattggctaatcctggcatcgctatg |
| | NPEIQYTSNFEKQTGVDFAVDSQGVY | gcaacacacaaagacgacgaggagcgtttttttcccagtaacgggatcctgatt |
| | SEPRPIGTRYLTRNL SEQ ID NO: | tttggcaaacaaaatgctgccagagacaatgcggattacagcgatgtcatgctc |
| | 100 | accagcgaggaagaaatcaaaaccactaaccctgtcgctacagaggaatacggt |
| | | atcgtggcagataacttgcagcagcaaaacacggcccctcaaattggaactgtc |
| | | aacagccagggggccttacccggtatggtctggcagaaccgggacgtgtacctg |
| | | cagggtcccatctgggccaagattcctcacacggacggcaacttccacccgtct |
| | | ccgctgatgggcggctttggcctgaaacatcctccgcctcagatcctgatcaag |
| | | aacacgcccgttcccgctaatcctccggaggtgcttactcctgccaagtttgct |
| | | tcgttcatcacacactacagcaccggacaagtcagcgtggaaatcgagtgggag |
| | | ctgcagaaggaaaacagcaagcgctggaacccggagattcagtacacctccaac |
| | | tttgaaaagcagactgctgtggacttcgccgttgacagccagggcgtctactct |
| | | gagcctcgccctattggcactcgttacctcacccgcaacctg-3′ SEQ ID |
| | | NO: 124 |
| KJ05 | MAAGGGAPMADNNEGADGVGNASGNW | 5′-atggcttcaggtggtggcgcaccagtggcagacaataacgaaggtgccgac |
| (VP3) | HCDSTWLGDRVITTSTRTWALPTYNN | ggagtgggtaatgcctcaggaaatcggcattgcgattccacatggctggccgac |
| | HLYKQISNGTSGGATNDNTYFGYSTP | agagtcatcaccaccaccacccgaacatgggccctgcccacctacaacaaccac |
| | GFRPKRLSFKLFNIQVKEVTQNEGTK | ctctacaagcaaatctccaacagcacatctggaggatcttcaaatgacaacgcc |
| | TIANNLTSTIQVFTDSEYQLPYVLGS | tacttcggctacagcaccccctgggggtattttgacttcaacggattccactgc |
| | AHQGCLPPFPADVFMIPQYGYLTLNN | catttctcaccacgtgactggcagcgactcatcaacaacaattggggattccgg |
| | GSQAVGRSSFYCLEYFPSQMLRTGNN | cccaagagactcggcttcaagctcttcaacatccaggtcaaggaggtcacgcag |
| | FQFTYTFEDVPFHSSYAHSQSLDRLM | aatgaaggcaccaagaccatcgccaataacctcaccagcaccatccaggtgttt |
| | NPLIDQYLYYSLRTQTTGGTANTQTL | acggactcggagtaccagctgccgtacgttctcggctctgcccaccagggctgc |
| | GFSQGGPNTMANQAKNWLPGPCYRQQ | ctgcctccgttcccggcggacgtgttcatgattcctcagtacggctacctgact |
| | RVSTTTGQNNNSNFAWTAGTKYHLNG | ctgaacaatggcagtcaggccgtgggccgttcctccttctactgcctggagtac |
| | RNSLANPGIAMATHKDDEERFFPSNG | tttccttctcaaatgctgcgaactggaaacaattttgaattcagctacaccttc |
| | ILIFGKQNAARDNADYSDVMLTSEEE | gaggacgtgcctctccacagcagctacgcgcacagccagagcctggacaggctg |
| | IKTTNPVATEEYGIVADNLQQQNTAP | atgaatcccctcatcgactactacctgtactacttgtctcggactcaaacaaca |
| | QIGTVNSQGALPGMVWQNRDVYLQGP | ggaggcacggcaaatacgcagactctgggcttcagccaaggtgggcctaataca |
| | IWAKIPHTDGNEHPSPLMGGFGLKHP | atcgccaatcaggcaaagaactgcctgccaggaccctcttaccgccaacaacgc |
| | PPQILIKNTPVPANPPEVFTPAKFAS | gtctcaacgacaaccgggcaaaacaacaatagcaactttgcctggactgctggg |
| | FITQYSTGQVSVEIEWELQKENSKRW | accaaataccatctgaacggaagaaattcattggctaatcctggcatcgctatg |
| | NPEIQYTSNFEKQTGVDFAVDSQGVY | gcaacacacaaacacgacgaggaccgtttttttcccagtaacgggatcctgatt |
| | SEPRPIGTRYLTRNL SEQ ID NO: | tttggcaaacaaaatgctgccagagacaatgcggattacagcgatgtcatgctc |
| | 101 | accagcgaggaagaaatcaaaaccactaaccctgtcgctacacaggaatacggt |
| | | atcgtggcagataacttgcagcagcaaaacacggctcctcaaattggaactgtc |
| | | gacagccagggggccttacccggtatggtctggcagaaccgggacgtgtacctg |
| | | cagggtcccatctgggccaagattcctcacacggacggcaacttccacccgtcc |
| | | ccgctgatgggcggctttggcctgaaacatcctccgcctcagatcctgatcaag |
| | | aacacgcctgtacctgcggatcctccgaccaccttcaaccagtcaaagctgaac |
| | | tctttcatcacgcaatacagcacccgacaggtcagcgtggaaattgaatgggag |
| | | ctacagaaggaaaacagcaagcgctggaaccccgagatccagtacacctccaac |
| | | tactacaaatctacaagtgtggactttgctgttaatacagaaggcgtttactct |
| | | gagcctcgccctattgggactcgttacctcacccgtaatctg-3′ SEQ ID |
| | | NO: 125 |
| HW01 | MAAGGGAPMADNNEGADGVGNSSGNW | 5′-atggctgcaggcggtggcgctccaatggcagacaataacgaaggcgccgac |
| (VP3) | HCDSTWLGDRVITTSTRTWALPTYNN | ggagtgggtaattcctcgggaaattggcattgcgattccacatggctgggggac |
| | HLYKQISNSTSGGSSNDNAYFGYSTP | agagtcatcaccaccagcacccgaacctggcccctgcccacctacaacaaccac |
| | WGYFDFNRFHCHRSPRDWQRLINNNW | ctctacaagcaaatctccaacagcacatctggaggatcttcaaatgacaacgcc |
| | GFRPKRLNFKLFNIQVKEVTQNEGTK | cacttcggctacagcaccccctgggggtattctgacttcaacagattccactgc |
| | TIANNLTSTIQVFTDSEYQLPYVLGS | cacttctcaccacgtgactggcagcgactcatcaacaacaactggggattccgg |
| | AHQGCLPPFPADVFMIPQYGYLTLNN | cccaagagactcaacttcaagctcttcaacatccaggtcaaggaggtcacgcag |
| | GSQAVGRSSFYCLEYFPSQMLRTGNN | aatgaagccaccaacaccatccccaataacctcaccagcaccatccaggcgttt |
| | FQFTYTFEDVPFHSSYAHSQSLDRLM | aaggactcggagtaccagctgccgtacgttctcggctctgcccacaagggctgc |
| | NPLIDQYLYYLSRTQSTGGTAGTQQL | ctgcctccgttcccggcggacgtgttcatgattccccagtacggctacctaaca |
| | LFSQAGPNNMSAQAKNWLPGPCYRQQ | ctcaacaacggtagtcaggccgtgggacgctcctccttctactgcctggaatac |
| | RVSKTSADNNNSEYSETGATKYHLNG | tttccttcgcagatgctgagaaccggcaacaacttccagtttacttacaccttc |
| | RDSLVNPGPAMASHKDDEEKFFPQSG | gaggacgtgcctctccacagcagctacgcccacagccagagcttggaccggctg |
| | VLIFGKQGSEKTNVDIEKMVITDEEE | atgaaccccctcatcgaccagtacctgtactacctctctcggactcagtccacg |
| | IRTTNPVATEQYGSVSTNLQRGNRQA | ggaggtaccgcaggaactcagcagttgctattttctcaggccgggcctaataac |
| | ATADVNTQGVLPGMVWQDRDVYLQGP | atatcggctcaggccaaaaactggctacccgggccctgctaccggcagcagcga |
| | IWAKIPHTDGHFHFSPLMGGFGLKHP | gtatcaaagacatctgcggataacaacaacagtgaatactcgtggactgcagct |
| | PPQILIKNTPVPADPPTTFNQSKLNS | accaagtaccacctcaatggcagagactctctggtgaatccgggcccggccatg |
| | FITQYSTGQVSVEIEWELQKENSKRW | gcaagccacaaggacgatgaagaaaagttttttcctcagagcggggtcctcatc |
| | NPEIQYTSNYYKSTSVDFAVNTEGVY | tttgggaagcaaggctcagagaaaacaaatgtggacattgaaaaggtcatgatt |
| | SEPRPIGTRYLTRNL SEQ ID NO: | acagacgaagaggaaatcaggacaaccaatcccgtggctacggagcagtatggt |
| | 102 | tctgtatctaccaacctccagagaggcaacagacaagcagctaccgcagatctc |
| | | aacacacaaggcgttcttccaggcatggtctggcaggacagagatgtgtacctt |
| | | caggggcccatctgggcaaagattccacacacggacggacattttcacccctct |
| | | cccctcatgggtggattcggacttaaacaccctcctccacagatcctgatcaag |
| | | aacacgcctgtacctgcggatcctccgaccaccttcaaccagtcaaagctgaac |
| | | cctttcttcacgcaatacagcaccggacaggtcagcgtggaaattgaatcggag |
| | | ctgcagaaggaaaacacccagcgctggaaccccgagatccagtacacctccaac |
| | | tactacaaatctacaagtgtggactttgctgttaatacagaaggcgtgtactct |
| | | gaaccccgccccattggcacccgttacctcacccgcaacctg-3′ SEQ ID |
| | | NO: 126 |
| HW02 | MATGSGAFMADNNEGADGVGNSSGNW | 5′-atggctacaggcagtggcgcaccaatggcagacaataacgagggcgccgac |
| (VP3) | HCDSTWMGDRVITTSTRTWALPTYNN | ggagtgggtaattcctcgggaaattggcattgcgattccacatggatgggcgac |
| | HLYKQISSQSGASNDNHYFGYSTPWG | agagtcatcaccaccagcacccgaacctgggccctgcccacctacaacaaccac |
| | YFDFNRFHCHFSPRDWQRLINNNWGF | ctctacaaacaaatttccagccaatcaggagcctcgaacgacaatcactacttt |
| | RPKRLNFKLFNIQVKEVTQNDGTTTI | ggctacagcaccccttgggggtattttgacttcaacagattccactgccacttt |
| | ANNLTSTVQVFTDSEYQLPYVLGSAH | tcaccacgtgaccggcaaagactcatcaacaacaactggggattccgacccaag |
| | QGCLPPFPADVFMVPQYGYLTLNNGS | agactcaacttcaagccctttaacattcaagtcaaagaggtcacgcagaatgac |
| | QAVGRSSFYCLEYFPSQMLRTGNNFT | ggtacgacgacgatcgccaataaccttaccagcacggtccaggtgtttactgac |
| | FSYTFEDVPFHSSYAHSWSLDRLMNP | tccgagtaccagctcccgtacgtcctcggctcggcgcatcaaggatgcctcccg |
| | LIDQYLYYLSRTNTPSGTTTQSRLQF | ccgttcccagcagacgtcttcatggtgccacagtatggatacctcaccctgaac |
| | SQAGASDIRDQSRNWLPGPCYRQQFV | aacgggagtcaggcagtaggacgctcttcattttactgcctggagtactttcct |
| | SKTSADNNNSEYSWTGATKYHLNGRD | tctcagatgctgcgtaccggaaacaactttaccttcagctacacttttgaggac |
| | SLVNPGPAMASHKDDEEKFFPQSGVL | gttcctttccacagcagctacgctcacagccagagtctggaccgtctcatgaat |
| | IFGKQGSEKTNVDIEKVMITDEEEIR | cctctcatcgaccagtacctgtatcacttgagcagaacaaacactccaactgga |
| | TTNPVATEQYGSVSTNLQRGNRQAAT | accaccacgcagtcaacgcttcagttttctcaggccgcagcgagtgacattcgg |
| | ADVNTQGVLPGMVWQDRDVYLQGPIW | gaccagtctaggaactggcctcctggaccctgttaccgccagcagcgagtatca |
| | AKIPHTDGHFHPSPLMGGFGLKHPPP | aagacatctgcggacaacaacaacagtgaatactcctggactggagctaccaag |
| | QILIKNTPVPADPPTTFNQSKLNSFI | taccacctcaatggcagagactctctggtgaatccgggcccggccatggcaagc |
| | TQYSTGQVSVEIEWELQKENSKRWNP | cacaaggacgatgaagaaaagttttttcctcagagcggggttctcatctttggg |
| | EIQYTSNYYKSTSVDFAVNTEGVYSE | aagcaaggctcagacaaaacaaatgtggacattgaaaaggtcatgattacagac |
| | PRPIGTRYLTRNL SEQ ID NO: | gaagaggaaatcaggacaaccaatcccgcggctacggagcagtatggttctgta |
| | 103 | tccaccaacctccagacaggcaacagacaagcagctaccgcagatgtcaacaca |
| | | caaggcgttcttccaggcatggtctggcaggacagagatgtgtaccttcagggg |
| | | cccatctgggcaaagattccacacacggacggacattttcacccctctcccctc |
| | | atgggtggattcggacctaaacaccctcctccacagaccctgatcaagaacacg |
| | | cccgtacctgcggatcctccgaacaccttcaaccagtcaaagctgaactctttc |
| | | atcacgcaatacagcaccggacaggtcagcgtggaaaatgaatgggagctgcag |
| | | aaggaaaacagcaagccctggaaccccgagatccagtacacctccaactaccac |
| | | aaatctacaagtgtggactttgctgttaatacagaaggcgtgtactctgaaccc |
| | | cgccccattggcacccgttacctcacccgcaacctg-3′ SEQ ID NO: 127 |
| HW03 | MAAGGGAPMADNNEGADGVGNSSGNW | 5′-atggctgcaggcggtggcgctccaatggcagacaataacgaaggcgccgac |
| (VP3) | HCDSTWLGDRVITTSTRTWALPTYNN | ggagtgggtaattcctcgggaaattggcattgcgattccacctggctgggggac |
| | HLYKQISNSTSGGSSNDNAYFGYSTP | agagtcatcaccaccagcacccgaacctgggccctgcccacctacaacaaccac |
| | WGYFDFNRFHCHFSPRDWQRLINNNW | ctctacaagcaaatctccaacagcacatctggaggatcttcaaatgacaacgcc |
| | GFRFKRLNFKLFNIQVKEVTQNEGTK | tacttcggctacagcaccccctgggggtattttgacttcaacagattccactgc |
| | TIANNLTSTIQVFTDSEYQLPYVLGS | cacttctcaccacgtgactggcagcgactcatcaacaacaactggggattccgg |
| | AHQGCLPPFPADVFMIPQYGYLTLNN | cccaagagactcaacttcaagctcttcaacatccaggtcaaggaggtcacgcag |
| | GSQAVGRSSFYCLEYFPSQMLRTGNN | aatgaaggcaccaacaccatccccaataacctcaccagcaccatccaggtgttt |
| | FQFTYTFEDVPFHSSYAHSQSLDRLM | acggactcggagtaccagctgccgtacgttctcggctctgcccaccagggctgc |
| | NPLIDQYLYYLSRTQSTGGTAGTQQL | ctgcctccgttcccggcggacgtcttcatgattccccagtacggctacctaaca |
| | LFSQAGPNNMSAQAKNWLPGPCYRQQ | ctcaacaacggtagtcaggccgtgggacgctcctccttctactgcctggaatac |
| | RVSKTSADNNNSEYSWTGATKYHLNG | tttccttcgcagatgctgagaaccggcaacaacttccagtttacttacaccttc |
| | RDSLVNPGPAMASHKDDEEKFFPQSG | gaggacgtgcctctccacagcagctacgcccacagccagagcttcgaccggctg |
| | VLIFGKQGSEKTNVDIEKVMITDEEE | atgaaccccctcatcgaccagtacctgtactacctgtctcggaatcagtccacg |
| | IRTTNPVATEGYGSVSTNLQRGNRQA | ggaggtaccgcaggaactcagcagttgctattttctcaggccgggcctaataac |
| | ATADVNTQGVLPGMVWQDRDVYLQFP | atgtcggctcaggccaaaaactgcctacccgggccctgctaccggcagcagcga |
| | IWAKIPHTDGHFHPSPLMGGFGLKHP | gtatcaaagacatctgcggataacaacaacagtgaatactcgtggactggagct |
| | PPQILIKNTPVPADPPTTFNQSKLNS | accaagtaccacctcaatggcagagactctctggtgaacccgggcccggccatg |
| | FITQYSTGQVSVEIEWELQKENSKRW | gcaagccacaagcacgatgaagaaaagttttttcctcagagcggcgttctcatc |
| | NPEIQYTSNYYKSTSVDEAVNTEGVY | tttgggaagcaaggctcagagaaaacaaatgtggacattgaaaaggtcatgatt |
| | SEPRPIGTRYLTRNL SEQ ID NO: | acagacgaagaggaaatcaggacaaccaatcccgtggctacgcagcagtatggt |
| | 104 | tctgtatctaccaacctccagagaggcaacagacaagcagctaccgcagatgtc |
| | | aacacacaaggcgttcttccaggcatggtctggcaggacagagatgtgtacctt |
| | | caggggcccatctgggcaaagattccacacacggacggacattttcacccctct |
| | | cccctcatgggtggattcggacttaaacaccctcctccacagatcctgatcaag |
| | | aacacgcctgtacctgcggatcctccgaccaccttcaaccagtcaaagctgaac |
| | | tctttcatcacgcaatacagcaccgaacaggtcagcgtggaaattgaatgggag |
| | | ctgcagaaggaaaacagcaagcgctggaaccccgagatccagtacacctccaac |
| | | tactacaaatctacaagtgtggactttgctgttaatacagaaggcgtgtactct |
| | | gaaccccgccccattggcacccgttacctcacccgcaacctg-3′ SEQ ID |
| | | NO: 123 |
| HW04 | MAAGGGAPMADNNEGADGVGNSSGNW | 5′-atggcttcaggcggtggcgcaccaatggcagacaataacgaaggcgccgac |
| (VP3) | HCDSTWLGDRVITTSTRTWALPTYNN | ggagtgggtaatgcctcaggaaattggcattgcgattccacatggctgggcgat |
| | HLYKQISNSTSGGSSNDNAYFGYSTP | agagtcatcaccaccagcacccgaacctgggccctccccacctacaacaatcac |
| | WGYFDFNRFHCHFSPRDWQRLINNNW | ctctacaagcaaatctccaacagcacatctggaggatcttcaaatgacaacgcc |
| | GFRFKRLNFKLFNIQVKEVTQNEGTK | tacttcggctacagcaccccctgggggtattttgacttcaacagattccactgc |
| | TIANNLTSTIQVFTDSEYQLPYVLGS | catttctcaccacgtgactggcagcgactcatcaacaacaattggggattccgg |
| | AHQGCLPPFPADVFMIPQYGYLTLNN | cccaagagactcaacttcaagctcttcaacatccaagtcaaggaggtcacgacg |
| | GSQAVGRSSFYCLEYFPSQMLRTGNN | aatgatggcgtcacgaccatcgctaataaccttaccagcacggttcaagtcttc |
| | FQFTYTFEDVPFHSSYAHSQSLDRLM | ccggactcggagtaccagttgccgcacgtcctcggctctgcgcaccagggctgc |
| | NPLIDQYLYYLSRTQSTGGTAGTQQL | ctccctccgttcccggcggacgtgttcatgattcctcagtacggctacctgact |
| | LFSQAGPNNMSAQAKNWLPGPCYRQQ | ctcaacaatggcagtcagtctgtgggccgttcctcctcctactgcctggaatat |
| | RVSKTSADNNNSEYSWTGATKYHLNG | ttcccatcgcagatgctgagaacgggcaataactttaccttcagctacaccttc |
| | RDSLVNPGPAMASHKDDEEKFFPQSG | gaggacgtgcctttccacagcagctacgcccacagccagagcttggaccggctg |
| | VLIFGKQGSEKTNVDIEKVMITDEEE | atcaatcctctcatcgaccagtacctgtactacttatccagaactcagtccaca |
| | IRTTNPVATEGYGSVSTNLQRGNRQA | ggaggaactcaaggtacccagcaactgttatcttctcaagctgggcctgcaaac |
| | ATADVNTQGVLPGMVWQDRDVYLQFP | atgtcggctcaggctaagaactggctacctggaccttgctaccggcagcagcga |
| | IWAKIPHTDGHFHPSPLMGGFGLKHP | gtatcaaagacatcagcggataacaacaacagtgaatactcgtggactggagct |
| | PPQILIKNTPVPADPPTTFNQSKLNS | accaagtaccacctcaatggcagagactctctggtcaatccgggcccggccatg |
| | FITQYSTGQVSVEIEWELQKENSKRW | gcaagccacaaggacgatgaagaaaagttttttcctcagagcggggttctcatc |
| | NPEIQYTSNYYKSTSVDEAVNTEGVY | ttcgggaagcaaggctcagagaaaacaaatgtggacactgaaaaggtcatgatt |
| | SEPRPIGTRYLTRNL SEQ ID NO: | acagacgaagaggaaatcaggacaaccaatcccgtggctacggagcagtatggt |
| | 105 | tctgtatctaccaacctccagagaggcaacagacaagcagctaccgcagatgtc |
| | | aacacacaaggcgttcttccaggcatggtctggcaggacagagatgtgtacctt |
| | | caggggcccatctgggcaaagattccacacacggacggacattttcacccctct |
| | | cccctcatgggtggattcggacttaaacaccctcctccacagattctcatcaag |
| | | aacaccccggtacctgcgaatccttcgaccaccctcaaccagtcaaagctgaac |
| | | tctttcatcacgcaatacagcaacggacaagtcagcgtggaaatcgagtgggag |
| | | ctgcagaaggaaaacagcaagcgctggaatccagagattcaatacacttccaac |
| | | tactacaaatctacaaatgtggactttgctgtcaacacggagggcgtttatagt |
| | | gagcctcgccccattggeacccgttacctcacccgtaatctg-3′ SEQ ID |
| | | NO: 129 |
| HW05 | MAAGGGAPMADNNEGADGVGSSSGNW | 5′-atggctgcaggcggtggcgcaccaatggcagacaataacgaaggcgccgac |
| (VP3) | HCDSTWLGDRVITTSTRTWALPTYNN | ggagtgggtagttcctcgggaaattggcattgcgattccacatggctggccgac |
| | HLYKQISSASTGASNDNTYFGYSTPW | agagtcatcaccaccagcacccgaacatgggccttgcccacctacaacaaccac |
| | GYFDFNRFHCHFSPRDWQRLINNNWG | ctctacaagcaaatctccagtgcttcaacgggggccagcaacgacaacacccac |
| | FRPKKLRFKLFNIQVKEVTQNEGTKT | ttcggctacagcaccccctgggggtattttgacttcaacagattccactgtcac |
| | IANNLTSTVQVFTDSEYQLPYVLGSA | ttttcaccacgtgactggcaacgactcatcaacaacaactggggattccggccc |
| | HQGCLPPFPADVFMVPQYGYLTLNNG | aacaagctgcggctcaagctcttcaacatccaggtcaaggaggtcacgcagaat |
| | SQALGRSSFYCLEYFPSQMLRTGNNF | gaaggcaccaagaccatcgccaataatctcaccagcaccgtgcaggtctttacg |
| | QFTYTFEDVPFHSSYAHSQSLDRLMN | gactcggagtaccagttaccgtacgtgccaggatccgctcaccagggatgtctg |
| | PLIDQYLYYLSRTQSTGGTAGTQQLL | cctccgttcccggcggacgccttcatggttcctcagtacggctacttaactcta |
| | FSQAGPNNMSAQAKNWLPGPCYRQQR | aacaatggaagccaagccccgggacgttcctccttctactgtctggagtatttc |
| | VSKTSADNNNSEYSWTGATKYHLNGR | ccatcgcagatgctgagaaccggcaacaactttcagtttacttacaccttcgag |
| | DSLVNPGPAMASHKDDEEKFFPQSGV | gacgtgcctttccacagcagctacgcgcacagccagagcctggacaggctgatg |
| | LIFGKQGSEKTNVDIEKVMITDEEEI | aatcccctcatcgaccagtacctgtactacctgtctcggactcagtccacggga |
| | RTTNPVATEQYGSVSTNLQRGNRQAA | ggtaccgcaggaacccagcagctgctatcttctcaggccgggcctaataacatg |
| | TADVNTQGVLPGMVWQDRDVYLQGPI | tccgctcaggccaaaaactgactacccgggccctgctaccggcagcagcgagta |
| | WAKIPHTDGNFHPSPLMGGFGMKHPP | tcaaagacatctgcggataacaacaacagtgaatactcgtggactggagctacc |
| | PQILIKNTPVPADPPTTFSQAKLASF | aagtaccacctcaacggcagagaccctccggtgaacccgggcccggccatggca |
| | ITQYSTGQVSVEIEWELQKENSKRWN | agccacaaggacgatgaagaaaagttttttcctcagagcggggttctcatcttt |
| | PEIQYTSNYYKSTNVDFAVNTEGTYS | gggaagcaaggctcagagaaaacaaatgtggacattgaaaaggtcatgattaca |
| | EPRPIGTRYLTRNL SEQ ID NO: | gacgaagaggaaatcaggacaaccaatcccgtggctacggagcagtatgcttct |
| | 106 | gtatctaccaacctccagagaggcaacagacaagcagctaccgcagatgtcaac |
| | | acacaaggcgttcttccaggcatggtctggcaggacagagatgtgtaccttcag |
| | | gggcccatctgggcaaagattccacacacggacggcaactttcacccttctccg |
| | | ctgatgggagggtttggaatgaagcacccacctcctcagatcctgatcaagaac |
| | | acgccggtacctgcggatcctccaacaacgttcagccaggcgaaattggcttcc |
| | | ttcattacgcagtacagcaccggacaggtcagcgtggaaatccagtgggagctg |
| | | cagaaggagaacagcaaacgctggaacccagagattcagtacacttcaaactac |
| | | tacaaatctacaaatgtggacttcgctgtcaatacagagggaacctattctcag |
| | | cctcgccccattggtactcgttaccteacccgtactctg-3′ SEQ ID NO: |
| | | 130 |
| HW05 | MAAGGGAPMADNNEGADGVGSSSGNW | 5′-atggctgcaggcggtggcgcaccaatggcagacaataacgaaggcgcccac |
| (VP3) | HCDSTWLGDVITTSTRTWALPTYNN | ggagtggatagttcctcgggaaattggcattgcgartccacatggctggccgac |
| | HLYKQISSASTGASNDNHYFGYSTPW | agagtcatcaccaccagaacccgaacatgggccttgcccacctataacaaccac |
| | GYFDFNRFHCHFSPRDWQRLINNNWG | ctctacaagcaaatctccagtgcttcaacgggggccagcaacgacaaccactac |
| | FRPKKLRFKLFNIQVKEVTDNNGVKT | ctcggctacagcaccccctgggggcattctgacttcaacagactccactgccac |
| | IANNLTSTIQVFTDSEYQLPYVLGSA | ttttcaccacgtgactggcagcgactcatcaacaacaactggggattccggccc |
| | HQGCLPPFPADVFMIPQYGYLTLNNG | aagaagctgcggttcaagctcttcaacattcaggtcaaagaggtcacggacaac |
| | SQAVGRSSFYCLEYFPSQMLRTGNNF | aatggagtcaaaaccatcgccaataacctcaccagcaccatccaggtgtttacg |
| | QFSYEFENVPFHSSYAHSQSLDRLMN | gactcggagtaccagctgccgtacgttcccggctctgcccaccagggctgcctg |
| | PLIDQYLYYLSRTQTTGGTANTQTLG | cccccgttcccggcggacgtcttcatgattccgcagtacggctaccttacactg |
| | FSQGGPNTMANQAKNWLPGPCYRQQR | aacaatggaagtcaagccgtaagccgttcctccttctactgcctggaatacttt |
| | VSTTTGQNNNSNFAWTAGTKYHLNGR | ccttcgcagatgctgagaaccggcaacaacttccagttcagctacgagtttgag |
| | NSLANPGIAMATHKDDEERFFPSNGI | aacgtacctttccatagcagctacgctcacagccaaagcctggaccgactaatg |
| | LIFGKQNAARDNADYSDVMLTSEEEI | aatccactgattgaccagtacctgtactacttgtctcggactcaaacaacagga |
| | KTTNPVATEEYGIVADNLQQQNTAPQ | ggcacggcaaatacgcagactctgggcttcagccaaggtgggcctaatacaatg |
| | IGTVNSQGALPGMVWQNRDVYLQGPI | gccaatcaggcaaagaactggctgccaggaccctgttaccgccaacaacgcgtc |
| | WAKIPHTDGNFHPSPLMGGFGLKHPP | tcaacgacaaccgggcaaaacaacaatagcaactttgcctggactgctgggacc |
| | PQILIKNTPVPANPPEVFTPAKFASF | aaataccatctgaatggaagaaattcattggctaatcctggcatcgctacggca |
| | ITQYSTGQVSVEIEWELQKENSKRWN | acacacaaagacgacgaggagcgtttttttcccagtaacgggatcctgattttt |
| | PEIQYTSNFEKQTGVDFAVDSQGVYS | ggcaaacaaaatgctgccagagacaatgcggattacagcgatgtcatgctcacc |
| | EPRPIGTRYLTRNL SEQ ID NO: | agcgaggaagaaatcaaaaccactaaccctgcggctacagaggaatacgctatc |
| | 107 | gtcgcagataacttgcagcagcaaaacacggctcctcaaattggaactgtcaac |
| | | agccagggggccttacccggtatggtctggcagaaccgggacgtgtacctgcag |
| | | ggtcccatctgggccaagattcctcacacggacggcaactttcacccttctccg |
| | | ctgatgggcggctttggcctgaaacacccgcctcctcagatcctgattaagaat |
| | | acacctgttcccgctaatcctccggaggtgtttactcctgccaagtttgctccg |
| | | ctcatcacacagtacagcaccggacaagccagcgtggaaatcgagtgggagctg |
| | | cagaaggaaaacagcaagcgctggaacccggagattcagtacacctccaacttt |
| | | gaaaagcagactggtgcggactttgccgttgacagccagggtgtttactctgag |
| | | cctcgccctattggcactcgttacctcacccgtaatctg-3′ SEQ ID NO: |
| | | 131 |
| HW07 | VAAGGGAPMADNNEGADGVGNASGNW | 5′-gtggctgcaggcggtggcgcaccaatggcagacaataacgaaggtgccgac |
| (VP3) | HCDSTWLGDRVITTSTRTWALPTYNN | ggagtgggtaatgcctcaggaaattggcattgcgattccacatggctgggcgac |
| | HLYKQISSETAGSTNDNTYFGYSTPW | agagtcattaccaccaccacccgaacctgggccctgcccacctacaacaaccac |
| | GYFDFNRFHCHFSPRDWQRLINNNWG | ctctacaagcaaatctccagtgaaactgcaggtagtaccaacgacaacacctac |
| | FRPKRLNFKLFNIQVKEVTTNEGTKT | ttcggctacagcaccccctgggggcattttgacttcaacagattccactgtcac |
| | IANNLTSTVQVFTDSEYQLPYVLGSA | ttttcaccacgtgactggcaacgactcatcaacaacaattggggattccggccc |
| | HQGCLPPFPADVFMIPQYGYLTLNNG | aaaagactcaacttcaagctgttcaacatccaggtcaaggaagtcacgacgaac |
| | SQAVGRSSFYCLEYFPSQMLRTGNNF | gaaggcaccaagaccatcgccaataatctcaccagcaccgtgcaggtctttacg |
| | EFSYSFEDVPFHSSYAHSQSLDRLMN | gactcggaataccagctcccgtacgtcctcggctctgcgcaccacggctgcctg |
| | PLIDQYLYYLSRTNTPSGTTTQSRLQ | cctccgttcccggcggacgtcttcatgattcctcagtacgggtacctgactctg |
| | FSQAGASDIRDQSRNWLPGPCYRQQR | aacaatggaagtcaagccgtaagccgttcctccttctactgcctggaatatttc |
| | VSKTSADNNNSEYSWTGATKYHLNGR | ccatcgcagatgctgagaacgggcaacaactttgagttcagctacagcttcgag |
| | DSLVNPGPAMASHKDDEEKFFPQSGV | gacgttcctttccacagcagctacgctcacagccagagtctggaccgtctcatg |
| | LIFGKQGSEKTNVDIEKVMITDEEEI | aatcctctcatcgaccagtacctgtattacttgagcagaacaaacactccaagt |
| | RTTNPVATEQYGSVSTNLQRGNRAQQ | ggaaccaccacgcagtcaaggcttcagttttctcaggccggagcgagtgacatt |
| | TADVNTQGVLPGMVWQDRDVYLQGPI | cgcgaccagtctaggaactggcttcctggaccctgttaccgccagcagcgacta |
| | WAKIPHTDGHFHPSPLMGGFGLKHPP | tcaaagacatctgcggataacaacaacagtgaatactcgtggactggagctacc |
| | PQILIKNTPVPADFPTTFSQAKLASF | aagtaccacctcaatggcagagactctctggtgaatccgggcccggccatggca |
| | ITQYSTGQVSVEIEWELQKENSKRWN | agccacaagcacgatgaagaaaagttctttcctcagagcggggtcctcatcctt |
| | PEIQYTSNYYKSNTVDFAVNTEGVYS | gggaagcaaggctcagagaaaacaaatgtggacattgaaaaggtcatgattaca |
| | EPRPIGTRYLTRNL SEQ ID NO: | gacgaagaggaaatcaggacaaccaatcccgtggctacggagcagtatggttct |
| | 108 | gtatctaccaacctccagagaggcaacagacaagcagctaccgcagacgtcaac |
| | | acacaaggcgttcttccaggcatggtctggcaggacagagatgtgtaccttcag |
| | | gggcccatctgggcaaagattccacacacggacggacattttcacccctctcct |
| | | ctcatgggcggctttggacttaagcacccgcctcctcagatcctcatcaaaaac |
| | | acgcctgttcccgcggatcctccaactaccttcagtcaagctaagctggcgtcg |
| | | ctcatcacgcagtacagcacccgacaggtcagcgtcgaaatcgagtgggagctg |
| | | cacaaggagaacagcaaacgctgcaacccagagattcaatacacttccaactac |
| | | tacaaatctacaaatgtggactttgctgttaatacagaaggcgtgtactctgaa |
| | | ccccgccccattggcacccgtcacctcacccgtaatctg-3′ SEQ ID NO: |
| | | 132 |
| HW08 | MAAGGGAPMADNNEGADGVGSSSGNW | 5′-atggctgcaggcggtggccctccaatggcagacaataacgaagacgccgac |
| (VP3) | HCDSTWLGDRVITTSTRTWALPTYNN | ggagtgggtagttcctcaggaaattggcattgcgactccacatggctgggcgac |
| | HLYKQISSQSGASNDNHYFGYSTPWG | agagtcatcaccaccagcacccgaacctgggccctccccacctacaacaaccac |
| | YFDFNRFHCHRSPRDWQRLINNNWGF | ctctacaaacaaatttccagccaatcaggagcctcgaacgacaatcactacttt |
| | YFDFNRFHCHFSPRDWQRLINNNWGF | ggctacagcaccccttgggggtattttgacttcaacagattccaccgccacttt |
| | RPKRLNFKLFNIQVKEVTDNNGVKTI | tcaccacgtgaccggcagcgactcatcaacaacaactggggattccggcctaag |
| | ANNLTSTIQVFTDSEYQLPYVLGSAH | cgactcaacttcaagctcttcaacattcaggtcaaagaggttacggacaaccat |
| | QGCLPPFPADVFMIPQYGYLTLNNGS | ggagtcaagaccatcgccaataacctcaccagcaccatccaggtgtttacggac |
| | QSVGRSSFYCLEYFPSQMLRTGNNFE | tcggagtaccagctgccgtacgtcctcggctctgcgcaccagggctgcctgcct |
| | FSYTFEDVPFHSSYAHSQSLDRLMNP | ccgttcccggcggacgtcttcatgattcctcagtacggctacctgactctcaac |
| | LIDQYLYYLSRTQSTGGTAGTQQLLF | aatggcagtcagtccgtgggacgttcctccttctactgcctggaatattttcca |
| | SQAGPNNMSAQAKNWLPGPCYRQQRV | tctcaaatgctgcgaactggaaacaattttcaattcagctacaccttcgaggac |
| | SKTSADNNNSEYSWTGATKYHLNGRD | gtgcctttccacagcagctacgcacacagccagagcttggacggactgatgaat |
| | SLVNPGPAMASHKDDEEKFFPQSGVL | cctctcatcgaccagtacctgtaccacttatccagaactcagtccacggcaggt |
| | IFGKQGSEKTNVDIEKVMITDEEEIR | accgcaggaaatcagcagttgctattttctcaggccgggcctaataacacgtcg |
| | TTNPVATEQYGSVSTNLQRGNRQAAT | gctcaggccaaaaactggctacccgggccctgctaccgccagcagcgagtatca |
| | ADVNTQGVLPGMVWQDRDVYLQGPIW | aagacatctgcggataacaacaacagtgaatactcgtggactggagctaccaag |
| | AKIPHTDGHFHPSPLMGGFGLKHPPP | taccacctcaatggcagagactctctggtgaatccgggcccggccatggcaagc |
| | QILIKNTPVPANPSTTFSAAKFASFI | cacaaggacgatgaagaaaagttttttcctcagagcggggttctcatctttggg |
| | TQYSTGQVSVEIEWELQKENSKRWNP | aagcaagcctcagagaaaacaaatctggacattgaaaaggtcatgattacagac |
| | EIQYTSNYNKSVNVDFTVDTNGVYSE | gaagaggaaatcaggacaaccaatcccgtggctacggagcagtatggttctgta |
| | PHPIGTRYLTRNL SEQ ID NO: | tctaccaacctccagagaggcaacagacaagcagctaccgcagatgtcaacaca |
| | 109 | caaggcgttcttccaggcatggtctggcaggacagagatgtgtaccttcagggg |
| | | cccatctgggcaaagattccacacacggacggacattttcacccctctcccctc |
| | | atcggcggattcggacctaaacaccctcctccacagactctcatcaagaacacc |
| | | ccggtacctgcgaatccttcgaccaccttcagtgccgcaaagtttgcttccttc |
| | | atcacacagtactccacgggacaggtcagcgtggagatcgagtgggagctgcag |
| | | aaggaaaacagcaaaccctggaaccccgagatccagtacacttccaactacaac |
| | | aagtctgttaatgtggactttactgtggacactaatggcgtgtattcagagcct |
| | | caccccattggcaccagatacctcacccgtaatctg-3′ SEQ ID NO: 133 |
| HW09 | MATGSPAPMADNNEGADGVGNSSGNW | 5′-atggctacaggcagtggcgcaccaatggcagacaataacgagggcgccgac |
| (VP3) | HCDSTWLGDRVITTSTRTWALPTYNN | ggagtgggtaattcctcgggaaatcggcattgcgattccacatggctgggcgac |
| | HLYKQISSQSSPRTTITTLATAPPGG | agagtcattaccaccagcacccgaacctgggccctgcccacctacaacaaccac |
| | YFDFNRFHCHFSPRDWQRLINNNWGF | ctctacaaacaaatttccagccaatcnnagcctcgaacgacaatcactactttg |
| | RPKRLSFKLFNIQVKEVTQNEGTKTI | gctacagcaccccctggggggtatcttgactttaacagattccactgccacttt |
| | ANNLTSTIQVFTDSEYQLPYVLGSAH | tcaccacgtgactggcagcgactcatcaacaacaactggggattccggccaaaa |
| | QGCLPPFPADVFMVPQYGYLTLNNGS | agactcagcttcaagctcttcaacatccaggtcaaggaggtcacgcagaatgaa |
| | QALGRSSFYCLEYFPSQMLRTGNNFQ | ggcaccaagaccatcgccaataaccttaccagcacgattcaggtatttacggac |
| | FSYTFEDVPFHSSYAHSQSLDRLMNP | tcggaataccagctgccgtacgtcctcggctctgcgcaccagggctgcctgcct |
| | LIDQYLYYLSRTQSTGGTAGTQQLLF | ccgttcccggcggacgtcttcatggtccctcagcacggctatttaaccttaaac |
| | SQAGASDIRDQSRNWLPGPCYRQQRV | aatggaagccaagccctgggacgttcctccttctactgtctggagtatttccca |
| | AKTSTDNNNSEYSWTGATKYHLNGRD | tcgcagatgctgagaaccggcaacaactttcagttcagctacaccttcgaggac |
| | SLVNPGPAMASHKDDEEKFFPQSGVL | gtgcctttccacagcagctacgcccacagccagagtccggaccggctgatgaac |
| | IFGEQGSEKTNVDIEKMVITSEEEIR | cccctcatcgaccagtacctgtactacctgtctcggactcagtccacgggaggt |
| | TTNPVATEQYGSVSTNLQRGNRQAAT | accgcaggaactcagcagttgctattttctcaggccggagcgagtgacattcgg |
| | ADVNTQGVLPGMVWQDRDVYLQGPIW | gaccagtctaggaactggcttcctggaccctgttaccgccagcagcgagtatca |
| | AKIPHTDGNFHPSPLMGGFGLKHPPP | aagacatctacggataacaacaacagtgaatactcgtggactggagctaccaag |
| | QILIKNTPVPADPPTTFNQSKLNSFI | taccacctcaatggcagagaccctctggcgaatccgggcccggccatggcaagc |
| | TQYSTGQVSVEIEWELQKENSKRWNP | cacaaggacgatgaagaaaagttttttcctcagagcggggttctcatctttggg |
| | EIQYTSNYYKSTNVDFAVNTEGVYSE | gagcaaggctcagagaaaacaaatgtggacattgaaaaggtcatgattacagac |
| | PRPIGTRYLTRNL SEQ ID NO: | gaagaggaaatcaggacaaccaatcccgtggctacggagcagtatggttctgta |
| | 110 | tctaccaacctccagagaggcaacagacaaccagctaccgcagatgtcaacaca |
| | | caaggcgttcttccaggcatggtctggcaggacagagatgtgtaccttcagggg |
| | | cccatctgggcaaagattccacacacggacggcaacttccacccgtctccgctg |
| | | ttgggcggctttggcctgaaacatcctccgcctcagatcctgatcaagaacacg |
| | | cctgtacctgcggatcctccgaccaccttcaaccagtcaaagctgaactccttc |
| | | attacgcagtacagcaccggacaggtcagcgtggaaatcgagtgggagctgcag |
| | | aaggagaacagcaaacgctggaacccagagactcagtacacttcaaactactac |
| | | aaatccacaaatgtggactttgctgtcaacacggaggcggtttatagcgagcct |
| | | cgccccattggcacccgttacctcacccgcaacctg-3′ SEQ ID NO: 134 |
| HW10 | MASGGGAPMADNNEGADGVGNASGNW | 5′-atggcttcacgcggtggcgcaccaatggcagacaataacgaaggcgccaac |
| (VP3) | HCDSTWMGDRVITTSTRTWALPTYNN | ggagtgggtaatgcctcaggaaattggcattgcgattccacatggatgggcgac |
| | HLYKQISSQSGASNDNHYFGYSTPWG | agagtcatcaccaccagcacccgaacctgggccctgcccacccacaacaaccac |
| | YFDFNRFHCHRSPRDWQRLINNNWGF | ctctacaaacaaatttccagccaatcaggagcctcgaacgacaatcactacttt |
| | RPKRLSFKLFNIQVKEVTQNEGTKTI | ggctacagcaccccctgggggtattttgactttaacagattccactgccacttt |
| | ANNLTSTIQVFTDSEYQLPYVLGSAH | tcaccacgtgactggcagcgactcatcaacaacaactggggattccggcccaag |
| | QGCLPPFPADVFMIPQYGLYTLNNGS | agactcagcttcaagctcttcaacatccaggccaaggaggtcacgcagaatgaa |
| | QAVGRSSFYCLEYFPSQMLRTGNNFE | ggcaccaagaccatcgccaataacctcaccagcaccatccaggtgttcacggac |
| | FSYFTEDVPFHSSYAHSQSLDRLMNP | tcggagtaccagctcccgtacctcctcggctctgcgcaccagcgctgcctgcct |
| | LIDQYLYYLSRTNTPSGTTTQSRLQF | ccgttcccggcggacgtgttcatgattcctcagtacgggtacctgactctgaac |
| | SQAGASDIRDQSRNWLPGPCYRQQRV | aacggcagccaggccgtgggccgctcctccttccaccgcccggaatacttccca |
| | SKTSADNNNSEYSWTGATKYHLNGRD | tctcaaatgctgcgaactggaaacaattttgaattcagctacaccttcgaggac |
| | SLVNPGPAMASHKDDEEKFFPQSGVL | gtgccttcccacagcagctacgcacacagccagagcttggaccgactgatgaat |
| | IFGKQGSEKTNVDIEKVMITDEEEIR | cccctcatcgaccagtacctgtactacttgagcagaacaaacaccccaagtgga |
| | TTNPVATEQYGSVSTNLQRGNRQAAT | accaccacgcagtcaaggcttcagttttctcaggccggagcgagtgacattcgg |
| | ADVNTQGVLPGMVWQDRDVYLQGPIW | gaccagtctaggaactggcttcctggaccctgttaccgccagcagcgagtatca |
| | AKIPHTDGHFHPSPLMGGFGLKHPPP | aacacatctccggataacaacaacagcgaacactcgtcgactggagctaccaag |
| | QILIKNTPVPADPPLTFNQAKLNSFI | taccacctcaatggcagagactctctggtgaatccgggcccggccatggcaagc |
| | TQYSTGQVSVEIEWELQKENSKRWNP | cacaaggacgatgaagaaaagttttttcctcagagcggggttctcatctttggg |
| | EIQYTSNYYKSTSVDFAVNTEGVYSE | aaccaaggctcagagaaaacaaacgtggacattgaaaaggtcatcatcacagac |
| | PRPIGTRYLTRNL SEQ ID NO: | gaagaggaaatcaggacaaccaatcccgtggctacggagcagtacggttctgta |
| | 111 | tctaccaacctccagagaggcaacagacaagcagccaccgcagatgtcaacaca |
| | | caaggcgttcttccaggcatggtctggcaggacagagatgtgtaccttcagggg |
| | | cccatctgggcaaagattccacacacggacggacattctcacccctctcccctc |
| | | atgggtggattcggacttaaacaccctcctccacagatcctcatcaagaacaca |
| | | ccggttccagcggacccgccgcttaccttcaaccaggccaagctgaactctttc |
| | | atcacgcagtacagcaccggacaggtcagcgtggaaatcgagtgggagctgcag |
| | | aaagaaaacagcaagcgctggaacccggagattcagtacacctccaactactac |
| | | aaatctacaagtgtggactttgctgttaatacagaaggcgtgtactctgaaccc |
| | | cgccccattggcacccgttacctcacccgtaatctg-3′ SEQ ID NO: 135 |
| HW11 | VAAGGGAPMADNNEGADGVGNASGNW | 5′-gtggctgcaggcggtggcgcaccaatggcagacaataacgaaggtgccgac |
| (VP3) | HCDSTWMGDRVITTSTRTWALPTYNN | ggagtgggtaatgcctcaggaaattggcattgcgattccacatgcatgggcgac |
| | HLYKQISSQSGASNDNHYFGYSTPWG | agagtcatcaccaccagcacccgaacctgggccctgcccacctacaacaaccac |
| | YFDFNRFHCHFSPRDWQRLINNNWGF | ctctacaaacaaatttccagccaatcaggagcctcgaacgacaatgactacttt |
| | RPKRLNFKLFNIQVKEVTDNNGVKTI | ggctacagcaccccctgggggtattttgacttcaacagattccactgccatttc |
| | ANNLTSTVQVFTDSDYQLPYVLGSAH | tcaccacgtgactggcagcgactcatcaacaacaactggggattccggcctaag |
| | EGCLPPFPADVFMIPQYGYLTLNDGS | cgactcaacttcaagctcttcaacattcaggtcaaagaggttacggacaacaat |
| | QAVGRSSFYCLEYFPSQMLRTGNNFQ | ggagtcaagaccatcgccaataaccttaccagcacggtccaggtcttcacggac |
| | FTYTFEDVPFHSSYAHSQSLDRLMNP | tcagactatcagctcccgtacgtgctcgggtcggctcacgagggctgcctcccg |
| | LIDQYLYYSLRTNTPSGTTTQSRLQF | ccgttcccagcggacgttttcatgattcctcagtacgggtatctgacgcttaat |
| | SQAGASDIRDQSRNWLPGPCYRQQRV | gatggaagccaggccgtgggtcgttcgtccttttactgcctggaatattcccca |
| | SKTSADNNNSEYSWTGATKYHLNGRD | tcgcagatgctgagaaccggcaacaacttccagcttacttacaccttcgagcac |
| | SLVNPGPAMASHKDDEEKFFPQSGVL | gtgcctttccacagcagctacgcccacagccagagcttggaccggctgatgaat |
| | IFGKQGSEKTNVDIEKMVITDEEEIR | cctgtgattgaccagtacctgcactacttgagcagaacaaacactccaagtgga |
| | TTNPVATEQYGSVSTNLQRGNRQAAT | accaccacgcagtcaaggcttcagttttctcaggccggagcgagtgacattcgg |
| | ADVNTQGVLPGMVWQDRDVYLQGPIW | gaccagtctaggaactggcttcctggaccccgttaccgccagcagcgagtatca |
| | AKIPHTDGHFHPSPLMGGFGLKHPPP | aagacatctgcggataacaacaacagtgaatactcctggactcgagctaccaag |
| | QILIKNTPVPANPSTTFSAAKFASFI | taccacctcaatcgcacagactctctggtgaatccggccccggccatcgcaagc |
| | TQYSTGQVSVEIEWELQKENSKRWNP | cacaaggacgatgaagaaaagttttttcctcagagcggggttctcatctttggg |
| | EIQYTSNYNKSVNVDFTVDTNGVYSE | aagcaaggctcagagaaaacaaatgtggacattgaaaaggtcatgattacagac |
| | PRPIGTRYLTRNL SEQ ID NO: | gaagaggaaatcaggacaaccaatcccgtggctacggagcagtatggttctgta |
| | 112 | tctaccaacctccagagaggcaacagacaagcagctaccgcagacgtcaacaca |
| | | caaggcgctcttccaggcatgctccggcaggacagagacgtgcaccttcagggg |
| | | cccatctgggaaaagattccacacacggacggacactttcacccctctcccctc |
| | | atgggtggattcggacttaaacaccctcctccacagattctcatcaagaacacc |
| | | ccggtacctgcgaatccttcgaccaccttcagtgcggcaaagtttgcttccttc |
| | | atcacacagtactccacgggacaggtcagcgtggagatcgagtgggagctgcag |
| | | aaggaaaacagcaaacgctggaaccccgaaattcagtacacttccaactacaac |
| | | aagtctgttaatgtcgactttactgtggacactaatggcgtgtattcagagcct |
| | | cgccccattggcacccgttacctcacccgtaatctg-3′ SEQ ID NO: 136 |
| HW12 | MASGGGAPMADNNEGADGVGNSSGNW | 5′-atggcttcaggcggtggcgcaccaatggcagacaataacgaaggcgccgac |
| (VP3) | HCDSTWLGDRVITTSTRTWALPTYNN | ggagtgggtaattcctcgggaaattggcattgcgactccacatggctgggggac |
| | HLYKQISSQSGASNDNHYFGYSTPWG | agagtcatcaccaccagcacccgaacctgggccctgcccacctacaacaaccac |
| | YFDFNRFHCHFSPRDWQRLINNNWGF | ctctacaaacaaatttccagccaatcaggagccccgaacgacaaccactacctt |
| | RPKRLNPKLFNIQVKEVTQNDGTTTI | ggctacagcaccccctgggggcatcttgactccaacagattccactgccacttt |
| | ANNLTSTVQVFTDSEYQLPVYLGSAH | tcaccacgtcactggcaaagactcatcaacaacaactcgggattccgacccaag |
| | QGCLPPFPADVFMIPQYGYLTLNNGS | agactcaacttcaagctctttaacattcaagtcaaagaggccacgcagaatgac |
| | QAVGRSSFYCLEYFPSQMLRTGNNFT | ggtacgacgacgatcgccaataaccttaccagcaccgttcaggtgtttacggac |
| | FSYTFEDVPFHSSYAHSQSLDRLMNP | tcggagtaccagctgccgtacgctctcggctctgcccaccagggctgcctgcct |
| | LIDQYLYYLSRTNTPSGTTTQSRLQF | ccgttcccggcggacgtgttcatgattccccagtacggctacctaacactcaac |
| | SQAGASDIRDQSRNWLPGPCYRQQRV | aacggtagtcaggccgtgggacggtcatccttttactgcctggaatatttccca |
| | SKTSADNNNSEYSWTGATKYHLNGRD | tctcagatgctgagaacgggcaatacctttaccttcagctacaccttcgaggac |
| | SLVNPGPAMASHKDDEEKFFPQSGVL | gtgcctttccacagcagctacgcccacagccagagtccggaccgcctcatgaat |
| | IFGKQGSEKTNVKIEKVMITDEEEIR | cctctcatcgaccagtacctgtattacttgagcagaacaaacactccaagtgga |
| | TTNPVATEQYGSVSTNLQRGNRQAAT | accaccacgcagtcaaggcttcagctttctcaggccggagcgagtgacattcgg |
| | ADVNTQGVLPGMVWQDRDVYLQGPIW | gaccagtctaggaactggcttcccggaccccgttaccgccagcagcgagtacca |
| | AKIPHTDGNFHPSPLMGGFGLKHPPP | aagacatctgcggataacaacaacagtgaatactcgtggactggagctaccaag |
| | QILIKNTPVPANPSTTFSAAKFASFI | caccacctcaatggcagagactctctggtgaatccgggcccggccatggcaagc |
| | TQYSTGQVSVEIEWELQKENSKRWNP | cacaaggacgatgaagaaaagttcttccctcagagcggggctctcatctttggg |
| | EIQYTSNYNKSVNVDVTVDTNGVYSE | aagcaaggctcagagaaaacaaatgtggacattgaaaaggtcatgattacagac |
| | PRPIGTRYLTRNL SEQ ID NO: | gaagaggaaatcaggacaaccaatcccgtggctacggagcagcatggttctgca |
| | 113 | tctaccaacctccagacaggcaacagacaagcagctaccgcagatgtcaacaca |
| | | caaggcgttcttccaggcacggtctggcaggacagagatgtgtaccttcagggg |
| | | cccatctcggccaacattcctcacacggacggcaacttccacccctctcccctc |
| | | atcggtagattcgaacttcaacaccctcctccacagattctcatcaagaacacc |
| | | ccggtacctgcgaatccttcgaccaccttcagtgcggcaaagtttgcttccttc |
| | | atcacacagtactccacgggacaggtcagcgtggagatcgagcgggagccgcag |
| | | aaggaaaacagcaaacgctggaatcccgaaattcagtacacttccaactacaac |
| | | aagtccgtcaatgtggactctaccgtggacactaatggcgcgtactcagagcct |
| | | cgccccattggtactcgttacctcacccgtaatctg-3′ SEQ ID NO: 137 |
| HW13 | MASGGGAPMADNNEGADGVGNASGNW | 5′-atggcttcacgcgctggcgctccaatggcagacaataacgaaggcgccgac |
| (VP3) | HCDSTWLGDRVITTSTRTWALPTYNN | ggagtgggtaatgcctcaggaaattggcattgcgattccacatggctgggcgac |
| | HLYKQISSQSGASNDNHYFGYSTPWG | agagtcatcaccaccagcacccgaacctgggccctgcccacctacaacaaccac |
| | YFDFNRFHCHFSPRDWQRLINNNWGF | ctctacaaacaaatttccagccaatcaggagcctcgaacgacaaccactacctt |
| | RPKRLSFKLFNIQVKEVTQNEGTKTI | ggctacagcaccccttgggggtatttcgacttcaacagattccactgccacttt |
| | ANNLTSTIQVFTDSEYQLPVYLGSAH | tcaccacgtgactggcagcgactcatcaacaacaactggggattccggcccaag |
| | QGCLPPFPADVFMIPQYGYLTLNNGS | agactcagcttcaagctcttcaacatccaggtcaaggaggtcacgcagaatgaa |
| | QSVGRSSFYCLEYFPSQMLRTGNNFT | ggcaccaagaccatcgccaataaccttaccagcacgattcaggtctttacggac |
| | FSYTFEDVPFHSSYAHSQSLDRLMNP | ccggaataccagctcccgtacgtcctcggctctgcgcaccagggctgcccccct |
| | LIDQYLYYLSRTNTPSGTTTQSRLQF | cccttcccggcggacgtgttcatcattcctcagtacggctacctgactctcaac |
| | SQAGASDIRDQSRNWLPGPCYRQQRV | aatggcagtcagtctgtgggacgttcctccttctactgccgggagtacttcccc |
| | SKTSADNNNSEYSWTGATKYHLNGRD | tctcagatgctgcgtaccggaaacaactttaccttcagctacacttttgaggac |
| | SLVNPGPAMASHKDDEEKFFPQSGVL | gttcctttccacagcagctacgctcacagccagagtctggaccgtctcatgaat |
| | IFGKQGSKETNVDIEKVMITSEEEIR | cctctcatcgaccagtacctgtattacttgagcagaacaaacactccaagtgga |
| | TTNFVATEQYGSVSTNLQRGNRQAAT | accaccacgcagtcaaggcttcagctttctcaggccggagcgagtgacactcgg |
| | ADVNTQGVLPGMVWQDRDVYLQGPIW | gaccagtctaggaactggcttcctggaccctgttaccgccagcagcgagtatca |
| | AKIPHTDGNFHPSPLMGGFGLKHPPP | aagacatctgcggataacaacaacagtgaatactcgtggactggagctaccaag |
| | QILIKNTPVPADPPTTFNQSKLNSFI | taccacctcaatggcagagactctctggtgaatcccggcccggccatggcaagc |
| | TQYSTGQVSVEIEWELQKENSKRWNP | cacaaggacgatgaagaaaagttttttcctcagagcggggttctcatctttggg |
| | EIQYTSNYYKSTSVDFAVNTEGVYSE | aagcaaggctcagagaaaacaaacgtggacattgaaaaggtcatgatcacagac |
| | PRPIGTRYLTRNL SEQ ID NO: | gaagaggaaatcaggacaaccaatcccgtggctacggagcagtatggttctgta |
| | 114 | tctaccaacctccagagaggcaacagacaagcagctaccgcagatgtcaacaca |
| | | caaggcgttcttccaggcatggtctggcaggacagagatgtgtacctccagggg |
| | | cccatctgggccaaaattcctcacacggacggcaacttccacccgtctcccctg |
| | | atgggcggctttggactgaagcacccgcctcctcagatcctgatcaagaacacg |
| | | cctgtacctgcggatcctccgaccaccttcaaccagtcaaagctcaactctttc |
| | | atcacgcaatacagcaccggacaggtcagcgtggaaattgaatgggagctgcag |
| | | aaagaaaaaagcaagcgctggaaccccgagatccagtacacatccaactactac |
| | | aaatctacaagtgtggactttgctgtcaacacggagggggtttatagcgagcct |
| | | cgccccattggcacccgttacctcacccgtaatctg-3′ SEQ ID NO: 138 |
| HW14 | MAAGGGAPMADNNEGADGVGNASGNW | 5′-atggctgaaggcggtggcgcaccaatggcagacaataacgaaggcgccgac |
| (VP3) | HCDSTWLGDRVITTSTRTWALPTYNN | ggagtgggtaatgcctcaggaaattggcattgcgattccacatggctgggcgac |
| | HLYKQISSETAGSTNDNTYFGYSTPW | agagtcatcaccaccagcacccgaacatgggccttgcccacctataacaaccac |
| | GYFDFNRFHCHFSPRDWQRLINNNWG | ctctacaagcaaatctccagtgaaactgcaggtagtaccaacgacaacacctac |
| | FRPKRLSFKLFNIQVKEVTTNDGVTT | ttcggctacagcaccccctgggggtattttgactttaacagattccactgccac |
| | IANNLTSTVQVFSDSEYQLPYVLGSA | ttctcaccacgtgactggcagcgactcatcaacaacaactggagattcccgcca |
| | HQGCLPPFPADVFMIPQYGYLTLNNG | aaaagactcagcttcaagctcttcaacatccaagtcaaggaggtcacgacgaat |
| | SQAVGRSSFYCLEYFPSQMLRTGNNF | gacggcgtcacgaccatcgctaataaccttaccagcacggttcaagtcttctcg |
| | QFSYTFEDVPFHSSYAHSQSLDRLMN | gactcggagtaccagttgccgtacgtcctcggctctgcgcaccagggctgcctg |
| | PLIDQYLYYLSRTNTPSGTTTQSRLQ | cctccgttcccggcggacgtcttcatgattccccagtacggctacctaacactc |
| | FSQAGASDIRDQSRNWLPGPCYRQQR | aacaacggtagtcaggccgtgggacgctcctccttctactgtctggagtatctc |
| | VSKTSADNNNSEYSWTGATKYHLNGR | ccatcgcagatgctgagaaccggcaacaactttcagcccagctacaccttcgag |
| | DSLVNPGPAMASHKDDEEKFFPQSGV | gacgtgcctttccacagcagctacgcgcacagccagagcctggacaggccgatg |
| | LIFGKQGSEKTNVDIEKVMITDEEEI | aatcccctcatcgaccagtacctctattacttgagcacaacaaacactccaagt |
| | RTTNPVATEQYGSVSTNLQRGNRQAA | ggaaccaccacgcagtcaaggcttcagttttctcaggccggagcgagtgacatt |
| | TADVNTQGVLPGMVWQDRDVYLQGPI | cgggaccagtctaggaactggcttcctggaccctgctaccgccagcagcgagta |
| | WAKIPHTDGHFHPSPLMGGFGLKHPP | tcaaagacatctgcggataacaacaacagtgaatactcgtggactggagctacc |
| | PQILIKNTPVPANPSTTFSAAKFASF | aagtaccacctcaatggcagagactctctggtgaatccgggcccggccatggca |
| | ITQYSTGQVSVEIEWELQKENSKRWN | agccacaaggacgacgaagaaaagctttctcctcagagcgggcttctcaccttt |
| | PEIQYTSNYYKSTSVDFAVNTEGVYS | ggcaagcaacgctcagagaaaacaaatgtggacattgaaaaggtcatgattaca |
| | ERPRIGTRYLTRNL SEQ ID NO: | gacgaagaggaaatcaggacaaccaatcccgtggctacggagcagtatggttct |
| | 115 | gtatctaccaacctccagagaggcaacagacaagcagctaccgcagatgtcaac |
| | | acacaaggcgttctcccaggcatggtctggcaggacagagatgtgtaccttcag |
| | | gggcccatctgggcaaagactccacacacggacggacattttcacccctctccc |
| | | ctcatggctggattcggacttaaacaccctcctccacagatcctcatcaaaaac |
| | | acaactgcacctgcgaatccttcgaccaccttcagtgcggcaaagtttgcttcc |
| | | ttcatcacacagcactccacgggacaggtcagcgtggaaatcgagtgggagctg |
| | | cagaaagaaaacagcaaacgctggaacccagagattcaatacacttccaactac |
| | | cacaaatctacaagtgtggactttgctgttaatacagaaggcgtgtactctgaa |
| | | ccccgccccattggcacccgttacctcacccgtaatccg-3′ SEQ ID NO: |
| | | 139 |
| HW15 | MAAGGGAPMADNNEGADGVGNSSGNW | 5′-atggctccaggcgctggcgctccaatggcagacaataacgaaggcgccgac |
| (VP3) | HCDSTWLGDRVITTSTRTWALPTYNN | ggagtgggtaattcctcgggaaattggcattgcgattccacatggctgggcgac |
| | HLYKQISSASTGASNDNTYFGYSTPW | agagtcatcaccaccagcacccgaacctgggccctgcccacctacaacaaccac |
| | GYFDFNRFHCHFSPRDWQRLINNNWG | ctctacaagcaaatctccagtgcttcaacgggggccagcaacgacaacacctac |
| | FRPKRLSFKLFNIQVKEVTQNEGTKT | ttcggctacagcaccccctgggggtattttgactttaacagattccactgccac |
| | IANNLTSTIQVFTDSEYQLPYVLGSA | ttttcaccacgtgactggcagcgactcatcaacaacaactggggattcccgcca |
| | HQGGLPPFPADVFMIPQYGYLTLNNG | aaaagactcagcttcaagctcttcaacatccaggtcaaggaggtcacgcagaat |
| | SQAVGRSSFYCLEYFPSQMLRTGNNF | gaaggcaccaagaccatcgccaataaccttaccagcacgactcaggtctttacg |
| | EFSYQFEDVPFHSSYAHSQSLDRLMN | gactcggaataccagctgccgtacgtcctcggctctgcccaccagggctgcctg |
| | PLIDQYLYYLSRTQTTGGTANTQTLG | cctccgttcccggcggacgtgttcatgattccccagtacggttacctaacactc |
| | FSQGGPNTMANQAKNWLPGPCYRQQR | aacaacggtagtcaggccgtgggacgctcctccttctactgcctggaatacttt |
| | VSTTTGQNNNSNFAWTAGTKYHLNGR | ccttcgcagatgctcagaacgcgcaacaactttgagttcagctaccagtttgag |
| | NSLANPGIAMATHKDDEERFFPSNGI | gacgtgccttttcacagcagctacgcgcacagccagagcctggaccggctgatg |
| | LIFGKQNAARDNADYSDMVLTSEEEI | aaccctctcatcgaccagtacctctactacttgtctcggactcaaacaacacga |
| | KTTNPVATEEYGIVADNLQQTNTGPI | ggcacggcaaatacgcagactctgggcttcagccaaggtgggcctaatacaatg |
| | VGNVNSQGALPGMVWQNRDVYLQGPI | gccaatcaggcaaacaactggctgccaggaccctgttaccgccaacaacgcgtc |
| | WAKIPHTDGNFHPSPLMGGFGLKHPP | tcaacgacaaccgggcaaaacaacaacagcaaccttgcctggactgccgggacc |
| | PQILIKNTPVPADPPTTFNQSKLNSF | aaataccatctgaatggaagaaattcattggctaaacctggcatcgctatggca |
| | ITQYSTGQVSVEIEWELQKENSKRWN | acacacaaagacgacgaggagcgtttttttcccagtaacgggatcctgattttt |
| | PEIQYTSNYYKSNNVEFAVNTEGVYS | ggcaaacaaaatgctgccagagacaatgcggattacagcgatgtcatgctcacc |
| | EPRPIGTRYLTRNL SEQ ID NO: | agcgaggaagaaatcaaaaccactaaccctgtggctacagaggaatacggtatc |
| | 116 | gtggcagataacttgcagcaaaccaatacggggcctatcgtgggaaatgccaac |
| | | agccaaggagccttacctggcatcgtctggcagaaccgagacgtgtacctgcag |
| | | ggtcccatctgggccaagattcctcacacggacggcaacttccacccgtctccg |
| | | ctgatgggcggcttcggcctgaaacatcctccgcctcagatcctgatcaagaac |
| | | acgcctgtacctgcggatcctccgaccaccttcaaccagtcaaagctgaactct |
| | | ttcatcacgcaatacagcaccggacaggtcagcgtggaaattgaatgggagctg |
| | | cagaaggaaaacagcaagcgctggaacccggagatccagtacacttccaactat |
| | | tacaagtctaataatgttgaatttgctgttaatacagaaggcgtgtactctgaa |
| | | ccccgccccattggcacccgttacctcacccgtaatetg-3′ SEQ ID NO: |
| | | 140 |
| HW16 | MATGSGAPMADNNEGADGVGNSSGNW | 5′-atggctacaggcactggcgcaccaatggcagacaataacgacggcgcccac |
| (VP3) | HCDSTWLGNRVITTSTRTWALPTYNN | ggagtgggtaattcctcgggaaattggcattgcgattccacatggctgggcaac |
| | HLYKQISNGTSGGSTNDNTYFGYSTP | agagtcatcaccaccagcacccgaacctgggccctgcccacctacaacaaccac |
| | WGYFDFNRFHCHRSPRDWQRLINNNW | ctctacaagcaaatatccaatgggacatcgggaggaagcaccaacgacaacacc |
| | GFRPKRLSFKLFNIQVKEVTQNEGTK | tactttggctacagcaccccctgggggtattttcacttcaacagattccaccgt |
| | TIANNLTSTVQVFTDSDYQLPYVLGS | cacttttcaccacgtgactggcaacgactcatcaacaacaactgggaattccgg |
| | AHEGCLPPFPADVFMIPQYGYLTLND | cccaagagactcagcttcaagctcttcaacattcaggtcaaggaggtcacgcag |
| | GSQAVGRSSFYCLEYFPSQMLRTGNN | aatgaaggcaccaagaccatcgccaataaccttaccagcacggtccaggtcttc |
| | FQFTYTFEDVPFHSSYAHSQSLDRLM | acggactcagactatcagctcccgtacgtgctcgggtcggctcacgagggctgc |
| | NPLIDQYLYYLSRTQTTGGTANTQTL | ctcccgccgttcccagcggacgttttcacgattccccagtacgggtatctgacg |
| | GFSQGGPNTMANQAKNWLPGPCYRQQ | cttaatgatggaagccaggccgtgggtcgttcgtccttttactgcctggaatat |
| | RVSTTTGQNNNSNFAWTAGTKYHLNG | ttcccgtcgcaaatgctaagaacgggtaacaacttccagtttacttacaccttc |
| | RNSLANPGIAMATHKDDEERFFPSNG | gaggacgtgcctttccacagcagctacgcccacagccagagcttggaccggctg |
| | ILIFGKQNAARDNADYSDVMLTSEEE | atgaatcctctgattgaccagtacctgtactacttgtctcggactcaaacaaca |
| | IKTTNPVATEEYGIVADNLQQRNTAP | ggaggcacggcaaatacgcagactctgggcttcagccaaggtgggcctaataca |
| | QIGTVNSQGALPGMVWQNRDVYLQGP | atggccaatcaggcaaagaactggctgccaggaccctgttaccgccaacaacgc |
| | IWAKIPHTDGNFHPSPLMGGFGLKHP | gtctcaacgacaaccgggcaaaacaacaatagcaactttgcctggactgctggg |
| | PPQILIKNTPVPADPPTTFSAAKFAS | accaaataccatctgaatggaagaaattcattggctaatcctggcatcgctatg |
| | FITQYSTGQVSVEIEWELQKENSKRW | gcaacacacaaagacgacgagcagcgttcttttcccagtaacgggatcctgatt |
| | NPEIQYTSNYYKSNNVEFAVNTEGVY | tttggcaaacaaaatgctgccagagacaatgcggattacagcgatgtcatgctc |
| | SEPRPIGTRYLTRNL SEQ ID NO: | accagcgaggaagaaatcaaaaccactaaccctgtggctacagaggaatacggt |
| | 117 | atcgtggcagataacttgcagcagcgaaacacggctcctcaaattggaactgtc |
| | | aacagccagggggccttacccggtatggtctggcagaaccgggacgtgtacctg |
| | | cagggtcccatctgggccaagattcctcacacggacggcaacttccacccgtct |
| | | ccgctgatgggcggctttggcctgaaacatcctccgcctcagatcctgatcaag |
| | | aacacgcctgtacctgcggatcctccaactaccttcagtgcggcaaagtttgct |
| | | tccttcatcacacagtactccacgggacagctcagcgtggagatcgagtggcag |
| | | ctgcagaaggaaaacagcaaacgctggaatcccgaaattcagtacacttccaac |
| | | tattacaagtctaataatgttgaatttgctgttaatactgaaggtgtatatagt |
| | | gaaccccgccccattggcaccagatacctgactcgtaatctg-3′ SEQ ID |
| | | NO: 141 |
| HW17 | MASGGGAPVADNNEGADGVGSSSGNW | 5′-atggcttcaggtggtggcgcaccagtggcagacaataacgaaggtgccgat |
| (VP3) | HCDSTWLGDRVITTSTRTWALPTYNN | ggagtgggtagttcctcgggaaattggcattgcgattccacatggctgggcgac |
| | HLYKQISNGTSGGSTNDNTYFGYSTP | agagtcatcaccaccagcacccgaacctgggccctgcccacctacaacaaccac |
| | WGYFDFNRFHCHFSPRDWQRLINNNW | ctctacaagcaaatatccaatgggacatcgggaggaagcaccaacgacaacacc |
| | GFRPKRLSFKLFNIQVKEVTQNDGTT | tacttcggctacagcaccccctgggggtattttgactttaacagattccactgc |
| | TIANNLTSTVQVFTDSEYQLPYVLGS | cacttttcaccacgtgactggcagcgactcatcaccaacaactggggattccgg |
| | AHQGCLPPFPADVFMIPQYGYLTLNN | cccaagagactcagcttcaagctcttcaacatccaggtcaaagaggtcacgcag |
| | GSQAVGRSSFYCLEYFPSQMLRTGNN | aacgacggtacgacgacgattgccaataaccttaccagcacggttcaggtgttt |
| | FEFSYTFEDVPFHSSYAHSQSLDRLM | actgactcggagtaccagctcccgtacgtcctcggctcggcgcatcaaggatgc |
| | NPLIDQYLYYLSRTQTTGGTANTQTL | ctcccgccgttcccggcggacgtcttcatgattcctcagtacgggtacctgact |
| | FFSQGGPNTMANXAKNWLPGPCYRQQ | ctgaacaatcgcagtcaggccgtgggccgttcctccttctactgcctggagtac |
| | RVSTTTGQNNNSNFAWTGATKYHLNG | tttccttctcaaatgctgagaacgggcaacaactttgagttcagctaccccttc |
| | RDSLVNPGVAMATHKDDDDRFFPSSG | gaggacgtgcctttccacagcagctacgcacacagccagagcttggaccgactg |
| | VLIFGKQGAGNDGVDYSQVLITDEEE | atcaatcctctcatcgaccagtacctgtactacttgtctcggactcaaacaaca |
| | IRTTNPVATEQYGSVSTNLQRGNRQA | ggaggcacggcaaatacgcagactctgggctttagccaaggtgggcctaataca |
| | ATADVNTQGVLPGMVWQDRDVYLQGP | atggccaatcangcaaagaactggctgccaggaccctgttaccggcagcagcga |
| | IWAKIPHTDGHFHPSPLMGGFGLKHP | gtctctacgacaaccgggcaaaacaacaacagcaactttgcttggactggtgcc |
| | PPQILIKNTPVPANPSTTFSAAKFAS | accaaatatcacctgaacggaagagactctctgtaaatccccggtgtcgctatg |
| | FITQYSTGQVSVEIEWELQKENSKRW | gcaacccacaaggatgacgacgaccgcttcttcccttcgagcggggtcctgatt |
| | NPEIQYTSNFEKQTGVDFAVNTEGTY | tttggcaagcaaggagccgggaacgatggagtggattacagccaagtgctgatt |
| | SEPRPIGTRYLTRNL SEQ ID NO: | acagacgaagaggaaatcaggacaaccaatcccgtggctacggagcagtatggt |
| | 118 | tctgtatctaccaacctccagagaggcaacagacaagcagctaccgcagatgtc |
| | | aacacacaaggcgttcttccaggcatggtctggcaggacagagatgtgtacctt |
| | | caggggcccatctgggcaaagattccacacacggacggacattttcacccctct |
| | | cccctcatgggtggattcggacttaaacaccctcctccacagattctcatcaag |
| | | aacaccccggtacctgcgaatccttcgaccaccttcagtgcggcaaagtttgct |
| | | tccttcatcacacagtactccacgggacaggtcagcgtggagatcgagtgggag |
| | | ctgcagaaggaaaacagcaagcgctggaacccggagattcagtacacctccaac |
| | | tttgaaaagcagactggtgtggactttgctgtcaatacagagggaacttattct |
| | | gagcctcgccccattgctactcgttacctcacccgtaatctg-3′ SEQ ID |
| | | NO: 142 |
| HW18 | MASGGGAPMADNNEXADGVGSSSGNW | 5′-atggcttcaggcggtggcgcaccaatggcagacaataacgaangtgccgat |
| (VP3) | HCDSQWLGDRVITTSTRTWALPTYNN | ggagtgggtagttcctcgggaaattggcattgcgattcccaatggctgggggac |
| | HLYKQISSETAGSTNDNTYFGYSTPW | agagtcatcaccaccagcacccgaacctgggccctgcccacctacaacaatcac |
| | GYFDFNRFHCHFSPRDWQRLINNNWG | ctctacaagcaaatctccagtgaaactgcaggtagtaccaacgacaacacctac |
| | FRPKRLSFKLFNIQVKEVTQNDGTTT | ttcggctacagcaccccctgggggtattttgactttaacagattccactgccac |
| | IANNLTSTVQVFTDSEYQLPYVLGSA | ttctcaccacgtgactggcagcgactcatcaacaacaactggggattccagccc |
| | HQGCLPPFPADVFMIPQYGYLTLNNG | aagagactcagcttcaagctctttaacattcaagtcaaagaggtcacgcagaat |
| | SQAVGRSSFYCLEYFPSQMLRTGNNF | gacggtacgacgacgattgccaataaccttaccagcacggttcaggtgtttact |
| | TFSYTFEDVPFHSSYAHSQSLDRLMN | gactcggagtaccagctcccgtacgtcctcggctccgcgcaccagggctgcctc |
| | PLIDQYLYYLSRTNTPSGTTTQSRLQ | cctccgttcccggcggacgtgttcatgattccgcagtacggctacctaacgctc |
| | FSQAGASDIRDQSRNWLPGPCYRQQR | aacaatggcagccaggcagtgggacggtcatccttttactgcctggaatatttc |
| | VSKTSADNNNSEYSWTGATKYHLNGR | ccatcgcagatgctgagaacgcgcaataactttaccttcagctacaccttcgag |
| | DSLVNPGPAMASHKDDEEKFFPQSGV | gacgtgcctttccacagcagctacgcccacagccagagtctggaccgtctcatg |
| | LIFGKQGSEKTNVDIEKVMITDEEEI | aatcctctcatcgaccagtacctgtattacttgagcagaacaaacactccaagt |
| | RTTNPVATEQYGSVSTNLQRGNRQAA | ggaaccaccacgcagtcaaggcttcagttttctcacgccggagcgagtgacatt |
| | TADNVTQGVLPGMVWQDRDVYLQGPI | cgggaccagtctaggaactggcttcctggaccctgttaccgccagcagcgagta |
| | WAKIPHTDGHFHPSPLMGGFGLKHPP | tcaaagacatctgcggataacaacaacagtgaatactcgtcgactggagctacc\| |
| | PQILIKNTPVPANPSTTFSAAKFASF | aagtaccacctcaatggcagagactctctggtgaatccgggcccggccatggca\| |
| | ITQYSTGQVSVEIEWELQKENSKRWN | agccacaaggacgatgaagaaaagttttttcctcagagcggggttctcatcttt |
| | PEIQYTSNYYKSTSVDFAVNTEGVYS | gggaagcaacgctcagagaaaacaaatgtggacattgaaaaggtcatgattaca |
| | EPRPIGTRYLTRNL SEQ ID NO: | gacgaagaggaaatcaggacaaccaatcccgtggctacggagcagtatggttct |
| | 119 | gtatctaccaacctccagagaggcaacagacaagcagctaccgcagatgtcaac |
| | | acacaaggcgttcttccaggcatggtctggcaggacagagatgtgtaccttcag |
| | | gggcccatctgggcaaagattccacacacggacggacattttcacccctctccc |
| | | ctcatgggtggattcggacttaaacaccctcctccacagattctcatcaagaac |
| | | accccggtacctgcgaatccttcgaccaccttcagtgcggcaaagtttgcttcc |
| | | ttcatcacacagtactccacgggacaggtcagcgtggaaattgaatgggagctg |
| | | cagaaggaaaacagcaagcgctggaaccccgagatccagtacacctccaactac |
| | | tacaaatctacaagtgtggactttgctgttaatacagaaggcgtgtactctgaa |
| | | ccccgccccattggcacccgttacctcacccgtaatctg-3′ SESQ ID NO: |
| | | 143 |
| HW19 | MATGGGAPMADNNEGADGVGNASGNW | 5′-atggctacaggcggtggcgcaccaatgccagacaataacgaaggtgccgac |
| (VP3) | HCDSTWLGDRVITTSTRTWALPTYNN | ggagtgggtaatgcctcaggaaattggcattgcgattccacatggctgggcgac |
| | HLYKQISSETAGSTNDNTYFGYSTPW | agagtcattaccaccagcacccgaacctgggccctgcccacctacaacaaccac |
| | GYFDFNRFHCHFSPRDWQRLINNNWG | ctctacaagcaaatctccagtgaaactgcaggtagtaccaacgacaacacctac |
| | FRPKRLSFKLFNIQVKEVTQNEGTKT | ttcggctacagcaccccctgggggtattttgactttaacagattccactgccac |
| | IANNLTSTIQVFTDSEYQLPYVLGSA | ttttcaccacgtgactggcagcgactcatcaacaacaactggggattccggccc |
| | HQGCLPPFPADVFMIPQYGYLTLNNG | aagagactcagcttcaagctcttcaacatccaggtcaaggaggtcacgcagaat |
| | SQSVGRSSFYCLEYFPSQMMRTGNNF | gaaggcaccaagaccatcgccaataaccttaccagcacgattcaggtgtttacg |
| | EFSYSFEDVPFHSSYAHSQSLDRLMN | gactcggagtaccagctgccgtacgttctcggctccgcccaccagggctgcctg |
| | PLIDQYLYYLSRTQSTGGTAGTQQLL | cctccgttcccggcggacgtgttcatgattcctcagtacggctacctgactctc |
| | FSQAGASDIRDQSRNWLPGPCYRQQR | aacaatggcagtcagtctgtgggacgttcctccttctactgcctggagtacttc |
| | VSKTSADNNNSEYSWTGATKYHLNGR | ccctctcagatgatgagaacgggcaacaactttgacttcagccacagcttcgag |
| | DSLVNPGPAMASHKDDEEKFFPQSGV | gacgtgcctttccacagcagctacgcacacagccagagcctggaccggctgatg |
| | LIFGKQGSEKTNVDIEKVMITDEEEI | aatcccctcatcgaccagtacctgtactacctgtctcggactcagtccacggga |
| | RTTNPVATEQYGSVSTNLQRGNRQAA | ggtaccgcaggaactcagcagttgctattttctcacgccggagcgagtgacatt |
| | TADVNTQGVLPGMVWQDRDVYLQGPI | cgggaccagtctaggaactggcttcctggaccctgttaccgccagcagcgagta |
| | WAKIPHTDGNFHPSPLMGGFGLKHPP | tcaaagacacctgcggataacaacaacagtgaacactcgtggaccggagccacc |
| | PQILIKNTPVPADPPTTFSQAKLASF | aagtaccacctcaatggcagagactctctggtgaatccgggcccggccatggca |
| | ITQYSTGQVSVEIEWELQKENSKRWN | agccacaaggacgatgaagaaaagttttttcctcagagcggggttctcatcttt |
| | PEIQYTSNYYKSTNVDFAVNTEGVYS | gggaagcaaggctcagagaaaacaaatgtggacattgaaaaggtcatgattaca |
| | EPRPIGTRYLTRNL SEQ ID NO: | gacgaagaggaaatcaggacaaccaatcccgtggctacggagcagtatggttct |
| | 120 | gtatctaccaacctccagagaggcaacagacaagcagctaccgcagatgtcaac |
| | | acacaaggcgttcttccaggcatggtctggcaggacacagatgtataccttcag |
| | | gggcccatctgggcaaagattcctcacacggacggcaacttccacccttcaccg |
| | | ctaatgggaggattcggactgaagcacccacctcctcagatcctgatcaagaac |
| | | accccggtacctgcggatcctccaacaacgttcagtcaagctaagctggcgtcg |
| | | ttcatcacgcagtacagcaccggacaggtcagcgtggaaattgaatgggagctg |
| | | cagaaggaaaacagcaagcgctggaacccggagattcaatacacttccaactac |
| | | tacaaatctacaaatgtggactttgctgtcaacacggagggggtttatagcgag |
| | | cctcgccccattggcacccgttacctcacccgcaacctg-3′ SEQ ID NO: |
| | | 144 |

In some embodiments, a capsid protein may be the VP3 protein KJ01. In some embodiments, a capsid protein may be the VP3 protein KJ02. In some embodiments, a capsid protein may be the VP3 protein KJ03. In some embodiments, a capsid protein may be the VP3 protein KJ04. In some embodiments, a capsid protein may be the VP3 protein KJ05. In some embodiments, a capsid protein may be the VP3 protein HW01. In some embodiments, a capsid protein may be the VP3 protein HW02. In some embodiments, a capsid protein may be the VP3 protein HW03. In some embodiments, a capsid protein may be the VP3 protein HW04. In some embodiments, a capsid protein may be the VP3 protein HW05. In some embodiments, capsid protein may be the VP3 protein HW06. In some embodiments, a capsid protein may be the VP3 protein HW07. In some embodiments, capsid protein may be the VP3 protein HW08. In some embodiments, capsid protein may be the VP3 protein HW09. In some embodiments, a capsid protein may be the VP3 protein HW10. In some embodiments, a capsid protein may be the VP3 protein HW11. In some embodiments, a capsid protein may be the VP3 protein HW12. In some embodiments, a capsid protein may be the VP3 protein HW13. In some embodiments, a capsid protein may be the VP3 protein HW14. In some embodiments, a capsid protein may be the VP3 protein HW15. In some embodiments, a capsid protein may be the VP3 protein HW16. In some embodiments, a capsid protein may be the VP3 protein HW17. In some embodiments, a capsid protein may be the VP3 protein HW18. In some embodiments, a capsid protein may be the VP3 protein HW19.

In some embodiments, the AAV particle comprises VP1, VP2 and VP3 capsid proteins, as shown in Tables 1-3.

In some embodiments, the capsid protein is a KJ01 capsid protein, or variant thereof. In some embodiments, the capsid protein is a KJ01 VP1 protein, or variant thereof. In some embodiments, the capsid protein is a KJ01 VP2 protein, or variant thereof. In some embodiments, the capsid protein is a KJ01 VP3 protein, or variant thereof. In some embodiments, described herein is an AAV particle comprising a KJ01 VP1 capsid protein, or variant thereof. In some embodiments, described herein is an AAV particle comprising a KJ01 VP2 protein, or variant thereof. In some embodiments, described herein is an AAV particle comprising a KJ01 VP3 protein, or variant thereof. In some embodiments, described herein is an AAV particle comprising a KJ01 VP1 protein, or variant thereof, and a KJ01 VP2 protein, or variant thereof. In some embodiments, described herein is an AAV particle comprising a KJ01 VP1 protein, or variant thereof, a KJ01 VP2 protein, or variant thereof, and a KJ01 VP3 protein, or variant thereof. In some embodiments, described herein is an AAV particle comprising a KJ01 VP1 protein, or variant thereof, and a KJ01 VP3 protein, or variant thereof. In some embodiments, described herein is an AAV particle comprising a KJ01 VP2 protein, or variant thereof, and a KJ01 VP3 protein, or variant thereof. In some embodiments, described herein is an AAV particle comprising a KJ01 VP1 protein, or variant thereof, a KJ01 VP2 protein, or variant thereof and a KJ01 VP3 protein, or variant thereof, wherein the VP1. VP2 and VP3 proteins or variants are present in the particle in a ratio of 1-2:1:10, respectively. In some embodiments, described herein is an AAV particle comprising a KJ01 VP1 protein, or variant thereof, and a KJ01 VP3 protein, or variant thereof wherein the VP1 and VP3 proteins or variants are present in the particle in a ratio of 1-2:10, respectively.

In some embodiments, the capsid protein is a KJ02 capsid protein, or variant thereof. In some embodiments, the capsid protein is a KJ02 VP1 protein, or variant thereof. In some embodiments, the capsid protein is a KJ02 VP2 protein, or variant thereof. In some embodiments, the capsid protein is a KJ02 VP3 protein, or variant thereof. In some embodiments, described herein is an AAV particle comprising a KJ02 VP1 capsid protein, or variant thereof. In some embodiments, described herein is an AAV particle comprising a KJ02 VP2 protein, or variant thereof. In some embodiments, described herein is an AAV particle comprising a KJ02 VP3 protein, or variant thereof. In some embodiments, described herein is an AAV particle comprising a KJ02 VP1 protein, or variant thereof, and a KJ02 VP2 protein, or variant thereof. In some embodiments, described herein is an AAV particle comprising a KJ02 VP1 protein, or variant thereof, a KJ02 VP2 protein, or variant thereof, and a KJ02 VP3 protein, or variant thereof. In some embodiments, described herein is an AAV particle comprising a KJ02 VP1 protein, or variant thereof, and a KJ02 VP3 protein, or variant thereof. In some embodiments, described herein is an AAV particle comprising a KJ02 VP2 protein, or variant thereof, and a KJ02 VP3 protein, or variant thereof. In some embodiments, described herein is an AAV particle comprising a KJ02 VP1 protein, or variant thereof, a KJ02 VP2 protein, or variant thereof and a KJ02 VP3 protein, or variant thereof, wherein the VP1. VP2 and VP3 proteins or variants are present in the particle in a ratio of 1-2:1:10, respectively. In some embodiments, described herein is an AAV particle comprising a KJ02 VP1 protein, or variant thereof, and a KJ02 VP3 protein, or variant thereof wherein the VP1 and VP3 proteins or variants are present in the particle in a ratio of 1-2:10, respectively.

In some embodiments, the capsid protein is a KJ03 capsid protein, or variant thereof. In some embodiments, the capsid protein is a KJ03 VP1 protein, or variant thereof. In some embodiments, the capsid protein is a KJ03 VP2 protein, or variant thereof. In some embodiments, the capsid protein is a KJ03 VP3 protein, or variant thereof. In some embodiments, described herein is an AAV particle comprising a KJ03 VP1 capsid protein, or variant thereof. In some embodiments, described herein is an AAV particle comprising a KJ03 VP2 protein, or variant thereof. In some embodiments, described herein is an AAV particle comprising a KJ03 VP3 protein, or variant thereof. In some embodiments, described herein is an AAV particle comprising a KJ03 VP1 protein, or variant thereof, and a KJ03 VP2 protein, or variant thereof. In some embodiments, described herein is an AAV particle comprising a KJ03 VP1 protein, or variant thereof, a KJ03 VP2 protein, or variant thereof, and a KJ03 VP3 protein, or variant thereof. In some embodiments, described herein is an AAV particle comprising a KJ03 VP1 protein, or variant thereof, and a KJ03 VP3 protein, or variant thereof. In some embodiments, described herein is an AAV particle comprising a KJ03 VP2 protein, or variant thereof, and a KJ03 VP3 protein, or variant thereof. In some embodiments, described herein is an AAV particle comprising a KJ03 VP1 protein, or variant thereof, a KJ03 VP2 protein, or variant thereof and a KJ03 VP3 protein, or variant thereof, wherein the VP1, VP2 and VP3 proteins or variants are present in the particle in a ratio of 1-2:1:10, respectively. In some embodiments, described herein is an AAV particle comprising a KJ03 VP1 protein, or variant thereof, and a KJ03 VP3 protein, or variant thereof wherein the VP1 and VP3 proteins or variants are present in the particle in a ratio of 1-2:10, respectively.

In some embodiments, the capsid protein is a KJ04 capsid protein, or variant thereof. In some embodiments, the capsid protein is a KJ04 VP1 protein, or variant thereof. In some embodiments, the capsid protein is a KJ04 VP2 protein, or variant thereof. In some embodiments, the capsid protein is a KJ04 VP3 protein, or variant thereof. In some embodiments, described herein is an AAV particle comprising a KJ04 VP1 capsid protein, or variant thereof. In some embodiments, described herein is an AAV particle comprising a KJ04 VP2 protein, or variant thereof. In some embodiments, described herein is an AAV particle comprising a KJ04 VP3 protein, or variant thereof. In some embodiments, described herein is an AAV particle comprising a KJ04 VP1 protein, or variant thereof, and a KJ04 VP2 protein, or variant thereof. In some embodiments, described herein is an AAV particle comprising a KJ04 VP1 protein, or variant thereof, a KJ04 VP2 protein, or variant thereof, and a KR04 VP3 protein, or variant thereof. In some embodiments, described herein is an AAV particle comprising a KJ04 VP1 protein, or variant thereof, and a KJ04 VP3 protein, or variant thereof. In some embodiments, described herein is an AAV particle comprising a KJ04 VP2 protein, or variant thereof, and a KJ04 VP3 protein, or variant thereof. In some embodiments, described herein is an AAV particle comprising a KJ04 VP1 protein, or variant thereof, a KJ04 VP2 protein, or variant thereof and a KJ04 VP3 protein, or variant thereof, wherein the VP1. VP2 and VP3 proteins or variants are present in the particle in a ratio of 1-2:1:10, respectively. In some embodiments, described herein is an AAV particle comprising a KJ04 VP1 protein, or variant thereof, and a KJ04 VP3 protein, or variant thereof wherein the VP1 and VP3 proteins or variants are present in the particle in a ratio of 1-2:10, respectively.

In some embodiments, the capsid protein is a KJ05 capsid protein, or variant thereof. In some embodiments, the capsid protein is a KJ05 VP1 protein, or variant thereof. In some embodiments, the capsid protein is a KJ05 VP2 protein, or variant thereof. In some embodiments, the capsid protein is a KJ05 VP3 protein, or variant thereof. In some embodiments, described herein is an AAV particle comprising a KJ05 VP1 capsid protein, or variant thereof. In some embodiments, described herein is an AAV particle comprising a KJ05 VP2 protein, or variant thereof. In some embodiments, described herein is an AAV particle comprising a KJ05 VP3 protein, or variant thereof. In some embodiments, described herein is an AAV particle comprising a KJ05 VP1 protein, or variant thereof, and a KJ05 VP2 protein, or variant thereof. In some embodiments, described herein is an AAV particle comprising a KJ05 VP1 protein, or variant thereof, a KJ05 VP2 protein, or variant thereof, and a KJ05 VP3 protein, or variant thereof. In some embodiments, described herein is an AAV particle comprising a KJ05 VP1 protein, or variant thereof, and a KJ05 VP3 protein, or variant thereof. In some embodiments, described herein is an AAV particle comprising a KJ05 VP2 protein, or variant thereof, and a KJ05 VP3 protein, or variant thereof. In some embodiments, described herein is an AAV particle comprising a KJ05 VP1 protein, or variant thereof, a KJ05 VP2 protein, or variant thereof and a KJ05 VP3 protein, or variant thereof, wherein the VP1, VP2 and VP3 proteins or variants are present in the particle in a ratio of 1-2:1:10, respectively. In some embodiments, described herein is an AAV particle comprising a KJ05 VP1 protein, or variant thereof, and a KJ05 VP3 protein, or variant thereof wherein the VP1 and VP3 proteins or variants are present in the particle in a ratio of 1-2:10, respectively.

In some embodiments, the capsid protein is a HW01 capsid protein, or variant thereof. In some embodiments, the capsid protein is a HW01 VP protein, or variant thereof. In some embodiments, the capsid protein is a HW01 VP2 protein, or variant thereof. In some embodiments, the capsid protein is a HW01 VP3 protein, or variant thereof. In some embodiments, described herein is an AAV particle comprising a HW01 VP1 capsid protein, or variant thereof. In some embodiments, described herein is an AAV particle comprising a HW01 VP2 protein, or variant thereof. In some embodiments, described herein is an AAV particle comprising a HW01 VP3 protein, or variant thereof. In some embodiments, described herein is an AAV particle comprising a HW01 VP1 protein, or variant thereof, and a HW01 VP2 protein, or variant thereof. In some embodiments, described herein is an AAV particle comprising a HW01 VP1 protein, or variant thereof, a HW01 VP2 protein, or variant thereof, and a HW01 VP3 protein, or variant thereof. In some embodiments, described herein is an AAV particle comprising a HW01 VP1 protein, or variant thereof, and a HW01 VP3 protein, or variant thereof. In some embodiments, described herein is an AAV particle comprising a HW01 VP2 protein, or variant thereof, and a HW01 VP3 protein, or variant thereof. In some embodiments, described herein is an AAV particle comprising a HW01 VP1 protein, or variant thereof, a HW01 VP2 protein, or variant thereof and a HW01 VP3 protein, or variant thereof, wherein the VP1, VP2 and VP3 proteins or variants are present in the particle in a ratio of 1-2:1:10, respectively. In some embodiments, described herein is an AAV particle comprising a HW01 VP1 protein, or variant thereof, and a HW01 VP3 protein, or variant thereof wherein the VP1 and VP3 proteins or variants are present in the particle in a ratio of 1-2:10, respectively.

In some embodiments, the capsid protein is a HW02 capsid protein, or variant thereof. In some embodiments, the capsid protein is a HW02 VP1 protein, or variant thereof. In some embodiments, the capsid protein is a HW02 VP2 protein, or variant thereof. In some embodiments, the capsid protein is a HW02 VP3 protein, or variant thereof. In some embodiments, described herein is an AAV particle comprising a HW02 VP1 capsid protein, or variant thereof. In some embodiments, described herein is an AAV particle comprising a HW02 VP2 protein, or variant thereof. In some embodiments, described herein is an AAV particle comprising a HW02 VP3 protein, or variant thereof. In some embodiments, described herein is an AAV particle comprising a HW02 VP1 protein, or variant thereof, and a HW02 VP2 protein, or variant thereof. In some embodiments, described herein is an AAV particle comprising a HW02 VP1 protein, or variant thereof, a HW02 VP2 protein, or variant thereof, and a HW02 VP3 protein, or variant thereof. In some embodiments, described herein is an AAV particle comprising a HW02 VP1 protein, or variant thereof, and a HW02 VP3 protein, or variant thereof. In some embodiments, described herein is an AAV particle comprising a HW02 VP2 protein, or variant thereof, and a HW02 VP3 protein, or variant thereof. In some embodiments, described herein is an AAV particle comprising a HW02 VP1 protein, or variant thereof, a HW02 VP2 protein, or variant thereof and a HW02 VP3 protein, or variant thereof, wherein the VP1, VP2 and VP3 proteins or variants are present in the particle in a ratio of 1-2:1:10, respectively. In some embodiments, described herein is an AAV particle comprising a HW02 VP1 protein, or variant thereof, and a HW02 VP3 protein, or variant thereof wherein the VP1 and VP3 proteins or variants are present in the particle in a ratio of 1-2:10, respectively.

In some embodiments, the capsid protein is a HW03 capsid protein, or variant thereof. In some embodiments, the capsid protein is a HW03 VP1 protein, or variant thereof. In some embodiments, the capsid protein is a HW03 VP2 protein, or variant thereof. In some embodiments, the capsid protein is a HW03 VP3 protein, or variant thereof. In some embodiments, described herein is an AAV particle comprising a HW03 VP1 capsid protein, or variant thereof. In some embodiments, described herein is an AAV particle comprising a HW03 VP2 protein, or variant thereof. In some embodiments, described herein is an AAV particle comprising a HW03 VP3 protein, or variant thereof. In some embodiments, described herein is an AAV particle comprising a HW03 VP1 protein, or variant thereof, and a HW03 VP2 protein, or variant thereof. In some embodiments, described herein is an AAV particle comprising a HW03 VP1 protein, or variant thereof, a HW03 VP2 protein, or variant thereof, and a HW03 VP3 protein, or variant thereof. In some embodiments, described herein is an AAV particle comprising a HW03 VP1 protein, or variant thereof, and a HW03 VP3 protein, or variant thereof. In some embodiments, described herein is an AAV particle comprising a HW03 VP2 protein, or variant thereof, and a HW03 VP3 protein, or variant thereof. In some embodiments, described herein is an AAV particle comprising a HW03 VP1 protein, or variant thereof, a HW03 VP2 protein, or variant thereof and a HW03 VP3 protein, or variant thereof, wherein the VP1. VP2 and VP3 proteins or variants are present in the particle in a ratio of 1-2:1:10, respectively. In some embodiments, described herein is an AAV particle comprising a HW03 VP1 protein, or variant thereof, and a HW03 VP3 protein, or variant thereof wherein the VP1 and VP3 proteins or variants are present in the particle in a ratio of 1-2:10, respectively.

In some embodiments, the capsid protein is a HW04 capsid protein, or variant thereof. In some embodiments, the capsid protein is a HW04 VP1 protein, or variant thereof. In some embodiments, the capsid protein is a HW04 VP2 protein, or variant thereof. In some embodiments, the capsid protein is a HW04 VP3 protein, or variant thereof. In some embodiments, described herein is an AAV particle comprising a HW04 VP1 capsid protein, or variant thereof. In some embodiments, described herein is an AAV particle comprising a HW04 VP2 protein, or variant thereof. In some embodiments, described herein is an AAV particle comprising a HW04 VP3 protein, or variant thereof. In some embodiments, described herein is an AAV particle comprising a HW04 VP1 protein, or variant thereof, and a HW04 VP2 protein, or variant thereof. In some embodiments, described herein is an AAV particle comprising a HW04 VP1 protein, or variant thereof, a HW04 VP2 protein, or variant thereof, and a HW04 VP3 protein, or variant thereof. In some embodiments, described herein is an AAV particle comprising a HW04 VP1 protein, or variant thereof, and a HW04 VP3 protein, or variant thereof. In some embodiments, described herein is an AAV particle comprising a HW04 VP2 protein, or variant thereof, and a HW04 VP3 protein, or variant thereof. In some embodiments, described herein is an AAV particle comprising a HW04 VP1 protein, or variant thereof, a HW04 VP2 protein, or variant thereof and a HW04 VP3 protein, or variant thereof, wherein the VP1. VP2 and VP3 proteins or variants are present in the particle in a ratio of 1-2:1:10, respectively. In some embodiments, described herein is an AAV particle comprising a HW04 VP1 protein, or variant thereof, and a HW04 VP3 protein, or variant thereof wherein the VP1 and VP3 proteins or variants are present in the particle in a ratio of 1-2:10, respectively.

In some embodiments, the capsid protein is a HW05 capsid protein, or variant thereof. In some embodiments, the capsid protein is a HW05 VP protein, or variant thereof. In some embodiments, the capsid protein is a HW05 VP2 protein, or variant thereof. In some embodiments, the capsid protein is a HW05 VP3 protein, or variant thereof. In some embodiments, described herein is an AAV particle comprising a HW05 VP1 capsid protein, or variant thereof. In some embodiments, described herein is an AAV particle comprising a HW05 VP2 protein, or variant thereof. In some embodiments, described herein is an AAV particle comprising a HW05 VP3 protein, or variant thereof. In some embodiments, described herein is an AAV particle comprising a HW05 VP1 protein, or variant thereof, and a HW05 VP2 protein, or variant thereof. In some embodiments, described herein is an AAV particle comprising a HW05 VP1 protein, or variant thereof, a HW05 VP2 protein, or variant thereof, and a HW05 VP3 protein, or variant thereof. In some embodiments, described herein is an AAV particle comprising a HW05 VP1 protein, or variant thereof, and a HW05 VP3 protein, or variant thereof. In some embodiments, described herein is an AAV particle comprising a HW05 VP2 protein, or variant thereof, and a HW05 VP3 protein, or variant thereof. In some embodiments, described herein is an AAV particle comprising a HW05 VP1 protein, or variant thereof, a HW05 VP2 protein, or variant thereof and a HW05 VP3 protein, or variant thereof, wherein the VP1, VP2 and VP3 proteins or variants are present in the particle in a ratio of 1-2:1:10, respectively. In some embodiments, described herein is an AAV particle comprising a HW05 VP1 protein, or variant thereof, and a HW05 VP3 protein, or variant thereof wherein the VP1 and VP3 proteins or variants are present in the particle in a ratio of 1-2:10, respectively.

In some embodiments, the capsid protein is a HW06 capsid protein, or variant thereof. In some embodiments, the capsid protein is a HW06 VP1 protein, or variant thereof. In some embodiments, the capsid protein is a HW06 VP2 protein, or variant thereof. In some embodiments, the capsid protein is a HW06 VP3 protein, or variant thereof. In some embodiments, described herein is an AAV particle comprising a HW06 VP1 capsid protein, or variant thereof. In some embodiments, described herein is an AAV particle comprising a HW06 VP2 protein, or variant thereof. In some embodiments, described herein is an AAV particle comprising a HW06 VP3 protein, or variant thereof. In some embodiments, described herein is an AAV particle comprising a HW06 VP1 protein, or variant thereof, and a HW06 VP2 protein, or variant thereof. In some embodiments, described herein is an AAV particle comprising a HW06 VP1 protein, or variant thereof, a HW06 VP2 protein, or variant thereof, and a HW06 VP3 protein, or variant thereof. In some embodiments, described herein is an AAV particle comprising a HW06 VP1 protein, or variant thereof, and a HW06 VP3 protein, or variant thereof. In some embodiments, described herein is an AAV particle comprising a HW06 VP2 protein, or variant thereof, and a HW06 VP3 protein, or variant thereof. In some embodiments, described herein is an AAV particle comprising a HW06 VP1 protein, or variant thereof, a HW06 VP2 protein, or variant thereof and a HW06 VP3 protein, or variant thereof, wherein the VP1, VP2 and VP3 proteins or variants are present in the particle in a ratio of 1-2:1:10, respectively. In some embodiments, described herein is an AAV particle comprising a HW06 VP1 protein, or variant thereof, and a HW06 VP3 protein, or variant thereof wherein the VP1 and VP3 proteins or variants are present in the particle in a ratio of 1-2:10, respectively.

In some embodiments, the capsid protein is a HW07 capsid protein, or variant thereof. In some embodiments, the capsid protein is a HW07 VP protein, or variant thereof. In some embodiments, the capsid protein is a HW07 VP2 protein, or variant thereof. In some embodiments, the capsid protein is a HW07 VP3 protein, or variant thereof. In some embodiments, described herein is an AAV particle comprising a HW07 VP1 capsid protein, or variant thereof. In some embodiments, described herein is an AAV particle comprising a HW07 VP2 protein, or variant thereof. In some embodiments, described herein is an AAV particle comprising a HW07 VP3 protein, or variant thereof. In some embodiments, described herein is an AAV particle comprising a HW07 VP1 protein, or variant thereof, and a HW07 VP2 protein, or variant thereof. In some embodiments, described herein is an AAV particle comprising a HW07 VP1 protein, or variant thereof, a HW07 VP2 protein, or variant thereof, and a HW07 VP3 protein, or variant thereof. In some embodiments, described herein is an AAV particle comprising a HW07 VP1 protein, or variant thereof, and a HW07 VP3 protein, or variant thereof. In some embodiments, described herein is an AAV particle comprising a HW07 VP2 protein, or variant thereof, and a HW07 VP3 protein, or variant thereof. In some embodiments, described herein is an AAV particle comprising a HW07 VP1 protein, or variant thereof, a HW07 VP2 protein, or variant thereof and a HW07 VP3 protein, or variant thereof, wherein the VP1. VP2 and VP3 proteins or variants are present in the particle in a ratio of 1-2:1:10, respectively. In some embodiments, described herein is an AAV particle comprising a HW07 VP1 protein, or variant thereof, and a HW07 VP3 protein, or variant thereof wherein the VP1 and VP3 proteins or variants are present in the particle in a ratio of 1-2:10, respectively.

In some embodiments, the capsid protein is a HW08 capsid protein, or variant thereof. In some embodiments, the capsid protein is a HW08 VP protein, or variant thereof. In some embodiments, the capsid protein is a HW08 VP2 protein, or variant thereof. In some embodiments, the capsid protein is a HW08 VP3 protein, or variant thereof. In some embodiments, described herein is an AAV particle comprising a HW08 VP1 capsid protein, or variant thereof. In some embodiments, described herein is an AAV particle comprising a HW08 VP2 protein, or variant thereof. In some embodiments, described herein is an AAV particle comprising a HW08 VP3 protein, or variant thereof. In some embodiments, described herein is an AAV particle comprising a HW08 VP1 protein, or variant thereof, and a HW08 VP2 protein, or variant thereof. In some embodiments, described herein is an AAV particle comprising a HW08 VP1 protein, or variant thereof, a HW08 VP2 protein, or variant thereof, and a HW08 VP3 protein, or variant thereof. In some embodiments, described herein is an AAV particle comprising a HW08 VP1 protein, or variant thereof, and a HW08 VP3 protein, or variant thereof. In some embodiments, described herein is an AAV particle comprising a HW08 VP2 protein, or variant thereof, and a HW08 VP3 protein, or variant thereof. In some embodiments, described herein is an AAV particle comprising a HW08 VP1 protein, or variant thereof, a HW08 VP2 protein, or variant thereof and a HW08 VP3 protein, or variant thereof, wherein the VP1. VP2 and VP3 proteins or variants are present in the particle in a ratio of 1-2:1:10, respectively. In some embodiments, described herein is an AAV particle comprising a HW08 VP1 protein, or variant thereof, and a HW08 VP3 protein, or variant thereof wherein the VP1 and VP3 proteins or variants are present in the particle in a ratio of 1-2:10, respectively.

In some embodiments, the capsid protein is a HW09 capsid protein, or variant thereof. In some embodiments, the capsid protein is a HW09 VP1 protein, or variant thereof. In some embodiments, the capsid protein is a HW09 VP2 protein, or variant thereof. In some embodiments, the capsid protein is a HW09 VP3 protein, or variant thereof. In some embodiments, described herein is an AAV particle comprising a HW09 VP1 capsid protein, or variant thereof. In some embodiments, described herein is an AAV particle comprising a HW09 VP2 protein, or variant thereof. In some embodiments, described herein is an AAV particle comprising a HW09 VP3 protein, or variant thereof. In some embodiments, described herein is an AAV particle comprising a HW09 VP1 protein, or variant thereof, and a HW09 VP2 protein, or variant thereof. In some embodiments, described herein is an AAV particle comprising a HW09 VP1 protein, or variant thereof, a HW09 VP2 protein, or variant thereof, and a HW09 VP3 protein, or variant thereof. In some embodiments, described herein is an AAV particle comprising a HW09 VP1 protein, or variant thereof, and a HW09 VP3 protein, or variant thereof. In some embodiments, described herein is an AAV particle comprising a HW09 VP2 protein, or variant thereof, and a HW09 VP3 protein, or variant thereof. In some embodiments, described herein is an AAV particle comprising a HW09 VP1 protein, or variant thereof, a HW09 VP2 protein, or variant thereof and a HW09 VP3 protein, or variant thereof, wherein the VP1, VP2 and VP3 proteins or variants are present in the particle in a ratio of 1-2:1:10, respectively. In some embodiments, described herein is an AAV particle comprising a HW09 VP1 protein, or variant thereof, and a HW09 VP3 protein, or variant thereof wherein the VP1 and VP3 proteins or variants are present in the particle in a ratio of 1-2:10, respectively.

In some embodiments, the capsid protein is a HW10 capsid protein, or variant thereof. In some embodiments, the capsid protein is a HW10 VP1 protein, or variant thereof. In some embodiments, the capsid protein is a HW10 VP2 protein, or variant thereof. In some embodiments, the capsid protein is a HW10 VP3 protein, or variant thereof. In some embodiments, described herein is an AAV particle comprising a HW10 VP1 capsid protein, or variant thereof. In some embodiments, described herein is an AAV particle comprising a HW10 VP2 protein, or variant thereof. In some embodiments, described herein is an AAV particle comprising a HW10 VP3 protein, or variant thereof. In some embodiments, described herein is an AAV particle comprising a HW10 VP1 protein, or variant thereof, and a HW10 VP2 protein, or variant thereof. In some embodiments, described herein is an AAV particle comprising a HW10 VP1 protein, or variant thereof, a HW10 VP2 protein, or variant thereof, and a HW10 VP3 protein, or variant thereof. In some embodiments, described herein is an AAV particle comprising a HW10 VP1 protein, or variant thereof, and a HW10 VP3 protein, or variant thereof. In some embodiments, described herein is an AAV particle comprising a HW10 VP2 protein, or variant thereof, and a HW10 VP3 protein, or variant thereof. In some embodiments, described herein is an AAV particle comprising a HW10 VP1 protein, or variant thereof, a HW10 VP2 protein, or variant thereof and a HW10 VP3 protein, or variant thereof, wherein the VP1, VP2 and VP3 proteins or variants are present in the particle in a ratio of 1-2:1:10, respectively. In some embodiments, described herein is an AAV particle comprising a HW10 VP1 protein, or variant thereof, and a HW10 VP3 protein, or variant thereof wherein the VP1 and VP3 proteins or variants are present in the particle in a ratio of 1-2:10, respectively.

In some embodiments, the capsid protein is a HW11 capsid protein, or variant thereof. In some embodiments, the capsid protein is a HW11 VP protein, or variant thereof. In some embodiments, the capsid protein is a HW11 VP2 protein, or variant thereof. In some embodiments, the capsid protein is a HW11 VP3 protein, or variant thereof. In some embodiments, described herein is an AAV particle comprising a HW11 VP capsid protein, or variant thereof. In some embodiments, described herein is an AAV particle comprising a HW11 VP2 protein, or variant thereof. In some embodiments, described herein is an AAV particle comprising a HW11 VP3 protein, or variant thereof. In some embodiments, described herein is an AAV particle comprising a HW11 VP protein, or variant thereof, and a HW11 VP2 protein, or variant thereof. In some embodiments, described herein is an AAV particle comprising a HW11 VP1 protein, or variant thereof, a HW11 VP2 protein, or variant thereof, and a HW11 VP3 protein, or variant thereof. In some embodiments, described herein is an AAV particle comprising a HW11 VP1 protein, or variant thereof, and a HW11 VP3 protein, or variant thereof. In some embodiments, described herein is an AAV particle comprising a HW11 VP2 protein, or variant thereof, and a HW11 VP3 protein, or variant thereof. In some embodiments, described herein is an AAV particle comprising a HW11 VP1 protein, or variant thereof, a HW11 VP2 protein, or variant thereof and a HW11 VP3 protein, or variant thereof, wherein the VP1. VP2 and VP3 proteins or variants are present in the particle in a ratio of 1-2:1:10, respectively. In some embodiments, described herein is an AAV particle comprising a HW11 VP1 protein, or variant thereof, and a HW11 VP3 protein, or variant thereof wherein the VP1 and VP3 proteins or variants are present in the particle in a ratio of 1-2:10, respectively.

In some embodiments, the capsid protein is a HW12 capsid protein, or variant thereof. In some embodiments, the capsid protein is a HW12 VP protein, or variant thereof. In some embodiments, the capsid protein is a HW12 VP2 protein, or variant thereof. In some embodiments, the capsid protein is a HW12 VP3 protein, or variant thereof. In some embodiments, described herein is an AAV particle comprising a HW12 VP1 capsid protein, or variant thereof. In some embodiments, described herein is an AAV particle comprising a HW12 VP2 protein, or variant thereof. In some embodiments, described herein is an AAV particle comprising a HW12 VP3 protein, or variant thereof. In some embodiments, described herein is an AAV particle comprising a HW12 VP1 protein, or variant thereof, and a HW12 VP2 protein, or variant thereof. In some embodiments, described herein is an AAV particle comprising a HW12 VP1 protein, or variant thereof, a HW12 VP2 protein, or variant thereof, and a HW12 VP3 protein, or variant thereof. In some embodiments, described herein is an AAV particle comprising a HW12 VP1 protein, or variant thereof, and a HW12 VP3 protein, or variant thereof. In some embodiments, described herein is an AAV particle comprising a HW12 VP2 protein, or variant thereof, and a HW12 VP3 protein, or variant thereof. In some embodiments, described herein is an AAV particle comprising a HW12 VP1 protein, or variant thereof, a HW12 VP2 protein, or variant thereof and a HW12 VP3 protein, or variant thereof, wherein the VP1. VP2 and VP3 proteins or variants are present in the particle in a ratio of 1-2:1:10, respectively. In some embodiments, described herein is an AAV particle comprising a HW12 VP1 protein, or variant thereof, and a HW12 VP3 protein, or variant thereof wherein the VP1 and VP3 proteins or variants are present in the particle in a ratio of 1-2:10, respectively.

In some embodiments, the capsid protein is a HW13 capsid protein, or variant thereof. In some embodiments, the capsid protein is a HW13 VP1 protein, or variant thereof. In some embodiments, the capsid protein is a HW13 VP2 protein, or variant thereof. In some embodiments, the capsid protein is a HW13 VP3 protein, or variant thereof. In some embodiments, described herein is an AAV particle comprising a HW13 VP1 capsid protein, or variant thereof. In some embodiments, described herein is an AAV particle comprising a HW13 VP2 protein, or variant thereof. In some embodiments, described herein is an AAV particle comprising a HW13 VP3 protein, or variant thereof. In some embodiments, described herein is an AAV particle comprising a HW13 VP1 protein, or variant thereof, and a HW13 VP2 protein, or variant thereof. In some embodiments, described herein is an AAV particle comprising a HW13 VP1 protein, or variant thereof, a HW13 VP2 protein, or variant thereof, and a HW13 VP3 protein, or variant thereof. In some embodiments, described herein is an AAV particle comprising a HW13 VP1 protein, or variant thereof, and a HW13 VP3 protein, or variant thereof. In some embodiments, described herein is an AAV particle comprising a HW13 VP2 protein, or variant thereof, and a HW13 VP3 protein, or variant thereof. In some embodiments, described herein is an AAV particle comprising a HW13 VP1 protein, or variant thereof, a HW13 VP2 protein, or variant thereof and a HW13 VP3 protein, or variant thereof, wherein the VP1, VP2 and VP3 proteins or variants are present in the particle in a ratio of 1-2:1:10, respectively. In some embodiments, described herein is an AAV particle comprising a HW13 VP1 protein, or variant thereof, and a HW13 VP3 protein, or variant thereof wherein the VP1 and VP3 proteins or variants are present in the particle in a ratio of 1-2:10, respectively.

In some embodiments, the capsid protein is a HW14 capsid protein, or variant thereof. In some embodiments, the capsid protein is a HW14 VP protein, or variant thereof. In some embodiments, the capsid protein is a HW14 VP2 protein, or variant thereof. In some embodiments, the capsid protein is a HW14 VP3 protein, or variant thereof. In some embodiments, described herein is an AAV particle comprising a HW14 VP1 capsid protein, or variant thereof. In some embodiments, described herein is an AAV particle comprising a HW14 VP2 protein, or variant thereof. In some embodiments, described herein is an AAV particle comprising a HW14 VP3 protein, or variant thereof. In some embodiments, described herein is an AAV particle comprising a HW14 VP1 protein, or variant thereof, and a HW14 VP2 protein, or variant thereof. In some embodiments, described herein is an AAV particle comprising a HW14 VP1 protein, or variant thereof, a HW14 VP2 protein, or variant thereof and a HW14 VP3 protein, or variant thereof. In some embodiments, described herein is an AAV particle comprising a HW14 VP1 protein, or variant thereof, and a HW14 VP3 protein, or variant thereof. In some embodiments, described herein is an AAV particle comprising a HW14 VP2 protein, or variant thereof, and a HW14 VP3 protein, or variant thereof. In some embodiments, described herein is an AAV particle comprising a HW14 VP1 protein, or variant thereof, a HW14 VP2 protein, or variant thereof and a HW14 VP3 protein, or variant thereof, wherein the VP1, VP2 and VP3 proteins or variants are present in the particle in a ratio of 1-2:1:10, respectively. In some embodiments, described herein is an AAV particle comprising a HW14 VP1 protein, or variant thereof, and a HW14 VP3 protein, or variant thereof wherein the VP1 and VP3 proteins or variants are present in the particle in a ratio of 1-2:10, respectively.

In some embodiments, the capsid protein is a HW15 capsid protein, or variant thereof. In some embodiments, the capsid protein is a HW15 VP1 protein, or variant thereof. In some embodiments, the capsid protein is a HW15 VP2 protein, or variant thereof. In some embodiments, the capsid protein is a HW15 VP3 protein, or variant thereof. In some embodiments, described herein is an AAV particle comprising a HW15 VP1 capsid protein, or variant thereof. In some embodiments, described herein is an AAV particle comprising a HW15 VP2 protein, or variant thereof. In some embodiments, described herein is an AAV particle comprising a HW15 VP3 protein, or variant thereof. In some embodiments, described herein is an AAV particle comprising a HW15 VP1 protein, or variant thereof, and a HW15 VP2 protein, or variant thereof. In some embodiments, described herein is an AAV particle comprising a HW15 VP1 protein, or variant thereof, a HW15 VP2 protein, or variant thereof, and a HW15 VP3 protein, or variant thereof. In some embodiments, described herein is an AAV particle comprising a HW15 VP1 protein, or variant thereof, and a HW15 VP3 protein, or variant thereof. In some embodiments, described herein is an AAV particle comprising a HW15 VP2 protein, or variant thereof, and a HW15 VP3 protein, or variant thereof. In some embodiments, described herein is an AAV particle comprising a HW15 VP1 protein, or variant thereof, a HW15 VP2 protein, or variant thereof and a HW15 VP3 protein, or variant thereof, wherein the VP1, VP2 and VP3 proteins or variants are present in the particle in a ratio of 1-2:1:10, respectively. In some embodiments, described herein is an AAV particle comprising a HW15 VP1 protein, or variant thereof, and a HW15 VP3 protein, or variant thereof wherein the VP1 and VP3 proteins or variants are present in the particle in a ratio of 1-2:10, respectively.

In some embodiments, the capsid protein is a HW16 capsid protein, or variant thereof. In some embodiments, the capsid protein is a HW16 VP protein, or variant thereof. In some embodiments, the capsid protein is a HW16 VP2 protein, or variant thereof. In some embodiments, the capsid protein is a HW16 VP3 protein, or variant thereof. In some embodiments, described herein is an AAV particle comprising a HW16 VP1 capsid protein, or variant thereof. In some embodiments, described herein is an AAV particle comprising a HW16 VP2 protein, or variant thereof. In some embodiments, described herein is an AAV particle comprising a HW16 VP3 protein, or variant thereof. In some embodiments, described herein is an AAV particle comprising a HW16 VP1 protein, or variant thereof, and a HW16 VP2 protein, or variant thereof. In some embodiments, described herein is an AAV particle comprising a HW16 VP1 protein, or variant thereof, a HW16 VP2 protein, or variant thereof, and a HW16 VP3 protein, or variant thereof. In some embodiments, described herein is an AAV particle comprising a HW16 VP1 protein, or variant thereof, and a HW16 VP3 protein, or variant thereof. In some embodiments, described herein is an AAV particle comprising a HW16 VP2 protein, or variant thereof, and a HW16 VP3 protein, or variant thereof. In some embodiments, described herein is an AAV particle comprising a HW16 VP1 protein, or variant thereof, a HW16 VP2 protein, or variant thereof and a HW16 VP3 protein, or variant thereof, wherein the VP1, VP2 and VP3 proteins or variants are present in the particle in a ratio of 1-2:1:10, respectively. In some embodiments, described herein is an AAV particle comprising a HW16 VP1 protein, or variant thereof, and a HW16 VP3 protein, or variant thereof wherein the VP1 and VP3 proteins or variants are present in the particle in a ratio of 1-2:10, respectively.

In some embodiments, the capsid protein is a HW17 capsid protein, or variant thereof. In some embodiments, the capsid protein is a HW17 VP1 protein, or variant thereof. In some embodiments, the capsid protein is a HW17 VP2 protein, or variant thereof. In some embodiments, the capsid protein is a HW17 VP3 protein, or variant thereof. In some embodiments, described herein is an AAV particle comprising a HW17 VP1 capsid protein, or variant thereof. In some embodiments, described herein is an AAV particle comprising a HW17 VP2 protein, or variant thereof. In some embodiments, described herein is an AAV particle comprising a HW17 VP3 protein, or variant thereof. In some embodiments, described herein is an AAV particle comprising a HW17 VP1 protein, or variant thereof, and a HW17 VP2 protein, or variant thereof. In some embodiments, described herein is an AAV particle comprising a HW17 VP1 protein, or variant thereof, a HW17 VP2 protein, or variant thereof, and a HW17 VP3 protein, or variant thereof. In some embodiments, described herein is an AAV particle comprising a HW17 VP1 protein, or variant thereof, and a HW17 VP3 protein, or variant thereof. In some embodiments, described herein is an AAV particle comprising a HW17 VP2 protein, or variant thereof, and a HW17 VP3 protein, or variant thereof. In some embodiments, described herein is an AAV particle comprising a HW17 VP1 protein, or variant thereof, a HW17 VP2 protein, or variant thereof and a HW17 VP3 protein, or variant thereof, wherein the VP1, VP2 and VP3 proteins or variants are present in the particle in a ratio of 1-2:1:10, respectively. In some embodiments, described herein is an AAV particle comprising a HW17 VP1 protein, or variant thereof, and a HW17 VP3 protein, or variant thereof wherein the VP1 and VP3 proteins or variants are present in the particle in a ratio of 1-2:10, respectively.

In some embodiments, the capsid protein is a HW18 capsid protein, or variant thereof. In some embodiments, the capsid protein is a HW18 VP protein, or variant thereof. In some embodiments, the capsid protein is a HW18 VP2 protein, or variant thereof. In some embodiments, the capsid protein is a HW18 VP3 protein, or variant thereof. In some embodiments, described herein is an AAV particle comprising a HW18 VP1 capsid protein, or variant thereof. In some embodiments, described herein is an AAV particle comprising a HW18 VP2 protein, or variant thereof. In some embodiments, described herein is an AAV particle comprising a HW18 VP3 protein, or variant thereof. In some embodiments, described herein is an AAV particle comprising a HW18 VP1 protein, or variant thereof, and a HW18 VP2 protein, or variant thereof. In some embodiments, described herein is an AAV particle comprising a HW18 VP1 protein, or variant thereof, a HW18 VP2 protein, or variant thereof, and a HW18 VP3 protein, or variant thereof. In some embodiments, described herein is an AAV particle comprising a HW18 VP1 protein, or variant thereof, and a HW18 VP3 protein, or variant thereof. In some embodiments, described herein is an AAV particle comprising a HW18 VP2 protein, or variant thereof, and a HW18 VP3 protein, or variant thereof. In some embodiments, described herein is an AAV particle comprising a HW18 VP1 protein, or variant thereof, a HW18 VP2 protein, or variant thereof and a HW18 VP3 protein, or variant thereof, wherein the VP1, VP2 and VP3 proteins or variants are present in the particle in a ratio of 1-2:1:10, respectively. In some embodiments, described herein is an AAV particle comprising a HW18 VP1 protein, or variant thereof, and a HW18 VP3 protein, or variant thereof wherein the VP1 and VP3 proteins or variants are present in the particle in a ratio of 1-2:10, respectively.

In some embodiments, the capsid protein is a HW19 capsid protein, or variant thereof. In some embodiments, the capsid protein is a HW19 VP1 protein, or variant thereof. In some embodiments, the capsid protein is a HW19 VP2 protein, or variant thereof. In some embodiments, the capsid protein is a HW19 VP3 protein, or variant thereof. In some embodiments, described herein is an AAV particle comprising a HW19 VP1 capsid protein, or variant thereof. In some embodiments, described herein is an AAV particle comprising a HW19 VP2 protein, or variant thereof. In some embodiments, described herein is an AAV particle comprising a HW19 VP3 protein, or variant thereof. In some embodiments, described herein is an AAV particle comprising a HW19 VP1 protein, or variant thereof, and a HW19 VP2 protein, or variant thereof. In some embodiments, described herein is an AAV particle comprising a HW19 VP1 protein, or variant thereof, a HW19 VP2 protein, or variant thereof, and a HW19 VP3 protein, or variant thereof. In some embodiments, described herein is an AAV particle comprising a HW19 VP1 protein, or variant thereof, and a HW19 VP3 protein, or variant thereof. In some embodiments, described herein is an AAV particle comprising a HW19 VP2 protein, or variant thereof, and a HW19 VP3 protein, or variant thereof. In some embodiments, described herein is an AAV particle comprising a HW19 VP1 protein, or variant thereof, a HW19 VP2 protein, or variant thereof and a HW19 VP3 protein, or variant thereof, wherein the VP1, VP2 and VP3 proteins or variants are present in the particle in a ratio of 1-2:1:10, respectively. In some embodiments, described herein is an AAV particle comprising a HW19 VP1 protein, or variant thereof, and a HW19 VP3 protein, or variant thereof wherein the VP1 and VP3 proteins or variants are present in the particle in a ratio of 1-2:10, respectively.

In some embodiments, a first capsid protein is considered a variant of a second capsid protein if the amino acid sequence of the first capsid protein has at least 90%, 91%, 92%, 93%, 94%, 95%, 96%, 97%, 98% or 99% identity to the amino acid sequence of the second capsid protein. Differences between amino acid sequence of a capsid protein and a variant of the capsid protein can comprise amino acid substitutions (e.g., conservative amino acid substitutions), deletions and insertions. A first capsid protein is not considered a variant of a second capsid protein if the amino acid sequence of the first capsid protein is identical to the amino acid sequence of any one of the AAV2, AAV6, AAV7, AAV8, AAV9, AAVrh8, AAVrh10, or AAVrh43 serotypes.

In one embodiment, the initiation codon for translation of the AAV VP1 capsid protein may be CTG, TTG, or GTG as described in U.S. Pat. No. 8,163,543, the contents of which are herein incorporated by reference in their entirety.

The present disclosure refers to structural capsid proteins (including VP1. VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence.

As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Capsid Engineering and Directed Evolution

Recombinant or engineered AAV vectors have shown promise for use in therapy for the treatment of human disease. However, a need still exists for AAV particles with more specific and/or enhanced tropism for target tissues. Capsid engineering methods have been used to try to identify capsids with enhanced transduction of target tissues (e.g., brain, spinal cord, DRG). A variety of methods have been used, including mutational methods, DNA barcoding, directed evolution, random peptide insertions, and capsid shuffling and/or chimeras.

Directed evolution involves the generation of AAV capsid libraries (˜10⁴-10⁸) by any of a variety of mutagenesis techniques and selection of lead candidates based on response to selective pressure by properties of interest (e.g., tropism), as described in Grimm et al. Molecular Therapy 23(12): 1819-1831 (2015), the contents of which are herein incorporated by reference in their entirety. Directed evolution of AAV capsids allows for positive selection from a pool of diverse mutants without necessitating extensive prior characterization of the mutant library. Directed evolution libraries may be generated by any molecular biology technique known in the art, and may include, DNA shuffling, random point mutagenesis, insertional mutagenesis (e.g., targeting peptides), random peptide insertions, or ancestral reconstructions. AAV capsid libraries may be subjected to more than one round of selection using directed evolution for further optimization. Directed evolution methods are most commonly used to identify AAV capsid proteins with enhanced transduction of a target tissue. Capsids with enhanced transduction of a target tissue have been identified for the targeting of human airway epithelium, neural stem cells, human pluripotent stem cells, retinal cells, and other in vitro and in vivo cells.

In one embodiment, directed evolution methods are used to identify AAV capsids having enhanced transduction of a target tissue (e.g., CNS or PNS). Multiple strategies of directed evolution, including random-point mutagenesis, insertional mutagenesis, and capsid shuffling are available for the selection of AAV capsids with the desired properties. Random point mutagenesis, site-directed mutagenesis and/or randomized mutagenesis may be used to alter the viral genome, and ultimately the viral capsid, as described in Grimm et al. Molecular Therapy 23(12): 1819-1831 (2015). Santiago-Ortiz et al. Gene Therapeutics 22(12): 934-946 (2015), and Wu et al. Journal of J Virology 74(18): 8635-8647 (2000), the contents of each of which are herein incorporated by reference in their entirety. Insertional mutagenesis may involve the insertion of a peptide sequence into the capsid to enhance the desired tropism, or any other characteristic for which AAV can be screened, as described in Grimm et al. Molecular Therapy 23(12): 1819-1831 (2015) and Michelfelder et al PLoS One 4(4):e5122 (2009), the contents of each of which are herein incorporated by reference in their entirety. Lastly, capsid shuffling, and/or chimeras describe a method in which fragments of at least two parent AAV capsids are combined to generate a new recombinant capsid protein. After library production, the resulting AAV may then be screened for desired properties.

### Capsid Shuffling

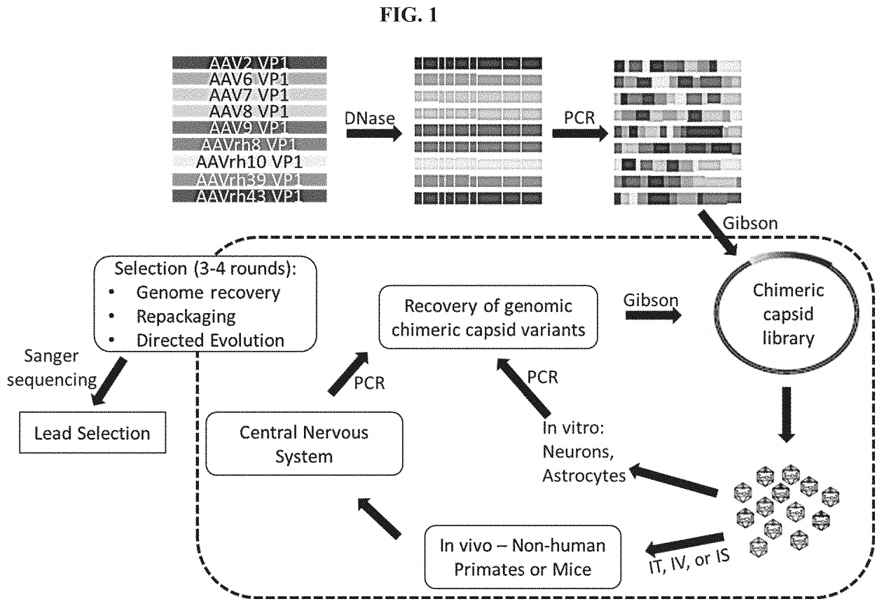

In one embodiment, capsid shuffling may be used to identify AAV capsids having enhanced transduction of a target tissue (e.g., CNS or PNS), as described in Lisowski et al. Nature 506(7488): 382-386 (2014), Grimm et al. Molecular Therapy 23(12): 1819-1831 (2015), Koerber et al. Molecular Therapy 16(10): 1703-1709 (2008), and Grosse et al. Journal of Virology 91(20): e01198-17 (2017), the contents of each of which are herein incorporated by reference in their entirety. The number of parent AAV capsids used may be 2-20, or more than 20. Parental serotypes may be used to amplify full length cap genes via PCR, and then purified and fragmented (e.g. with DNAse I), as seen in FIG. 1. The resulting fragments may be reassembled into full-length cap variants by primerless PCR. The shuffled cap library may then be subcloned into wild type ITR-rep vector. The method of incorporation into the wild type ITR-rep vector may comprise the Gibson Assembly method, as described in Gibson et al., Nt. Methods., 7(11):901-903 (2010), the contents of which are herein incorporated by reference in their entirety.

The shuffled capsid library may then be transformed into cells (e.g. Escherichia coli) (e.g. by electroporation). The integrity and genetic diversity of a shuffled capsid library may then be assessed by any method, including colony number and 100% homology of cap variants in hundreds of colonies (e.g. by Sanger sequencing). To produce a large amount of viruses for directed evolution, the library plasmid and adenoviral helper plasmid may also be transduced into other cells (e.g. HEK-293T). The resulting hybrid viruses in cells and culture medium may then be collected and purified and assessed. The libraries may be assessed in vitro, in vivo, or ex vivo for subsequent directed evolution.

The AAV particles described herein may be generated by any method known in the art. In some embodiments, directed evolution methods are used to identify AAV capsids proteins. In some embodiments, AAV particles described herein may be used to encapsidate one or more viral genomes. In some embodiments, AAV particles described herein may be used to deliver a viral genome to a target tissue.

A viral genome as described herein, may comprise, but is not limited to comprising, at least one inverted terminal repeat (ITR) region, a promoter region, an untranslated region (UTR), a polyadenylation sequence (polyA), an intron, a stuffer sequence, a miRNA or miRNA binding sequence region, and/or a payload region.

### Viral Genome Component: Inverted Terminal Repeats (ITRs)

The AAV particles that have one or more capsid proteins described herein can comprise a viral genome with at least one ITR region and a payload region. In some embodiments, the viral genome has two ITRs. These two ITRs flank the payload region at the 5′ and 3′ ends. The ITRs function as origins of replication comprising recognition sites for replication. ITRs comprise sequence regions that can be complementary and symmetrically arranged. ITRs incorporated into viral genomes described herein may be comprised of naturally occurring polynucleotide sequences or recombinantly derived polynucleotide sequences.

In some embodiments, the AAV particle comprising one or more capsid proteins described herein has more than one ITR. In a non-limiting example, the AAV particle has a viral genome comprising two ITRs. In some embodiments, the viral genome comprises a polynucleotide sequence that encodes a payload molecule described herein that is positioned between the two ITRs. In some embodiments, the ITRs are of the same serotype as one another. In another embodiment, the ITRs are of different serotypes. In some embodiments both ITRs of the viral genome of the AAV particle are AAV2ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 105, 130, 140, 141, 142, 145 nucleotides in length, and those comprising at least 95% identity thereto.

### Viral Genome Component: Promoters

In some embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art will recognize that expression of the polypeptides described herein in a target cell may require a specific promoter, including but not limited to, a promoter that is species specific, inducible, tissue-specific, or cell cycle-specific (Parr et al., Nat. Med 3:1145-9 (1997); the contents of which are herein incorporated by reference in their entirety).

In some embodiments, the promoter is deemed to be efficient when it drives expression of the polypeptide(s) encoded in the payload region of the viral genome of the AAV particle comprising one or more capsid proteins described herein.

In some embodiments, the promoter is a promoter deemed to be efficient when it drives expression in the cell being targeted.

In some embodiments, the promoter is a promoter comprising a tropism for the cell being targeted.

In some embodiments, the promoter drives expression of the payload for a period in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, I week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a weak promoter for sustained expression of a payload in nervous tissues.

In some embodiments, the promoter drives expression of the polypeptides described herein for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated or mutated.

Promoters that drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, p glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters that can be used to restrict expression to neurons or subtypes of neurons, astrocytes, or oligodendrocytes.

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2). Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EF1α, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EF1α promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, a HSV-ILAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter that are both absent in the liver but NFH is abundant in the sensory proprioceptive neurons, brain and spinal cord and NFH is present in the heart. SCN8A is a 470 nucleotide promoter that expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A. Mamm Genome (2007) 18:723-731; and Raymond et al. Expression of Alternatively Spliced Sodium Channel α-subunit genes. Journal of Biological Chemistry (2004) 279(44) 46234-46241; the contents of each of which are herein incorporated by reference in their entireties).

Any of the promoters taught by the aforementioned Yu, Soderblom, Gill, Husain, Passini, Xu, Drews or Raymond may be used in connection with the present disclosure.

In some embodiments, the promoter is not cell specific.

In some embodiments, the promoter is an ubiquitin c (UBC) promoter. The UBC promoter may have a size of 300-350 nucleotides. As a non-limiting example, the UBC promoter is 332 nucleotides.

In some embodiments, the promoter is a β-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides.

In some embodiments, the promoter is a neurofilament heavy (NFH) promoter. The NFH promoter may have a size of 900-950 nucleotides. As a non-limiting example, the NFH promoter is 920 nucleotides.

In some embodiments, the promoter is a SCN8A promoter. The SCN8A promoter may have a size of 450-500 nucleotides. As a non-limiting example, the SCN8A promoter is 470 nucleotides.

92 In some embodiments, the promoter is a frataxin (FXN) promoter.

In some embodiments, the promoter is a phosphoglycerate kinase 1 (PGK) promoter.

In some embodiments, the promoter is a chicken β-actin (CBA) promoter.

In some embodiments, the promoter is a cytomegalovirus (CMV) promoter.

In some embodiments, the promoter is a H1 promoter.

In some embodiments, the promoter is an engineered promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In some embodiments, the promoter is a RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In some embodiments, the viral genome comprises an enhancer element, a promoter and/or a 5′UTR intron. The enhancer element, also referred to herein as an enhancer, may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron: (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In some embodiments, the viral genome comprises an engineered promoter.

In another embodiment, the viral genome comprises a promoter from a naturally expressed protein.

### Viral Genome Component: Untranslated Regions (UTTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another “G”.

In some embodiments, the 5′UTR in the viral genome comprises a Kozak sequence.

In some embodiments, the 5′UTR in the viral genome does not comprise a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may comprise an oligo(dT) sequence for templated addition of a poly-A tail.

In some embodiments, the viral genome may comprise at least one miRNA seed, binding site or full sequence. MicroRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence includes a “seed” region, e.g., a sequence in the region of positions 2-8 of the mature microRNA, which sequence has perfect Watson-Crick complementarity to the miRNA target sequence of the nucleic acid.

In some embodiments, the viral genome may be engineered to comprise, alter or remove at least one miRNA binding site, sequence or seed region.

Any UTR from any gene known in the art may be incorporated into the viral genome of the AAV particle comprising one or more capsid proteins described herein. These UTRs, or portions thereof, may be placed in the same orientation as in the gene from which they were selected or they may be altered in orientation or location. In some embodiments, the UTR used in the viral genome of the AAV particle may be inverted, shortened, lengthened, made with one or more other 5′ UTRs or 3′ UTRs known in the art. Altered as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprising one or more capsid proteins described herein comprises at least one artificial UTR that is not a variant of a wild type UTR.

In some embodiments, the viral genome of the AAV particle comprising one or more capsid proteins described herein comprises UTRs that have been selected from a family of transcripts whose proteins share a common function, structure, feature or property.

### Viral Genome Component: Polyadenylation Sequence

In some embodiments, the viral genome of the AAV particles comprising one or more capsid proteins described herein comprise at least one polyadenylation sequence. The viral genome of the AAV particle may comprise a polyadenylation sequence between the 3′ end of the payload coding sequence and the 5′ end of the 3′ITR.

In some embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287.288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493.494, 495, 496, 497, 498, 499, and 500 nucleotides in length.

In some embodiments, the polyadenylation sequence is 50-100 nucleotides in length.

In some embodiments, the polyadenylation sequence is 50-150 nucleotides in length.

In some embodiments, the polyadenylation sequence is 50-160 nucleotides in length.

In some embodiments, the polyadenylation sequence is 50-200 nucleotides in length.

In some embodiments, the polyadenylation sequence is 60-100 nucleotides in length.

In some embodiments, the polyadenylation sequence is 60-150 nucleotides in length.

In some embodiments, the polyadenylation sequence is 60-160 nucleotides in length.

In some embodiments, the polyadenylation sequence is 60-200 nucleotides in length.

In some embodiments, the polyadenylation sequence is 70-100 nucleotides in length.

In some embodiments, the polyadenylation sequence is 70-150 nucleotides in length.

In some embodiments, the polyadenylation sequence is 70-160 nucleotides in length.

In some embodiments, the polyadenylation sequence is 70-200 nucleotides in length.

In some embodiments, the polyadenylation sequence is 80-100 nucleotides in length.

In some embodiments, the polyadenylation sequence is 80-150 nucleotides in length.

In some embodiments, the polyadenylation sequence is 80-160 nucleotides in length.

In some embodiments, the polyadenylation sequence is 80-200 nucleotides in length.

In some embodiments, the polyadenylation sequence is 90-100 nucleotides in length.

In some embodiments, the polyadenylation sequence is 90-150 nucleotides in length.

In some embodiments, the polyadenylation sequence is 90-160 nucleotides in length.

In some embodiments, the polyadenylation sequence is 90-200 nucleotides in length.

### Viral Genome Component: Introns

In some embodiments, the viral genome comprises at least one element to enhance the transgene target specificity and expression (See e.g., Powell et al. Viral Expression Cassette Elements to Enhance Transgene Target Specificity and Expression in Gene Therapy, 2015: the contents of which are herein incorporated by reference in its entirety) such as an intron. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80450, 80-500, 200-300, 200-400, 200-500, 300-400, 300-500, or 400-500.

### Viral Genome Component: Stuffer Sequences

In some embodiments, the viral genome comprises at least one element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In some embodiments, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000.

Viral Genome Component: miRNA

In some embodiments, the viral genome comprises at least one sequence encoding a miRNA to reduce the expression of the transgene is a specific tissue. miRNAs and their targeted tissues are well known in the art. As a non-limiting example, a miR-122 miRNA may be encoded in the viral genome to reduce the expression of the viral genome in the liver.

### AAV Production

The present disclosure provides methods for the generation of AAV particles comprising one or more capsid proteins described herein by viral genome replication in a viral replication cell.

In accordance with the present disclosure, the viral genome comprising a payload region will be incorporated into the AAV particle comprising one or more capsid proteins described herein produced in a viral replication cell. Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948: or International Publication Nos. WO1996039530, WO1998010088, WO1999014354, WO1999015685, WO1999047691, WO2000055342. WO2000075353 and WO2001023597; Methods In Molecular Biology, ed. Richard. Humana Press, NJ (1995); O'Reilly et al., Baculovirus Expression Vectors, A Laboratory Manual, Oxford Univ. Press (1994); Samulski et al., J. Vir. 63:3822-8 (1989): Kajigaya et al., Proc. Nat'l. Acad. Sci. USA 88: 4646-50 (1991); Ruffing et al., J. Vir. 66:6922-30 (1992): Kimbauer et al., Vir., 219:37-44 (1996): Zhao et al., Vir. 272:382-93 (2000): the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AAV particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties.

In some embodiments, the present disclosure provides a method for producing an AAV particle comprising one or more capsid proteins described herein wherein the particle has enhanced (increased, improved) transduction efficiency comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying the AAV particle comprising a viral genome.

In some embodiments, the present disclosure provides a method for producing an AAV particle comprising one or more capsid proteins described herein, wherein the method comprises the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In some embodiments, the viral genome of the AAV particle comprising one or more capsid proteins described herein optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes as described in International application No. WO 96/23810: Heim et al., Current Biology 2:178-182 (1996): Heim et al., Proc. Natl. Acad. Sci. USA (1995); or Heim et al., Science 373:663-664 (1995); WO 96/30540, the contents of each of which are incorporated herein by reference in their entiretics).

In some embodiments, provided herein is a method for producing an AAV particle comprising one or more capsid proteins described herein whereby the particle is produced by insect cells, for example, by using an Sf9/baculovirus insect cell system.

In some embodiments, the present disclosure provides a method of making AAV particles comprising one or more capsid proteins described herein, wherein the method comprises: (a) culturing insect cells to produce the AAV particles; and (b) harvesting the particles produced by the insect cells. For example, in some embodiments, the present disclosure provides a method of AAV particles comprising one or more capsid proteins described herein, wherein the method comprises: (a) culturing insect cells comprising one or more baculovirus expression vectors, or BEVs, to produce the AAV particles; and (b) harvesting the AAV particles produced by the insect cells.

A BEV is a baculovirus plasmid or bacmid comprising a viral construct for expression of non-structural and structural proteins and/or a payload construct as described herein. In this context, “non-structural proteins” refer to proteins involved in AAV replication, including site specific endonuclease and helicase activity, DNA replication and activation of promoters during transcription, or proteins that are required for assembly of the capsid of an AAV particle. Also in this context, “structural proteins” refer to capsid proteins, such as VP1, VP2 and VP3 capsid proteins described herein, of an AAV particle.

In the context of AAV, the rep gene encodes the non-structural Rep proteins of Rep78, Rep68, Rep52 and Rep40, which in the plasmid(s) or bacmid(s) can be expressed via single or multiple, separate, coding sequences and the ORF2 of the cap gene encodes the non-structural Assembly-Activating Protein (AAP) for introducing such constructs into a baculovirus plasmid or bacmid are well known in the art, which can include use of a transposon donor/acceptor system. Accordingly, in some embodiments, an insect cell for producing an AAV particle comprising one or more capsids described herein can comprise a polynucleotide sequence (e.g. a rep gene) that encodes a Rep protein, such as a polynucleotide sequence encoding a Rep78, Rep68, Rep52 and/or Rep40 protein. In some embodiments, the insect cell comprises a polynucleotide sequence encoding Rep78. In some embodiments, the insect cell comprises a polynucleotide sequence encoding Rep52. In some embodiments, the insect cell comprises a polynucleotide sequence encoding Rep78 and Rep52.

The polynucleotide sequences encoding the Rep protein, in some embodiments, can be part of the same nucleic acid molecule that encodes the one or more capsid protein described herein. Accordingly, in some embodiments, an insect cell for producing AAV particles comprising one or more capsids described herein comprises a nucleic acid molecule comprising a polynucleotide sequence encoding a Rep78, Rep68, Rep52 and/or Rep40 protein and a polynucleotide sequence encoding one or more capsids described in Tables 1-3. In some embodiments, the nucleic acid molecule comprises polynucleotide sequence encoding Rep78 and a polynucleotide sequence encoding the capsid protein of SEQ ID NO: 6. In some embodiments, the nucleic acid molecule comprises polynucleotide sequence encoding Rep52 and a polynucleotide sequence encoding the capsid protein of SEQ ID NO: 6. In some embodiments, the nucleic acid molecule comprises polynucleotide sequence encoding Rep78 and Rep 52 and a polynucleotide sequence encoding the capsid protein of SEQ ID NO: 6. In some embodiments, the nucleic acid molecule comprises polynucleotide sequence encoding Rep78 and a polynucleotide sequence encoding the capsid protein of SEQ ID NO: 8. In some embodiments, the nucleic acid molecule comprises polynucleotide sequence encoding Rep52 and a polynucleotide sequence encoding the capsid protein of SEQ ID NO: 8. In some embodiments, the nucleic acid molecule comprises polynucleotide sequence encoding Rep78 and Rep 52 and a polynucleotide sequence encoding the capsid protein of SEQ ID NO: 8. In some embodiments, the nucleic acid molecule comprises polynucleotide sequence encoding Rep78 and a polynucleotide sequence encoding the capsid protein of SEQ ID NO: 9. In some embodiments, the nucleic acid molecule comprises polynucleotide sequence encoding Rep52 and a polynucleotide sequence encoding the capsid protein of SEQ ID NO: 9. In some embodiments, the nucleic acid molecule comprises polynucleotide sequence encoding Rep78 and Rep 52 and a polynucleotide sequence encoding the capsid protein of SEQ ID NO: 9.

In one embodiment, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG. TTG, or GTG, as described in U.S. Pat. No. 8,163,543, the contents of which are herein incorporated by reference in its entirety.

In one embodiment, the viral construct vector(s) used for AAV production may contain a nucleotide sequence encoding the AAV rep proteins where the initiation codon of the AAV rep protein or proteins is a non-ATG. In one embodiment, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the present disclosure provides a method for producing AAV particles comprising one or more capsid proteins described herein, wherein the method comprises: (a) culturing insect cells: (b) infecting the insect cells with a first BIIC and a second BIIC, wherein the first BIIC comprises a baculovirus expression vector comprising a polynucleotide sequence that produces an AAV viral genome described herein, and wherein the second BIIC comprises a baculovirus expression vector comprising a nucleotide sequence that produces AAV non-structural and structural proteins necessary for AAV particle formation in the insect cells; and (c) harvesting the AAV particles produced by the insect cells following the infection step. A BIIC is a “baculovirus infected insect cell” and refers to an insect cell that has been infected with a BEV.

Growing conditions for insect cells in culture, and production of heterologous products in insect cells in culture are well-known in the art, see U.S. Pat. No. 6,204,059, the contents of which are herein incorporated by reference in their entirety.

Any insect cell that allows for replication of a parvovirus (e.g., AAV) and that can be maintained in culture can be used in accordance with the present disclosure. Cell lines can be used from Spodoptera frugiperda, including, but not limited to the pupal ovarian Sf9 or Sf21 cell lines, drosophila cell lines, or mosquito cell lines, such as, Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, NJ (1995), O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad Sci. USA 88: 4646-50 (1991): Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety.

Baculovirus expression vectors for producing AAV particles in insect cells, including but not limited to Spodoptera frugiperda (Sf9) cells, provide high titers of AAV particle product. Recombinant baculovirus encoding the viral construct expression vector and payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety.

In some embodiments, a genetically stable baculovirus can be used to produce the source of one or more of the components for producing AAV particles in invertebrate cells. In some embodiments, defective baculovirus expression vectors can be maintained episomally in insect cells. In such an embodiment, the bacmid vector is engineered with replication control elements, including but not limited to promoters, enhancers, and/or cell-cycle regulated replication elements.

In some embodiments, baculoviruses can be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In some embodiments, stable viral replication cells permissive for baculovirus infection are engineered with at least one stable integrated copy of any of the elements necessary for AAV replication and AAV particle production including, but not limited to, i) the entire AAV genome, ii) rep genes and polynucleotide sequences that express capsid protein coding sequences described herein (either as a single or separate open reading frames), iii) rep genes, iv) polynucleotide sequences that express capsid protein coding sequences (either as single or separate open reading frames), v) polynucleotides that express each Rep protein coding sequence as a separate transcription cassette, vi) polynucleotides that express each capsid VP protein coding sequence as a separate transcription/expression cassette, vii) polynucleotides that express the AAP (assembly activation protein), and/or viii) at least one of the baculovirus helper genes with native or non-native promoters.

In some embodiments, the polynucleotide sequence described herein that encodes a Rep protein and/or a capsid protein is linked to a sequence that promotes expression of the Rep protein and/or capsid protein in insect cells. Accordingly, in some embodiments, a nucleic acid molecule described herein comprising a polynucleotide sequence encoding a capsid protein of Tables 1-3 is linked to a second polynucleotide sequence that promotes expression in insect cells. In some embodiments, an insect cell described herein comprises a polynucleotide sequence encoding a Rep protein (e.g., Rep 78, Rep 68, Rep 40 or Rep 52) linked to a polynucleotide sequence that promotes expression in insect cells. Non-limiting examples of polynucleotide sequences that promote expression in insect cells include promoters, enhancers, and/or cell-cycle regulated replication elements. Exemplary promoters include the Baculovirus immediate-early gene (ie 1) promoter, truncated promoter for the immediate-early 1 gene of Orgyia pseudotsugata nuclear polyhedrosis virus (deltalE1 promoter), Actin 5c gene promoter, polyhedrin gene promoter, and p10 gene promoter.

In some embodiments, large-scale viral production methods can include the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats can be carried out according to any methods known in the art. For large-scale adherent cell cultures, transfection methods can include, but are not limited to the use of inorganic compounds (e.g. calcium phosphate) organic compounds [e.g. polyethyleneimine (PEI)] or the use of non-chemical methods (e.g. electroporation). With cells grown in suspension, transfection methods can include, but are not limited to the use of calcium phosphate and the use of PEI. In some cases, transfection of large scale suspension cultures can be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes can be formed for introduction of plasmids to be transfected. In some cases, cells being transfected with PEI-DNA complexes can be ‘shocked’ prior to transfection. This includes lowering cell culture temperatures to 4° C. for a period of about 1 hour. In some cases, cell cultures can be shocked for a period of from about 10 minutes to about 5 hours. In some cases, cell cultures can be shocked at a temperature of from about 0° C. to about 20° C.

In some cases, transfections can comprise one or more vectors for expression of an RNA effector molecule to reduce expression of nucleic acids from one or more payload constructs. Such methods can enhance the production of AAV particles by reducing cellular resources wasted on expressing payload constructs. In some cases, such methods can be carried according to those taught in US Publication No. US2014/0099666, the contents of which are herein incorporated by reference in their entirety.

Cells described herein, including, but not limited to viral production cells, can be subjected to cell lysis according to any methods known in the art. Cell lysis can be carried out to obtain one or more agents (e.g. AAV particles) present within any cells described herein. In some embodiments, cell lysis can be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258.595, 6,261,551, 6,270,996, 6,281,010, 6,365.394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods can be chemical or mechanical. Chemical cell lysis typically comprises contacting one or more cells with one or more lysis agents. Mechanical lysis typically comprises subjecting one or more cells to one or more lysis conditions and/or one or more lysis forces.

In some embodiments, chemical lysis can be used to lyse cells. As used herein, the term lysis agent refers to any agent that can aid in the disruption of a cell. In some cases, lysis agents are introduced in solutions, termed lysis solutions or lysis buffers. As used herein, the term lysis solution refers to a solution (typically aqueous) including one or more lysis agents. In addition to lysis agents, lysis solutions can include one or more buffering agents, solubilizing agents, surfactants, preservatives, cryoprotectants, enzymes, enzyme inhibitors and/or chelators. Lysis buffers are lysis solutions including one or more buffering agents. Additional components of lysis solutions can include one or more solubilizing agents. As used herein, the term solubilizing agent refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents can include any of those described in U.S. Pat. Nos. 8,685,734, 7,901,921, 7,732,129, 7,223.585, 7,125,706, 8,236,495, 8,110,351, 7,419.956, 7,300,797, 6,699,706 and 6,143,567, the contents of each of which are herein incorporated by reference in their entirety. In some cases, lysis agents can be selected from lysis salts, amphoteric agents, cationic agents, ionic detergents and non-ionic detergents. Lysis salts can include, but are not limited to sodium chloride (NaCl) and potassium chloride (KC). Further lysis salts can include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts can be increased or decreased to obtain an effective concentration for rupture of cell membranes. Amphoteric agents, as referred to herein, are compounds capable of reacting as an acid or a base. Amphoteric agents can include, but are not limited to lysophosphatidylcholine, 3-((3-Cholamidopropyl)dimethylammonium)-1-propanesulfonate (CHAPS), ZWITTERGENT® and the like. Cationic agents can include, but are not limited to cetyltrimethylammonium bromide (C(16)TAB) and Benzalkonium chloride. Lysis agents including detergents can include ionic detergents or non-ionic detergents. Detergents can function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents can include, but are not limited to sodium dodecyl sulfate (SDS), cholate and deoxycholate. In some cases, ionic detergents can be included in lysis solutions as a solubilizing agent. Non-ionic detergents can include, but are not limited to octylglucoside, digitonin, lubrol, C12E8, TWEEN®-20, TWEEN®-80, Triton X-100 and Noniodet P-40. Non-ionic detergents are typically weaker lysis agents, but can be included as solubilizing agents for solubilizing cellular and/or viral proteins. Further lysis agents can include enzymes and urea. In some cases, one or more lysis agents can be combined in a lysis solution in order to enhance one or more of cell lysis and protein solubility. In some cases, enzyme inhibitors can be included in lysis solutions in order to prevent proteolysis that can be triggered by cell membrane disruption.

In some embodiments, mechanical cell lysis is carried out. Mechanical cell lysis methods can comprise the use of one or more lysis conditions and/or one or more lysis forces. As used herein, the term lysis condition refers to a state or circumstance that promotes cellular disruption. Lysis conditions can comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments can comprise freeze-thaw lysis. As used herein, the term freeze-thaw lysis refers to cellular lysis in which a cell solution is subjected to one or more freeze-thaw cycles. According to freeze-thaw lysis methods, cells in solution are frozen to induce a mechanical disruption of cellular membranes caused by the formation and expansion of ice crystals. Cell solutions used according to freeze-thaw lysis methods, can further comprise one or more lysis agents, solubilizing agents, buffering agents, cryoprotectants, surfactants, preservatives, enzymes, enzyme inhibitors and/or chelators. Once cell solutions subjected to freezing are thawed, such components can enhance the recovery of desired cellular products. In some cases, one or more cyroprotectants are comprised in cell solutions undergoing freeze-thaw lysis. A cryoprotectant refers to an agent used to protect one or more substances from damage due to freezing. Cryoprotectants described herein can comprise any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants can comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis can be carried out according to any of the methods described in U.S. Pat. No. 7,704,721, the contents of which are herein incorporated by reference in their entirety.

As used herein, the term lysis force refers to a physical activity used to disrupt a cell. Lysis forces can include, but are not limited to mechanical forces, sonic forces, gravitational forces, optical forces, electrical forces and the like. Cell lysis carried out by mechanical force is referred to herein as mechanical lysis. Mechanical forces that can be used according to mechanical lysis can comprise high shear fluid forces. According to such methods of mechanical lysis, a microfluidizer can be used. Microfluidizers typically include an inlet reservoirs where cell solutions can be applied. Cell solutions can then be pumped into an interaction chamber via a pump (e.g. high-pressure pump) at high speed and/or pressure to produce shear fluid forces. Resulting lysates can then be collected in one or more output reservoir. Pump speed and/or pressure can be adjusted to modulate cell lysis and enhance recovery of products (e.g. AAV particles). Other mechanical lysis methods can comprise physical disruption of cells by scraping.

Cell lysis methods can be selected based on the cell culture format of cells to be lysed. For example, with adherent cell cultures, some chemical and mechanical lysis methods can be used. Such mechanical lysis methods can comprise freeze-thaw lysis or scraping. In another example, chemical lysis of adherent cell cultures can be carried out through incubation with lysis solutions including surfactant, such as Triton-X-100. In some cases, cell lysates generated from adherent cell cultures can be treated with one more nucleases to lower the viscosity of the lysates caused by liberated DNA.

Cell lysates comprising AAV particles comprising one or more capsid proteins described herein can be subjected to clarification. Clarification refers to initial steps taken in purification of AAV particles from cell lysates. Clarification serves to prepare lysates for further purification by removing larger, insoluble debris. Clarification steps can include, but are not limited to centrifugation and filtration. During clarification, centrifugation can be carried out at low speeds to remove larger debris, only. Similarly, filtration can be carried out using filters with larger pore sizes so that only larger debris is removed. In some cases, tangential flow filtration can be used during clarification. Objectives of viral clarification include high throughput processing of cell lysates and to optimize ultimate viral recovery. Advantages of including a clarification step include scalability for processing of larger volumes of lysate. In some embodiments, clarification can be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491.508. US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and can be carried out according to a variety of available methods including, but not limited to passive filtration and flow filtration. Filters used can comprise a variety of materials and pore sizes. For example, cell lysate filters can comprise pore sizes of from about 1 μM to about 5 μM, from about 0.5 μM to about 2 μM, from about 0.1 μM to about 1 μM, from about 0.05 μM to about 0.5 μM and from about 0.001 μM to about 0.1 μM. Exemplary pore sizes for cell lysate filters can comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In some embodiments, clarification can comprise filtration through a filter with 2.0 μM pore size to remove large debris, followed by passage through a filter with 0.45 μM pore size to remove intact cells.

Filter materials can be composed of a variety of materials. Such materials can include, but are not limited to polymeric materials and metal materials (e.g. sintered metal and pored aluminum). Exemplary materials can include, but are not limited to nylon, cellulose materials (e.g. cellulose acetate), polyvinylidene fluoride (PVDF), polyethersulfone, polyamide, polysulfone, polypropylene and polyethylene terephthalate. In some cases, filters useful for clarification of cell lysates can include, but are not limited to ULTIPLEAT PROFILE™ filters (Pall Corporation, Port Washington, N.Y.), SUPOR™ membrane filters (Pall Corporation, Port Washington, N.Y.)

In some cases, flow filtration can be carried out to increase filtration speed and/or effectiveness. In some cases, flow filtration can comprise vacuum filtration. According to such methods, a vacuum is created on the side of the filter opposite that of cell lysate to be filtered. In some cases, cell lysates can be passed through filters by centrifugal forces. In some cases, a pump is used to force cell lysate through clarification filters. Flow rate of cell lysate through one or more filters can be modulated by adjusting one of channel size and/or fluid pressure.

According to some embodiments, cell lysates can be clarified by centrifugation. Centrifugation can be used to pellet insoluble particles in the lysate. During clarification, centrifugation strength [expressed in terms of gravitational units (g), which represents multiples of standard gravitational force] can be lower than in subsequent purification steps. In some cases, centrifugation can be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation can be carried out in order to partition particulates in the cell lysate by sedimentation rate. Gradients used according to methods of the present disclosure can comprise, but are not limited to cesium chloride gradients and iodixanol step gradients.

In some cases, AAV particles comprising one or more capsid proteins described herein can be purified from clarified cell lysates by one or more methods of chromatography. Chromatography refers to any number of methods known in the art for separating out one or more elements from a mixture. Such methods can comprise, but are not limited to ion exchange chromatography (e.g. cation exchange chromatography and anion exchange chromatography) immunoaffinity chromatography and size-exclusion chromatography. In some embodiments, methods of viral chromatography can comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

In some embodiments, ion exchange chromatography can be used to isolate AAV particles comprising one or more capsid proteins described herein. Ion exchange chromatography is used to bind AAV particles based on charge-charge interactions between capsid proteins and charged sites present on a stationary phase, typically a column through which viral preparations (e.g. clarified lysates) are passed. After application of viral preparations, bound AAV particles can then be eluted by applying an elution solution to disrupt the charge-charge interactions. Elution solutions can be optimized by adjusting salt concentration and/or pH to enhance recovery of bound AAV particles, and can comprise cation or anion exchange chromatography methods. Methods of ion exchange chromatography can comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, size-exclusion chromatography (SEC) can be used. SEC can include the use of a gel to separate particles according to size. In AAV particle purification, SEC filtration is sometimes referred to as “polishing.” In some cases. SEC can be carried out to generate a final product that is near-homogenous. Such final products can in some cases be used in pre-clinical studies and/or clinical studies (Kotin, R. M. 2011. Human Molecular Genetics. 20(1):R2-R6, the contents of which are herein incorporated by reference in their entirety). In some cases, SEC can be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, AAV particles comprising one or more capsid proteins described herein can be isolated or purified using the methods described in U.S. Pat. Nos. 6,146,874, 6,660,514, 8,283,151, or 8,524,446, the contents of each of which is herein incorporated by reference in its entirety.

### Genome Size

In some embodiments, the AAV particle which comprises a payload described herein may be single stranded or double stranded viral genome. The size of the viral genome may be small, medium, large or the maximum size. Additionally, the viral genome may comprise a promoter and a polyA tail.

In some embodiments, the viral genome that comprises a payload described herein may be a small single stranded viral genome. A small single stranded viral genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded viral genome may be 3.2 kb in size. As another non-limiting example, the small single stranded viral genome may be 2.2 kb in size. Additionally, the viral genome may comprise a promoter and a polyA tail.

In some embodiments, the viral genome that comprises a payload described herein may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may comprise a promoter and a polyA tail.

In some embodiments, the viral genome that comprises a payload described herein e.g., polynucleotide, siRNA or dsRNA, may be a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may comprise a promoter and a polyA tail.

In some embodiments, the viral genome that comprises a payload described herein may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may comprise a promoter and a polyA tail.

In some embodiments, the viral genome that comprises a payload described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may comprise a promoter and a polyA tail.

In some embodiments, the viral genome that comprises a payload described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may comprise a promoter and a polyA tail.

### Payloads

The AAV particles of the present disclosure comprise at least one payload region. Payloads described herein typically encode polypeptides or fragments or variants thereof, or modulatory polynucleotides (e.g., miRNAs).

An RNA encoded by the payload region can, for example, comprise an mRNA, tRNA, rRNA, tmRNA, miRNA, siRNA, piRNA, shRNA antisense RNA, double stranded RNA, snRNA, snoRNA, or long non-coding RNA (lncRNA).

The payload region may be constructed in such a way as to reflect a region similar to or mirroring the natural organization of an mRNA.

The payload region may comprise a combination of coding and non-coding nucleic acid sequences. In some embodiments, the AAV payload region may encode a coding or non-coding RNA.

In some embodiments, the AAV payload region encodes one or more microRNAs (or miRNA) that are 19-25 nucleotide long noncoding RNAs that bind to the 3′UTR of nucleic acid molecules and down-regulate gene expression either by reducing nucleic acid molecule stability or by inhibiting translation. The payload region can comprise one or more microRNA target sequences, microRNA sequences, or microRNA seeds. Such sequences can correspond to any known microRNA such as those taught in US Publication US2005/0261218 and US Publication US2005/0059005, the contents of which are incorporated herein by reference in their entirety. A microRNA sequence includes a seed region, e.g., a sequence in the region of positions 2-8 of the mature microRNA, which has perfect Watson-Crick complementarity to the miRNA target sequence. A microRNA seed can comprise positions 2-8 or 2-7 of the mature microRNA. In some embodiments, a microRNA seed can comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed can comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

In some embodiments, the payload region comprises more than one nucleic acid sequence encoding more than one payload molecule of interest. In some embodiments, the AAV particle comprises a viral genome with a payload region comprising nucleic acid sequences encoding more than one polypeptide of interest. In such an embodiment, a viral genome encoding more than one polypeptide may be replicated and packaged into a viral (e.g., an AAV) particle comprising one or more capsid proteins as described herein. A target cell transduced with such a viral particle comprising more than one polypeptide may express each of the polypeptides in a single cell.

In some embodiments, the payload region may comprise the components a payload region located within the viral genome. At the 5′ and/or the 3′ end of the payload region, there may be at least one inverted terminal repeat (ITR). In some embodiments, within the payload region, there is a promoter region, an intron region and a coding region.

Where the AAV particle payload region encodes a polypeptide, the polypeptide may be a peptide or protein. As a non-limiting example, the payload region may encode at least one allele of apolipoprotein E (APOE) such as, but not limited to ApoE2, ApoE3 and/or ApoE4. As a second non-limiting example, the payload region may encode a human or a primate frataxin protein, or fragment or variant thereof. As another non-limiting example, the payload region may encode an antibody, or a fragment thereof. As another non-limiting example, the payload region may encode SMN, or fragment or variant thereof. As another non-limiting example, the payload region may encode GCase, or fragment or variant thereof. As another non-limiting example, the payload region may encode N-sulfoglucosamine sulfohydrolase, or fragment or variant thereof. As another non-limiting example, the payload region may encode N-acetyl-alpha-glucosaminidase, or fragment or variant thereof. As another non-limiting example, the payload region may encode iduronate 2-sulfatase, or fragment or variant thereof. As another non-limiting example, the payload region may encode alpha-L-iduronidase, or fragment or variant thereof. As another non-limiting example, the payload region may encode palmitoyl-protein thioesterase 1, or fragment or variant thereof. As another non-limiting example, the payload region may encode tripeptidyl peptidase 1, or fragment or variant thereof. As another non-limiting example, the payload region may encode battenin, or fragment or variant thereof. As another non-limiting example, the payload region may encode CLN5, or fragment or variant thereof. As another non-limiting example, the payload region may encode CLN6 (linclin), or fragment or variant thereof. As another non-limiting example, the payload region may encode MFSD8, or fragment or variant thereof. As another non-limiting example, the payload region may encode CLN8, or fragment or variant thereof. As another non-limiting example, the payload region may encode ASPA, or fragment or variant thereof. As another non-limiting example, the payload region may encode GRN, or fragment or variant thereof. As another non-limiting example, the payload region may encode MeCP2, or fragment or variant thereof. As another non-limiting example, the payload region may encode GLB1, or fragment or variant thereof. As another non-limiting example, the payload region may encode GAN, or fragment or variant thereof. The AAV viral genomes encoding polypeptides described herein may be useful in the fields of human disease, viruses, infections veterinary applications and a variety of in vivo and in vitro settings.

In some embodiments, AAV particles comprising one or more capsid proteins described herein are useful in the field of medicine for the treatment, prophylaxis, palliation or amelioration of neurological diseases and/or disorders.

In some embodiments. AAV particles comprising one or more capsid proteins described herein are useful in the field of medicine for the treatment, prophylaxis, palliation or amelioration of tauopathy.

In some embodiments, AAV particles comprising one or more capsid proteins described herein are useful in the field of medicine for the treatment, prophylaxis, palliation or amelioration of Alzheimer's Disease.

In some embodiments, AAV particles comprising one or more capsid proteins described herein are useful in the field of medicine for the treatment, prophylaxis, palliation or amelioration of Friedreich's ataxia, or any disease stemming from a loss or partial loss of frataxin protein.

In some embodiments, AAV particles comprising one or more capsid proteins described herein are useful in the field of medicine for the treatment, prophylaxis, palliation or amelioration of Parkinson's Disease.

In some embodiments, AAV particles comprising one or more capsid proteins described herein are useful in the field of medicine for the treatment, prophylaxis, palliation or amelioration of Amyotrophic lateral sclerosis.

In some embodiments, AAV particles comprising one or more capsid proteins described herein are useful in the field of medicine for the treatment, prophylaxis, palliation or amelioration of Huntington's Disease.

Amino acid sequences encoded by payload regions of the viral genomes described herein may be translated as a whole polypeptide, a plurality of polypeptides or fragments of polypeptides, which independently may be encoded by one or more nucleic acids, fragments of nucleic acids or variants of any of the aforementioned.

Sequence tags or amino acids, such as one or more lysines, can be added to the peptide sequences described herein (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble, or linked to a solid support.

### Payloads: Nucleic Acids Encoding a Protein of Interest

In some embodiments, the payload region of the AAV particle comprising one or more capsid proteins described herein comprises one or more nucleic acid sequences encoding a protein or polypeptide of interest.

### Apolipoprotein E (APOE)

In some embodiments, the payload region of the AAV particle comprises one or more nucleic acid sequences encoding an allele of the apolipoprotein E (APOE) gene (e.g., ApoE2, ApoE3, and/or ApoE4), for example, an allele of the human APOE gene. In a non-limiting example, the payload region of the AAV particle comprises a nucleic acid sequence, or fragment thereof, as found at NCBI reference numbers NP_00032.1, NP_001289618.1, NP_0, NP_001289617.1, NM_000041.3, NM_001302689.1, NM_001302690.1, or NM_001302688.1, or Ensembl reference numbers ENSP00000252486, ENSP00000413135, ENSP00000413653, ENSP00000410423, ENST00000252486.8, ENST0000044699.5, ENST0000045628.2, ENST00000434152.5, or ENST00000425718.1.

### Frataxin (FXA)

In some embodiments, the payload region of the AAV particle comprises one or more nucleic acid sequences encoding frataxin (FXN) for example, human frataxin. In a non-limiting example, the payload region of the AAV particle comprises a nucleic acid sequence, or fragment thereof, as found at NCBI reference numbers NP_000135.2, NP_852090.1, NP_001155178.1, NM_000144.4, NM_181425.2, or NM_001161706.1.

### Aromatic L-Amino Acid Decarboxylase (AADC)

In some embodiments, the payload region of the AAV particle comprises one or more nucleic acid sequences encoding Aromatic L-Amino Acid Decarboxylase (AADC), for example, human AADC. In a non-limiting example, the payload region of the AAV particle comprises a nucleic acid sequence, or fragment thereof, as found at NCBI reference numbers NP_00078.1 or NM_000790.3.

### tau Antibody

In some embodiments, the payload region of the AAV particle comprises one or more nucleic acid sequences encoding the heavy chain and/or light chain of an antibody directed against a tau protein, for example, a human tau protein. In some embodiments, the tau antibody is the Paired Helical Filamentous 1 (PHF-1) antibody.

### Payloads: Modulatory Polynucleotides as Payloads

In some embodiments, the present disclosure relates to AAV particles comprising one or more capsid proteins, wherein the AAV particles encode modulatory polynucleotides, e.g., RNA or DNA molecules, as therapeutic agents. RNA interference mediated gene silencing can specifically inhibit targeted gene expression. The present disclosure then provides small double stranded RNA (dsRNA) molecules (small interfering RNA, siRNA) targeting a gene of interest, pharmaceutical compositions comprising such siRNAs, as well as processes of their design. The present disclosure also provides methods of their use for inhibiting gene expression and protein production of a gene of interest, for treating a neurological disease.

In some embodiments, the present disclosure provides small interfering RNA (siRNA) duplexes (and modulatory polynucleotides encoding them) that target the mRNA of a gene of interest to interfere with the gene expression and/or protein production.

In some embodiments, the siRNA duplexes described herein may target the gene of interest along any segment of their respective nucleotide sequence.

In some embodiments, the siRNA duplexes described herein may target the gene of interest at the location of a SNP or variant within the nucleotide sequence.

In some embodiments, expression of the siRNA duplexes described herein inhibits or suppresses the expression of a gene of interest in a cell.

In some embodiments, a nucleic acid sequence encoding such siRNA molecules, or a single strand of the siRNA molecules, is inserted into a viral gnome of an AAV particle, which is introduced into cells, specifically cells in the central nervous system.

AAV particles have been investigated for siRNA delivery because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the particle and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

siRNA duplex sequences generally contain an antisense strand and a sense strand hybridized together forming a duplex structure, wherein the antisense strand is complementary to the nucleic acid sequence of the targeted gene, and wherein the sense strand is homologous to the nucleic acid sequence of the targeted gene. In some aspects, the 5′ end of the antisense strand has a 5′ phosphate group and the 3′ end of the sense strand contains a 3′ hydroxyl group. In other aspects, there are none, one or 2 nucleotide overhangs at the 3′ end of each strand.

In one aspect, each strand of the siRNA duplex targeting a gene of interest is about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length. In some aspects, the siRNAs may be unmodified RNA molecules.

In other aspects, the siRNAs may contain at least one modified nucleotide, such as base, sugar or backbone modification.

In some embodiments, an siRNA or dsRNA comprises at least two sequences that are complementary to each other. The dsRNA comprises a sense strand comprising a first sequence and an antisense strand comprising a second sequence. The antisense strand comprises a nucleotide sequence that is substantially complementary to at least part of an mRNA encoding the target gene, and the region of complementarity is 30 nucleotides or less and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length.

The dsRNA, whether directly administered or encoded by AAV particles described herein upon contacting with a cell expressing the target protein, inhibits the expression of the protein by at least 10%, at least 20%, at least 25%, at least 30%, at least 35% or at least 40% or more, such as when assayed by a method as described herein.

The siRNA molecules comprised in the compositions featured herein comprise a dsRNA comprising an antisense strand (the antisense strand) comprising a region that is 30 nucleotides or less, generally 19 to 25, 19 to 24 or 19 to 21 nucleotides in length, that is substantially complementary to at least part of an mRNA transcript of a target gene.

In one aspect, AAV particles described herein comprise one or more capsid proteins described herein and a viral genome comprising nucleic acids that encode siRNA duplexes. For example, in some embodiments, such an AAV particle has one or more of the capsid proteins in Table 1, Table 2, and/or Table 3, or variants thereof.

In one aspect, the siRNA molecules are designed and tested for their ability in reducing target gene mRNA levels in cultured cells.

The present disclosure also provides pharmaceutical compositions comprising an AAV particle comprising one or more capsid proteins described herein and a viral genome that encodes at least one siRNA duplex targeting a gene of interest and a pharmaceutically acceptable carrier.

In some embodiments, an siRNA duplex encoded by an AAV particle comprising one or more capsid proteins described herein may be used to reduce the expression of target protein and/or mRNA in at least one region of the CNS or PNS. The expression of target protein and/or mRNA can, for example, be reduced by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS or PNS. As a non-limiting example, the expression of target protein and mRNA in the neurons (e.g., cortical neurons) is reduced by 50-90%. As a non-limiting example, the expression of target protein and mRNA in the neurons (e.g., cortical neurons) is reduced by 40-50%.

In some embodiments, the present disclosure provides methods for treating, or ameliorating neurological disorders associated with target gene and/or target protein in a subject in need of treatment, the method comprising administering to the subject a pharmaceutically effective amount of an AAV particle comprising one or more capsid proteins described herein that encodes at least one siRNA duplex targeting the gene of interest, delivering the particle to targeted cells, inhibiting target gene expression and protein production, and ameliorating symptoms of neurological disorder in the subject.

In some embodiments, an AAV particle comprising one or more capsid proteins described herein and comprising a nucleic acid sequence encoding at least one siRNA duplex targeting a gene of interest is administered to the subject in need for treating and/or ameliorating a neurological disorder. The AAV particle can comprise one or more capsid proteins in Table 1, 2 and/or 3, or variants thereof.

In some embodiments, AAV particles comprising one or more capsid proteins described herein and comprising a nucleic acid encoding such siRNA molecules may be introduced directly into the central nervous system of the subject, for example, by infusion into the putamen, by infusion to the thalamus, or by infusion to the white matter of a subject.

In some embodiments, AAV particles comprising one or more capsid proteins described herein and comprising a nucleic acid encoding such siRNA molecules may be introduced to the central nervous system of the subject, for example, by intravenous administration to a subject.

In some embodiments, a pharmaceutical composition described herein is used as a solo therapy. In other embodiments, a pharmaceutical composition described herein is used in combination therapy. The combination therapy may be in combination with one or more neuroprotective agents such as small molecule compounds, growth factors and hormones that have been tested for their neuroprotective effect on motor neuron degeneration.

In some embodiments, the present disclosure provides methods for treating, or ameliorating a neurological disorder, whether manifesting peripherally (PNS) or centrally (CNS) by administering to a subject in need thereof a therapeutically effective amount of an AAV particle comprising one or more capsid proteins described herein and one or more nucleic acid sequences encoding a selected payload (e.g., an siRNA molecule) described herein.

### Target Genes

Non-limiting examples of the neurological diseases that may be treated by administration of AAV particles comprising one or more capsid proteins described herein, wherein the AAV particles encode one or more modulatory polynucleotides described herein, include tauopathies, Alzheimer Disease, Huntington's Disease, and/or Amyotrophic Lateral Sclerosis. Target genes may be any of the genes associated with any neurological disease such as, but not limited to, those listed herein.

In some embodiments, the target gene is an allele of the APOE gene (e.g., ApoE2, ApoE3, and/or ApoE4), for example, an allele of human APOE.

In some embodiments, the target gene is an allele of the C9ORF72, for example, human C9ORF72.

In some embodiments, the target gene is an allele of the TARDBP, for example, human TARDBP.

In some embodiments, the target gene is an allele of the ATXN3, for example, human ATXN3.

In some embodiments, the target gene is an allele of the APP, for example, human APP.

In some embodiments, the target gene is an allele of the SNCA, for example, human SNCA.

In some embodiments, the target gene is an allele of the SCN9A for example, human SCN9A.

In some embodiments, the target gene is an allele of the SCN0A for example, human SCN10A.

In another embodiment, the target gene is SOD1, for example, human SOD1. In one non-limiting example, the SOD1 target gene has a sequence as found at NCBI reference number NM_00454.4.

In another embodiment, the target gene is HTT, for example, human HTT. As a non-limiting example, the HTT target gene has a sequence as found at NCBI reference number NM_002111.7. As another non-limiting example, the HIT target gene is HTT and the target gene encodes an amino acid sequence as found at NCBI reference number NP_002102.4.

In yet another embodiment, the target gene is MAPT. As a non-limiting example, the target gene is MAPT and the target gene has a sequence of any of the nucleic acid sequences or amino acid sequences found at NCBI reference numbers NP_058519.3, NP_005901.2, NP_058518.1, NP_058525.1, NP_001116539.1, NP_001116538.2, NP_001190180.1, NP_001190181.1, NM_016835.4, NM_005910.5, NM_016834.4, NM_016841.4, NM_001123067.3, NM_001123066.3, NM_001203251.1, or NM_001203252.1.

Some guidelines for designing siRNAs have been proposed in the art. These guidelines generally recommend generating a 19-nucleotide duplexed region, symmetric 2-3 nucleotide 3′ overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

In one aspect, siRNA molecules (e.g., siRNA duplexes or encoded dsRNA) that target a gene of interest are designed. Such siRNA molecules can specifically, suppress target gene expression and protein production. In some aspects, the siRNA molecules are designed and used to selectively “knock out” target gene variants in cells (e.g., transcripts that are identified in neurological disease). In some aspects, the siRNA molecules are designed and used to selectively “knock down” target gene variants in cells.

In some embodiments, an siRNA molecule described herein comprises a sense strand and a complementary antisense strand in which both strands are hybridized together to form a duplex structure. The antisense strand has sufficient complementarity to the target mRNA sequence to direct target-specific RNAi, e.g., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

In one aspect, the siRNA molecule has a length from about 10-50 or more nucleotides, e.g., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In some embodiments, the siRNA molecule has a length from about 19 to 25, 19 to 24 or 19 to 21 nucleotides.

In some embodiments, the siRNA molecules described herein may comprise an antisense sequence and a sense sequence, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

### Molecular Scaffold

In some embodiments, described herein are AAV particles comprising one or more capsid proteins described herein, wherein the AAV particles encode the siRNA molecules in a modulatory polynucleotide that also comprises a molecular scaffold. A molecular scaffold is a framework or starting molecule that forms the sequence or structural basis against which to design or make a subsequent molecule.

In some embodiments, the modulatory polynucleotide that comprises the payload (e.g., siRNA, miRNA or other RNAi agent described herein) comprises a molecular scaffold that comprises a leading 5′ flanking sequence that may be of any length and may be derived in whole or in part from wild type microRNA sequence or be completely artificial. A 3′ flanking sequence may mirror the 5′ flanking sequence in size and origin. Either flanking sequence may be absent. The 3′ flanking sequence may optionally contain one or more CNNC motifs, where “N” represents any nucleotide.

In some embodiments, one or both of the 5′ and 3′ flanking sequences are absent.

In some embodiments the 5′ and 3′ flanking sequences are the same length.

In some embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 5′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351.352, 353, 354, 355, 356, 357, 358.359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468.469, 470, 471, 472, 473, 474, 475, 476, 477.478, 479, 480, 481, 482, 483, 484.485, 486, 487, 488, 489, 490, 491, 492, 493, 494, 495, 496, 497, 498, 499, or 500 nucleotides in length.

In some embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 3′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314.315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387.388, 389, 390, 391, 392, 393, 394.395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments the 5′ and 3′ flanking sequences are the same sequence. In some embodiments they differ by 2%, 3%, 4%, 5%, 10%, 20% or more than 30% when aligned to each other.

Forming the stem of a stem loop structure is a minimum of at least one payload sequence. In some embodiments, the payload sequence comprises at least one nucleic acid sequence that is in part complementary or will hybridize to the target sequence. In some embodiments, the payload is an siRNA molecule or fragment of an siRNA molecule.

In some embodiments, the 5′ arm of the stem loop comprises a sense sequence.

In some embodiments, the 3′ arm of the stem loop comprises an antisense sequence. The antisense sequence, in some instances, comprises a “G” nucleotide at the 5′ most end.

In other embodiments, the sense sequence may reside on the 3′ arm while the antisense sequence resides on the 5′ arm of the stem of the stem loop structure.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

Neither the identity of the sense sequence nor the homology of the antisense sequence need be 100% complementary to the target.

Separating the sense and antisense sequence of the stem loop structure is a loop (also known as a loop motif). The loop may be of any length, between 4-30 nucleotides, between 4-20 nucleotides, between 4-15 nucleotides, between 5-15 nucleotides, between 6-12 nucleotides, 6 nucleotides, 7, nucleotides, 8 nucleotides, 9 nucleotides, 10 nucleotides, 11 nucleotides, and/or 12 nucleotides.

In some embodiments, the loop comprises at least one UGUG motif. In some embodiments, the UGUG motif is located at the 5′ terminus of the loop.

Spacer regions may be present in the modulatory polynucleotide to separate one or more modules from one another. There may be one or more such spacer regions present.

In some embodiments, a spacer region of between 8-20, e.g., 8, 9, 10, 11, 12, 13, 14, 15, 16, 17, 18, 19, or 20 nucleotides may be present between the sense sequence and a flanking sequence.

In some embodiments, the spacer is 13 nucleotides and is located between the 5′ terminus of the sense sequence and a flanking sequence. In some embodiments, a spacer is of sufficient length to form approximately one helical turn of the sequence.

In some embodiments, a spacer region of between 8-20, e.g., 8, 9, 10, 11, 12, 13, 14, 15, 16, 17, 18, 19, or 20 nucleotides may be present between the antisense sequence and a flanking sequence.

In some embodiments, the spacer sequence is between 10-13, e.g., 10, 11, 12 or 13 nucleotides and is located between the 3′ terminus of the antisense sequence and a flanking sequence. In some embodiments, a spacer is of sufficient length to form approximately one helical turn of the sequence.

In some embodiments, the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a sense sequence and the 3′ arm comprises the antisense sequence. In another non-limiting example, the 5′ arm comprises the antisense sequence and the 3′ arm comprises the sense sequence.

In some embodiments, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In some embodiments, the molecular scaffold of the modulatory polynucleotides is aligned in order to have the rate of excision of the guide strand be greater than the rate of excision of the passenger strand. The rate of excision of the guide or passenger strand may be, independently, 1%, 2%, 3%, 4%, 5%, 10%, 15%, 20%, 25%, 30%, 35%, 40%, 450, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In some embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In some embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In some embodiments, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In some embodiments, the molecular scaffold comprises a dual-function targeting modulatory polynucleotide. A dual-function targeting modulatory polynucleotide is a polynucleotide where both the guide and passenger strands knock down the same target or the guide and passenger strands knock down different targets.

In some embodiments, the molecular scaffold of the modulatory polynucleotides described herein comprise a 5′ flanking region, a loop region and a 3′ flanking region.

In some embodiments, the molecular scaffold may comprise one or more linkers known in the art. The linkers may separate regions or one molecular scaffold from another. As a non-limiting example, the molecular scaffold may be polycistronic.

In some embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In some embodiments, AAV particles comprising one or more capsid proteins described herein may be introduced into cells that are relevant to the disease to be treated. As a non-limiting example, the disease is a tauopathy and/or Alzheimer's Disease and the target cells are entorhinal cortex, hippocampal or cortical neurons.

In some embodiments, AAV particles comprising one or more capsid proteins described herein may be introduced into cells that have a high level of endogenous expression of the target sequence.

In some embodiments, AAV particles comprising one or more capsid proteins described herein may be introduced into cells that have a low level of endogenous expression of the target sequence.

In some embodiments, the cells may be those that have a high efficiency of AAV transduction.

In other embodiments, AAV particles comprising one or more capsid proteins described herein and comprising a nucleic acid sequence encoding the siRNA molecules described herein may be used to deliver siRNA molecules to the central nervous system.

In some embodiments, an AAV particle comprising one or more capsid proteins described herein that comprises a nucleic acid sequence encoding siRNA molecules described herein may encode siRNA molecules that are polycistronic molecules. The siRNA molecules may additionally comprise one or more linkers between regions of the siRNA molecules.

In some embodiments, an AAV particle comprising one or more capsid proteins described herein and comprising a nucleic acid sequence encoding a payload of interest (e.g., one expressing or targeting an antibody, AADC, APOE2, Frataxin, SMN, GCase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, ASPA, GRN, MeCP2, GLB1, and/or GAN) described herein may be formulated for CNS or PNS delivery.

In some embodiments, an AAV particle comprising one or more capsid proteins described herein and comprising a nucleic acid sequence encoding an siRNA molecule described herein may be administered directly to the CNS. As a non-limiting example, the AAV particle comprises a nucleic acid sequence encoding a siRNA molecules targeting ApoE, for example, ApoE2, ApoE3, or ApoE4. As a non-limiting example, the AAV particle comprises a nucleic acid sequence encoding the siRNA molecules targeting SOD1. As a non-limiting example, the AAV particle comprises a nucleic acid sequence encoding the siRNA molecules targeting HTT. As a non-limiting example, the AAV particle comprises a nucleic acid sequence encoding the siRNA molecules targeting Tau. As a non-limiting example, the AAV particle comprises a nucleic acid sequence encoding the siRNA molecules targeting C9ORF72. As a non-limiting example, the AAV particle comprises a nucleic acid sequence encoding the siRNA molecules targeting TARDBP. As a non-limiting example, the AAV particle comprises a nucleic acid sequence encoding the siRNA molecules targeting ATXN3. As a non-limiting example, the AAV particle comprises a nucleic acid sequence encoding the siRNA molecules targeting APP. As a non-limiting example, the AAV particle comprises a nucleic acid sequence encoding the siRNA molecules targeting SNCA. As a non-limiting example, the AAV particle comprises a nucleic acid sequence encoding the siRNA molecules targeting SCN9A. As a non-limiting example, the AAV particle comprises a nucleic acid sequence encoding the siRNA molecules targeting SCN10A.

### II. FORMULATION AND DELIVERY

### Pharmaceutical Compositions

In one aspect, AAV particles comprising one or more capsid proteins described herein may be prepared as pharmaceutical compositions. It will be understood that such compositions necessarily comprise one or more active ingredients and, most often, a pharmaceutically acceptable excipient.

In some embodiments, AAV particle pharmaceutical compositions described herein may comprise at least one payload. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions that are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

### Formulations

Formulations described herein can comprise, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. In general, such preparatory methods comprise the step of associating the active ingredient with an excipient and/or one or more other accessory ingredients.

and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. A unit dose refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

In some embodiments, the AAV particles described herein may be formulated in PBS with 0.001% of pluronic acid (F-68) at a pH of about 7.0.

In some embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

In one aspect, AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In one aspect, AAV particles may be formulated for PNS delivery.

### Excipients and Diluents

The AAV particles described herein can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload described herein.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia. and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins. Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, AAV particle formulations may comprise at least one inactive ingredient. An inactive ingredient refers to one or more agents that do not contribute to the activity of the active ingredient of the pharmaceutical composition comprised in formulations. In some embodiments, all, none or some of the inactive ingredients that may be used in the formulations described herein may be approved by the US Food and Drug Administration (FDA).

Pharmaceutical composition formulations of AAV particles disclosed herein may comprise cations or anions. In some embodiments, the formulations comprise metal cations such as, but not limited to, Zn2+, Ca2+, Cu2+, Mn2+, Mg+ and combinations thereof. As a non-limiting example, formulations may comprise polymers and complexes with a metal cation (See e.g., U.S. Pat. Nos. 6,265,389 and 6,555,525, each of which is herein incorporated by reference in its entirety).

Formulations described herein may also comprise one or more pharmaceutically acceptable salts. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

### III. ADMINISTRATION AND DOSING

### Administration

In some embodiments, an AAV particle comprising one or more capsid proteins described herein may be administered to a subject (e.g., to the CNS of a subject) in a therapeutically effective amount to reduce the symptoms of neurological disease of a subject (e.g., determined using a known evaluation method).

The AAV particles described herein may be administered by any delivery route that results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), sub-pial (between pia and CNS parenchyma), intracarotid arterial (into the intracarotid artery), epicutaneous (application onto the skin), intradermal, (into the skin itself), subcutaneous (under the skin), nasal administration (through the nose), intravenous (into a vein), intravenous bolus, intravenous drip, intra-arterial (into an artery), systemic, intramuscular (into a muscle), intracardiac (into the heart), intraosseous infusion (into the bone marrow), intrathecal (into the spinal canal), intraparenchymal (into the substance of a tissue. e.g., CNS tissue), intraperitoneal, (infusion or injection into the peritoneum), intravesical infusion, intravitreal, (through the eye), intracavemous injection (into a pathologic cavity) intracavitarv (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavemosum (within the dilatable spaces of the corporus cavemosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration that is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In some embodiments, compositions may be administered in a way that allows them to cross the blood-brain barrier, vascular barrier, or other epithelial barrier. The AAV particles described herein may be administered in any suitable form, as either a liquid solution or suspension, as a solid form suitable for liquid solution or suspension in a liquid solution. The AAV particles may be formulated with any appropriate and pharmaceutically acceptable excipient.

In some embodiments, the AAV particles described herein may be delivered to a subject via a single route administration.

In some embodiments, the AAV particles described herein may be delivered to a subject via a multi-site route of administration. AAV particles may be administered at 2, 3, 4, 5 or more than 5 sites.

In some embodiments, a subject may be administered the AAV particles described herein using a bolus infusion.

In some embodiments, a subject may be administered the AAV particles described herein using sustained delivery over a period of minutes, hours or days. The infusion rate may be changed depending on the subject, distribution, formulation or another delivery parameter.

In some embodiments, the AAV particles described herein may be delivered by intramuscular delivery route (see, e.g., U.S. Pat. No. 6,506,379; the content of which is incorporated herein by reference in its entirety). Non-limiting examples of intramuscular administration comprise an intravenous injection or a subcutaneous injection.

In some embodiments, the AAV particles described herein may be delivered by oral administration. Non-limiting examples of oral administration comprise a digestive tract administration and a buccal administration.

In some embodiments, the AAV particles described herein may be delivered by intraocular delivery route. A non-limiting example of intraocular administration comprises an intravitreal injection.

In some embodiments, the AAV particles described herein may be delivered by intranasal delivery route. Non-limiting examples of intranasal delivery comprise administration of nasal drops or nasal sprays.

In some embodiments, the AAV particles described herein may be administered to a subject by peripheral injections. Non-limiting examples of peripheral injections comprise intraperitoneal, intramuscular, intravenous, conjunctival or joint injection. It was disclosed in the art that the peripheral administration of AAV particles can be transported to the central nervous system, for example, to the motor neurons (see, e.g., U.S. Patent Publication Nos. 20100240739; and 20100130594; the content of each of which is incorporated herein by reference in their entirety).

In some embodiments, the AAV particles described herein may be delivered by injection into the CSF pathway. Non-limiting examples of delivery to the CSF pathway comprise intrathecal and intracerebroventricular administration.

In some embodiments, the AAV particles described herein may be delivered by systemic delivery. As a non-limiting example, the systemic delivery may be by intravascular administration.

In some embodiments, the AAV particles described herein may be administered to a subject by intracranial delivery (see, e.g., U.S. Pat. No. 8,119,611; the content of which is incorporated herein by reference in its entirety).

In some embodiments, the AAV particles described herein may be administered by injection. As a non-limiting example, the AAV particles described herein may be administered to a subject by injection.

In some embodiments, the AAV particles described herein may be administered by muscular injection. As a non-limiting example, the AAV particles described herein may be administered to a subject by muscular administration.

In some embodiments, the AAV particles described herein may be administered by intramuscular administration. As a non-limiting example, the AAV particles described herein may be administered to a subject by intramuscular administration.

In some embodiments, the AAV particles described herein are administered to a subject and transduce muscle of a subject. As a non-limiting example, the AAV particles are administered by intramuscular administration.

In some embodiments, the AAV particles described herein may be administered via intraparenchymal injection. As a non-limiting example, the AAV particles described herein may be administered to a subject by intraparenchymal administration.

In some embodiments, the AAV particles described herein may be administered by intravenous administration. As a non-limiting example, the AAV particles described herein may be administered to a subject by intravenous administration.

In some embodiments, the AAV particles described herein may be administered via intravenous delivery.

In some embodiments, the AAV particles described herein may be administered via a single dose intravenous delivery. As a non-limiting example, the single dose intravenous delivery may be a one-time treatment.

In some embodiments, the AAV particles described herein may be administered via intravenous delivery to the DRG nociceptive neurons.

In some embodiments, the AAV particles described herein may be administered via a single dose intravenous delivery to the DRG nociceptive neurons. As a non-limiting example, the single dose intravenous delivery may be a one-time treatment.

In some embodiments, the AAV particles described herein may be administered by intrathecal injection. As a non-limiting example, the AAV particles described herein may be administered by intrathecal injection.

In some embodiments, the AAV particles described herein may be administered to the cisterna magna in a therapeutically effective amount to transduce a central nervous system cell (e.g., a cell of the caudate, hippocampus, thalamus, putamen, brain stem, cortex (frontal, motor, occipital or cingulate cell), purkinje fiber, substanita nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia).

As a non-limiting example, the AAV particles described herein may be administered intrathecally.

In some embodiments, the AAV particles described herein may be administered using intrathecal infusion in a therapeutically effective amount to transduce a central nervous system cell (e.g., a cell of the caudate, hippocampus, thalamus, putamen, brain stem, cortex (frontal, motor, occipital or cingulate cell), purkinje fiber, substanita nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia).

In some embodiments, the AAV particles described herein may be administered via a single dose intrathecal injection. As a non-limiting example, the single dose intrathecal injection may be a one-time treatment. In some embodiments, the AAV particles described herein may be administered via intrathecal injection to the DRG nociceptive neurons.

In some embodiments, the AAV particles described herein may be administered via a single dose intrathecal injection to the DRG nociceptive neurons. As a non-limiting example, the single dose intrathecal injection may be a one-time treatment.

In some embodiments, the AAV particles described herein is administered via intrathecal (IT) infusion at Cl. The infusion may be for 1, 2, 3, 4, 6, 7, 8, 9, 10, 11, 12, 13, 14, 15 or more than 15 hours.

In some embodiments, the AAV particles described herein may be administered by intraparenchymal injection. As a non-limiting example, the AAV particles described herein may be administered to a subject by intraparenchymal injection.

In some embodiments, the AAV particles described herein may be administered by intraparenchymal injection and intrathecal injection. As a non-limiting example, the AAV particles described herein may be administered via intraparenchymal injection and intrathecal injection.

In some embodiments, the AAV particles described herein may be administered by subcutaneous injection. As a non-limiting example, the AAV particles described herein may be administered to a subject by subcutaneous injection.

In some embodiments, the AAV particles described herein may be administered topically. As a non-limiting example, the AAV particles described herein may be administered to a subject topically.

In some embodiments, the AAV particles may be delivered by direct injection into the brain. As a non-limiting example, the brain delivery may be by intrastriatal administration.

In some embodiments, the AAV particles described herein may be administered via intrastriatal injection.

In some embodiments, the AAV particles described herein may be administered via intrastriatal injection and another route of administration described herein.

In some embodiments, the AAV particles described herein may be delivered by more than one route of administration. As non-limiting examples of combination administrations, AAV particles described herein may be delivered by intrathecal and intracerebroventricular, or by intravenous and intraparenchymal administration.

In some embodiments, the AAV particles described herein may be administered to the CNS or PNS in a therapeutically effective amount to improve function and/or survival for a subject with a neurological disease. As a non-limiting example, the AAV particles described herein may be administered intravenously.

The AAV particles described herein may be administered in a therapeutically effective amount (e.g., an amount that is sufficient to alleviate and/or prevent at least one symptom associated with the disease, or provide improvement in the condition of the subject).

In some embodiments, the catheter may be located at more than one site in the spine for multi-site delivery. The AAV particles described herein may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the orientation of the spine of the subject during delivery of the AAV particles described herein may be vertical to the ground.

In another embodiment, the orientation of the spine of the subject during delivery of the AAV particles described herein may be horizontal to the ground.

In some embodiments, the spine of the subject may be at an angle as compared to the ground during the delivery of the AAV particles. The angle of the spine of the subject as compared to the ground may be at least 10, 20, 30, 40, 50, 60, 70, 80, 90, 100, 110, 120, 130, 140, 150 or 180 degrees.

In some embodiments, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. Yet another non-limiting example, prolonged infusions provide a more uniform transduction along the rostral-caudal length of the spinal cord.

### Parenteral and Injectable Administration

In some embodiments, pharmaceutical compositions, AAV particles described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can comprise adjuvants such as wetting agents, emulsifying and suspending agents, sweetening, flavoring, and/or perfuming agents. In some embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions that can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions that are compatible with body tissues.

### Rectal and Vaginal Administration

In some embodiments, pharmaceutical compositions, AAV particles described herein may be administered rectally and/or vaginally. Compositions for rectal or vaginal administration are typically suppositories that can be prepared by mixing compositions with suitable non-irritating excipients such as cocoa butter, polyethylene glycol or a suppository wax that are solid at ambient temperature but liquid at body temperature and therefore melt in the rectum or vaginal cavity and release the active ingredient.

### Oral Administration

In some embodiments, pharmaceutical compositions, AAV particles described herein may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, pharmaceutical compositions, AAV particles described herein may be formulated for administration topically. The skin may be an ideal target site for delivery as it is readily accessible. Three routes are commonly considered to deliver pharmaceutical compositions, AAV particles described herein to the skin: (i) topical application (e.g. for local/regional treatment and/or cosmetic applications), (ii) intradermal injection (e.g. for local/regional treatment and/or cosmetic applications); and (iii) systemic delivery (e.g. for treatment of dermatologic diseases that affect both cutaneous and extracutaneous regions). Pharmaceutical compositions, AAV particles described herein can be delivered to the skin by several different approaches known in the art.

In some embodiments, the disclosure provides for a variety of dressings (e.g., wound dressings) or bandages (e.g., adhesive bandages) for conveniently and/or effectively carrying out methods described herein. Typically dressing or bandages may comprise sufficient amounts of pharmaceutical compositions, AAV particles described herein to allow users to perform multiple treatments.

Dosage forms for topical and/or transdermal administration may comprise ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of pharmaceutical compositions, AAV particles described herein to the body. Such dosage forms may be prepared, for example, by dissolving and/or dispensing pharmaceutical compositions, AAV particles in the proper medium. Alternatively or additionally, rates may be controlled by either providing rate controlling membranes and/or by dispersing pharmaceutical compositions, AAV particles in a polymer matrix and/or gel.

Formulations suitable for topical administration comprise, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, pharmaceutical compositions, AAV particles described herein are formulated in depots for extended release. Generally, specific organs or tissues (“target tissues”) are targeted for administration.

In some aspects described herein, pharmaceutical compositions comprising AAV particles described herein are spatially retained within or proximal to target tissues. Provided are methods of providing pharmaceutical compositions comprising AAV particles to target tissues of mammalian subjects by contacting target tissues (which include one or more target cells) with pharmaceutical compositions comprising AAV particles under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of AAV particles that enter one or more target cells. For example, at least 1%, 5%, 10%, 20%, 30%, 40%, 50%, 60%, 70% 80%, 85%, 90%, 95%, 96%, 97% 98%, 99%, 99.9%, 99.99% or greater than 99.99% of the AAV particles administered to subjects are present intracellularly at a period of time following administration. For example, intramuscular injection to mammalian subjects may be performed using aqueous compositions comprising pharmaceutical compositions comprising AAV particles described herein and one or more transfection reagents, and retention is determined by measuring the amount of AAV particles present in target cells.

Certain aspects described herein are directed to methods of providing pharmaceutical compositions comprising AAV particles described herein to target tissues of mammalian subjects, by contacting target tissues (including one or more target cells) with pharmaceutical compositions comprising AAV particles under conditions such that they are substantially retained in such target tissues. Pharmaceutical compositions comprising AAV particles comprise enough active ingredient such that the effect of interest is produced in at least one target cell. In some embodiments, pharmaceutical compositions comprising AAV particles generally comprise one or more cell penetration agents, although “naked” formulations (such as without cell penetration agents or other agents) are also contemplated, with or without pharmaceutically acceptable carriers.

### Pulmonary Administration

In some embodiments, pharmaceutical compositions comprising AAV particles described herein may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may comprise a solid fine powder diluent such as sugar and are conveniently provided in a unit dose form.

Low boiling propellants generally comprise liquid propellants comprising a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles including active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, including active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methyl hydroxybenzoate. Droplets provided by this route of administration may have an average diameter in the range from about 0.1 nm to about 200 nm.

### Intranasal, Nasal and Buccal Administration

In some embodiments, pharmaceutical compositions comprising AAV particles described herein may be administered nasally and/or intranasal. In some embodiments, formulations described herein useful for pulmonary delivery may also be useful for intranasal delivery. In some embodiments, formulations for intranasal administration comprise a coarse powder including the active ingredient and comprising an average particle size from about 0.2 μm to 500 μm. Such formulations are administered in the manner in which snuff is taken, e.g. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, include 0.1% to 20% (w/w) active ingredient, the balance including an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may include average particle and/or droplet sizes in the range of from about 0.1 nm to about 200 nm, and may further include one or more of any additional ingredients described herein.

### Ophthalmic or Otic Administration

In some embodiments, pharmaceutical compositions comprising AAV particles described herein may be prepared, packaged, and/or sold in formulations suitable for ophthalmic and/or otic administration. Such formulations may, for example, be in the form of eye and/or ear drops comprising, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations that are useful include those that include active ingredients in microcrystalline form and/or in liposomal preparations. Subretinal inserts may also be used as forms of administration.

### Delivery, Dose and Regimen

In one aspect, the present disclosure provides methods of administering AAV particles described herein to a subject in need thereof. The pharmaceutical, diagnostic, or prophylactic AAV particles and compositions described herein may be administered to a subject using any amount and any route of administration effective for preventing, treating, managing, or diagnosing diseases, disorders and/or conditions.

In some embodiments, the AAV particles may be delivered in a multi-dose regimen. The multi-dose regimen may be 2, 3, 4, 5, 6, 7, 8, 9, 10 or more than 10 doses.

In some embodiments, the AAV particles may be delivered to a subject via a multi-site route of administration. A subject may be administered the AAV particles at 2, 3, 4, 5 or more than 5 sites.

The desired dosage of the AAV particles described herein may be delivered only once, three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. A split dose is the division of single unit dose or total daily dose into two or more doses, e.g., two or more administrations of the single unit dose. A single unit dose is a dose of any therapeutic administered in one dose/at one time/single route/single point of contact, e.g., single administration event.

The desired dosage of the AAV particles described herein may be administered as a pulse dose or as a continuous flow. A pulse dose is a series of single unit doses of any therapeutic administered with a set frequency over a period of time. A continuous flow is a dose of therapeutic administered continuously for a period of time in a single route/single point of contact, e.g., continuous administration event. A total daily dose, an amount given or prescribed in 24 hour period, may be administered by any of these methods, or as a combination of these methods, or by any other methods suitable for a pharmaceutical administration.

In some embodiments, delivery of the AAV particles described herein to a subject provides regulating activity of a target gene in a subject. The regulating activity may be an increase in the production of the target protein in a subject or the decrease of the production of target protein in a subject. The regulating activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, the AAV particles described herein may be administered to a subject using a single dose, one-time treatment. The dose of the one-time treatment may be administered by any methods known in the art and/or described herein. A one-time treatment refers to a composition that is only administered one time. If needed, a booster dose may be administered to the subject to ensure the appropriate efficacy is reached. A booster may be administered 1 month, 2 months, 3 months, 4 months, 5 months, 6 months, 7 months, 8 months, 9 months, 10 months, 11 months, 12 months, 1 year, 13 months, 14 months, 15 months, 16 months, 17 months, 18 months, 19 months, 20 months, 21 months, 22 months, 23 months, 24 months, 2 years, 3 years, 4 years, 5 years, 6 years, 7 years, 8 years, 9 years, 10 years, or more than 10 years after the one-time treatment.

### Delivery Methods

In some embodiments, the AAV particles or pharmaceutical compositions described herein may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions described herein may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions described herein may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particle or pharmaceutical compositions described herein may be administered or delivered using the methods for delivering proteins using AAV particles described in European Patent Application No. EP2678433, the contents of which are herein incorporated by reference in their entirety.

In some embodiments, the AAV particle or pharmaceutical compositions described herein may be administered or delivered using the methods for delivering DNA to the bloodstream described in U.S. Pat. No. 6,211,163, the contents of which are herein incorporated by reference in their entirety.

In some embodiments, the AAV particle or pharmaceutical compositions described herein may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions described herein may be administered or delivered using the methods for delivering a payload described in U.S. Pat. No. 8,283,151, the contents of which are herein incorporated by reference in their entirety.

In some embodiments, the AAV particle or pharmaceutical compositions described herein may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions described herein may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV particles, comprising contacting the cell or tissue with the AAV particles or contacting the cell or tissue with a formulation comprising the AAV particles, or contacting the cell or tissue with any of the described compositions, comprising pharmaceutical compositions. The method of delivering the AAV particles to a cell or tissue can be accomplished in vitro, ex vivo, or in vivo.

### Delivery to Subjects

The present disclosure additionally provides a method of delivering to a subject, comprising a mammalian subject, any of the above-described AAV particles comprising administering to the subject the AAV particle, or administering to the subject a formulation comprising the AAV particle, or administering to the subject any of the described compositions, comprising pharmaceutical compositions.

### Measurement of Expression

Expression of payloads from viral genomes may be determined using various methods known in the art such as, but not limited to immunochemistry (e.g., IHC), n situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoclectrophoresis, Western blot, SDS-PAGE, protein immunoprecipitation, and/or PCR.

### IV. METHODS AND USES OF THE COMPOSITIONS OF THE DISCLOSURE

### Analysis of Chimeric Capsid Library

### Directed Evolution

In some embodiments, AAV capsid libraries are prepared via capsid shuffling. These libraries may be generated with high complexity and diversity. The AAV capsid libraries may be prepared from any parental AAV serotype. In some embodiments, the AAV capsid libraries are produced from 2, 3, 4, 5, 6, 7, 8, 9, 10, 11, 12, 13, 14, 15, 16, 17, 18, 19, or 20 or more parental serotypes. In some embodiments, AAV capsid libraries may be produced from nine parental AAV serotypes. These parental AAV serotypes may include, but are not limited to, AAV2, AAV6, AAV7, AAV8, AAV9, AAVrh8, AAVrh10, AAVrh39 and AAVrh43.

In some embodiments, the parental AAV serotypes may be used to amplify the full length of cap genes by any method known to one skilled in the art, including PCR. Equal amounts of cap PCR products may be pooled and fragmented by DNase I incubation to obtain a pool of fragments between 0.2 and 1.0 kb in size. Fragments may be between from about 0.2 kb to about 0.4 kb, from about 0.4 kb to about 0.6 kb, from about 0.6 kb to about 0.8 kb, or from about 0.8 kb to about 1.0 kb. Differently sized fragments may be reassembled into full-length cap variants by primerless PCR. PCR may then be carried out a second time to amplify the shuffled cap library using any primer known to one of skill in the art. In some embodiments, the primers contain HindIII (forward primer: 5′-CAGTGACGCAGATATAAGTGAGCCC-3′: SEQ ID NO: 145) or ClaI site (reverse primer: 5′-GAAACGAATTAAACGGTTATfGATTAACAATCGATTA-3′; SEQ ID NO: 146). The band may then be purified and subcloned into linearized wild-type ITR-rep vector (digested by HindIII and ClaI). In some embodiments, the band may be subloned via the Gibson Assembly method (FIG. 1) (Gibson et al., Nat. Methods., 7(11):901-903 (2010)). The shuffled capsid library may then be transformed into cells. The method of transformation may comprise electroporation. Those cells may be bacterial cells (e.g. Escherichia coli). The integrity and genetic diversity of a shuffled capsid library may be assessed by total colony number and/or 100% homology of cap variants in hundreds of colonies. The homology of cap variants may be determined by chain-termination sequencing. The maximal diversity of an original chimeric capsid library may be between from about 0.1×10⁷ to about 0.5×10⁷, from about 0.5×10⁷ to about 1.0×10⁷, from about 10×10⁷ to about 1.5×10⁷, from about 1.5×10⁷ to about 2.0×10⁷, from about 2.0×10⁷ to about 2.5×10⁷, from about 2.5×10⁷ to about 3.0×10⁷, from about 3.0×10⁷ to about 3.5×10⁷, from about 3.5×10⁷ to about 4.0×10⁷, from about 4.0×10⁷ to about 4.5×10⁷, or from about 4.5×10⁷ to about 5×10⁷. In some embodiments, the maximal diversity of the original chimeric capsid library may be about 3.4×10⁷.

Library plasmids and adenoviral helper plasmids may be transduced into cells. Those cells may be HEK-293T cells. The resulting hybrid viruses in cells and culture medium may then be collected and purified by any method, including an iodixanol gradient. The complexity and diversity of a shuffled capsid library recovered from hybrid viruses may then be assessed by any method, including, but not limited to, qPCR and chain-termination sequencing (e.g. Sanger sequencing).

To begin the first round of directed evolution, a subject may be injected with the hybrid viruses, which may be prepared in any buffer (e.g. PBS). Subjects may be a mammal, including but not limited to mice, rats, rabbits, non-human primates, and humans. The subject may be a non-human primate (e.g. monkey). The subject may be injected via any method described in the present disclosure. In some embodiments, the subject may be injected intrathecally, intravenously, intrastriatally, or via cisterna magna. Subjects may be injected with doses from about 1.0×10¹⁰ vg to about 1.0×10¹⁵ vg of hybrid viruses. Subjects may be injected with doses from about 1.0×10¹⁰ vg to about 5.0×10¹⁰ vg, from about 5.0×10¹⁰ vg to about 1.0×10¹¹ vg, from about 1.0×10¹¹ vg to about 5.0×10¹¹ vg, from about 5.0×10¹¹ vg to about 1.0×10¹² vg, from about 1.0×10¹² vg to about 5.0×10¹² vg, from about 5.0×10¹² vg to about 1.0×10¹³ vg, from about 1.0×10¹³ vg to about 5.0×10¹³ vg, from about 5.0×10¹³ vg to about 1.0×10¹⁴ vg, from about 1.0×10¹⁴ vg to about 5.0×10¹⁴ vg, or from about 5.0×10¹⁴ vg to about 1.0×10¹⁵ vg of hybrid viruses. In some embodiments, the subjects may be injected with 3.0×10¹³ vg of hybrid viruses. In some embodiments, the subjects may be injected with 1.5×10¹¹ vg of hybrid viruses.

In some embodiments, the injection volume may be from about 0.1 μL to about 10 mL. The injection volume may be from about 0.1 μL to about 5.0 μL, from about 5.0 μL to about 10.0 μL, from about 10.0 μL to about 50.0 μL, from about 50.0 μL to about 100.0 μL, from about 100.0 μL to about 500.0 μL, from about 500.0 μL to about 1 mL, from about 1 mL to about 2 mL, from about 2 mL to about 3 mL, from about 3 mL to about 4 mL, from about 4 mL to about 5 mL, from about 5 mL to about 6 mL, from about 6 mL to about 7 mL, from about 7 mL to about 8 mL, from about 8 mL to about 9 mL, or from about 9 mL to about 10 mL.

After administration of the hybrid viruses, the subject may be sacrificed, and DNA may be extracted, via any method or kit, from sample CNS tissues. The sacrifice and/or DNA isolation may be performed up to 1 week, 2 weeks, 3 weeks, 4 weeks, 5 weeks, 6 weeks, 7 weeks, 8 weeks, 9 weeks, 6 months, or 1 year after administration. Sample CNS tissues may include, but are not limited to, the dentate nucleus, the hippocampus, the thalamus, the putamen, the brain stem, the cortex (frontal, motor, occipital, and cingulate), the purkinje fibers, the substantia nigra, the striatum, the spinal cord (cervical, thoracic, and lumbar), the caudate, the dorsal root ganglion (DRG), the cerebellum, and the peripheral organs (including liver, heart, lung, muscle). The chimeric capsid library may be recovered from the tissues via PCR, and purified via any method known to one skilled in the art.

For the next round of directed evolution, the recovered capsid library may be subcloned into linearized wild-type ITR-rep plasmid and transformed into cells. Those cells may be bacterial cells (e.g. Escherichia coli). The production, purification and quality control of hybrid viruses may be performed as for the first-round preparation. Directed evolution may be performed for at least two rounds, at least three rounds, at least four rounds, at least five rounds, or at least six rounds. In some embodiments, the dose of hybrid viruses may decrease with each round of directed evolution.

Directed evolution may also be performed in cells. Those cells may include, but are not limited to, astrocytes, glia, microglia, neurons, and oligodendrocytes. In some embodiments, the cells may be infected with MOI at between from about 100 to about 10,000 hybrid viruses. The cells may be infected at about 1000 hybrid viruses. The hybrid viruses may be preincubated with human intravenous immunoglobulin prior to infection. The cells may be infected for up to 10 hours, up to 24 hours, up to 36 hours, up to 72 hours, or up to one week. The chimeric capsid library may then be recovered from genomic DNA of cells for next round of directed evolution. The production, purification and quality control of hybrid viruses may be performed with the methods described for animal studies. Directed evolution in cells may be performed for at least two rounds, at least three rounds, at least four rounds, at least five rounds, or at least six rounds.

### Lead Capsid Selection

In some embodiments, selection of lead capsid variants may be performed after at least two rounds, at least three rounds, at least four rounds, at least five rounds, or at least six rounds of directed evolution. After the desired round of directed evolution, CNS tissues and/or cells may be harvested, and the chimeric capsid library may be recovered (e.g. by PCR), and then subcloned and transformed. A chimeric capsid library may be transformed into bacterial cells (e.g. Escherichia coli) with standard transformation techniques. Random colonies may then be sequenced via next-generation sequencing, chain termination sequencing (Sanger sequencing), bridge PCR, shotgun sequencing, pyrosequencing, nanopore sequencing, sequencing by ligation, sequencing by combinatorial probe anchor synthesis, sequencing by synthesis, and any other sequencing method known to one of skill in the art. In some embodiments, at least 10, at least 20, at least 50, at least 100, at least 200, at least 300, at least 400, at least 500, at least 600, at least 700, at least 800, at least 900, or at least 1000 random colonies may be sequenced.

The identified capsid variants may be any of those described in Table 1, Table 2, and/or Table 3. The sequenced capsid variants may then be aligned and compared to the parental capsids. Lead capsid variants may be selected based on their abundance rate. In some embodiments, the abundance rate was at least 1%, at least 5%, at least, 10%, at least 20%, or at least 30%.

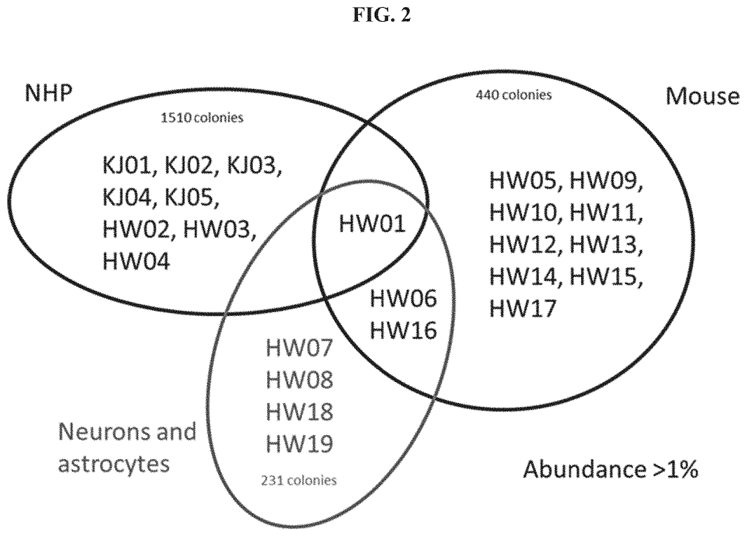

In some embodiments, leads identified from selections in non-human primates may include, but are not limited to, KJ01, KJ02, KJ03, KJ04, KJ05, HW01, HW02, HW03 and HW04, as seen in FIG. 2.

In some embodiments, leads identified from selections in mice may include, but are not limited to, HW01, HW05, HW06, HW09, HW10, HW11, HW12, HW13, HW14, HW15, HW16, and HW17, as seen in FIG. 2.

In some embodiments, leads identified from selections in neurons and astrocytes may include but are not limited to, HW01, HW06, HW07, HW08, HW16, HW18, and HW19, as seen in FIG. 2.

In some embodiments, leads identified from selections in neurons, astrocytes, and mice may include but are not limited to, HW01, HW06 and HW16, as seen in FIG. 2.

In some embodiments, HW01 may be identified as a lead in any tested subject or cell line.

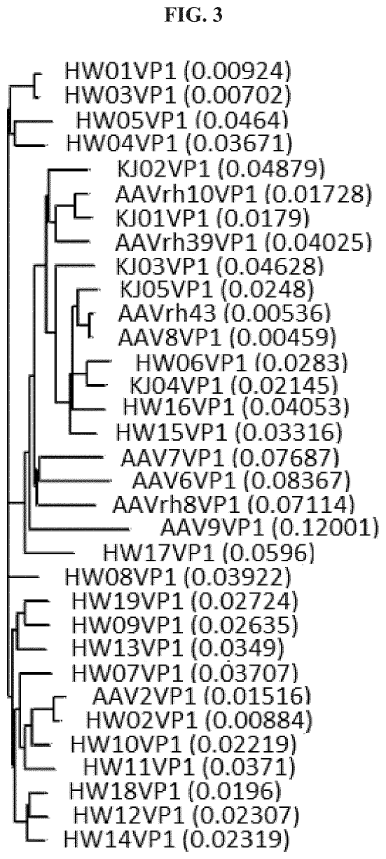

In some embodiments, the Neighbor Joining method (NJ) of Saitou et al. Molecular Biology and Evolution, 4(4): 406-425 (1987), the contents of which are herein incorporated by reference in their entirety, may be used to analyze relationships between hybrid viruses and the parent AAV serotypes. The NJ method works on a matrix of distances between all pairs of sequences to be analyzed. These distances are related to the degree of divergence between the sequences. The calculated distance values are calculated after the sequences are aligned, and they represent a measure of complexity. The smaller a value is, the less divergence between the sequences, e.g. the value “0” represents completely identical. In some embodiments, the calculated distance values are from about 0.001 to about 0.500. In some embodiments, the calculated distance values are from about 0.001 to about 0.005, from about 0.005 to about 0.010, from about 0.010 to about 0.015, from about 0.015 to about 0.020, from about 0.020 to about 0.025, from about 0.025 to about 0.030, from about 0.030 to about 0.035, from about 0.035 to about 0.040, from about 0.040 to about 0.045, from about 0.045 to about 0.050, from about 0.050 to about 0.055, from about 0.055 to about 0.060, from about 0.060 to about 0.065, from about 0.065 to about 0.070, from about 0.070 to about 0.075, from about 0.075 to about 0.080, from about 0.080 to about 0.085, from about 0.085 to about 0.090, from about 0.090 to about 0.095, from about 0.095 to about 0.100, from about 0.100 to about 0.150, from about 0.150 to about 0.200, or from about 0.200 to about 0.500. In some embodiments, the calculated distance values may be used to prepare a guide tree (FIG. 3) and they are as listed in the parenthesis following the molecule names in FIG. 3.

Transduction with Lead Capsids

In some embodiments, the transduction efficiency of one or more lead capsids may be identified via directed evolution. Transgenes may be prepared for the delivery of any of the payloads described in the present disclosure. In some embodiments, human frataxin-HA (hFXN-HA) may be used as transgene. In some embodiments, a transgene vector (2,828 bp from 5′-ITR to 3′-ITR; SEQ ID NO: 147; 5′CCTGCAGGCAGCTGCGCGCTCGCTCGCTCACTGAGGCCGCCCGGGCAAAGCCC GGGCGTCGGGCGACCTTTGGTCGCCCGGCCTCAGTGAGCGAGCGAGCGCGCAGA GAGGGAGTGGCCAACTCCATCACTAGGGGTTCCTTGTAGTTAATGATTAACCCGC CATGCTACTTATCTACGTAGCCATGCGTCGACATAACGCGTCGACATTGATTATT GACTAGTTATTAATAGTAATCAATTACGGGGTCATTAGTTCATAGCCCATATATG GAGTTCCGCGTTACATAACTTACGGTAAATGGCCCGCCTGGCTGACCGCCCAACG ACCCCCGCCCATTGACGTCAATAATGACGTATGTTCCCATAGTAACGCCAATAGG GACTTTCCATTGACGTCAATGGGTGGAGTATTACGGTAAACTGCCCACTTGGCA GTACATCAAGTGTATCATATGCCAAGTACGCCCCCTATTGACGTCAATGACGGTA AATGGCCCGCCTGGCATTATGCCCAGTACATGACCTTATGGGACTTTCCTACTTG GCAGTACATCTACGTATTAGTCATCGCTATTACCATGTCGAGGCCACGTTCTGCT TCACTCTCCCCATCTCCCCCCCCTCCCCACCCCCAATTTTGTATTTATTTATTTTTT AATTATTTTGTGCAGCGATGGGGGCGGGGGGGGGCGCGCGCCAGGCGGGGC GGGGCGGGGCGAGGGGCGGGGCGGGGCGAGGCGGAGAGGTGCGGCGGCAGCCA ATCAGAGCGGCGCGCTCCGAAAGTTTCCTTTTATGGCGAGGCGGCGGCGGCGGC GGCCCTATAAAAAGCGAAGCGCGCGGCGGGCGGGAGCAAGCTTCGTTTAGTGAA CCGTCAGATCGCCTGGAGACGCCATCCACGCTGTTTTGACCTCCATAGAAGACAC CGGGACCGATCCAGCCTCCGCGGATTCGAATCCCGGCCGGGAACGGTGCATTGG AACGCGGATTCCCCGTGCCAAGAGTGACGTAAGTACCGCCTATAGAGTCTATAG GCCCACAAAAAATGCTTTCTTCTTTTAATATACTTTTTTGTTTATCTTATTTCTAAT ACTTTCCCTAATCTCTTTCTTTCAGGGCAATAATGATACAATGTATCATGCCTCTT TGCACCATTCTAAAGAATAACAGTGATAATTTCTGGGTTAAGGCAATAGCAATAT TTCTGCATATAAATATTTCTGCATATAAATTGTAACTGATGTAAGAGGTTTCATAT TGCTAATAGCAGCTACAATCCAGCTACCATTCTGCTTTTATTTTATGGTTGGGATA AGGCTGGATTATTCTGAGTCCAAGCTAGGCCCTTTTGCTAATCATGTTCATACCTC TTATCTTCCTCCCACAGCTCCTGGGCAACGTGCTGGTCTGTGTGCTGGCCCATCAC TTTGGCAAAGAATTGGGATTCGAACCGGTATGTGGACTTTCGGGCGCCGCGCAGT TGCCGGCCTCCTGGCGTCCCCGAGCCCGGCCCAGGCCCAGACCCTCACCCGGGCC CCGCGGCTGGCAGAGTTGGCCCAGCTCTGCAGCCGCCGGGGCCTGCGCACCGGC ATCAATGCGACCTGCACAACCCACCACACCAGTTCGAACCTCCGTGGCCTCAACC AGATTCGGAATGTCAAAAGGCAGAGTGTCTACTTGATGAATTTGAGGAAATCGG GAACTTTGGGCCACCCAGGCTCTCTAGATGACACCACCTATGAAAGACTAGCAG AGGAAACGCTGGACTCTAGCAGAGTTTTTTGAAGACCTTGCAGACAAGCCATA CACCTTTGAGGACTATGATGTTTCCTTTGGGAGTGGTGTCTTAACTGTTAAACTGG GTGGAGATCTAGGAACCTACGTGATCAACAAGCAGACGCCAAACAAGCAAATCT GGTTATCTTCTCCATCCAGTGGACCCAAGCGTTATGACTGGACTGGGAAAAACTG GGTGTATTCCCACGACGGCGTTTCCCTCCATGAGCTGCTGGGCGCAGAGCTCACT AAAGCCTTAAAAACCAAACTGGACTTGTCTTCCTTGGCCTATTCCGGAAAAGACG CTTATCCTTATGACGTGCCTGACTATGCCTGATGACTCGAGGACGGGGTGAACTA CGCCTGAGGATGGGTGGCATCCCTGTGACCCCTCCCCAGTGCCTCTCCTGGCCCT GGAAGTTGCCACTCCAGTGCCCACCAGCCTTGTCCTAATAAAATTAAGTTGCATC ATTTTGTCTGACTAGGTGTCCTTCTATAATATTATGGGGTGGAGGGGGGTGGTAT GGAGCAAGGGGCAAGTTGGGAAGACAACCTGTAGGGCCTGCGGGGTCTATTGGG AACCAAGCTGGAGTGCAGTGGCACAATCTTGGCTCACTGCAATCTCCGCCTCCTG GGTTCAAGCGATTCTCCTGCCTCAGCCTCCCGAGTTGTTGGATTCCAGGCATGC ATGACCAGGCTCAGCTAATTTTTGTTTTTTTGGTAGAGACGGGGTTTCACCATATT GGCCAGGCTGGTCTCCAACTCCTAATCTCAGGTGATCTACCCACCTTGGCCTCCC AAATTGCTGGGATTACAGGCGTGAACCACTGCTCCCTTCCCTGTCCTTGGCCTAG GTATCGATGCTACGTAGATAAGTAGCATGGCGGGTTAATCATTAACTACAGAGG AACCCCTAGTGATGGAGTTGGCCACTCCCTCTCTGCGCGCTCGCTCGCTCACTGA GGCCGGGCGACCAAAGGTCGCCCGACGCCCGGGCTTGCCCGGGCGGCCTCAGT GAGCGAGCGAGCGCGCAGCTGCCTGCAGG3′) may be used to prepare vectors with hFXN-HA as a representative transgene. The resulting transgene vector may be rAAV.CBA.hBglobin.hFXN-HA.hGHpA (includes sequences of or encoding CBA promoter, hBglobin, human frataxin with HA flag, human growth hormone (hGH) polyA). Transgene vectors may be prepared in any cell line described herein (e.g. HEK-293T cells), and purified by any method known to one of skill in the art (e.g. iodixanol gradient). Any of the lead capsids from Table 1, Table 2, and Table 3 may be used to prepare the vectors. In some embodiments, the lead capsids may include, but are not limited to HW01, HW2, HW03 and HW04.

Vectors may be formulated in any one or more buffers, including, but not limited to citric acid buffer, Gly-Gly, HEPES, imidazole, MES, MOPS, phosphate buffered saline (PBS), phosphate buffer, Pluronic F-68, TEA, tricine, Trizma buffer, sodium acetate, and sodium carbonate. Buffers may be present in the formulation at a concentration (by weight or by volume) of from about 0.0001% to about 99.9%. Buffers may be present in the formulation at a concentration (by weight or by volume) of from about 0.0001% to about 0.001%, from about 0.001% to about 0.01%, from about 0.01% to about 0.1%, from about 0.1% to about 1%, from about 1% to about 10%, from about 10% to about 20%, from about 20% to about 50%, from about 50% to about 75%, or from about 75% to about 99.9%.

Quality of the formulations may be assessed by rAAV titer (ddPCR), vector purity (by silver staining), genome integrity (by denaturing gel staining), and endotoxin level. rAAV titer (via ddPCR) may be from about 1.0×10¹⁰ vg/mL to about 1.0×10¹⁵ vg/mL. rAAV titer (via ddPCR) may be from about 1.0×10¹⁰ vg/mL to about 5.0×10¹⁰ vg/mL, from about 5.0×10¹⁰ vg/mL to about 1.0×10¹¹ vg/mL, from about 1.0×10¹¹ vg/mL to about 5.0×10¹¹ vg/mL, from about 5.0×10¹¹ vg/mL to about 1.0×10¹² vg/mL, from about 1.0×10¹² vg/mL to about 5.0×10¹² vg/mL, from about 5.0×10¹² vg/mL to about 1.0×10¹³ vg/mL, from about 1.0×10¹³ vg/mL to about 5.0×10¹³ vg/mL, from about 5.0×10¹³ vg/mL to about 1.0×10¹⁴ vg/mL, from about 1.0×10¹⁴ vg/mL to about 5.0×10¹⁴ vg/mL, or from about 5.0×10¹⁴ vg/mL to about 1.0×10¹⁵ vg/mL. Endotoxin levels may be from less than about 0.5 EU/mL to about 4.0 EU/mL. Endotoxin levels may be from less than about 0.5 EU/mL to about 1.0 EU/mL, from about 1.0 EU/mL to about 1.5 EU/mL, from about 1.5 EU/mL to about 2.0 EU/mL, from about 2.0 EU/mL to about 2.5 EU/mL, from about 2.5 EU/mL to about 3.0 EU/mL, from about 3.0 EU/mL to about 3.5 EU/mL, or from about 3.5 EU/mL to about 4.0 EU/mL.

rAAV vectors may then be administered to a subject. That subject may be any subject described herein (e.g. a mouse). The rAAV vectors may be administered via any method described herein (e.g. injected intrathecally and/or intravenously). Vectors may be administered at a concentration of from about 1.0×10¹⁰ vg/mL to about 1.0×10¹⁵ vg/mL in the formulation buffer. Vectors may be administered at a concentration of from about 1.0×10¹⁰ vg/mL to about 5.0×10¹⁰ vg/mL, from about 5.0×10¹⁰ vg/mL to about 1.0×10¹¹ vg/mL, from about 1.0×10¹¹ vg/mL to about 5.0×10¹¹ vg/mL, from about 5.0×10¹¹ vg/mL to about 1.0×10¹² vg/mL, from about 1.0×10¹² vg/mL to about 5.0×10¹² vg/mL, from about 5.0×10¹² vg/mL to about 1.0×10¹³ vg/mL, from about 1.0×10¹³ vg/mL to about 5.0×10¹³ vg/mL, from about 5.0×10¹³ vg/mL to about 1.0×10¹⁴ vg/mL, from about 1.0×10¹⁴ vg/mL to about 5.0×10¹⁴ vg/mL, or from about 5.0×10¹⁴ vg/mL to about 1.0×10¹⁵ vg/mL in the formulation buffer. The injection volume may be from about 0.1 μL to about 10.0 mL. The injection volume may be from about 0.1 μL to about 5.0 μL, from about 5.0 μL to about 10.0 μL, from about 10.0 μL to about 50.0 μL, from about 50.0 μL to about 100.0 μL, from about 100.0 μL to about 500.0 μL, from about 500.0 μL to about 1 mL, from about 1 mL to about 2 mL, from about 2 mL to about 3 mL, from about 3 mL to about 4 mL, from about 4 mL to about 5 mL, from about 5 mL to about 6 mL, from about 6 mL to about 7 mL, from about 7 mL to about 8 mL, from about 8 mL to about 9 mL, or from about 9 mL to about 10 mL.

After administration of the rAAV vectors, the subject may be sacrificed and DNA may be extracted, via any method or kit, from sample CNS tissues. Sacrifice and DNA isolation may be performed at up to 1 week, 2 week, 3 weeks, 4 weeks, 5 weeks, 6 weeks, 7 weeks, 8 weeks, 9 weeks, 6 months, or 1 year after administration. Sample CNS tissues may include, but are not limited to, cortex, striatum, brain stem, cerebellum, spinal cord, and peripheral tissues (liver, heart and lung).

The expression of mRNA and protein in the tissues may be evaluated by real-time quantitative reverse transcription-polymerase chain reaction (qRT-PCR) and enzyme-linked immunosorbent assay (ELISA), or any other methods known to one of skill in the art. The fold change in mRNA expression and/or protein expression in the described tissues or cells, as compared to AAV9, may be determined via the results of PCR and ELISA respectively. The fold change in mRNA expression and/or protein expression may be from about 0.01 to about 15.0. The fold change in mRNA expression and/or protein expression may be from about 0.01 to about 0.05, about 0.05 to about 0.10, about 0.10 to about 0.20, about 0.20 to about 0.30, about 0.30 to about 0.40, about 0.40 to about 0.50, about 0.50 to about 0.60, about 0.60 to about 0.70, about 0.70 to about 0.80, about 0.80 to about 0.90, about 0.90 to about 1.0, about 1.0 to about 2.0, about 2.0 to about 3.0, about 3.0 to about 4.0, about 4.0 to about 5.0, about 5.0 to about 6.0, about 6.0 to about 7.0, about 7.0 to about 8.0, about 8.0 to about 9.0, about 9.0 to about 10.0, about 10.0 to about 11.0, about 11.0 to about 12.0, about 12.0 to about 13.0, about 13.0 to about 14.0, about 14.0 to about 15.0, or greater than 15.0.

In some embodiments, the protein levels in the described tissues or cells may be from about 0.01 pg/μg Total protein to about 25000 pg/μg Total protein. In some embodiments, the protein levels in the described tissues or cells may be from about 0.01 pg/μg Total protein to about 5 pg/μg Total protein, from about 5 pg/μg Total protein to about 20 pg/μg Total protein, from about 20 pg/μg Total protein to about 50 pg/μg Total protein, from about 50 pg/μg Total protein to about 200 pg/μg Total protein, from about 200 pg/μg Total protein to about 1000 pg/μg Total protein, from about 1000 pg/μg Total protein to about 5000 pg/μg Total protein, or from about 5000 pg/μg Total protein to about 25000 pg/μg Total protein.

In one embodiment, HW01 may provide higher mRNA expression in the cortex and spinal cord as compared to AAV9.

In one embodiment, HW03 may provide higher mRNA expression in the cortex, brain stem, cerebellum, and spinal cord as compared to AAV9.

In one embodiment, HW04 may provide higher mRNA expression in the cortex, striatum, brain stem, cerebellum, and spinal cord as compared to AAV9.

In one embodiment, HW01, HW03 and HW04 may provide mRNA expression levels that were lower in the liver and in the heart after IT injection as compared to AAV9.

Total protein from different tissues may be determined by any method, including a BCA protein assay. With IT injection HW01 may display enhanced protein expression in the cortex and spinal cord as compared to AAV9. IT injection of HW03 may provide higher protein expression in the cortex, brain stem, cerebellum and spinal cord as compared with AAV9. IT injection of HW04 may provide higher protein expression in the cortex, striatum, brain stem, cerebellum, and spinal cord as compared with AAV9. In some embodiments, HW01, HW03 and HW04 may provide lower protein expression in the liver after IT injection as compared to AAV9. With IV injection, HW03 and HW04 may provide increased protein expression in the brain stem, while HW01, HW02, HW03 and HW04 may provide less protein expression in the cortex, striatum, cerebellum, spinal cord and liver, compared to AAV9.

### Neurological Disease

Various neurological diseases may be treated with pharmaceutical compositions and AAV particles described herein. For example, the present disclosure provides a method for treating neurological disorders in a mammalian subject, comprising a human subject, comprising administering to the subject any of the AAV particles or pharmaceutical compositions described herein. In some embodiments, the AAV particle is a blood brain barrier crossing particle. In some embodiments, neurological disorders treated according to the methods described herein include tauopathies. Alzheimer's disease (AD), Amyotrophic lateral sclerosis (ALS), Huntington's Disease (HD), Parkinson's Disease (PD), and/or Friedreich's Ataxia (FA).

The present disclosure provides a method for administering to a subject in need thereof, comprising a human subject, a therapeutically effective amount of the AAV particles described herein to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

In some embodiments, delivery of AAV particles described herein, comprising ApoE2, ApoE3 or ApoE4 polynucleotides, may be used to treat subjects suffering from tauopathy.

In some embodiments, delivery of AAV particles described herein comprising modulatory polynucleotides for the silencing of ApoE2, ApoE3 or ApoE4 gene and/or protein expression may be used to treat subjects suffering from tauopathy.

In some embodiments, delivery of AAV particles described herein comprising modulatory polynucleotides for the silencing of tau gene and/or protein expression may be used to treat subjects suffering from tauopathy.

In some embodiments, delivery of AAV particles described herein comprising a nucleic acid encoding an anti-tau antibody may be used to treat subjects suffering from tauopathy.

In some embodiments, the compositions described herein are used in combination with one or more known or exploratory treatments for tauopathy. Non-limiting examples of such treatments include inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK30 (lithium) or PP2A, and/or immunization with tau phospho-epitopes or treatment with anti-tau antibodies.

In some embodiments, delivery of AAV particles described herein, comprising ApoE2, ApoE3 or ApoE4 polynucleotides, may be used to treat subjects suffering from AD and other tauopathies. In some embodiments, delivery of AAV particles described herein comprising modulatory polynucleotides for the silencing of the ApoE2, ApoE3 or ApoE4 gene and/or protein may be used to treat subjects suffering from AD and other tauopathies.

In some embodiments, delivery of AAV particles described herein comprising modulatory polynucleotides for the silencing of the tau gene and/or protein may be used to treat subjects suffering from AD and other tauopathies. In some embodiments, delivery of AAV particles described herein comprising a nucleic acid encoding an anti-tau antibody may be used to treat subjects suffering from AD and other tauopathies.

AAV particles and methods of using the AAV particles described herein may be used to prevent, manage and/or treat ALS. As non-limiting examples, the AAV particles described herein that may be used for the treatment, prevention or management of ALS may comprise modulatory polynucleotides targeting SOD1, C9ORF72, TARDBP and/or Tau.

AAV particles and methods of using the AAV particles described herein may be used to prevent, manage and/or treat HD. As a non-limiting example, the AAV particles described herein used to treat, prevent and/or manage HD may comprise a modulatory polynucleotide targeting HTT.

In some embodiments, methods described herein may be used to treat subjects suffering from PD and other synucleinopathies. In some cases, methods described herein may be used to treat subjects suspected of developing PD and other synucleinopathies such as Parkinson's Disease Dementia (PDD), multiple system atrophy (MSA), dementia with Lewy bodies, juvenile-onset generlized neuroaxonal dystrophy (Hallervorden-Spatz disease), pure autonomic failure (PAF), neurodegeneration with brain iron accumulation type-1 (NBIA-1) and combined Alzheimer's and Parkinson's disease.

In some embodiments, delivery of AAV particles described herein, comprising frataxin polynucleotides, may be used to treat subjects suffering from Friedreich's Ataxia. In some embodiments, the AAV particles described herein, comprising frataxin polynucleotides, may be delivered to the dentate nucleus of the cerebellum, brainstem nuclei and/or Clarke's column of the spinal cord. Delivery to one or more of these regions may treat and/or reduce the effects of Friedreich's Ataxia in a subject. In some embodiments, the AAV particles described herein, comprising frataxin polynucleotides, may be delivered by intravenous administration to the central nervous system, peripheral nervous system, and/or peripheral organs for the treatment of Friedreich's Ataxia in a subject.

### Methods of Treatment of Neurological Disease

### AAV Particles Encoding Protein Payloads

In one aspect, disclosed herein are methods for treating neurological disease associated with insufficient function/presence of a target protein (e.g., AADC, APOE2, Frataxin, SMN, GCase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, ASPA, GRN, MeCP2, GLB1, and/or GAN) in a subject in need of treatment. The method optionally comprises administering to the subject a therapeutically effective amount of a composition comprising AAV particles described herein. As a non-limiting example, the AAV particles can increase target gene expression, increase target protein production, and thus reduce one or more symptoms of neurological disease in the subject such that the subject is therapeutically treated.

In some embodiments, the AAV particle described herein comprising a nucleic acid encoding a protein payload comprises an AAV capsid that allows for transmission across the blood brain barrier after intravenous administration.

In some embodiments, the composition comprising the AAV particles described herein is administered to the central nervous system of the subject via systemic administration. In some embodiments, the systemic administration is intravenous injection.

In some embodiments, the composition comprising the AAV particles described herein is administered to the central nervous system of the subject. In other embodiments, the composition comprising the AAV particles described herein is administered to a tissue of a subject (e.g., brain of the subject).

In some embodiments, the composition comprising the AAV particles described herein is administered to the central nervous system of the subject via intraparenchymal injection. Non-limiting examples of intraparenchymal injections include intrathalamic, intrastriatal, intrahippocampal or targeting the entorhinal cortex.

In some embodiments, the composition comprising the AAV particles described herein is administered to the central nervous system of the subject via intraparenchymal injection and intrathecal injection.

In some embodiments, the AAV particles described herein may be delivered into specific types of targeted cells, including, but not limited to, a central nervous system cell (e.g., a cell of the caudate, hippocampus, thalamus, putamen, brain stem, cortex (frontal, motor, occipital or cingulate cell), purkinje fiber, substantia nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia).

In some embodiments, the AAV particles described herein may be delivered to neurons in the striatum and/or cortex.

In some embodiments, the AAV particles described herein may be used as a therapy for neurological disease.

In some embodiments, the AAV particles described herein may be used as a therapy for tauopathies.

In some embodiments, the AAV particles described herein may be used as a therapy for Alzheimer's Disease.

In some embodiments, the AAV particles described herein may be used as a therapy for Amyotrophic Lateral Sclerosis.

In some embodiments, the AAV particles described herein may be used as a therapy for Huntington's Disease.

In some embodiments, the AAV particles described herein may be used as a therapy for Parkinson's Disease.

In some embodiments, the AAV particles described herein may be used as a therapy for Friedreich's Ataxia.

In some embodiments, the AAV particles described herein may be used to increase target protein expression in a central nervous system cell (e.g., a cell of the caudate, hippocampus, thalamus, putamen, brain stem, cortex (frontal, motor, occipital or cingulate cell), purkinje fiber, substantia nigra, spinal cord (cervical, thoracic, lumbar cell), dorsal root ganglion, cerebellum, or striatum), a neuron (e.g., medium spiny neuron or cortical neuron), or an astrocyte in order to treat a neurological disease. Target protein in these cells may be increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles described herein may be used to increase target protein expression in astrocytes in order to treat a neurological disease. Target protein in astrocytes may be increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in microglia. The increase of target protein in microglia may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in cortical neurons. The increase of target protein in the cortical neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in hippocampal neurons. The increase of target protein in the hippocampal neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in DRG and/or sympathetic neurons. The increase of target protein in the DRG and/or sympathetic neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles described herein may be used to increase target protein in sensory neurons in order to treat neurological disease. Target protein in sensory neurons may be increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the composition described herein for treating neurological disease is administered to the subject in need intravenously, intra-arterially, intramuscularly, subcutaneously, intraperitoneally, intraparenchymally, intrathecally and/or intraventricularly, allowing the AAV particles to pass through one or both the blood-brain barrier and the blood spinal cord barrier. In some aspects, the method comprises administering (e.g., intraparenchymal administration, intraventricular administration and/or intrathecally administration) directly to the central nervous system (CNS) of a subject (using, e.g., an infusion pump and/or a delivery scaffold) a therapeutically effective amount of a composition comprising AAV particles described herein. The AAV particles may be used to increase target gene expression, and/or reducing one or more symptoms of neurological disease in the subject such that the subject is therapeutically treated.

In some embodiments, administration of the AAV particles described herein to a subject may increase target protein levels in a subject. The target protein levels may be increased by about 10%, 20%, 30%, 40%, 50%, 60%, 70%, 80%, 85%, 90%, 95% and 100% in a subject such as, but not limited to, the CNS or PNS, a region of the CNS or PNS, or a specific cell of the CNS or PNS of a subject. As a non-limiting example, a subject may have an increase of 10% of target protein. As a non-limiting example, the AAV particles may increase the protein levels of a target protein by fold increases over baseline. In some embodiments, AAV particles lead to 5-6 times higher levels of a target protein.

In some embodiments, administration of the AAV particles described herein to a subject may increase the expression of a target protein in a subject. The expression of the target protein may be increased by about 10%, 20%, 30%, 40%, 50%, 60%, 70%, 80%, 85%, 90%, 95% and 100% in a subject such as, but not limited to, the CNS or PNS, a region of the CNS or PNS, or a specific cell of the CNS or PNS of a subject.

In some embodiments, intravenous administration of the AAV particles described herein to a subject may increase the CNS expression of a target protein in a subject. The expression of the target protein may be increased by about 10%, 20%, 30%, 40%, 50%, 60%, 70%, 80%, 85%, 90%, 95% and 100%, in a subject such as, but not limited to, the CNS, a region of the CNS, or a specific cell of the CNS of a subject.

In some embodiments, administration of the AAV particles to a subject will increase the expression of a target protein in a subject and the increase of the expression of the target protein will reduce the effects and/or symptoms of neurological disease in a subject.

### AAV Particles Comprising Modulatory Polynucleotides

In one aspect, provided herein are methods for introducing the AAV particles, comprising a nucleic acid sequence encoding the siRNA molecules described herein into cells, the method comprising introducing into the cells any of the AAV particles in an amount sufficient for degradation of a target mRNA to occur, thereby activating target-specific RNAi in the cells. In some aspects, the cells may be muscle cells, stem cells, a central nervous system cell (e.g., a cell of the caudate, hippocampus, thalamus, putamen, brain stem, cortex (frontal, motor, occipital or cingulate cell), purkinje fiber, substanita nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia).

In one aspect, provided herein are methods for treating neurological diseases associated with dysfunction of a target protein in a subject in need of treatment. The method optionally comprises administering to the subject a therapeutically effective amount of a composition comprising AAV particles comprising a nucleic acid sequence encoding the siRNA molecules described herein. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles described herein comprising a nucleic acid sequence encoding siRNA molecules comprise an AAV capsid that allows for transmission across the blood brain barrier after intravenous administration.

In some embodiments, the composition comprising the AAV particles comprising a nucleic acid sequence encoding the siRNA molecules described herein is administered to the central nervous system of the subject. In other embodiments, the composition comprising the AAV particles comprising a nucleic acid sequence encoding the siRNA molecules described herein is administered to a tissue of a subject (e.g., brain of the subject).

In some embodiments, the composition comprising the AAV particles comprising a nucleic acid sequence encoding the siRNA molecules described herein is administered to the central nervous system of the subject via systemic administration. In some embodiments, the systemic administration is intravenous injection.

In some embodiments, the composition comprising the AAV particles comprising a nucleic acid sequence encoding the siRNA molecules described herein is administered to the central nervous system of the subject via intraparenchymal injection. Non-limiting examples of intraparenchymal injections include intrathalamic, intrastriatal, intrahippocampal or targeting the entorhinal cortex.

In some embodiments, the composition comprising the AAV particles comprising a nucleic acid sequence encoding the siRNA molecules described herein is administered to the central nervous system of the subject via intraparenchymal injection and intrathecal injection.

In some embodiments, the AAV particles comprising a nucleic acid sequence encoding the siRNA molecules described herein may be delivered into specific types of targeted cells, including, but not limited to, a central nervous system cell (e.g., a cell of the caudate, hippocampus, thalamus, putamen, brain stem, cortex (frontal, motor, occipital or cingulate cell), purkinje fiber, substanita nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia).

In some embodiments, the AAV particles comprising a nucleic acid sequence encoding the siRNA molecules described herein may be delivered to neurons in the striatum and/or cortex.

In some embodiments, the AAV particles comprising a nucleic acid sequence encoding the siRNA molecules described herein may be used as a therapy for neurological disease.

In some embodiments, the AAV particles comprising a nucleic acid sequence encoding the siRNA molecules described herein may be used as a therapy for tauopathies.

In some embodiments, the AAV particles comprising a nucleic acid sequence encoding the siRNA molecules described herein may be used as a therapy for Alzheimer's Disease.

In some embodiments, the AAV particles comprising a nucleic acid sequence encoding the siRNA molecules described herein may be used as a therapy for Amyotrophic Lateral Sclerosis.

In some embodiments, the AAV particles comprising a nucleic acid sequence encoding the siRNA molecules described herein may be used as a therapy for Huntington's Disease.

In some embodiments, the AAV particles comprising a nucleic acid sequence encoding the siRNA molecules described herein may be used as a therapy for Parkinson's Disease.

In some embodiments, the AAV particles comprising a nucleic acid sequence encoding the siRNA molecules described herein may be used as a therapy for Friedreich's Ataxia.

In some embodiments, the AAV particles comprising a nucleic acid sequence encoding the siRNA molecules described herein may be used to suppress a target protein in a central nervous system cell (e.g., a cell of the caudate, hippocampus, thalamus, putamen, brain stem, cortex (frontal, motor, occipital or cingulate cell), purkinje fiber, substanita nigra, spinal cord (cervical, thoracic, lumbar cell), dorsal root ganglion, cerebellum, or striatum), a neuron (e.g., medium spiny neuron or cortical neuron), an astrocyte, and/or other cells surrounding neurons (e.g., T cells or microglia) in order to treat neurological disease. Target protein in such cells may be suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein in astrocytes in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein in microglia. The suppression of the target protein in microglia may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress target protein in cortical neurons. The suppression of a target protein in cortical neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein in hippocampal neurons. The suppression of a target protein in the hippocampal neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein in DRG and/or sympathetic neurons. The suppression of a target protein in the DRG and/or sympathetic neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein in sensory neurons in order to treat neurological disease. Target protein in sensory neurons may be suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a nucleic acid sequence encoding the siRNA molecules described herein may be used to suppress a target protein and reduce symptoms of neurological disease in a subject. The suppression of target protein and/or the reduction of symptoms of neurological disease may be, independently, reduced or suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the present composition is administered as a solo therapeutic or as combination therapeutic for the treatment of neurological disease.

The AAV particles encoding siRNA duplexes targeting the gene(s) of interest may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles encoding the nucleic acid sequence for the siRNA molecules described herein can be small molecule compounds that are antioxidants, anti-inflammatory agents, anti-apoptosis agents, calcium regulators, antiglutamatergic agents, structural protein inhibitors, compounds involved in muscle function, and compounds involved in metal ion regulation.

In some embodiments, the composition described herein for treating neurological disease is administered to the subject in need intravenously, intra-arterially, intramuscularly, subcutaneously, intraperitoneally, intraparenchymally, intrathecally and/or intraventricularly, allowing the siRNA molecules or AAV particles encoding the nucleic acid sequence for the siRNA molecules to pass through one or both the blood-brain barrier and the blood spinal cord barrier. In some aspects, the method comprises administering (e.g., intraparenchymal administration, intraventricular administration and/or intrathecally administration) directly to the central nervous system (CNS) of a subject (using, e.g., an infusion pump and/or a delivery scaffold) a therapeutically effective amount of a composition comprising AAV particles encoding the nucleic acid sequence for the siRNA molecules described herein. The AAV particles may be used to silence or suppress target gene expression, and/or reducing one or more symptoms of neurological disease in the subject such that the subject is therapeutically treated.

In some embodiments, administration of the AAV particles encoding a siRNA described herein, to a subject may lower target protein levels in a subject. The target protein levels may be lowered by about 10%, 20%, 50%, 60%, 70%, 80%, 85%, 90%, 95% and 100%.

In some embodiments, administration of the AAV particles encoding a siRNA described herein, to a subject may lower the expression of a target protein in a subject. The expression of a target protein may be lowered by about 10%, 20%, 30%, 40%, 50%, 60%, 70%, 80%, 85%, 90%, 95% and 100% in a subject such as, but not limited to, the CNS or PNS, a region of the CNS or PNS, or a specific cell of the CNS or PNS of a subject.

In some embodiments, intravenous administration of the AAV particles encoding a siRNA described herein, to a subject may lower the expression of a target protein in the CNS of a subject. The expression of a target protein may be lowered by about 10%, 20%, 30%, 40%, 50%, 60%, 70%, 80%, 85%, 90%, 95% and 100% in a subject such as, but not limited to, the CNS, a region of the CNS, or a specific cell of the CNS of a subject.

In some embodiments, administration of the AAV particles to a subject will reduce the expression of a target protein in a subject and the reduction of expression of the target protein will reduce the effects and/or symptoms of neurological disease in a subject.

In some embodiments, the AAV particles may be used to decrease target protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%.

The AAV particles encoding the target protein may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

### V. KITS AND DEVICES

### Kits

In some embodiments, the disclosure provides a variety of kits for conveniently and/or effectively carrying out methods described herein. Typically kits will comprise sufficient amounts and/or numbers of components to allow a user to perform multiple treatments of a subject(s) and/or to perform multiple experiments.

Any of the AAV particles described herein may be included in a kit. In some embodiments, kits may further comprise reagents and/or instructions for creating and/or synthesizing compounds and/or compositions described herein. In some embodiments, kits may also comprise one or more buffers. In some embodiments, kits described herein may comprise components for making protein or nucleic acid arrays or libraries and thus, may comprise, for example, solid supports.

In some embodiments, kit components may be packaged either in aqueous media or in lyophilized form. The container means of the kits will generally comprise at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be included in one or more vials. Kits described herein may also typically comprise means for containing compounds and/or compositions described herein, e.g., proteins, nucleic acids, and any other reagent containers in close confinement for commercial sale. Such containers may comprise injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits described herein. In such embodiments, dye may then be resuspended in any suitable solvent, such as DMSO.

In some embodiments, kits may comprise instructions for employing kit components as well as the use of any other reagent not included in the kit. Instructions may comprise variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in their entirety. The method may comprise: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane, and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which are herein incorporated by reference in their entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device comprises a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the content of which is herein incorporated by reference in its entirety.

In some embodiments, the AAV particles may be delivered using a cannula that is MRI-compatible. Non-limiting examples of MRI-compatible cannulas comprise those taught in International Patent Publication No. WO2011130107, the contents of which are herein incorporated by reference in its entirety.

In some embodiments, the AAV particles may be delivered using a catheter that is MRI-compatible. Non-limiting examples of MRI-compatible catheters comprise those taught in International Patent Publication No. WO2012116265, U.S. Pat. No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### VI. DEFINITIONS

At various places in the present specification, substituents of compounds of the present disclosure are disclosed in groups or in ranges. It is specifically intended that the present disclosure include each and every individual subcombination of the members of such groups and ranges.

Unless stated otherwise, the following terms and phrases have the meanings described below. The definitions are not meant to be limiting in nature and serve to provide a clearer understanding of certain aspects described herein.

About: As used herein, the term “about” means+/−10% of the recited value.

Active ingredient: As used herein, the phrase “active ingredient” generally refers either to an AAV particle carrying a payload region encoding the polypeptides described herein or to the end product encoded by a viral genome of an AAV particle as described herein.

Adeno-associated virus: The term “adeno-associated virus” or “AAV” as used herein refers to members of the dependovirus genus comprising any particle, sequence, gene, protein, or component derived therefrom.

AAV Capsid: As used herein, an “AAV capsid” is the protein shell of an AAV virus composed of structural subunits (e.g., capsid proteins). An AAV capsid can be composed of a mixture of AAV capsid proteins (e.g., VP1, VP2 and VP3). An AAV capsid can total 60 monomer proteins arranged in icosahedral symmetry. The ratio of VP1, VP2 and VP3 in a AAV capsid can vary depending upon the serotype, the method of production of a recombinant AAV capsid and/or other well-known variables.

AAV Particle: As used herein, an “AAV particle” is a virus that comprises a capsid and a viral genome with at least one payload region and at least one ITR region. AAV particles disclosed herein may be produced recombinantly and may be based on adeno-associated virus (AAV) parent or reference sequences. An AAV particle may be derived from any serotype described herein or known in the art, including combinations of serotypes (e.g., “pseudotyped” AAV), combinations of serotypes with the capsid proteins described herein, or from various genomes (e.g., single stranded or self-complementary). In addition, the AAV particle may be replication defective and/or targeted.

Activity: As used herein, the term “activity” refers to the condition in which things are happening or being done. Compositions described herein may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In some embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked.” “attached.” and “tethered.” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety that is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle described herein may be considered biologically active if even a portion of the encoded payload is biologically active or mimics an activity considered biologically relevant.

Capsid protein: As used herein, the phrase “capsid protein” refers to a structural protein that can be incorporated into the AAV capsid of an AAV particle, and can include a VP1, VP2 or VP3 protein.

Capsid shuffling: As used herein, the term “capsid shuffling” refers to a method of AAV library development in which the fragments of at least two parent AAV capsids are combined to generate a new recombinant capsid protein.

Capsid shuffled library: As used herein, the term “capsid shuffled library” refers to a collection of viral genomes prepared via the combination of fragments from two or more AAV capsids (capsid shuffling).

Central nervous system: As used herein, the term “central nervous system” or “CNS” refers to the tissues that control and coordinate the flow of information throughout the body of an organism. The central nervous system comprises nerve tissues, and it includes the brain and the spinal cord.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context described herein, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) that is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms comprising the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Conservative amino acid substitution: As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

Control Elements: As used herein, “control elements”. “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance that facilitates, at least in part, the m vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties that are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Directed evolution: As used herein, the term “directed evolution” refers to the generation of AAV capsid libraries (˜10⁴-10⁸) by any of a variety of mutagenesis techniques and selection of lead candidates based on response to selective pressure by properties of interest.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments described herein are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” comprises at least one AAV particle and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription. e.g., RNA or mRNA or of the amino acid product of translation, e.g., polypeptides or peptides. Methods of measuring the amount or levels of RNA, mRNA, polypeptides and peptides are well known in the art.

Hybrid virus: As used herein, the term “hybrid virus” refers to the resulting AAV particle upon the combination of fragments of at least two parent AAV capsids (capsid shuffling).

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 50%, 60%, 65%, 70%, 75%, 80%, 85%, 90%, 95%, or 99% identical or similar. The term “homologous” necessarily refers to a comparison between at least two sequences (polynucleotide or polypeptide sequences). In one aspect, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In one aspect, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region that would not be considered a homologous region.

Homologous Region: As used herein the term “homologous region” refers to a region that is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part 1, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Insect cell: As used herein, the term “insect cell” any insect cell that allows for replication of AAV. An insect cell, in some aspect, can be maintained in culture and infected with baculovirus expression vector in accordance with the present disclosure and standard techniques. Non-limiting examples of insect cell lines include Spodoptera frugiperda pupal ovarian cell lines (e.g., Sf9 or Sf21), drosophila cell lines, or mosquito cell lines, such as Aedes albopictus derived cell lines.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Library: As used herein, the term “library” refers to a collection of viral genomes and/or AAV particles with varying properties. This collection may comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 different AAV capsids. In some embodiments, libraries may comprise hundreds, thousands, or millions of different AAV capsids.

Linker: As used herein “linker” refers to a molecule or group of molecules that connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule described herein. Molecules may be modified in many ways including chemically, structurally, and functionally.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence that does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus comprised of at least two components, a capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient” as used herein, refers any ingredient other than the compounds described herein (for example, a vehicle capable of suspending or dissolving the active compound) and comprising the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines, alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentane propionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties. Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound described herein wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmaceutical composition: As used herein, the term “pharmaceutical composition” refers to a composition comprising at least one active ingredient and optionally one or more pharmaceutically acceptable excipients.

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation: (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also include single chain or multichain polypeptides and may be associated or linked. The term polypeptide may also apply to amino acid polymers in which one or more amino acid residues are an artificial chemical analogue of a corresponding naturally occurring amino acid.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify”, “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may include a linear sequence of amino acids along the protein or protein module or may include a three-dimensional area, an epitope and/or a cluster of epitopes. In some embodiments, regions include terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that comprises the N-terminus, but does not comprise the C-terminus. In some embodiments, C-terminal regions may comprise any length of amino acids, which comprise the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini, 5′ termini refer to the end of a polynucleotide including a nucleic acid with a free phosphate group. 3′ termini refer to the end of a polynucleotide including a nucleic acid with a free hydroxyl group. 5′ and 3′ regions may there for include the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that comprises the 5′ terminus, but does not comprise the 3′ terminus. In some embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (e.g., ssRNA or ssDNA, respectively) or multi-stranded (e.g., double stranded, e.g., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules that results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA that direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

2 ample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

3 Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) that is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing. e.g., enzymatic processing, to a short siRNA. siRNAs can be single stranded RNA molecules (ss-siRNAs) or double stranded RNA molecules (ds-siRNAs) comprising a sense strand and an antisense strand that hybridized to form a duplex structure called siRNA duplex.

Signal Sequences: As used herein, the phrase “signal sequences” refers to a sequence that can direct the transport or localization of a protein.

Single unit dose: As used herein, a “single unit dose” is a dose of any therapeutic administered in one dose/at one time/single route/single point of contact, e.g., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized. As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition: (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition: (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules described herein may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5 end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also comprise synthetically derived polynucleotides, such as those generated, for example, by using site-directed mutagenesis. Generally, a variant of a polynucleotide, including, but not limited to, a DNA, can have at least about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions (e.g., conservative amino acid substitutions), additions of one or more amino acids in comparison to the reference polypeptide. Similarities and/or differences in sequences between a variant and the reference polypeptide can be detected using conventional techniques known in the art, for example, Western blot. Generally, a variant of a polypeptide, can have at least about 90%, about 91%, about 92%, about 93%, about 94%, about 95%, about 96%, about 97%, about 98%, about 99% or more sequence identity to the reference polypeptide as determined by sequence alignment programs known by skilled artisans.

Vector: As used herein, a “vector” is any molecule or moiety that transports, transduces or otherwise acts as a carrier of a heterologous molecule. Vectors described herein may be produced recombinantly and may be based on and/or may include adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and that sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

### VII. EXAMPLES

### Example 1. Generation of Chimeric Capsid Library

The Example presented herein describes the successful generation of an AAV capsid shuffled library with high complexity and diversity produced from nine parental AAV serotypes (AAV2, AAV6, AAV7, AAV8, AAV9, AAVrh8, AAVrh10, AAVrh39 and AAVrh43).

The nine parental AAV serotypes were used to amplify full length cap genes by PCR. The PCR products were purified by Zymoclean™ Gel DNA Recovery Kit. Equal amounts of 9 cap PCR products were pooled and fragmented by DNase I incubation to obtain a pool of fragments between 0.2 and 1.0 kb in size. Differently sized fragments were reassembled into full-length cap variants by primerless PCR, then the second PCR was carried out to amplify shuffled cap library using the primers containing HindIII (forward primer: 5-CAGTGACGCAGATATAAGTGAGCCC-3′; SEQ ID NO: 145 or ClaI site (reverse primer: 5′-GAAACGAATTAAACGGTTTATTGATTAACAATCGATTA-3′; SEQ ID NO: 146). The band at the size of approximately 2.6 kb was purified and subcloned into linearized wild-type ITR-rep vector (digested by HindIII and ClaI) using Gibson Assembly method as shown in FIG. 1 (Gibson et al., Nat. Methods., 7(11):901-903 (2010)). The shuffled capsid library was transformed into ElectroMAX™ DH10B™ Cells by electroporation. The integrity and genetic diversity of the shuffled capsid library were assessed by total colony number and 100% homology of cap variants in hundreds of colonies by Sanger sequencing. The maximal diversity of original chimeric capsid library was about 3.4×10⁷.

To produce a large amount of viruses for directed evolution, the library plasmid and adenoviral helper plasmid were transduced into HEK-293T cells using CaCl₂ method. The resulting hybrid viruses in cells and culture medium were collected and purified by iodixanol gradient. The particle titer was assessed by qPCR and the purity of viruses was assessed by silver staining of PAGE gel. The complexity and diversity of shuffled capsid library recovered from hybrid viruses were assessed by qPCR and Sanger sequencing.

For the first round of directed evolution, two monkeys were intrathecally (IT) injected with 3.0×10¹³ vg of hybrid viruses in PBS and sacrificed after 28 days; and 5 mice were injected with 1.5×10¹¹ vg of hybrid viruses in PBS intrastriatally (IS), intravenously (IV), or via cisternae magna (CM), and sacrificed after 28 days (Table 4, FIG. 1). CNS tissues including dentate nucleus, hippocampus, thalamus, putamen, cortex, spinal cord, dorsalroot ganglion (DRG), cerebellum and peripheral organs (including liver, heart, muscle) were collected.

To recover the chimeric capsid library from CNS tissues after the first round of directed evolution, genomic DNA from individual CNS tissues was extracted by using DNeasv Blood & Tissue Kit. Chimeric capsid library was recovered from individual CNS tissues by PCR, purified by Zymolean™ Gel DNA Recovery Kit, and then pooled at equal amounts. For the next round of directed evolution, the recovered capsid library was subcloned into linearized wild-type ITR-rep plasmid and transformed into ElectroMAX™ DH10B™ Cells. The production purification and quality control of hybrid viruses were performed as for the first-round preparation.

Directed evolution and recovery of chimeric capsid library was similarly performed for three rounds in monkeys and mice (FIG. 1), but the dose of hybrid viruses for the second and third rounds was gradually reduced, as summarized in Table 4.

TABLE 4
Directed evolution of chimeric capsid variants in animal models

| | | | | | | In-life | |
|---|---|---|---|---|---|---|---|
| | | Number of | | Volume | Delivery | Duration | Tissues harvested |
| Species | Round | animals | Dose (vg) | (μl) | Route | (days) | included: |
| NHP | 1^{st} | 2 | 3.0 × 10¹³ | 3000 | IT | 28 | candate, hippocampus, |
| | 2^{nd} | 2 | 1.5 × 10¹³ | 3000 | IT | 28 | thalamus, putamen, |
| | 3^{rd} | 2 | 1.0 × 10¹³ | 3000 | IT | 28 | brain stem, cortex |
| | | | | | | | (frontal, motor, |
| | | | | | | | occipital, cingulate), |
| | | | | | | | purkinje fibers, |
| | | | | | | | substanita nigra, |
| | | | | | | | spinal cord (cervical, |
| | | | | | | | thoracic, lumbar), |
| | | | | | | | DRG, cerebellum, |
| | | | | | | | striatum, peripheral |
| | | | | | | | organs (including |
| | | | | | | | liver, heart, lung, |
| | | | | | | | muscle) |
| Mouse | 1^{st} | 5 | 1.5 × 10¹¹ | 5 | IS | 28 | dentate nucleus, |
| | | 5 | 1.5 × 10¹¹ | 100 | IV | 28 | hippocampus, |
| | | 5 | 1.5 × 10¹¹ | 5 | CM | 28 | thalamus, putamen, |
| | 2^{nd} | 5 | 5.0 × 10¹⁰ | 5 | IS | 28 | cortex, spinal cord |
| | | 5 | 5.0 × 10¹⁰ | 100 | IV | 28 | (cervical, thoracic, |
| | | 5 | 5.0 × 10¹⁰ | 5 | CM | 28 | lumbar), DRG, |
| | 3^{rd} | 5 | 1.5 × 10¹⁰ | 5 | IS | 28 | cerebellum, striatum, |
| | | 5 | 1.5 × 10¹⁰ | 100 | IV | 28 | peripheral organs |
| | | 5 | 1.5 × 10¹⁰ | 5 | CM | 28 | (including liver, |
| | | | | | | | heart, lung, muscle) |

For directed evolution in cells, neurons and astrocytes were infected with MOI at 1000 of hybrid viruses for 72 hours, then chimeric capsid library was recovered from genomic DNA of cells for next round of directed evolution. The production, purification and quality control of hybrid viruses for directed evolution were conducted using the same methods as the viruses used in animal studies. During the third and fourth rounds of directed evolution, the hybrid viruses were preincubated with human intravenous immunoglobulin (IVIG, 200 μg/ml for astrocytes and 1 mg/ml for neurons) for 1 hour before the infection with the cells.

### Example 2. Selection of Lead Capsid Variants from CNS Tissues and Cells

After 3 rounds of selection in non-human primate (NHP) with intrathecal (IT) injection, and in mice with intrastriatal, intravenous (IV), and IT injection, and 4 rounds of selection in human primary neurons and astrocytes in vitro, 24 lead capsid variants (>1% abundance) were selected. All selected capsid variants showed high complexity compared to parental capsids.

The selection of lead capsid variants from NHP CNS tissues was performed after the second and third rounds of directed evolution. The sequences of chimeric capsid variants were obtained by Sanger sequencing of random colonies from chimeric capsid library-transformed ElectroMAX® DH10B™ Cells on ampicillin-LB plate. After the second round of directed evolution in NHP, chimeric capsid library from individual CNS tissues was recovered, pooled at equal amount, and then subcloned and transformed. A total of 200 random colonies were sequenced and full-length sequences of capsid variants were obtained from 156 colonies. Using Vector NTI softwar, the sequences of 156 capsid variants were aligned and compared to parental capsids, five lead capsid variants including KJ01, KJ02, KJ03, KJ04 and KJ05 (Tables 1-3) were selected based on their abundance (KJ01: 23.9%; KJ02 and KJ03: 2.56%; KJ04 and KJ05: 1.28%).

After the third round of directed evolution in NHP, chimeric capsid library from individual CNS tissues was recovered by PCR. PCR products of approximately 2.6 kb could be recovered from all individual CNS tissues, and were individually purified, subcloned and transformed. Fifty random colonies from each CNS tissue of two monkeys were sequenced (total 1,800 colonies). The sequences of 1,354 capsid variants were obtained and aligned, 6 lead capsid variants (HW01, KJ01, HW02, HW03, KJ03 and HW04) were selected, according to their abundance rate (HW01: 20%; KJ01: 6.2%; HW02: 2.22%; HW03: 1.92%; KJ03: 1.48%; HW04: 1.04%). Tables 1-3 depict the KJ01, KJ03, HW01, HW02, HW03 and HW04 amino acid sequences and representative polynucleotide coding sequences.

Similar to NHP, the selection of lead capsid variants from mouse CNS tissues was performed after the second and third rounds of directed evolution. The recovery of chimeric capsid variants from individual mouse CNS tissues, subcloning and transformation were performed similarly as those for processing NHP samples (second round), but the samples from different injection methods (IS, IV, CM) were processed separately. The PCR products (approximately 2.6 kb) of chimeric capsid variants were recovered from individual CNS tissues, liver and heart of mice irrespective of injection route. After the second round of directed evolution, 100 random colonies from each injection method were sequenced, but no capsid variant showed duplicates out of the total 137 capsid variants. After the third round of directed evolution, full-length sequences of capsid variants were obtained from total 440 colonies (120 colonies from IS injection; 200 colonies from CM injection and 120 colonies from IV injection). Based on the alignment data and abundance rate, 12 lead capsid variants were selected as follows: HW05 (Tables 1-3; abundance: 1.36% from CM, IS and IV); HW15 (Tables 1-3; abundance: 2.5% from CM and IV); HW06 (Tables 1-3; abundance: 1.88% from CM and IV); HW13 (Tables 1-3; abundance: 1.88% from CM and IV); HW10 (Tables 1-3; abundance: 1.26% from CM and IV); HW01 (Tables 1-3; abundance: 5% from CM); HW12 (Tables 1-3; abundance: 2.5% from CM); HW11 (Tables 1-3; abundance: 1% from CM); HW14 (Tables 1-3; abundance: 1% from CM); HW16 (Tables 1-3; abundance: 1% from CM); HW17 (Tables 1-3; abundance: 1% from CM); and HW09 (Tables 1-3; abundance: 2.5% from IV).

The selection of lead capsid variants from neurons and astrocytes was performed after the third and fourth rounds of directed evolution, in which the hybrid viruses were preincubated with human (intravenous immunoglobulin) IVIG before infection with neurons and astrocytes. Based on the alignment of third-round capsid variants, 2 lead capsid variants including HW01 (Tables 1-3; abundance: 4.55%) and HW07 (Tables 1-3; abundance: 4.55%) were found in total 44 capsid variants recovered from astrocytes and 3 lead capsid variants including HW01 (Tables 1-3; abundance: 5.4%); HW18 (Tables 1-3; abundance: 5.4%) and HW19 (Tables 1-3; abundance: 10.8%) were selected from 37 capsid variants recovered from neurons. After the fourth round of directed evolution, 4 lead capsid variants including HW01 (Tables 1-3; abundance: 4%); HW06 (Tables 1-3; abundance: 8%), HW08 (Tables 1-3; abundance: 4%) and HW16 (Tables 1-3; abundance: 4%) were found in 100 capsid variants recovered from astrocytes while only HW01 (Tables 1-3; abundance: 5%) was enriched among 100 capsid variants recovered from neurons. As summarized in FIG. 2, HW01 was unique in being one of the best performing capsid variants in all 4 screening systems: NHP (third round), mouse (third round, CM injection), neurons (3^{rd} and 4^{th} rounds) and astrocytes (3^{rd} and 4^{th} rounds); HW06 and HW16 were among the best performing capsid variants from mouse (CM injection) and astrocytes (4^{th} round) screening systems.

The complexity of 24 lead capsid variants was analyzed by alignment with the 9 parental capsids (AAV2, AAV6, AAV7, AAV8, AAV9, AAVrh8, AAVrh10, AAVrh39 and AAVrh43). A Guide tree was made by Vector NTI software (FIG. 3), based on the Neighbor Joining method (NJ) of Saitou and Nei. The NJ method works on a matrix of distances between all pairs of sequences to be analyzed. These distances are related to the degree of divergence between the sequences. The calculated distance values in parenthesis following the molecule name are calculated after the sequences are aligned. The smaller the value is, the less divergence between the sequences, e.g. the value “0” represents completely identical. Based on the guide tree, all 24 capsid variants showed high complexity compared to the 9 parental capsids.

### Example 3. Characterization of HW01; HW02; HW03 and HW04 Variants in Mouse

As described herein, four of the 24 capsid variants (HW01, HW02, HW03, HW4) were characterized individually in mice using human frataxin-HA (hFXN-HA) as a representative transgene. To characterize the transduction efficiency of HW01, HW02, HW03 and HW04 in mouse CNS tissues, a transgene vector (2,828 bp from 5′-ITR to 3′-ITR; SEQ ID NO: 147; see below) was used to make rAAV.CBA.hBglobin.hFXN-HA.hGHpA vectors utilizing human frataxin (hFXN-HA) as a representative transgene. The vectors were prepared in 60 dishes (150-25 mm) of HEK-293T cells, purified by two rounds of iodixanol gradient, and formulated in PBS containing 0.001% Pluronic F-68 (formulation buffer). For quality control, the rAAV titer (by ddPCR; Table 5), vector purity (by silver staining), genome integrity (by denaturing gel staining), and endotoxin level (Table 5) were assessed.

All rAAV vectors except rAAVHW02 were intrathecally and intravenously injected into mice in separate groups (n=3 for each group), while rAAVHW02 vector was only delivered via IV injection. For IT injection, all vectors were diluted to a concentration of 2.5×10¹³ vg/ml in formulation buffer, then 10 μl of vector was injected. For IV injection, all vectors were diluted to a concentration of 2.5×10¹² vg/ml in formulation buffer, then 100 μl of vector was administered via tail vein injection. After 4 weeks, the mice were euthanized, and CNS (including cortex, striatum, brain stem, cerebellum and spinal cord) and peripheral tissues (liver, heart and lung) were harvested for further analyses.

The 5′-ITR to 3′-ITR CBA.hBglobin.hFXN-HA.hGHpA sequence used in the HW01-HW04 rAAV vectors characterized herein:

(SEQ ID NO: 147)
5′CCTGCAGGCAGCTGCGCGCTCGCTCGCTCACTGAGGCCGCCCGGGCA
AAGCCCGGGCGTCGGGCGACCTTTGGTCGCCCGGCCTCAGTGAGCGAGC
GAGCGCGCAGAGAGGGAGTGGCCAACTCCATCACTAGGGGTTCCTTGTA
GTTAATGATTAACCCGCCATGCTACTTATCTACGTAGCCATGCGTCGAC
ATAACGCGTCGACATTGATTATTGACTAGTTATTAATAGTAATCAATTA
CGGGGTCATTAGTTCATAGCCCATATATGGAGTTCCGCGTTACATAACT
TACGGTAAATGGCCCGCCTGGCTGACCGCCCAACGACCCCCGCCCATTG
ACGTCAATAATGACGTATGTTCCCATAGTAACGCCAATAGGGACTTTCC
ATTGACGTCAATGGGTGGAGTATTTACGGTAAACTGCCCACTTGGCAGT
ACATCAAGTGTATCATATGCCAAGTACGCCCCCTATTGACGTCAATGAC
GGTAAATGGCCCGCCTGGCATTATGCCCAGTACATGACCTTATGGGACT
TTCCTACTTGGCAGTACATCTACGTATTAGTCATCGCTATTACCATGTC
GAGGCCACGTTCTGCTTCACTCTCCCCATCTCCCCCCCCTCCCCACCCC
CAATTTTGTATTTATTTATTTTTTAATTATTTTGTGCAGCGATGGGGGC
GGGGGGGGGGGGCGCGCGCCAGGCGGGGCGGGGCGGGGCGAGGGGCGGG
GCGGGGCGAGGCGGAGAGGTGCGGCGGCAGCCAATCAGAGCGGCGCGCT
CCGAAAGTTTCCTTTTATGGCGAGGCGGCGGCGGCGGCGGCCCTATAAA
AAGCGAAGCGCGCGGCGGGCGGGAGCAAGCTTCGTTTAGTGAACCGTCA
GATCGCCTGGAGACGCCATCCACGCTGTTTTGACCTCCATAGAAGACAC
CGGGACCGATCCAGCCTCCGCGGATTCGAATCCCGGCCGGGAACGGTGC
ATTGGAACGCGGATTCCCCGTGCCAAGAGTGACGTAAGTACCGCCTATA
GAGTCTATAGGCCCACAAAAAATGCTTTCTTCTTTTAATATACTTTTTT
GTTTATCTTATTTCTAATACTTTCCCTAATCTCTTTCTTTCAGGGCAAT
AATGATACAATGTATCATGCCTCTTTGCACCATTCTAAAGAATAACAGT
GATAATTTCTGGGTTAAGGCAATAGCAATATTTCTGCATATAAATATTT
CTGCATATAAATTGTAACTGATGTAAGAGGTTTCATATTGCTAATAGCA
GCTACAATCCAGCTACCATTCTGCTTTTATTTTATGGTTGGGATAAGGC
TGGATTATTCTGAGTCCAAGCTAGGCCCTTTTGCTAATCATGTTCATAC
CTCTTATCTTCCTCCCACAGCTCCTGGGCAACGTGCTGGTCTGTGTGCT
GGCCCATCACTTTGGCAAAGAATTGGGATTCGAACCGGTATGTGGACTT
TCGGGCGCCGCGCAGTTGCCGGCCTCCTGGCGTCCCCGAGCCCGGCCCA
GGCCCAGACCCTCACCCGGGCCCCGCGGCTGGCAGAGTTGGCCCAGCTC
TGCAGCCGCCGGGGCCTGCGCACCGGCATCAATGCGACCTGCACAACCC
ACCACACCAGTTCGAACCTCCGTGGCCTCAACCAGATTCGGAATGTCAA
AAGGCAGAGTGTCTACTTGATGAATTTGAGGAAATCGGGAACTTTGGGC
CACCCAGGCTCTCTAGATCACACCACCTATGAAAGACTAGCAGAGGAAA
CGCTGGACTCTTTAGCAGAGTTTTTTGAAGACCTTGCAGACAAGCCATA
CACCTTTGAGGACTATGATGTTTCCTTTGGGAGTGGTGTCTTAACTGTT
AAACTGGGTGGAGATCTAGGAACCTACGTGATCAACAAGCAGACGCCAA
ACAAGCAAATCTGGTTATCTTCTCCATCCAGTGGACCCAAGCGTTATGA
CTGGACTGGGAAAAACTGGGTGTATTCCCACGACGGCGTTTCCCTCCAT
GAGCTGCTGGGCGCAGAGCTCACTAAAGCCTTAAAAACCAAACTGGACT
TGTCTTCCTTGGCCTATTCCGGAAAAGACGCTTATCCTTATGACGTGCC
TGACTATGCCTGATGACTCGAGGACGGGGTGAACTACGCCTGAGGATGG
GTGGCATCCCTGTGACCCCTCCCCAGTGCCTCTCCTGGCCCTGGAAGTT
GCCACTCCAGTGCCCACCAGCCTTGTCCTAATAAAATTAAGTTGCATCA
TTTTGTCTGACTAGGTGTCCTTCTATAATATTATGGGGTGGAGGGGGGT
GGTATGGAGCAAGGGGCAAGTTGGGAAGACAACCTGTAGGGCCTGCGGG
GTCTATTGGGAACCAAGCTGGAGTGCAGTGGCACAATCTTGGCTCACTG
CAATCTCCGCCTCCTGGGTTCAAGCGATTCTCCTGCCTCAGCCTCCCGA
GTTGTTGGGATTCCAGGCATGCGGCCAGGCTGGTCTCCAACTCCTAATC
TCAGGTGATCTACCCACCTTGGCCTCCCAAATTGCTGGGATTACAGGCG
TGAACCACTGCTCCCTTCCCTGTCCTTGGCCTAGGTATCGATGCTACGT
AGATAAGTAGCATGGCGGGTTAATCATTAACTACAGAGGAACCCCTAGT
GATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAG
TGAGCGAGCGAGCGCGCAGCTGCCTGCAGG3′

TABLE 5
rAAV.CBA.hBglobin.hFXN-HA.hGHpA Vectors
using AAV9, HW01, HW02, HW03 and HW04

| | Endotoxin | ddPCR |
|---|---|---|
| Vector name | (EU/ml) | (vg/ml) |
| AAV9.hFXN-HA | <0.5 | 3.32 × 10¹³ |
| HW01.hFXN-HA | 2.28 | 3.25 × 10¹³ |
| HW02.hFXN-HA | 1.99 | 4.49 × 10¹² |
| HW03.hFXN-HA | <0.5 | 3.2 × 10¹³ |
| HW04.hFXN-HA | <0.5 | 5.44 × 10¹³ |

After euthanizing the mice, CNS tissues and liver were collected. The expression of hFXN-HA mRNA and protein in the tissues was evaluated by real-time quantitative reverse transcription-polymerase chain reaction (qRT-PCR) and enzyme-linked immunosorbent assay (ELISA), respectively.

To compare hFXN mRNA expression among different vectors, total RNA in different tissues was extracted by RNeasy Mini Kit and cDNA was synthesized by High Capacity cDNA Reverse Transcription Kit. PCR reactions were run in duplicate for mRNA expression of hFXN and controls (TATA box binding protein (TBP) and X-prolyl aminopeptidase 1 (XPNPEP1)). The hFXN mRNA expression was normalized to TBP and XPNPEP1 mRNA expression, and then expressed relative to the level of hFXN mRNA with IT AAV9-delivery. These fold changes of hFXN mRNA expression relative to AAV9 in different tissues after transduction by HW01, HW02, HW03 and HW04 variants with IT injection are shown in Table 6. Compared to AAV9, HW01 provided higher hFXN mRNA expression in the cortex (6.26-fold) and spinal cord (2.47-fold); HW03 provided higher hFXN mRNA expression in the cortex (3.84-fold), brain stem (3.74-fold), cerebellum (1.4-fold), and spinal cord (6.68-fold); HW04 provided higher hFXN mRNA expression in the cortex (12.69-fold), striatum (1.7-fold), brain stem (2.83-fold), cerebellum (9.24-fold), and spinal cord (7.43-fold). Moreover, compared to AAV9, HW01, HW03 and HW04 provided hFXN mRNA expression levels that were 0.64-fold, 0.39-fold and 0.74-fold in the liver, and 0.55-fold, 0.44-fold and 0.62-fold in the heart, respectively, after IT injection (Table 6).

TABLE 6
The fold change of hFXN mRNA expression in different tissues transduced
by HW01, HW03 and HW04 after IT injection, compared to AAV9

| | | | | | Brain | | Spinal |
|---|---|---|---|---|---|---|---|
| Capsid | Liver | Heart | Cortex | Striatum | stem | Cerebellum | cord |
| HW01 | 0.64 | 0.55 | 6.26 | 0.67 | 0.47 | 0.26 | 2.47 |
| HW03 | 0.39 | 0.44 | 3.84 | 0.39 | 3.74 | 1.40 | 6.68 |
| HW04 | 0.74 | 0.62 | 12.69 | 1.70 | 2.83 | 9.24 | 7.43 |

The level of hFXN protein in different tissues was detected by ELISA using a Human Frataxin ELISA Kit (Abcam, ab176112). Total protein from different tissues was determined by a BCA protein assay, hFXN protein levels were normalized to total protein, and then expressed relative to hFXN protein levels with AAV9 delivery by IT administration (Table 7) or by IV dosing (Table 8). With IT injection, compared to AAV9, HW01 enhanced hFXN protein expression in the cortex (3.7-fold) and spinal cord (3.41-fold). IT HW03 provided higher hFXN protein expression in the cortex (4.16-fold), brain stem (3.13-fold), cerebellum (4.3-fold) and spinal cord (4.45-fold), compared with AAV9; HW04 provided higher hFXN protein expression in the cortex (4.72-fold), striatum (1.22-fold), brain stem (5.9-fold), cerebellum (4.46-fold), and spinal cord (1.63-fold), compared with AAV9. Additionally, HW01, HW03 and HW04 provided lower hFXN protein expression in the liver after IT injection compared to AAV9 (Table 7).

TABLE 7
The fold change of hFXN protein expression in
different tissues transduced by HW01, HW03 and
HW04 after IT injection, compared to AAV9

| | | | Brain | | Spinal | |
|---|---|---|---|---|---|---|
| Capsid | Cortex | Striatum | stem | Cerebellum | cord | Liver |
| HW01 | 3.70 | 0.12 | 0.32 | 0.11 | 3.41 | 0.49 |
| HW03 | 4.16 | 0.35 | 3.13 | 4.30 | 4.45 | 0.76 |
| HW04 | 4.72 | 1.22 | 5.90 | 4.46 | 1.63 | 0.63 |

With IV injection, HW03 and HW04 provided 4.5-fold and 5.58-fold higher hFXN protein expression, respectively, in the brain stem, while HW01, HW02, HW03 and HW04 provided less hFXN protein expression in the cortex, striatum, cerebellum, spinal cord and liver, compared to AAV9 (Table 8).

TABLE 8
The fold change of hFXN protein expression in different
tissues transduced by HW01, HW02, HW03 and HW04
after IV injection, compared to AAV9

| | | | Brain | | Spinal | |
|---|---|---|---|---|---|---|
| Capsid | Cortex | Striatum | stem | Cerebellum | cord | Liver |
| HW01 | 0.09 | 0.40 | 0.73 | 0.25 | 0.33 | 0.72 |
| HW02 | 0.03 | 0.05 | 0.07 | 0.04 | 0.01 | 0.05 |
| HW03 | 0.05 | 0.08 | 4.50 | 0.29 | 0.06 | 0.33 |
| HW04 | 0.10 | 0.73 | 5.58 | 1.07 | 0.18 | 0.59 |

### VIII. EQUIVALENTS AND SCOPE

Those skilled in the art will recognize, or be able to ascertain using no more than routine experimentation, many equivalents to the specific embodiments in accordance with the disclosure described herein. The scope described herein is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments described herein, to the tenth of the unit of the lower limit of the range, unless the context clearly dictates otherwise.

In addition, it is to be understood that any particular embodiment described herein that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions described herein (e.g., any antibiotic, therapeutic or active ingredient; any method of production: any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit described herein in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope described herein.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A capsid protein comprising a sequence selected from the group consisting of SEQ ID NO: 6, SEQ ID NO: 8, SEQ ID NO: 9, SEQ ID NO: 1-5, SEQ ID NO: 7, SEQ ID NO: 10-24, SEQ ID NO: 49-72, and SEQ ID NO: 97-120, or a fragment or variant thereof.

2. The capsid protein of claim 1 comprising a sequence selected from the group consisting of SEQ ID NO: 6, SEQ ID NO: 8, and SEQ ID NO: 9, or a fragment or variant thereof.

3. The capsid protein of claim 2 comprising SEQ ID NO: 6, or a fragment or variant thereof.

4. The capsid protein of claim 2 comprising SEQ ID NO: 8, or a fragment of variant thereof.

5. The capsid protein of claim 2 comprising SEQ ID NO: 9, or a fragment or variant thereof.

6. A nucleic acid molecule comprising a polynucleotide sequence that encodes the capsid protein of any of claims 1-5.

7. An insect cell comprising the nucleic acid molecule of claim 6.

8. The insect cell of claim 7, further comprising a rep-encoding polynucleotide sequence that encodes at least one Rep protein, wherein the at least one Rep protein is selected from the group consisting of Rep78, Rep68, Rep40, and Rep52 or a combination thereof.

9. The insect cell of claim 8, wherein the at least one Rep protein is Rep78.

10. The insect cell of claim 8, wherein the at least one Rep protein is Rep52.

11. The insect cell of claim 8, wherein the at least one Rep proteins are Rep78 and Rep 52.

12. The insect cell of any one of claims 8-11, wherein the nucleic acid molecule that encodes the capsid protein also comprises the rep-encoding polynucleotide sequence.

13. The insect cell of any one of claims 8-12, wherein the rep-encoding polynucleotide sequence is linked to a sequence that promotes expression in insect cells.

14. The insect cell of any one of claims 7-13, wherein the insect cell is an Sf9 insect cell.

15. An adeno-associated viral (AAV) particle, comprising one or more capsid proteins of any one of claims 1-5 and a viral genome, wherein the viral genome comprises at least one inverted terminal repeat (ITR) and at least one polynucleotide sequence that encodes at least one payload molecule.

16. The AAV particle of claim 15, wherein the at least one polynucleotide sequence that encodes at least one payload molecule is positioned between two ITRs.

17. The AAV particle of claim 15 or 16, wherein at least one of the at least one payload molecules is an siRNA duplex.

18. The AAV particle of claim 17, wherein the siRNA duplex, when expressed, inhibits or suppresses the expression of a gene of interest in a cell.

19. The AAV particle of claim 18, wherein the gene of interest is selected from the group consisting of superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A), and voltage-gated sodium channel alpha subunit 10 (SCN10A).

20. The AAV particle of claim 15 or 16, wherein at least one of the at least one payload molecule is a protein of interest.

21. The AAV particle of claim 20, wherein the protein of interest is selected from the group consisting of Frataxin, an antibody, Aromatic L-Amino Acid Decarboxylase (AADC), ApoE, survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1), and gigaxonin (GAN).

22. The AAV particle of claim 21, wherein the protein of interest is ApoE, and the ApoE is ApoE2.

23. A method of delivering a payload molecule to a cell, comprising contacting the cell with the AAV particle of any one of claims 15-22, wherein the AAV particle comprises a viral genome that encodes the payload molecule.

24. The method of claim 23, wherein the payload molecule encodes a protein of interest.

25. The method of claim 23, wherein the payload molecule is a modulatory polynucleotide.

26. The method of any one of claims 23-25, wherein the cell is a mammalian cell.

27. The method of claim 26, wherein the mammalian cell is a human cell.

28. The method of claim 26 or 27, wherein the mammalian cell is a nervous system cell.

29. The method of claim 28, wherein the nervous system cell is selected from the group consisting of a cell of the caudate, hippocampus, thalamus, putamen, brain stem, cortex, purkinje fiber, substantia nigra, spinal cord, dorsal root ganglion, cerebellum, and striatum.

30. The method of claim 29, wherein the nervous system cell is a cell of the cortex, and the cell of the cortex is selected from the group consisting of a frontal, motor, occipital and cingulate cortex cell.

31. The method of claim 29, wherein the nervous system cell is a cell of the spinal cord, and the cell of the spinal cord is selected from the group consisting of a cervical, thoracic, or lumbar spinal cord cell.

32. The method of claim 28, wherein the nervous system cell is a medium spiny neuron.

33. The method of claim 28, wherein the nervous system cell is a neuron.

34. The method of claim 33, wherein the neuron is a cortical neuron.

35. The method of claim 28, wherein the nervous system cell is an astrocyte.


SEQUENCE LISTING (147 sequences)

SEQ ID NO: 1 - protein, 738 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDADSVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 2 - protein, 736 aa, Artificial Sequence
  Description of Artificial Sequence Synthetic polypeptide
MAADGYLPDWLEDTLSEGIRQWWKLKPGPPPPKPAERHKDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLLNIQVKEVTDNNGVKTIANNLTSTIQVFTDSEYQLPYVLGSAHQGCPPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNTAPQIGTVNSQGALPGMVWQNRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTSVDFAVNTEGVYSEPRPIGTHYLTRNL

SEQ ID NO: 3 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLKEDTSFGGNLGRAVFQAKKRVLEPLGLVEEGAKTAPAKKRPVEPSPQRSPDSSTGIGKKGQQPARKRLNFGQTGDADSVPDPQPLGEPPAAPSGVGPNTMASGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 4 - protein, 738 aa, Artificial Sequence
  Description of Artificial Sequence Synthetic polypeptide
MAADGYLPDWLEDTLSEGIRQWWDLKPGAPKPKANQQKQDDGRGLVLPGYKYLGPFNGLDKGEPVNAADAEALEHDKAYDQQLKAGDNPYLKYNHADAEFQERLKEDTSFGGNLGRAVFQAKKRVLEPLGLVEEAAKTAPGKKRPVEPSPQRSPDSSTGIGKKGQQPAKKRLNFGQTGDSESVPDPQPIGEPPAAPSGLGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 5 - protein, 738 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDTESVPDPQPIGEPPAAPSGVGSLTMASGGGAPVADNNEGADGVGNASGNWHCDSTWLGDRVITTSTRTWALPTYNNHLYKQISNSTSGGSSNDNAYFGYSTPWGYFDFNGFHCHFSPRDWQRLINNNWGFRPKRLGFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDYYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVDSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDSESVPDPQPLGEPPATPAAVGPTTMAAGGGAPMADNNEGADGVGNSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - protein, 737 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDSESVPDPQPLGEPPATPAAVGPTTMAAGGGAPMADNNEGADGVGNSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 738 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDQQLKAGDNPYLKYNHADAEFQERLKEDTSFGGNLGRAVFQAKKRVLEPLGLVEEAAKTAPGKKRPVEPSPQRSPDSSSGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTQSTGGTQGTQQLLFSQAGPANMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTNVDFAVNTEGVYSEPRPIGTRYLTRNL

SEQ ID NO: 10 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEAAKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISSASTGASNDNTYFGYSTPWGYFDFNRFHCHFSPRDWQRLINNNWGFRPKKLRFKLFNIQVKEVTQNEGTKTIANNLTSTVQVFTDSEYQLPYVLGSAHQGCLPPFPADVFMVPQYGYLTLNNGSQALGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGMKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLQAGDNPYLKYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLRFKLFNIQVKEVTDNNGVKTIANNLTSTIQVFTDSEYQLPYVLGSAHQGCLPPFPADVFMIPQYGYLTLNNGSQAVGRSSFYCLEYFPSQMLRTGNNFQFSYEFENVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 12 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDQQLEAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 13 - protein, 736 aa, Artificial Sequence
  Description of Artificial Sequence Synthetic polypeptide
MAADGYLPDWLEDNLSEGIRQWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTIQVFTDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPHPIGTRYLTRNL

SEQ ID NO: 14 - protein, 736 aa, Artificial Sequence
  Description of Artificial Sequence Synthetic polypeptide
  MOD_RES (265)..(266): Any naturally occurring amino acid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDADSVPDPQPLGQPPAAPSGLGTNTMATGSGAPMADNNEGADGVGNSSGNWHCDSTWLGDRVITTSTRTWALPTYNNHLYKQISSQXXPRTTITTLATAPPGGYFDFNRFHCHFSPRDWQRLINNNWGFRPKRLSFKLFNIQVKEVTQNEGTKTIANNLTSTIQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLSRTQSTGGTAGTQQLLFSQAGASDIRDQSRNWLPGPCYRQQRVSKTSTDNNNSEYSWTGATKYHLNGRDSLVNPGPAMASHKDDEEKFFPQSGVLIFGEQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTNVDFAVNTEGVYSEPRPIGTRYLTRNL

SEQ ID NO: 15 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDQQLKAGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDAESVPDPQPLGEPPATPAAVGPTTMASGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPLTFNQAKLNSFITQYSTGQVSVEIEWELQKENSKRWNPEIQYTSNYYKSTSVDFAVNTEGVYSEPRPIGTRYLTRNL

SEQ ID NO: 16 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGSGTVAAGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 533 aa, Artificial Sequence
  Description of Artificial Sequence Synthetic polypeptide
VAAGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNEADAAALEHDKAYDRQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPLGEPPAAPSGLGPNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - protein, 736 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPIGEPPAAPSGVGPNTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 20 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSAGIGKSGAQPAKKRLNFGQTGDSESVPDPQPLGEPPAGPSGLGSGTMAAGGGAPMADNNEGADGVGNSSGNWHCDSTWLGDRVITTSTRTWALPTYNNHLYKQISSASTGASNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQTNTGPIVGNVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSNNVEFAVNTEGVYSEPRPIGTRYLTRNL

SEQ ID NO: 21 - protein, 738 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDADSVPDPQPLGQPPAAPSGLGTNTMATGSGAPMADNNEGADGVGNSSGNWHCDSTWLGNRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQRNTAPQIGTVNSQGALPGMVWQNRDVYLQGPIWAKIPHTDGNFHPSPLMGGFGLKHPPPQILIKNTPVPADPPTTFSAAKFASFITQYSTGQVSVEIEWELQKENSKRWNPEIQYTSNYYKSNNVEFAVNTEGVYSEPRPIGTRYLTRNL

SEQ ID NO: 22 - protein, 738 aa, Artificial Sequence
  Description of Artificial Sequence Synthetic polypeptide
  MOD_RES (476)..(476): Any naturally occurring amino acid
MAADGYLPDWLEDNLSEGIREWWDLKPGAPKPKANQQKQDDGRGLVLPGYKYLGPGNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSSGIGKSGAQPAKKRLNFGQTGDTESVPDPQPIGEPPAAPSGVGSLTMASGGGAPVADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTGGTANTQTLGFSQGGPNTMANXAKNWLPGPCYRQQRVSTTTGQNNNSNFAWTGATKYHLNGRDSLVNPGVAMATHKDDDDRFFPSSGVLIFGKQGAGNDGVDYSQVLITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNFEKQTGVDFAVNTEGTYSEPRPIGTRYLTRNL

SEQ ID NO: 23 - protein, 736 aa, Artificial Sequence
  Description of Artificial Sequence Synthetic polypeptide
  MOD_RES (217)..(217): Any naturally occurring amino acid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPQEPDSSSGIGKKGQQPARKRLNFGQTGDSESVPDPQPLGEPPATPAAVGPTTMASGGGAPMADNNEXADGVGSSSGNWHCDSQWLGDRVITTSTRTWALPTYNNHLYKQISSETAGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 24 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMMRTGNNFEFSYSFEDVPFHSSYAHSQSLDRLMNPLIDQYLYYLSRTQSTGGTAGTQQLLFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 25 - DNA, 2214 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgcaagaaaaagattgaattttggtcagactggagacgcagactcagtacctgacccacaacctctcggagaacctccagcagcgccctctagtgtgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacggcacctcgggaggaagcaccaacgacaacacctattttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaagtcagcgtggaaatcgagtgggagctgcagaaggaaaacagcaaacgctggaatccagagattcagtacacttcaaactactacaaatctacaaatgtggactttgctgttaacacagatggcacttattctgagcctcgccccatcggcacccgttacctcacccgtaatctg

SEQ ID NO: 26 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaagccaagaagcgggttctcgaacctctcggtctggttgaggaaggcgctaagacggctcctggaaagaaacgtccggtagagcagtcgccacaagagccagactcctcctcgggcatcggcaagacaggccaacagcccgctaaaaagagactcaattttggtcagactggcgactcagagtcagtccccgaccctcaaccaatcggagaaccaccagcaggcccctctggtctgggatctggtacaatggctgcaggcggtggcgctccaatggcagacaataacgagggcgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgccacttctcaccacgtgactggcagcgactcatcaacaacaactggggattccgacctaagcgactcaacttcaagctcctcaacattcaggtcaaagaagttacggacaacaatggagtcaagaccatcgccaataacctcaccagcaccatccaggtgtttacggactcggagtaccagctgccgtacgttctcggctctgcccaccagggctgcccgcctccgttcccggcggacgtcttcatgattcctcagtacggctacctgactctcaacaatggcagtcaggccgtgggccgttcctccttctactgcctggagtactttccttctcaaatgctgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacacggctcctcaaattggaactgtcaacagccagggggccttacccggtatggtctggcagaaccgggacgtgtacctgcagggtcccatctgggccaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccattacctcacccgcaacctg

SEQ ID NO: 27 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgccttaaagaagatacgtcttttgggggcaacctcggacgagcagtcttccaggcgaaaaagagggttctcgaacctctcggtctggttgaggaaggcgctaagacggctcctgcaaagaagagaccggtagagccgtcacctcagcgttcccccgactcctccacgggcatcggcaagaaaggccagcagcctgcaagaaaaagattgaattttggtcagactggagacgcagactcagtacctgacccccagcctctcggagaacctccagcagcgccctctggtgtgggacctaatacaatggcttcaggcggtggcgcaccaatggcagacaataacgaaggcgccgacggagtgggtagttcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtcttcatgattcctcagtacggctacctgacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggctaagaactggctacctggaccttgctaccggcagcagcgagtctctacgacactgtcgcaaaacaacaacagcaactttgcttggactggtgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcctatctgggccaagattcctcacacggacggaaactttcatccctcgccgctgatgggaggctttggactgaaacacccgcctcctcagatcctgattaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 28 - DNA, 2214 bp, Artificial Sequence
  Description of Artificial Sequence Synthetic polynucleotide
atggctgccgatggttatcttccagattggctcgaggacactctctctgaaggaataagacagtggtgggacttgaaacctggagccccgaaacccaaagccaaccagcaaaagcaggacgacggccggggcctggtgcttcctggctacaagtacctcggacccttcaacggactcgacaagggggagcccgtcaacgcggcagacgcggaggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctctacgggcatcggcaagaaaggccagcagcccgcgaaaaagagactcaactttgggcagactggcgactcagagtcagtgcccgaccctcaaccaatcggagaacctccagcagccccctcaggtctgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgacttcaacagattccactgccacttctcaccacgtgactggcaacgactcatcaacaacaattggggattccggcccaaaagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcgcaccagggctgcctgcctccgttcccggcggacgtcttcatgattcctcagtacgggtacctgactctgaacaatggcagtcaggccgtgggccgttcctccttctactgcctggaatattttccatcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgcaacctg

SEQ ID NO: 29 - DNA, 2214 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaaacccaaagccaaccagcaaaagcaggacaacggccggggtcttgtgcttcctgggtacaagtacctcggacccttcaacggactcgacaagggggagcccgtcaacgcggcggacgcagcggccctcgagcacgacaaggcctacgaccagcagctcaaagcgggtgacaacccgtacctgcggtacaaccacgccgacgcggagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgacggagtgggtaatgcctcaggaaattggcattgcgattccacatggctgggcgacagagtcatcaccaccagcacccgaacatgggccctgcccacctacaacaaccacctctacaagcaaatctccaacagcacatctggaggatcttcaaatgacaacgcctacttcggctacagcaccccctgggggtattttgacttcaacggattccactgccatttctcaccacgtgactggcagcgactcatcaacaacaattggggattccggcccaagagactcggcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattcctcagtacgggtacctgactctgaacaatggcagtcaggccgtgggccgttcctccttctactgcctggagtactttccttctcaaatgctgcgaactggaaacaattttgaattcagctacaccttcgaggacgtgcctttccacagcagctacgcgcacagccagagcctggacaggctgatgaatcccctcatcgactactacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcgacagccagggggccttacccggtatggtctggcagaaccgggacgtgtacctgcagggtcccatctgggccaagattcctcacacggacggcaacttccacccgtccccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtttactctgagcctcgccctattgggactcgttacctcacccgtaatctg

SEQ ID NO: 30 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaattttggtcagactggcgactcagagtcagtccccgatccacaacctctcggagaacctccagcaacccccgctgctgtgggacctactacaatggctgcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 31 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 32 - DNA, 2211 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaattttggtcagactggcgactcagagtcagtccccgatccacaacctctcggagaacctccagcaacccccgctgctgtgggacctactacaatggctgcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 33 - DNA, 2214 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggtgacaacccgtacctcaagtacaaccacgccgacgcggagtttcaggagcgccttaaagaagatacgtcttttgggggcaacctcggacgagcagtcttccaggccaagaagcgggttctcgaacctcttggtctggttgaggaagcggctaagacggctcctggaaagaagagaccggtagagccatcaccccagcgttctccagactcctcctcgggcatcggcaagaaaggccaacagcccgccagaaaaagactcaattttggccagactggcgactcagagtcagttccagaccctcaacctctcggagaacctccagcagcgccctctggtgtgggacctaatacaatggcttcaggcggtggcgcaccaatggcagacaataacgaaggcgccgacggagtgggtaatgcctcaggaaattggcattgcgattccacatggctgggcgatagagtcatcaccaccagcacccgaacctgggccctccccacctacaacaatcacctctacaagcaaatctccaacagcacatctggaggatcttcaaatgacaacgcctacttcggctacagcaccccctgggggtattttgacttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtgttcatgattcctcagtacggctacctgactctcaacaatggcagtcagtctgtgggccgttcctccttctactgcctggaatatttcccatcgcagatgctgagaacgggcaataactttaccttcagctacaccttcgaggacgtgcctttccacagcagctacgcccacagccagagcttggaccggctgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaagtcagcgtggaaatcgagtgggagctgcagaaggaaaacagcaagcgctggaatccagagattcaatacacttccaactactacaaatctacaaatgtggactttgctgtcaacacggagggggtttatagtgagcctcgccccattggcacccgttacctcacccgtaatctg

SEQ ID NO: 34 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagcccgtcaacgcggcggatgcagcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggccaagaagcgggttctcgaacctcttggtctggttgaggaagcggctaagacggctcctggaaagaagagaccggtagagccgtcacctcagcgttcccccgactcctccacgggcatcggcaagaaaggccagcagcccgctaaaaagagactgaactttggtcagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctacaacaaccacctctacaagcaaatctccagtgcttcaacgggggccagcaacgacaacacctacttcggctacagcaccccctgggggtattttgacttcaacagattccactgtcacttttcaccacgtgactggcaacgactcatcaacaacaactggggattccggcccaagaagctgcggttcaagctcttcaacatccaggtcaaggaggtcacgcagaatgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagtttacttacaccttcgaggacgtgcctttccacagcagctacgcgcacagccagagcctggacaggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaactttcacccttctccgctgatgggagggtttggaatgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 35 - DNA, 2208 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaagcccaaagccaaccagcaaaagcaggacgacggccggggcctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctcaagtacaaccacgccgacgcggagtttcaggagcggctgcaagaagatacgtcttttgggggcaacctcgggcgagcagtcttccaggccaagaagcgggttctcgaacctctcggtctggttgaggaaggcgctaagacggctcctggaaagaagagaccagtagagcagtcgccacaagagccagactcctcctcgggcatcggcaagaaaggccagcagcccgcgaaaaagagactcaactttgggcagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcagcgactcatcaacaacaactggggattccggcccaagaagctgcggttcaagctcttcaacattcaggtcaaagaggttacggacaacaatggagtcaagaccatcgccaataacctcaccagcaccatccaggtgtttacggactcggagtaccagctgccgtacgttctcggctctgcccaccagggctgcctgcctccgttcccggcggacgtcttcatgattccgcagtacggctaccttacactgaacaatggaagtcaagccgtaggccgttcctccttctactgcctggaatactttccttcgcagatgctgagaaccggcaacaacttccagttcagctacgagtttgagaacgtacctttccatagcagctacgctcacagccaaagcctggaccgactaatgaatccactgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaactttcacccttctccgctgatgggcggctttggcctgaaacacccgcctcctcagatcctgattaagaatacacctgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctg

SEQ ID NO: 36 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagcccgtcaacgcggcggacgcagcggccctcgagcacgacaaggcctacgaccagcagctcgaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggaataccagctcccgtacgtcctcggctctgcgcaccagggctgcctgcctccgttcccggcggacgtcttcatgattcctcagtacgggtacctgactctgaacaatggaagtcaagccgtaggccgttcctccttctactgcctggaatatttcccatcgcagatgctgagaacgggcaacaactttgagttcagctacagcttcgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcctctcatgggcggctttggacttaagcacccgcctcctcagatcctcatcaaaaacacgcctgttcccgcggatcctccaactaccttcagtcaagctaagctggcgtcgttcatcacgcagtacagcaccggacaggtcagcgtggaaatcgagtgggagctgcagaaggagaacagcaaacgctggaacccagagattcaatacacttccaactactacaaatctacaaatgtggactttgctgttaatacagaaggcgtgtactctgaaccccgccccattggcacccgttacctcacccgtaatctg

SEQ ID NO: 37 - DNA, 2208 bp, Artificial Sequence
  Description of Artificial Sequence Synthetic polynucleotide
atggctgccgatggttatcttccagattggctcgaggacaacctctctgaaggaataagacagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataacctcaccagcaccatccaggtgtttacggactcggagtaccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacgggaggtaccgcaggaactcagcagttgctattttctcaggccgggcctaataacatgtcggctcaggccaaaaactggctacccgggccctgctaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaaccccgagatccagtacacttccaactacaacaagtctgttaatgtggactttactgtggacactaatggcgtgtattcagagcctcaccccattggcaccagatacctcacccgtaatctg

SEQ ID NO: 38 - DNA, 2208 bp, Artificial Sequence
  Description of Artificial Sequence Synthetic polynucleotide
  modified_base (795)..(796): a, c, t or g
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagagccatcaccccagcgttctccagactcctctacgggcatcggcaagaaaggccaacagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggctgggcgacagagtcattaccaccagcacccgaacctgggccctgcccacctacaacaaccacctctacaaacaaatttccagccaatcnnagcctcgaacgacaatcactactttggctacagcaccccctggggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctctgcgcaccagggctgcctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgctcacagccagagtctggaccggctgatgaaccccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctacggataacaacaacagtgaatactcgtggactggagctaccaagtaccacctcaatggcagagactctctggtgaatccgggcccggccatggcaagccacaaggacgatgaagaaaagttttttcctcagagcggggttctcatctttggggagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaacttccacccgtctccgctgatgggcggctttggcctgaaacatcctccgcctcagatcctgatcaagaacacgcctgtacctgcggatcctccgaccaccttcaaccagtcaaagctgaactccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaacacggagggggtttatagcgagcctcgccccattggcacccgttacctcacccgcaacctg

SEQ ID NO: 39 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggacgcagcggccctcgagcacgacaaggcctacgaccagcagctcaaagcgggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctcccgtacgtcctcggctctgcgcaccagggctgcctgcctccgttcccggcggacgtgttcatgattcctcagtacgggtacctgactctgaacaatggcagtcaggccgtgggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaagcgctggaacccggagattcagtacacctccaactactacaaatctacaagtgtggactttgctgttaatacagaaggcgtgtactctgaaccccgccccattggcacccgttacctcacccgtaatctg

SEQ ID NO: 40 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagcccgtcaacgcggcggatgcagcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcagccccctctggtctgggatctggtacagtggctgcaggcggtggcgcaccaatggcagacaataacgaaggtgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgacttcaacagattccactgccatttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccatcgcagatgctgagaaccggcaacaacttccagtttacttacaccttcgaggacgtgcctttccacagcagctacgcccacagccagagcttggaccggctgatgaatcctctgattgaccagtacctgtactacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcacccgttacctcacccgtaatctg

SEQ ID NO: 41 - DNA, 2205 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccagtagagcagtcaccccaagaaccagactcctcctcgggcatcggcaagaaaggccaacagcccgccagaaaaagactcaattttggccagactggcgactcagagtcagttccagaccctcaacctctcggagaacctccagcaacccccgctgctgtgggacctactacaatggcttcaggcggtggcgcaccaatggcagacaataacgaaggcgccgacggagtgggtaattcctcgggaaattggcattgcgattccacatggctgggggacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacggtcatccttttactgcctggaatatttcccatctcagatgctgagaacgggcaataactttaccttcagctacaccttcgaggacgtgcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaagattcctcacacggacggcaacttccacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtactcgttacctcacccgtaatctg

SEQ ID NO: 42 - DNA, 2208 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaagcccaaagccaaccagcagaagcaggacgacggccggggtctggtgcttcctggctacaagtacctcggacccttcaacggactcgacaagggagagccggtcaacgaggcagacgccgcggccctcgagcacgacaaagcctacgaccggcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtatttcgacttcaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctccctccgttcccggcggacgtgttcatgattcctcagtacggctacctgactctcaacaatggcagtcagtctgtgggacgttcctccttctactgcctggagtacttcccctctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaaaattcctcacacggacggcaacttccacccgtctcccctgatgggcggctttggactgaagcacccgcctcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaagaaaacagcaagcgctggaaccccgagatccagtacacctccaactactacaaatctacaagtgtggactttgctgtcaacacggagggggtttatagcgagcctcgccccattggcacccgttacctcacccgtaatctg

SEQ ID NO: 43 - DNA, 2208 bp, Artificial Sequence
  Description of Artificial Sequence Synthetic polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctgggcctggttgaggaaggcgctaagacggctcctggaaagaagagaccagtagagcagtcaccccaagaaccagactcctcctcgggcatcggcaagaaaggccaacagcccgccagaaaaagactcaattttggccagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggccaaaaagactcagcttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtcttcatgattccccagtacggctacctaacactcaacaacggtagtcaggccgtgggacgctcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctcatcaaaaacacacctgtacctgcgaatccttcgaccaccttcagtgcggcaaagtttgcttccttcatcacacagtactccacgggacaggtcagcgtggaaatcgagtgggagctgcagaaagaaaacagcaaacgctggaacccagagattcaatacacttccaactactacaaatctacaagtgtggactttgctgttaatacagaaggcgtgtactctgaaccccgccccattggcacccgttacctcacccgtaatctg

SEQ ID NO: 44 - DNA, 2208 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtctcctcaggaaccggactcctccgcgggtattggcaaatcgggtgcacagcccgctaaaaagagactcaattttggtcagactggcgactcagagtcagtccccgatccacaacctctcggagaaccaccagcaggcccctctggtctgggatctggtacaatggctgcaggcggtggcgctccaatggcagacaataacgaaggcgccgacggagtgggtaattcctcgggaaattggcattgcgattccacatggctgggcgacagagtcatcaccaccagcacccgaacctgggccctgcccacctacaacaaccacctctacaagcaaatctccagtgcttcaacgggggccagcaacgacaacacctacttcggctacagcaccccctgggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtctttacggactcggaataccagctgccgtacgtcctcggctctgcccaccagggctgcctgcctccgttcccggcggacgtgttcatgattccccagtacggttacctaacactcaacaacggtagtcaggccgtgggacgctcctccttctactgcctggaatactttccttcgcagatgctgagaacgggcaacaactttgagttcagctaccagtttgaggacgtgccttttcacagcagctacgcgcacagccagagcctggaccggctgatgaatcctctcatcgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcaaaccaatacggggcctattgtgggaaatgtcaacagccaaggagccttacctggcatggtctggcagaaccgagacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaacccggagatccagtacacttccaactattacaagtctaataatgttgaatttgctgttaatacagaaggcgtgtactctgaaccccgccccattggcacccgttacctcacccgtaatctg

SEQ ID NO: 45 - DNA, 2214 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggctgggcaacagagtcatcaccaccagcacccgaacctgggccctgcccacctacaacaaccacctctacaagcaaatatccaatgggacatcgggaggaagcaccaacgacaacacctattttggctacagcaccccctgggggtattttgacttcaacagattccactgtcacttttcaccacgtgactggcaacgactcatcaacaacaactggggattccggcccaagagactcagcttcaagctcttcaacattcaggtcaaggaggtcacgcagaatgaaggcaccaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcgaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccaactaccttcagtgcggcaaagtttgcttccttcatcacacagtactccacgggacaggtcagcgtggagatcgagtgggagctgcagaaggaaaacagcaaacgctggaatcccgaaattcagtacacttccaactattacaagtctaataatgttgaatttgctgttaatactgaaggtgtatatagtgaaccccgccccattggcaccagatacctgactcgtaatctg

SEQ ID NO: 46 - DNA, 2214 bp, Artificial Sequence
  Description of Artificial Sequence Synthetic polynucleotide
  modified_base (1428)..(1428): a, c, t or g
atggctgccgatggttatcttccagattggctcgaggacaacctctctgagggcattcgcgagtggtgggacctgaaacctggagcccccaagcccaaggccaaccagcagaagcaggacgacggccggggtctggtgcttccgggttacaaataccttggacccggcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccatcaccccagcgttctccagactcctcctcgggcatcggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacaccttcgaggacgtgcctttccacagcagctacgcacacagccagagcttggaccgactgatgaatcctctcatcgaccagtacctgtactacttgtctcggactcaaacaacaggaggcacggcaaatacgcagactctgggctttagccaaggtgggcctaatacaatggccaatcangcaaagaactggctgccaggaccctgttaccggcagcagcgagtctctacgacaaccgggcaaaacaacaacagcaactttgcttggactggtgccaccaaatatcacctgaacggaagagactctctggtaaatcccggtgtcgctatggcaacccacaaggatgacgacgaccgcttcttcccttcgagcggggtcctgatttttggcaagcaaggagccgggaacgatggagtggattacagccaagtgctgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaagcgctggaacccggagattcagtacacctccaactttgaaaagcagactggtgtggactttgctgtcaatacagagggaacttattctgagcctcgccccattggtactcgttacctcacccgtaatctg

SEQ ID NO: 47 - DNA, 2208 bp, Artificial Sequence
  Description of Artificial Sequence Synthetic polynucleotide
  modified_base (649)..(649): a, c, t or g
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccagactcctcctcgggcatcggcaagaaaggccagcagcccgccagaaagagactcaatttcggtcagactggcgactcagagtcagtccccgaccctcaacctctcggagaacctccagcaacccccgctgctgtgggacctactacaatggcttcaggcggtggcgcaccaatggcagacaataacgaangtgccgatggagtgggtagttcctcgggaaattggcattgcgattcccaatggctgggggacagagtcatcaccaccagcacccgaacctgggccctgcccacctacaacaatcacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagagactcagcttcaagctctttaacattcaagtcaaagaggtcacgcagaatgacggtacgacgacgattgccaataaccttaccagcacggttcaggtgtttactgactcggagtaccagctcccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcccacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 48 - DNA, 2208 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtgtttacggactcggagtaccagctgccgtacgttctcggctctgcccaccagggctgcctgcctccgttcccggcggacgtgttcatgattcctcagtacggctacctgactctcaacaatggcagtcagtctgtgggacgttcctccttctactgcctggagtacttcccctctcagatgatgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattcctcacacggacggcaacttccacccttcaccgctaatgggaggatttggactgaagcacccacctcctcagatcctgatcaagaacacgccggtacctgcggatcctccaacaacgttcagtcaagctaagctggcgtcgttcatcacgcagtacagcaccggacaggtcagcgtggaaattgaatgggagctgcagaaggaaaacagcaagcgctggaacccggagattcaatacacttccaactactacaaatctacaaatgtggactttgctgtcaacacggagggggtttatagcgagcctcgccccattggcacccgttacctcacccgcaacctg

SEQ ID NO: 49 - protein, 601 aa, Artificial Sequence
  Description of Artificial Sequence Synthetic polypeptide
TAPGKKRPVEPSPQRSPDSSTGIGKKGQQPARKRLNFGQTGDADSVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 50 - protein, 599 aa, Artificial Sequence
  Description of Artificial Sequence Synthetic polypeptide
TAPGKKRPVEQSPQEPDSSSGIGKTGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLLNIQVKEVTDNNGVKTIANNLTSTIQVFTDSEYQLPYVLGSAHQGCPPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNTAPQIGTVNSQGALPGMVWQNRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTSVDFAVNTEGVYSEPRPIGTHYLTRNL

SEQ ID NO: 51 - protein, 600 aa, Artificial Sequence
  Description of Artificial Sequence Synthetic polypeptide
TAPAKKRPVEPSPQRSPDSSTGIGKKGQQPARKRLNFGQTGDADSVPDPQPLGEPPAAPSGVGPNTMASGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 52 - protein, 601 aa, Artificial Sequence
  Description of Artificial Sequence Synthetic polypeptide
TAPGKKRPVEPSPQRSPDSSTGIGKKGQQPAKKRLNFGQTGDSESVPDPQPIGEPPAAPSGLGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 53 - protein, 601 aa, Artificial Sequence
  Description of Artificial Sequence Synthetic polypeptide
TAPGKKRPVEPSPQRSPDSSTGIGKKGQQPAKKRLNFGQTGDTESVPDPQPIGEPPAAPSGVGSLTMASGGGAPVADNNEGADGVGNASGNWHCDSTWLGDRVITTSTRTWALPTYNNHLYKQISNSTSGGSSNDNAYFGYSTPWGYFDFNGFHCHFSPRDWQRLINNNWGFRPKRLGFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDYYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVDSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 54 - protein, 600 aa, Artificial Sequence
  Description of Artificial Sequence Synthetic polypeptide
TAPGKKRPVEQSPQEPDSSAGIGKSGAQPAKKRLNFGQTGDSESVPDPQPLGEPPATPAAVGPTTMAAGGGAPMADNNEGADGVGNSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 55 - protein, 598 aa, Artificial Sequence
  Description of Artificial Sequence Synthetic polypeptide
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 56 - protein, 600 aa, Artificial Sequence; identical to SEQ ID NO: 54
  Description of Artificial Sequence Synthetic polypeptide

SEQ ID NO: 57 - protein, 601 aa, Artificial Sequence
  Description of Artificial Sequence Synthetic polypeptide
TAPGKKRPVEPSPQRSPDSSSGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTQSTGGTQGTQQLLFSQAGPANMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTNVDFAVNTEGVYSEPRPIGTRYLTRNL

SEQ ID NO: 58 - protein, 600 aa, Artificial Sequence
  Description of Artificial Sequence Synthetic polypeptide
TAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISSASTGASNDNTYFGYSTPWGYFDFNRFHCHFSPRDWQRLINNNWGFRPKKLRFKLFNIQVKEVTQNEGTKTIANNLTSTVQVFTDSEYQLPYVLGSAHQGCLPPFPADVFMVPQYGYLTLNNGSQALGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGMKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 59 - protein, 599 aa, Artificial Sequence
  Description of Artificial Sequence Synthetic polypeptide
TAPGKKRPVEQSPQEPDSSSGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLRFKLFNIQVKEVTDNNGVKTIANNLTSTIQVFTDSEYQLPYVLGSAHQGCLPPFPADVFMIPQYGYLTLNNGSQAVGRSSFYCLEYFPSQMLRTGNNFQFSYEFENVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 60 - protein, 599 aa, Artificial Sequence
  Description of Artificial Sequence Synthetic polypeptide
TAPGKKRPVEQSPQEPDSSAGIGKSGAQPAKKRLNFGQTGDTESVPDPQPIGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 61 - protein, 599 aa, Artificial Sequence
  Description of Artificial Sequence Synthetic polypeptide
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTIQVFTDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPHPIGTRYLTRNL

SEQ ID NO: 62 - protein, 599 aa, Artificial Sequence
  Description of Artificial Sequence Synthetic polypeptide
  MOD_RES (128)..(129): Any naturally occurring amino acid
TAPGKKRPVEPSPQRSPDSSTGIGKKGQQPARKRLNFGQTGDADSVPDPQPLGQPPAAPSGLGTNTMATGSGAPMADNNEGADGVGNSSGNWHCDSTWLGDRVITTSTRTWALPTYNNHLYKQISSQXXPRTTITTLATAPPGGYFDFNRFHCHFSPRDWQRLINNNWGFRPKRLSFKLFNIQVKEVTQNEGTKTIANNLTSTIQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLSRTQSTGGTAGTQQLLFSQAGASDIRDQSRNWLPGPCYRQQRVSKTSTDNNNSEYSWTGATKYHLNGRDSLVNPGPAMASHKDDEEKFFPQSGVLIFGEQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTNVDFAVNTEGVYSEPRPIGTRYLTRNL

SEQ ID NO: 63 - protein, 598 aa, Artificial Sequence
  Description of Artificial Sequence Synthetic polypeptide
TAPGKKRPVEHSPVEPDSSSGTGKAGQQPARKRLNFGQTGDAESVPDPQPLGEPPATPAAVGPTTMASGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPLTFNQAKLNSFITQYSTGQVSVEIEWELQKENSKRWNPEIQYTSNYYKSTSVDFAVNTEGVYSEPRPIGTRYLTRNL

SEQ ID NO: 64 - protein, 598 aa, Artificial Sequence
  Description of Artificial Sequence Synthetic polypeptide
TAPGKKRPVEQSPQEPDSSSGIGKTGQQPAKKRLNFGQTGDSESVPDPQPLGEPPAAPSGLGSGTVAAGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 65 - protein, 598 aa, Artificial Sequence
  Description of Artificial Sequence Synthetic polypeptide
TAPAKKRPVEQSPQEPDSSSGIGKKGQQPARKRLNFGQTGDSESVPDPQPLGEPPATPAAVGPTTMASGGGAPMADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 66 - protein, 599 aa, Artificial Sequence
  Description of Artificial Sequence Synthetic polypeptide
TAPGKKRPVEPSPQRSPDSSTGIGKKGQQPAKKRLNFGQTGDSESVPDPQPLGEPPAAPSGLGPNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 67 - protein, 599 aa, Artificial Sequence
  Description of Artificial Sequence Synthetic polypeptide
TAPGKKRPVEQSPQEPDSSSGIGKKGQQPARKRLNFGQTGDSESVPDPQPIGEPPAAPSGVGPNTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 68 - protein, 599 aa, Artificial Sequence
  Description of Artificial Sequence Synthetic polypeptide
TAPGKKRPVEQSPQEPDSSAGIGKSGAQPAKKRLNFGQTGDSESVPDPQPLGEPPAGPSGLGSGTMAAGGGAPMADNNEGADGVGNSSGNWHCDSTWLGDRVITTSTRTWALPTYNNHLYKQISSASTGASNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQTNTGPIVGNVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSNNVEFAVNTEGVYSEPRPIGTRYLTRNL

SEQ ID NO: 69 - protein, 601 aa, Artificial Sequence
  Description of Artificial Sequence Synthetic polypeptide
TAPGKKRPVEPSPQRSPDSSTGIGKKGQQPARKRLNFGQTGDADSVPDPQPLGQPPAAPSGLGTNTMATGSGAPMADNNEGADGVGNSSGNWHCDSTWLGNRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQRNTAPQIGTVNSQGALPGMVWQNRDVYLQGPIWAKIPHTDGNFHPSPLMGGFGLKHPPPQILIKNTPVPADPPTTFSAAKFASFITQYSTGQVSVEIEWELQKENSKRWNPEIQYTSNYYKSNNVEFAVNTEGVYSEPRPIGTRYLTRNL

SEQ ID NO: 70 - protein, 601 aa, Artificial Sequence
  Description of Artificial Sequence Synthetic polypeptide
  MOD_RES (339)..(339): Any naturally occurring amino acid
TAPGKKRPVEPSPQRSPDSSSGIGKSGAQPAKKRLNFGQTGDTESVPDPQPIGEPPAAPSGVGSLTMASGGGAPVADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTGGTANTQTLGFSQGGPNTMANXAKNWLPGPCYRQQRVSTTTGQNNNSNFAWTGATKYHLNGRDSLVNPGVAMATHKDDDDRFFPSSGVLIFGKQGAGNDGVDYSQVLITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNFEKQTGVDFAVNTEGTYSEPRPIGTRYLTRNL

SEQ ID NO: 71 - protein, 599 aa, Artificial Sequence
  Description of Artificial Sequence Synthetic polypeptide
  MOD_RES (80)..(80): Any naturally occurring amino acid
TAPGKKRPVEQSPQEPDSSSGIGKKGQQPARKRLNFGQTGDSESVPDPQPLGEPPATPAAVGPTTMASGGGAPMADNNEXADGVGSSSGNWHCDSQWLGDRVITTSTRTWALPTYNNHLYKQISSETAGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 72 - protein, 599 aa, Artificial Sequence
  Description of Artificial Sequence Synthetic polypeptide
TAPGKKRPVEHSPVEPDSSSGTGKAGQQPARKRLNFGQTGDADSVPDPQPLGQPPAAPSGLGTNTMATGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMMRTGNNFEFSYSFEDVPFHSSYAHSQSLDRLMNPLIDQYLYYLSRTQSTGGTAGTQQLLFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 73 - DNA, 1803 bp, Artificial Sequence
  Description of Artificial Sequence Synthetic polynucleotide
acggctcctggaaagaagagaccggtagagccatcaccccagcgttctccagactcctctacgggcatcggcaagaaaggccaacagcccgcaagaaaaagattgaattttggtcagactggagacgcagactcagtacctgacccacaacctctcggagaacctccagcagcgccctctagtgtgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacggcacctcgggaggaagcaccaacgacaacacctattttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaagtcagcgtggaaatcgagtgggagctgcagaaggaaaacagcaaacgctggaatccagagattcagtacacttcaaactactacaaatctacaaatgtggactttgctgttaacacagatggcacttattctgagcctcgccccatcggcacccgttacctcacccgtaatctg

SEQ ID NO: 74 - DNA, 1797 bp, Artificial Sequence
  Description of Artificial Sequence Synthetic polynucleotide
acggctcctggaaagaaacgtccggtagagcagtcgccacaagagccagactcctcctcgggcatcggcaagacaggccaacagcccgctaaaaagagactcaattttggtcagactggcgactcagagtcagtccccgaccctcaaccaatcggagaaccaccagcaggcccctctggtctgggatctggtacaatggctgcaggcggtggcgctccaatggcagacaataacgagggcgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgccacttctcaccacgtgactggcagcgactcatcaacaacaactggggattccgacctaagcgactcaacttcaagctcctcaacattcaggtcaaagaagttacggacaacaatggagtcaagaccatcgccaataacctcaccagcaccatccaggtgtttacggactcggagtaccagctgccgtacgttctcggctctgcccaccagggctgcccgcctccgttcccggcggacgtcttcatgattcctcagtacggctacctgactctcaacaatggcagtcaggccgtgggccgttcctccttctactgcctggagtactttccttctcaaatgctgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacacggctcctcaaattggaactgtcaacagccagggggccttacccggtatggtctggcagaaccgggacgtgtacctgcagggtcccatctgggccaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccattacctcacccgcaacctg

SEQ ID NO: 75 - DNA, 1800 bp, Artificial Sequence
  Description of Artificial Sequence Synthetic polynucleotide
acggctcctgcaaagaagagaccggtagagccgtcacctcagcgttcccccgactcctccacgggcatcggcaagaaaggccagcagcctgcaagaaaaagattgaattttggtcagactggagacgcagactcagtacctgacccccagcctctcggagaacctccagcagcgccctctggtgtgggacctaatacaatggcttcaggcggtggcgcaccaatggcagacaataacgaaggcgccgacggagtgggtagttcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtcttcatgattcctcagtacggctacctgacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggctaagaactggctacctggaccttgctaccggcagcagcgagtctctacgacactgtcgcaaaacaacaacagcaactttgcttggactggtgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcctatctgggccaagattcctcacacggacggaaactttcatccctcgccgctgatgggaggctttggactgaaacacccgcctcctcagatcctgattaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 76 - DNA, 1803 bp, Artificial Sequence
  Description of Artificial Sequence Synthetic polynucleotide
acggctcctggaaagaagagaccggtagaaccgtcacctcagcgttcccccgactcctctacgggcatcggcaagaaaggccagcagcccgcgaaaaagagactcaactttgggcagactggcgactcagagtcagtgcccgaccctcaaccaatcggagaacctccagcagccccctcaggtctgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgacttcaacagattccactgccacttctcaccacgtgactggcaacgactcatcaacaacaattggggattccggcccaaaagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcgcaccagggctgcctgcctccgttcccggcggacgtcttcatgattcctcagtacgggtacctgactctgaacaatggcagtcaggccgtgggccgttcctccttctactgcctggaatattttccatcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgcaacctg

SEQ ID NO: 77 - DNA, 1803 bp, Artificial Sequence
  Description of Artificial Sequence Synthetic polynucleotide
acggctcctggaaagaagagaccggtagagccatcaccccagcgttctccagactcctctacgggcatcggcaagaaaggccagcagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgacggagtgggtaatgcctcaggaaattggcattgcgattccacatggctgggcgacagagtcatcaccaccagcacccgaacatgggccctgcccacctacaacaaccacctctacaagcaaatctccaacagcacatctggaggatcttcaaatgacaacgcctacttcggctacagcaccccctgggggtattttgacttcaacggattccactgccatttctcaccacgtgactggcagcgactcatcaacaacaattggggattccggcccaagagactcggcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattcctcagtacgggtacctgactctgaacaatggcagtcaggccgtgggccgttcctccttctactgcctggagtactttccttctcaaatgctgcgaactggaaacaattttgaattcagctacaccttcgaggacgtgcctttccacagcagctacgcgcacagccagagcctggacaggctgatgaatcccctcatcgactactacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcgacagccagggggccttacccggtatggtctggcagaaccgggacgtgtacctgcagggtcccatctgggccaagattcctcacacggacggcaacttccacccgtccccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtttactctgagcctcgccctattgggactcgttacctcacccgtaatctg

SEQ ID NO: 78 - DNA, 1800 bp, Artificial Sequence
  Description of Artificial Sequence Synthetic polynucleotide
acggctcctggaaagaagaggcctgtagagcagtctcctcaggaaccggactcctccgcgggtattggcaaatcgggtgcacagcccgctaaaaagagactcaattttggtcagactggcgactcagagtcagtccccgatccacaacctctcggagaacctccagcaacccccgctgctgtgggacctactacaatggctgcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 79 - DNA, 1794 bp, Artificial Sequence
  Description of Artificial Sequence Synthetic polynucleotide
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 80 - DNA, 1800 bp, Artificial Sequence; identical to SEQ ID NO: 78
  Description of Artificial Sequence Synthetic polynucleotide

SEQ ID NO: 81 - DNA, 1803 bp, Artificial Sequence
  Description of Artificial Sequence Synthetic polynucleotide
acggctcctggaaagaagagaccggtagagccatcaccccagcgttctccagactcctcctcgggcatcggcaagaaaggccaacagcccgccagaaaaagactcaattttggccagactggcgactcagagtcagttccagaccctcaacctctcggagaacctccagcagcgccctctggtgtgggacctaatacaatggcttcaggcggtggcgcaccaatggcagacaataacgaaggcgccgacggagtgggtaatgcctcaggaaattggcattgcgattccacatggctgggcgatagagtcatcaccaccagcacccgaacctgggccctccccacctacaacaatcacctctacaagcaaatctccaacagcacatctggaggatcttcaaatgacaacgcctacttcggctacagcaccccctgggggtattttgacttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtgttcatgattcctcagtacggctacctgactctcaacaatggcagtcagtctgtgggccgttcctccttctactgcctggaatatttcccatcgcagatgctgagaacgggcaataactttaccttcagctacaccttcgaggacgtgcctttccacagcagctacgcccacagccagagcttggaccggctgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaagtcagcgtggaaatcgagtgggagctgcagaaggaaaacagcaagcgctggaatccagagattcaatacacttccaactactacaaatctacaaatgtggactttgctgtcaacacggagggggtttatagtgagcctcgccccattggcacccgttacctcacccgtaatctg

SEQ ID NO: 82 - DNA, 1800 bp, Artificial Sequence
  Description of Artificial Sequence Synthetic polynucleotide
acggctcctggaaagaagagaccggtagagccgtcacctcagcgttcccccgactcctccacgggcatcggcaagaaaggccagcagcccgctaaaaagagactgaactttggtcagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctacaacaaccacctctacaagcaaatctccagtgcttcaacgggggccagcaacgacaacacctacttcggctacagcaccccctgggggtattttgacttcaacagattccactgtcacttttcaccacgtgactggcaacgactcatcaacaacaactggggattccggcccaagaagctgcggttcaagctcttcaacatccaggtcaaggaggtcacgcagaatgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagtttacttacaccttcgaggacgtgcctttccacagcagctacgcgcacagccagagcctggacaggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaactttcacccttctccgctgatgggagggtttggaatgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 83 - DNA, 1797 bp, Artificial Sequence
  Description of Artificial Sequence Synthetic polynucleotide
acggctcctggaaagaagagaccagtagagcagtcgccacaagagccagactcctcctcgggcatcggcaagaaaggccagcagcccgcgaaaaagagactcaactttgggcagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcagcgactcatcaacaacaactggggattccggcccaagaagctgcggttcaagctcttcaacattcaggtcaaagaggttacggacaacaatggagtcaagaccatcgccaataacctcaccagcaccatccaggtgtttacggactcggagtaccagctgccgtacgttctcggctctgcccaccagggctgcctgcctccgttcccggcggacgtcttcatgattccgcagtacggctaccttacactgaacaatggaagtcaagccgtaggccgttcctccttctactgcctggaatactttccttcgcagatgctgagaaccggcaacaacttccagttcagctacgagtttgagaacgtacctttccatagcagctacgctcacagccaaagcctggaccgactaatgaatccactgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaactttcacccttctccgctgatgggcggctttggcctgaaacacccgcctcctcagatcctgattaagaatacacctgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctg

SEQ ID NO: 84 - DNA, 1797 bp, Artificial Sequence
  Description of Artificial Sequence Synthetic polynucleotide
acggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggaataccagctcccgtacgtcctcggctctgcgcaccagggctgcctgcctccgttcccggcggacgtcttcatgattcctcagtacgggtacctgactctgaacaatggaagtcaagccgtaggccgttcctccttctactgcctggaatatttcccatcgcagatgctgagaacgggcaacaactttgagttcagctacagcttcgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcctctcatgggcggctttggacttaagcacccgcctcctcagatcctcatcaaaaacacgcctgttcccgcggatcctccaactaccttcagtcaagctaagctggcgtcgttcatcacgcagtacagcaccggacaggtcagcgtggaaatcgagtgggagctgcagaaggagaacagcaaacgctggaacccagagattcaatacacttccaactactacaaatctacaaatgtggactttgctgttaatacagaaggcgtgtactctgaaccccgccccattggcacccgttacctcacccgtaatctg

SEQ ID NO: 85 - DNA, 1797 bp, Artificial Sequence
  Description of Artificial Sequence Synthetic polynucleotide
acggctcctggaaagaagagaccggtagaaccgtcacctcagcgttcccccgactcctccacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataacctcaccagcaccatccaggtgtttacggactcggagtaccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacgggaggtaccgcaggaactcagcagttgctattttctcaggccgggcctaataacatgtcggctcaggccaaaaactggctacccgggccctgctaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaaccccgagatccagtacacttccaactacaacaagtctgttaatgtggactttactgtggacactaatggcgtgtattcagagcctcaccccattggcaccagatacctcacccgtaatctg

SEQ ID NO: 86 - DNA, 1797 bp, Artificial Sequence
  Description of Artificial Sequence Synthetic polynucleotide
  modified_base (384)..(385): a, c, t or g
acggctcctggaaagaagagaccggtagagccatcaccccagcgttctccagactcctctacgggcatcggcaagaaaggccaacagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggctgggcgacagagtcattaccaccagcacccgaacctgggccctgcccacctacaacaaccacctctacaaacaaatttccagccaatcnnagcctcgaacgacaatcactactttggctacagcaccccctggggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctctgcgcaccagggctgcctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgctcacagccagagtctggaccggctgatgaaccccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctacggataacaacaacagtgaatactcgtggactggagctaccaagtaccacctcaatggcagagactctctggtgaatccgggcccggccatggcaagccacaaggacgatgaagaaaagttttttcctcagagcggggttctcatctttggggagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaacttccacccgtctccgctgatgggcggctttggcctgaaacatcctccgcctcagatcctgatcaagaacacgcctgtacctgcggatcctccgaccaccttcaaccagtcaaagctgaactccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaacacggagggggtttatagcgagcctcgccccattggcacccgttacctcacccgcaacctg

SEQ ID NO: 87 - DNA, 1794 bp, Artificial Sequence
  Description of Artificial Sequence Synthetic polynucleotide
acggctccgggaaaaaagaggccggtagagcactctcctgtggagccagactcctcctcgggaaccggaaaggcgggccagcagcctgcaagaaaaagattgaattttggtcagactggagacgcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctcccgtacgtcctcggctctgcgcaccagggctgcctgcctccgttcccggcggacgtgttcatgattcctcagtacgggtacctgactctgaacaatggcagtcaggccgtgggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaagcgctggaacccggagattcagtacacctccaactactacaaatctacaagtgtggactttgctgttaatacagaaggcgtgtactctgaaccccgccccattggcacccgttacctcacccgtaatctg

SEQ ID NO: 88 - DNA, 1794 bp, Artificial Sequence
  Description of Artificial Sequence Synthetic polynucleotide
acggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcagccccctctggtctgggatctggtacagtggctgcaggcggtggcgcaccaatggcagacaataacgaaggtgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgacttcaacagattccactgccatttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccatcgcagatgctgagaaccggcaacaacttccagtttacttacaccttcgaggacgtgcctttccacagcagctacgcccacagccagagcttggaccggctgatgaatcctctgattgaccagtacctgtactacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcacccgttacctcacccgtaatctg

SEQ ID NO: 89 - DNA, 1794 bp, Artificial Sequence
  Description of Artificial Sequence Synthetic polynucleotide
acggctcctgcaaagaagagaccagtagagcagtcaccccaagaaccagactcctcctcgggcatcggcaagaaaggccaacagcccgccagaaaaagactcaattttggccagactggcgactcagagtcagttccagaccctcaacctctcggagaacctccagcaacccccgctgctgtgggacctactacaatggcttcaggcggtggcgcaccaatggcagacaataacgaaggcgccgacggagtgggtaattcctcgggaaattggcattgcgattccacatggctgggggacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacggtcatccttttactgcctggaatatttcccatctcagatgctgagaacgggcaataactttaccttcagctacaccttcgaggacgtgcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaagattcctcacacggacggcaacttccacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtactcgttacctcacccgtaatctg

SEQ ID NO: 90 - DNA, 1797 bp, Artificial Sequence
  Description of Artificial Sequence Synthetic polynucleotide
acggctcctggaaagaagagaccggtagaaccgtcacctcagcgttcccccgactcctccacgggcatcggcaagaaaggccagcagcccgctaaaaagagactgaactttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtatttcgacttcaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctccctccgttcccggcggacgtgttcatgattcctcagtacggctacctgactctcaacaatggcagtcagtctgtgggacgttcctccttctactgcctggagtacttcccctctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaaaattcctcacacggacggcaacttccacccgtctcccctgatgggcggctttggactgaagcacccgcctcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaagaaaacagcaagcgctggaaccccgagatccagtacacctccaactactacaaatctacaagtgtggactttgctgtcaacacggagggggtttatagcgagcctcgccccattggcacccgttacctcacccgtaatctg

SEQ ID NO: 91 - DNA, 1797 bp, Artificial Sequence
  Description of Artificial Sequence Synthetic polynucleotide
acggctcctggaaagaagagaccagtagagcagtcaccccaagaaccagactcctcctcgggcatcggcaagaaaggccaacagcccgccagaaaaagactcaattttggccagactggcgactcagagtcagtgcccgaccctcaaccaatcggagaacctccagcagcgccctctggtgtgggacctaatacaatggctgcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggccaaaaagactcagcttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtcttcatgattccccagtacggctacctaacactcaacaacggtagtcaggccgtgggacgctcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctcatcaaaaacacacctgtacctgcgaatccttcgaccaccttcagtgcggcaaagtttgcttccttcatcacacagtactccacgggacaggtcagcgtggaaatcgagtgggagctgcagaaagaaaacagcaaacgctggaacccagagattcaatacacttccaactactacaaatctacaagtgtggactttgctgttaatacagaaggcgtgtactctgaaccccgccccattggcacccgttacctcacccgtaatctg

SEQ ID NO: 92 - DNA, 1797 bp, Artificial Sequence
  Description of Artificial Sequence Synthetic polynucleotide
acggctcctggaaagaaacgtccggtagagcagtctcctcaggaaccggactcctccgcgggtattggcaaatcgggtgcacagcccgctaaaaagagactcaattttggtcagactggcgactcagagtcagtccccgatccacaacctctcggagaaccaccagcaggcccctctggtctgggatctggtacaatggctgcaggcggtggcgctccaatggcagacaataacgaaggcgccgacggagtgggtaattcctcgggaaattggcattgcgattccacatggctgggcgacagagtcatcaccaccagcacccgaacctgggccctgcccacctacaacaaccacctctacaagcaaatctccagtgcttcaacgggggccagcaacgacaacacctacttcggctacagcaccccctgggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtctttacggactcggaataccagctgccgtacgtcctcggctctgcccaccagggctgcctgcctccgttcccggcggacgtgttcatgattccccagtacggttacctaacactcaacaacggtagtcaggccgtgggacgctcctccttctactgcctggaatactttccttcgcagatgctgagaacgggcaacaactttgagttcagctaccagtttgaggacgtgccttttcacagcagctacgcgcacagccagagcctggaccggctgatgaatcctctcatcgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcaaaccaatacggggcctattgtgggaaatgtcaacagccaaggagccttacctggcatggtctggcagaaccgagacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaacccggagatccagtacacttccaactattacaagtctaataatgttgaatttgctgttaatacagaaggcgtgtactctgaaccccgccccattggcacccgttacctcacccgtaatctg

SEQ ID NO: 93 - DNA, 1803 bp, Artificial Sequence
  Description of Artificial Sequence Synthetic polynucleotide
acggctcctggaaagaagagaccggtagaaccgtcacctcagcgttcccccgactcctccacgggcatcggcaagaaaggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggctgggcaacagagtcatcaccaccagcacccgaacctgggccctgcccacctacaacaaccacctctacaagcaaatatccaatgggacatcgggaggaagcaccaacgacaacacctattttggctacagcaccccctgggggtattttgacttcaacagattccactgtcacttttcaccacgtgactggcaacgactcatcaacaacaactggggattccggcccaagagactcagcttcaagctcttcaacattcaggtcaaggaggtcacgcagaatgaaggcaccaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcgaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccaactaccttcagtgcggcaaagtttgcttccttcatcacacagtactccacgggacaggtcagcgtggagatcgagtgggagctgcagaaggaaaacagcaaacgctggaatcccgaaattcagtacacttccaactattacaagtctaataatgttgaatttgctgttaatactgaaggtgtatatagtgaaccccgccccattggcaccagatacctgactcgtaatctg

SEQ ID NO: 94 - DNA, 1803 bp, Artificial Sequence
  Description of Artificial Sequence Synthetic polynucleotide
  modified_base (1017)..(1017): a, c, t or g
acggctcctggaaagaagagaccggtagagccatcaccccagcgttctccagactcctcctcgggcatcggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacaccttcgaggacgtgcctttccacagcagctacgcacacagccagagcttggaccgactgatgaatcctctcatcgaccagtacctgtactacttgtctcggactcaaacaacaggaggcacggcaaatacgcagactctgggctttagccaaggtgggcctaatacaatggccaatcangcaaagaactggctgccaggaccctgttaccggcagcagcgagtctctacgacaaccgggcaaaacaacaacagcaactttgcttggactggtgccaccaaatatcacctgaacggaagagactctctggtaaatcccggtgtcgctatggcaacccacaaggatgacgacgaccgcttcttcccttcgagcggggtcctgatttttggcaagcaaggagccgggaacgatggagtggattacagccaagtgctgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaagcgctggaacccggagattcagtacacctccaactttgaaaagcagactggtgtggactttgctgtcaatacagagggaacttattctgagcctcgccccattggtactcgttacctcacccgtaatctg

SEQ ID NO: 95 - DNA, 1797 bp, Artificial Sequence
  Description of Artificial Sequence Synthetic polynucleotide
  modified_base (238)..(238): a, c, t or g
acggctcctggaaagaagaggcctgtagagcagtctcctcaggaaccagactcctcctcgggcatcggcaagaaaggccagcagcccgccagaaagagactcaatttcggtcagactggcgactcagagtcagtccccgaccctcaacctctcggagaacctccagcaacccccgctgctgtgggacctactacaatggcttcaggcggtggcgcaccaatggcagacaataacgaangtgccgatggagtgggtagttcctcgggaaattggcattgcgattcccaatggctgggggacagagtcatcaccaccagcacccgaacctgggccctgcccacctacaacaatcacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagagactcagcttcaagctctttaacattcaagtcaaagaggtcacgcagaatgacggtacgacgacgattgccaataaccttaccagcacggttcaggtgtttactgactcggagtaccagctcccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcccacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 96 - DNA, 1797 bp, Artificial Sequence
  Description of Artificial Sequence Synthetic polynucleotide
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtgtttacggactcggagtaccagctgccgtacgttctcggctctgcccaccagggctgcctgcctccgttcccggcggacgtgttcatgattcctcagtacggctacctgactctcaacaatggcagtcagtctgtgggacgttcctccttctactgcctggagtacttcccctctcagatgatgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattcctcacacggacggcaacttccacccttcaccgctaatgggaggatttggactgaagcacccacctcctcagatcctgatcaagaacacgccggtacctgcggatcctccaacaacgttcagtcaagctaagctggcgtcgttcatcacgcagtacagcaccggacaggtcagcgtggaaattgaatgggagctgcagaaggaaaacagcaagcgctggaacccggagattcaatacacttccaactactacaaatctacaaatgtggactttgctgtcaacacggagggggtttatagcgagcctcgccccattggcacccgttacctcacccgcaacctg

SEQ ID NO: 97 - protein, 535 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 98 - protein, 534 aa, Artificial Sequence
  Description of Artificial Sequence Synthetic polypeptide
MAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLLNIQVKEVTDNNGVKTIANNLTSTIQVFTDSEYQLPYVLGSAHQGCPPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNTAPQIGTVNSQGALPGMVWQNRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTSVDFAVNTEGVYSEPRPIGTHYLTRNL

SEQ ID NO: 99 - protein, 534 aa, Artificial Sequence
  Description of Artificial Sequence Synthetic polypeptide
MASGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 100 - protein, 535 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 101 - protein, 535 aa, Artificial Sequence
  Description of Artificial Sequence Synthetic polypeptide
MASGGGAPVADNNEGADGVGNASGNWHCDSTWLGDRVITTSTRTWALPTYNNHLYKQISNSTSGGSSNDNAYFGYSTPWGYFDFNGFHCHFSPRDWQRLINNNWGFRPKRLGFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDYYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVDSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 102 - protein, 535 aa, Artificial Sequence
  Description of Artificial Sequence Synthetic polypeptide
MAAGGGAPMADNNEGADGVGNSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 103 - protein, 533 aa, Artificial Sequence
  Description of Artificial Sequence Synthetic polypeptide
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 104 - protein, 535 aa, Artificial Sequence; identical to SEQ ID NO: 102
  Description of Artificial Sequence Synthetic polypeptide

SEQ ID NO: 105 - protein, 535 aa, Artificial Sequence
  Description of Artificial Sequence Synthetic polypeptide
MASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTQSTGGTQGTQQLLFSQAGPANMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFNQSKLNSFITQYSTGQVSVEIEWELQKENSKRWNPEIQYTSNYYKSTNVDFAVNTEGVYSEPRPIGTRYLTRNL

SEQ ID NO: 106 - protein, 534 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISSASTGASNDNTYFGYSTPWGYFDFNRFHCHFSPRDWQRLINNNWGFRPKKLRFKLFNIQVKEVTQNEGTKTIANNLTSTVQVFTDSEYQLPYVLGSAHQGCLPPFPADVFMVPQYGYLTLNNGSQALGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGMKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 107 - protein, 534 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLRFKLFNIQVKEVTDNNGVKTIANNLTSTIQVFTDSEYQLPYVLGSAHQGCLPPFPADVFMIPQYGYLTLNNGSQAVGRSSFYCLEYFPSQMLRTGNNFQFSYEFENVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 108 - protein, 534 aa, Artificial Sequence
  Description of Artificial Sequence Synthetic polypeptide
VAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 109 - protein, 533 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTIQVFTDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPHPIGTRYLTRNL

SEQ ID NO: 110 - protein, 533 aa, Artificial Sequence
  Description of Artificial Sequence Synthetic polypeptide
  MOD_RES (62)..(63): Any naturally occurring amino acid
MATGSGAPMADNNEGADGVGNSSGNWHCDSTWLGDRVITTSTRTWALPTYNNHLYKQISSQXXPRTTITTLATAPPGGYFDFNRFHCHFSPRDWQRLINNNWGFRPKRLSFKLFNIQVKEVTQNEGTKTIANNLTSTIQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLSRTQSTGGTAGTQQLLFSQAGASDIRDQSRNWLPGPCYRQQRVSKTSTDNNNSEYSWTGATKYHLNGRDSLVNPGPAMASHKDDEEKFFPQSGVLIFGEQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTNVDFAVNTEGVYSEPRPIGTRYLTRNL

SEQ ID NO: 111 - protein, 533 aa, Artificial Sequence
  Description of Artificial Sequence Synthetic polypeptide
MASGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPLTFNQAKLNSFITQYSTGQVSVEIEWELQKENSKRWNPEIQYTSNYYKSTSVDFAVNTEGVYSEPRPIGTRYLTRNL

SEQ ID NO: 112 - protein, 533 aa, Artificial Sequence; identical to SEQ ID NO: 17
  Description of Artificial Sequence Synthetic polypeptide

SEQ ID NO: 113 - protein, 533 aa, Artificial Sequence
  Description of Artificial Sequence Synthetic polypeptide
MASGGGAPMADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 114 - protein, 533 aa, Artificial Sequence
  Description of Artificial Sequence Synthetic polypeptide
MASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 115 - protein, 534 aa, Artificial Sequence
  Description of Artificial Sequence Synthetic polypeptide
MAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 116 - protein, 534 aa, Artificial Sequence
  Description of Artificial Sequence Synthetic polypeptide
MAAGGGAPMADNNEGADGVGNSSGNWHCDSTWLGDRVITTSTRTWALPTYNNHLYKQISSASTGASNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQTNTGPIVGNVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSNNVEFAVNTEGVYSEPRPIGTRYLTRNL

SEQ ID NO: 117 - protein, 535 aa, Artificial Sequence
  Description of Artificial Sequence Synthetic polypeptide
MATGSGAPMADNNEGADGVGNSSGNWHCDSTWLGNRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQRNTAPQIGTVNSQGALPGMVWQNRDVYLQGPIWAKIPHTDGNFHPSPLMGGFGLKHPPPQILIKNTPVPADPPTTFSAAKFASFITQYSTGQVSVEIEWELQKENSKRWNPEIQYTSNYYKSNNVEFAVNTEGVYSEPRPIGTRYLTRNL

SEQ ID NO: 118 - protein, 535 aa, Artificial Sequence
  Description of Artificial Sequence Synthetic polypeptide
  MOD_RES (273)..(273): Any naturally occurring amino acid
MASGGGAPVADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTGGTANTQTLGFSQGGPNTMANXAKNWLPGPCYRQQRVSTTTGQNNNSNFAWTGATKYHLNGRDSLVNPGVAMATHKDDDDRFFPSSGVLIFGKQGAGNDGVDYSQVLITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNFEKQTGVDFAVNTEGTYSEPRPIGTRYLTRNL

SEQ ID NO: 119 - protein, 534 aa, Artificial Sequence
  Description of Artificial Sequence Synthetic polypeptide
  MOD_RES (15)..(15): Any naturally occurring amino acid
MASGGGAPMADNNEXADGVGSSSGNWHCDSQWLGDRVITTSTRTWALPTYNNHLYKQISSETAGSTNDNTYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 120 - protein, 534 aa, Artificial Sequence
  Description of Artificial Sequence Synthetic polypeptide
MATGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMMRTGNNFEFSYSFEDVPFHSSYAHSQSLDRLMNPLIDQYLYYLSRTQSTGGTAGTQQLLFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGVYSEPRPIGTRYLTRNL

SEQ ID NO: 121 - DNA, 1605 bp, Artificial Sequence
  Description of Artificial Sequence Synthetic polynucleotide
atggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacggcacctcgggaggaagcaccaacgacaacacctattttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaagtcagcgtggaaatcgagtgggagctgcagaaggaaaacagcaaacgctggaatccagagattcagtacacttcaaactactacaaatctacaaatgtggactttgctgttaacacagatggcacttattctgagcctcgccccatcggcacccgttacctcacccgtaatctg

SEQ ID NO: 122 - DNA, 1602 bp, Artificial Sequence
  Description of Artificial Sequence Synthetic polynucleotide
atggctgcaggcggtggcgctccaatggcagacaataacgagggcgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgccacttctcaccacgtgactggcagcgactcatcaacaacaactggggattccgacctaagcgactcaacttcaagctcctcaacattcaggtcaaagaagttacggacaacaatggagtcaagaccatcgccaataacctcaccagcaccatccaggtgtttacggactcggagtaccagctgccgtacgttctcggctctgcccaccagggctgcccgcctccgttcccggcggacgtcttcatgattcctcagtacggctacctgactctcaacaatggcagtcaggccgtgggccgttcctccttctactgcctggagtactttccttctcaaatgctgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacacggctcctcaaattggaactgtcaacagccagggggccttacccggtatggtctggcagaaccgggacgtgtacctgcagggtcccatctgggccaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccattacctcacccgcaacctg

SEQ ID NO: 123 - DNA, 1602 bp, Artificial Sequence
  Description of Artificial Sequence Synthetic polynucleotide
atggcttcaggcggtggcgcaccaatggcagacaataacgaaggcgccgacggagtgggtagttcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtcttcatgattcctcagtacggctacctgacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggctaagaactggctacctggaccttgctaccggcagcagcgagtctctacgacactgtcgcaaaacaacaacagcaactttgcttggactggtgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcctatctgggccaagattcctcacacggacggaaactttcatccctcgccgctgatgggaggctttggactgaaacacccgcctcctcagatcctgattaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 124 - DNA, 1605 bp, Artificial Sequence
  Description of Artificial Sequence Synthetic polynucleotide
atggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgacttcaacagattccactgccacttctcaccacgtgactggcaacgactcatcaacaacaattggggattccggcccaaaagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcgcaccagggctgcctgcctccgttcccggcggacgtcttcatgattcctcagtacgggtacctgactctgaacaatggcagtcaggccgtgggccgttcctccttctactgcctggaatattttccatcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgcaacctg

SEQ ID NO: 125 - DNA, 1605 bp, Artificial Sequence
  Description of Artificial Sequence Synthetic polynucleotide
atggcttcaggtggtggcgcaccagtggcagacaataacgaaggtgccgacggagtgggtaatgcctcaggaaattggcattgcgattccacatggctgggcgacagagtcatcaccaccagcacccgaacatgggccctgcccacctacaacaaccacctctacaagcaaatctccaacagcacatctggaggatcttcaaatgacaacgcctacttcggctacagcaccccctgggggtattttgacttcaacggattccactgccatttctcaccacgtgactggcagcgactcatcaacaacaattggggattccggcccaagagactcggcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattcctcagtacgggtacctgactctgaacaatggcagtcaggccgtgggccgttcctccttctactgcctggagtactttccttctcaaatgctgcgaactggaaacaattttgaattcagctacaccttcgaggacgtgcctttccacagcagctacgcgcacagccagagcctggacaggctgatgaatcccctcatcgactactacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcgacagccagggggccttacccggtatggtctggcagaaccgggacgtgtacctgcagggtcccatctgggccaagattcctcacacggacggcaacttccacccgtccccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtttactctgagcctcgccctattgggactcgttacctcacccgtaatctg

SEQ ID NO: 126 - DNA, 1605 bp, Artificial Sequence
  Description of Artificial Sequence Synthetic polynucleotide
atggctgcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 127 - DNA, 1599 bp, Artificial Sequence
  Description of Artificial Sequence Synthetic polynucleotide
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgcaacctg

SEQ ID NO: 128 - DNA, 1605 bp, Artificial Sequence; identical to SEQ ID NO: 126
  Description of Artificial Sequence Synthetic polynucleotide

SEQ ID NO: 129 - DNA, 1605 bp, Artificial Sequence
  Description of Artificial Sequence Synthetic polynucleotide
atggcttcaggcggtggcgcaccaatggcagacaataacgaaggcgccgacggagtgggtaatgcctcaggaaattggcattgcgattccacatggctgggcgatagagtcatcaccaccagcacccgaacctgggccctccccacctacaacaatcacctctacaagcaaatctccaacagcacatctggaggatcttcaaatgacaacgcctacttcggctacagcaccccctgggggtattttgacttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtgttcatgattcctcagtacggctacctgactctcaacaatggcagtcagtctgtgggccgttcctccttctactgcctggaatatttcccatcgcagatgctgagaacgggcaataactttaccttcagctacaccttcgaggacgtgcctttccacagcagctacgcccacagccagagcttggaccggctgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcaaccagtcaaagctgaactctttcatcacgcaatacagcaccggacaagtcagcgtggaaatcgagtgggagctgcagaaggaaaacagcaagcgctggaatccagagattcaatacacttccaactactacaaatctacaaatgtggactttgctgtcaacacggagggggtttatagtgagcctcgccccattggcacccgttacctcacccgtaatctg

SEQ ID NO: 130 - DNA, 1602 bp, Artificial Sequence
  Description of Artificial Sequence Synthetic polynucleotide
atggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctacaacaaccacctctacaagcaaatctccagtgcttcaacgggggccagcaacgacaacacctacttcggctacagcaccccctgggggtattttgacttcaacagattccactgtcacttttcaccacgtgactggcaacgactcatcaacaacaactggggattccggcccaagaagctgcggttcaagctcttcaacatccaggtcaaggaggtcacgcagaatgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagtttacttacaccttcgaggacgtgcctttccacagcagctacgcgcacagccagagcctggacaggctgatgaatcccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaactttcacccttctccgctgatgggagggtttggaatgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 131 - DNA, 1602 bp, Artificial Sequence
  Description of Artificial Sequence Synthetic polynucleotide
atggctgcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttttcaccacgtgactggcagcgactcatcaacaacaactggggattccggcccaagaagctgcggttcaagctcttcaacattcaggtcaaagaggttacggacaacaatggagtcaagaccatcgccaataacctcaccagcaccatccaggtgtttacggactcggagtaccagctgccgtacgttctcggctctgcccaccagggctgcctgcctccgttcccggcggacgtcttcatgattccgcagtacggctaccttacactgaacaatggaagtcaagccgtaggccgttcctccttctactgcctggaatactttccttcgcagatgctgagaaccggcaacaacttccagttcagctacgagtttgagaacgtacctttccatagcagctacgctcacagccaaagcctggaccgactaatgaatccactgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaactttcacccttctccgctgatgggcggctttggcctgaaacacccgcctcctcagatcctgattaagaatacacctgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctg

SEQ ID NO: 132 - DNA, 1602 bp, Artificial Sequence
  Description of Artificial Sequence Synthetic polynucleotide
gtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggaataccagctcccgtacgtcctcggctctgcgcaccagggctgcctgcctccgttcccggcggacgtcttcatgattcctcagtacgggtacctgactctgaacaatggaagtcaagccgtaggccgttcctccttctactgcctggaatatttcccatcgcagatgctgagaacgggcaacaactttgagttcagctacagcttcgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcctctcatgggcggctttggacttaagcacccgcctcctcagatcctcatcaaaaacacgcctgttcccgcggatcctccaactaccttcagtcaagctaagctggcgtcgttcatcacgcagtacagcaccggacaggtcagcgtggaaatcgagtgggagctgcagaaggagaacagcaaacgctggaacccagagattcaatacacttccaactactacaaatctacaaatgtggactttgctgttaatacagaaggcgtgtactctgaaccccgccccattggcacccgttacctcacccgtaatctg

SEQ ID NO: 133 - DNA, 1599 bp, Artificial Sequence
  Description of Artificial Sequence Synthetic polynucleotide
atggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataacctcaccagcaccatccaggtgtttacggactcggagtaccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacgggaggtaccgcaggaactcagcagttgctattttctcaggccgggcctaataacatgtcggctcaggccaaaaactggctacccgggccctgctaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaaccccgagatccagtacacttccaactacaacaagtctgttaatgtggactttactgtggacactaatggcgtgtattcagagcctcaccccattggcaccagatacctcacccgtaatctg

SEQ ID NO: 134 - DNA, 1599 bp, Artificial Sequence
  Description of Artificial Sequence Synthetic polynucleotide
  modified_base (186)..(187): a, c, t or g
atggctacaggcagtggcgcaccaatggcagacaataacgagggcgccgacggagtgggtaattcctcgggaaattggcattgcgattccacatggctgggcgacagagtcattaccaccagcacccgaacctgggccctgcccacctacaacaaccacctctacaaacaaatttccagccaatcnnagcctcgaacgacaatcactactttggctacagcaccccctggggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctctgcgcaccagggctgcctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgctcacagccagagtctggaccggctgatgaaccccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctacggataacaacaacagtgaatactcgtggactggagctaccaagtaccacctcaatggcagagactctctggtgaatccgggcccggccatggcaagccacaaggacgatgaagaaaagttttttcctcagagcggggttctcatctttggggagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggcaacttccacccgtctccgctgatgggcggctttggcctgaaacatcctccgcctcagatcctgatcaagaacacgcctgtacctgcggatcctccgaccaccttcaaccagtcaaagctgaactccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaacacggagggggtttatagcgagcctcgccccattggcacccgttacctcacccgcaacctg

SEQ ID NO: 135 - DNA, 1599 bp, Artificial Sequence
  Description of Artificial Sequence Synthetic polynucleotide
atggcttcaggcggtggcgcaccaatggcagacaataacgaaggcgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctcccgtacgtcctcggctctgcgcaccagggctgcctgcctccgttcccggcggacgtgttcatgattcctcagtacgggtacctgactctgaacaatggcagtcaggccgtgggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaagcgctggaacccggagattcagtacacctccaactactacaaatctacaagtgtggactttgctgttaatacagaaggcgtgtactctgaaccccgccccattggcacccgttacctcacccgtaatctg

SEQ ID NO: 136 - DNA, 1599 bp, Artificial Sequence
  Description of Artificial Sequence Synthetic polynucleotide
gtggctgcaggcggtggcgcaccaatggcagacaataacgaaggtgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccctgggggtattttgacttcaacagattccactgccatttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccatcgcagatgctgagaaccggcaacaacttccagtttacttacaccttcgaggacgtgcctttccacagcagctacgcccacagccagagcttggaccggctgatgaatcctctgattgaccagtacctgtactacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcacccgttacctcacccgtaatctg

SEQ ID NO: 137 - DNA, 1599 bp, Artificial Sequence
  Description of Artificial Sequence Synthetic polynucleotide
atggcttcaggcggtggcgcaccaatggcagacaataacgaaggcgccgacggagtgggtaattcctcgggaaattggcattgcgattccacatggctgggggacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacggtcatccttttactgcctggaatatttcccatctcagatgctgagaacgggcaataactttaccttcagctacaccttcgaggacgtgcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaagattcctcacacggacggcaacttccacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggtactcgttacctcacccgtaatctg

SEQ ID NO: 138 - DNA, 1599 bp, Artificial Sequence
  Description of Artificial Sequence Synthetic polynucleotide
atggcttcaggcggtggcgctccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtatttcgacttcaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctccctccgttcccggcggacgtgttcatgattcctcagtacggctacctgactctcaacaatggcagtcagtctgtgggacgttcctccttctactgcctggagtacttcccctctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggccaaaattcctcacacggacggcaacttccacccgtctcccctgatgggcggctttggactgaagcacccgcctcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaagaaaacagcaagcgctggaaccccgagatccagtacacctccaactactacaaatctacaagtgtggactttgctgtcaacacggagggggtttatagcgagcctcgccccattggcacccgttacctcacccgtaatctg

SEQ ID NO: 139 - DNA, 1602 bp, Artificial Sequence
  Description of Artificial Sequence Synthetic polynucleotide
atggctgcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggccaaaaagactcagcttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctgcctccgttcccggcggacgtcttcatgattccccagtacggctacctaacactcaacaacggtagtcaggccgtgggacgctcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagatcctcatcaaaaacacacctgtacctgcgaatccttcgaccaccttcagtgcggcaaagtttgcttccttcatcacacagtactccacgggacaggtcagcgtggaaatcgagtgggagctgcagaaagaaaacagcaaacgctggaacccagagattcaatacacttccaactactacaaatctacaagtgtggactttgctgttaatacagaaggcgtgtactctgaaccccgccccattggcacccgttacctcacccgtaatctg

SEQ ID NO: 140 - DNA, 1602 bp, Artificial Sequence
  Description of Artificial Sequence Synthetic polynucleotide
atggctgcaggcggtggcgctccaatggcagacaataacgaaggcgccgacggagtgggtaattcctcgggaaattggcattgcgattccacatggctgggcgacagagtcatcaccaccagcacccgaacctgggccctgcccacctacaacaaccacctctacaagcaaatctccagtgcttcaacgggggccagcaacgacaacacctacttcggctacagcaccccctgggggtattttgactttaacagattccactgccacttttcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtctttacggactcggaataccagctgccgtacgtcctcggctctgcccaccagggctgcctgcctccgttcccggcggacgtgttcatgattccccagtacggttacctaacactcaacaacggtagtcaggccgtgggacgctcctccttctactgcctggaatactttccttcgcagatgctgagaacgggcaacaactttgagttcagctaccagtttgaggacgtgccttttcacagcagctacgcgcacagccagagcctggaccggctgatgaatcctctcatcgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcaaaccaatacggggcctattgtgggaaatgtcaacagccaaggagccttacctggcatggtctggcagaaccgagacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaacccggagatccagtacacttccaactattacaagtctaataatgttgaatttgctgttaatacagaaggcgtgtactctgaaccccgccccattggcacccgttacctcacccgtaatctg

SEQ ID NO: 141 - DNA, 1605 bp, Artificial Sequence
  Description of Artificial Sequence Synthetic polynucleotide
atggctacaggcagtggcgcaccaatggcagacaataacgagggcgccgacggagtgggtaattcctcgggaaattggcattgcgattccacatggctgggcaacagagtcatcaccaccagcacccgaacctgggccctgcccacctacaacaaccacctctacaagcaaatatccaatgggacatcgggaggaagcaccaacgacaacacctattttggctacagcaccccctgggggtattttgacttcaacagattccactgtcacttttcaccacgtgactggcaacgactcatcaacaacaactggggattccggcccaagagactcagcttcaagctcttcaacattcaggtcaaggaggtcacgcagaatgaaggcaccaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcgaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccaactaccttcagtgcggcaaagtttgcttccttcatcacacagtactccacgggacaggtcagcgtggagatcgagtgggagctgcagaaggaaaacagcaaacgctggaatcccgaaattcagtacacttccaactattacaagtctaataatgttgaatttgctgttaatactgaaggtgtatatagtgaaccccgccccattggcaccagatacctgactcgtaatctg

SEQ ID NO: 142 - DNA, 1605 bp, Artificial Sequence
  Description of Artificial Sequence Synthetic polynucleotide
  modified_base (819)..(819): a, c, t or g
atggcttcaggtggtggcgcaccagtggcagacaataacgaaggtgccgatggagtgggtagttcctcgggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacaccttcgaggacgtgcctttccacagcagctacgcacacagccagagcttggaccgactgatgaatcctctcatcgaccagtacctgtactacttgtctcggactcaaacaacaggaggcacggcaaatacgcagactctgggctttagccaaggtgggcctaatacaatggccaatcangcaaagaactggctgccaggaccctgttaccggcagcagcgagtctctacgacaaccgggcaaaacaacaacagcaactttgcttggactggtgccaccaaatatcacctgaacggaagagactctctggtaaatcccggtgtcgctatggcaacccacaaggatgacgacgaccgcttcttcccttcgagcggggtcctgatttttggcaagcaaggagccgggaacgatggagtggattacagccaagtgctgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaagcgctggaacccggagattcagtacacctccaactttgaaaagcagactggtgtggactttgctgtcaatacagagggaacttattctgagcctcgccccattggtactcgttacctcacccgtaatctg

SEQ ID NO: 143 - DNA, 1602 bp, Artificial Sequence
  Description of Artificial Sequence Synthetic polynucleotide
  modified_base (43)..(43): a, c, t or g
atggcttcaggcggtggcgcaccaatggcagacaataacgaangtgccgatggagtgggtagttcctcgggaaattggcattgcgattcccaatggctgggggacagagtcatcaccaccagcacccgaacctgggccctgcccacctacaacaatcacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagagactcagcttcaagctctttaacattcaagtcaaagaggtcacgcagaatgacggtacgacgacgattgccaataaccttaccagcacggttcaggtgtttactgactcggagtaccagctcccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcccacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 144 - DNA, 1602 bp, Artificial Sequence
  Description of Artificial Sequence Synthetic polynucleotide
atggctacaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtgtttacggactcggagtaccagctgccgtacgttctcggctctgcccaccagggctgcctgcctccgttcccggcggacgtgttcatgattcctcagtacggctacctgactctcaacaatggcagtcagtctgtgggacgttcctccttctactgcctggagtacttcccctctcagatgatgagaacgggcaacaactttgagttcagctacagcttcgaggacgtgcctttccacagcagctacgcacacagccagagcctggaccggctgatgaatcccctcatcgaccagtacctgtactacctgtctcggactcagtccacgggaggtaccgcaggaactcagcagttgctattttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattcctcacacggacggcaacttccacccttcaccgctaatgggaggatttggactgaagcacccacctcctcagatcctgatcaagaacacgccggtacctgcggatcctccaacaacgttcagtcaagctaagctggcgtcgttcatcacgcagtacagcaccggacaggtcagcgtggaaattgaatgggagctgcagaaggaaaacagcaagcgctggaacccggagattcaatacacttccaactactacaaatctacaaatgtggactttgctgtcaacacggagggggtttatagcgagcctcgccccattggcacccgttacctcacccgcaacctg

SEQ ID NO: 145 - DNA, 25 bp, Artificial Sequence
  Description of Artificial Sequence Synthetic oligonucleotide
cagtgacgcagatataagtgagccc

SEQ ID NO: 146 - DNA, 38 bp, Artificial Sequence
  Description of Artificial Sequence Synthetic oligonucleotide
gaaacgaattaaacggtttattgattaacaatcgatta

SEQ ID NO: 147 - DNA, 2828 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtatgtggactttcgggcgccgcgcagttgccggcctcctggcgtccccgagcccggcccaggcccagaccctcacccgggccccgcggctggcagagttggcccagctctgcagccgccggggcctgcgcaccggcatcaatgcgacctgcacaacccaccacaccagttcgaacctccgtggcctcaaccagattcggaatgtcaaaaggcagagtgtctacttgatgaatttgaggaaatcgggaactttgggccacccaggctctctagatgacaccacctatgaaagactagcagaggaaacgctggactctttagcagagttttttgaagaccttgcagacaagccatacacctttgaggactatgatgtttcctttgggagtggtgtcttaactgttaaactgggtggagatctaggaacctacgtgatcaacaagcagacgccaaacaagcaaatctggttatcttctccatccagtggacccaagcgttatgactggactgggaaaaactgggtgtattcccacgacggcgtttccctccatgagctgctgggcgcagagctcactaaagccttaaaaaccaaactggacttgtcttccttggcctattccggaaaagacgcttatccttatgacgtgcctgactatgcctgatgactcgaggacggggtgaactacgcctgaggatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcctaggtatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg